(19) United States
(12) Patent Application Publication
(10) Pub. No.: US 2017/0224758 A1
(43) Pub. Date: Aug. 10, 2017

(54) COMPOSITIONS AND METHODS OF TREATING MUSCULAR DYSTROPHY

(71) Applicants: THE BROAD INSTITUTE, INC., CAMBRIDGE, MA (US); CHILDREN'S MEDICAL CENTER CORPORATION, BOSTON, MA (US); UNIVERSITY OF SAO PAULO, SAO PAULO (BR)

(72) Inventors: KERSTIN LINDBLAD-TOH, CAMBRIDGE, MA (US); LOUIS M. KUNKEL, BOSTON, MA (US); NATASSIA M. VIEIRA, BOSTON, MA (US); MAYANA ZATZ, SAO PAULO (BR)

(73) Assignees: THE BROAD INSTITUTE, INC., CAMBRIDGE, MA (US); CHILDREN'S MEDICAL CENTER CORPORATION, BOSTON, MA (US); UNIVERSITY OF SAO PAULO, SAO PAULO (BR)

(21) Appl. No.: 15/519,100

(22) PCT Filed: Oct. 16, 2015
(86) PCT No.: PCT/US2015/056026

§ 371 Date: Apr. 13, 2017

Related U.S. Application Data
(60) Provisional application No. 62/065,559, filed on Oct. 17, 2014.

(51) Int. Cl. A61K 38/00; C07K 14/705; C12N 15/63; C12Q 1/68; C07K 14/47; C12N 15/67

(52) U.S. Cl. CPC A61K 38/00; C07K 14/4702; C12N 15/67; C12N 15/63; C12Q 1/686; C07K 14/4708; C07K 14/705

(57) ABSTRACT

Provided herein are compositions and methods of treating muscular dystrophy (MD), such as administering an effective amount of a composition that increases the expression of JAG1, a composition comprising a JAG1 agonist, or a composition that promotes JAG1 signaling. Also provided are methods of prognosing MD or evaluating responsiveness to treatment for MD, e.g., by measuring an expression level of JAG1, and methods of identifying a compound for the treatment of MD.

### CROSS-REFERENCE TO RELATED APPLICATIONS

This application claims the benefit under 35 U.S.C. §119(e) of U.S. provisional application No. 62/065,559, filed Oct. 17, 2014, the contents of which is incorporated by reference herein in their entirety.

### FEDERALLY SPONSORED RESEARCH OR DEVELOPMENT

This invention was made with U.S. Government support under 1RO1AR064300-01A1 awarded by the National Institutes of Health. The U.S. Government has certain rights in the invention.

### SEQUENCE LISTING

The Sequence Listing filed on Oct. 16, 2015 as an ASCII text file is incorporated by reference herein. The ASCII text file is named B1195.70031WO00-SEQ, was created on Oct. 16, 2015, and is 141,000 bytes in size.

### BACKGROUND OF THE INVENTION

Muscular dystrophy is a muscle degenerative disease in which the muscle at first forms normally, but starts to degenerate faster than it can be repaired. The most common form of muscular dystrophy is Duchenne Muscular Dystrophy (DMD) representing over 90% of the diagnosed cases. In 1986, mutations in the dystrophin gene were found to be the cause of both Duchenne and Becker Muscular Dystrophy. Shortly thereafter antibodies were developed against dystrophin and used to improve diagnosis of the disease. The predominant muscle dystrophin isoform is translated from the largest gene in the human genome. The gene encodes a large protein of 427 KDa that positions just inside of the sarcolemmal membrane and links the internal cytoskeleton with the muscle cell membrane. This linkage is vital to maintaining muscle membrane integrity during repeated cycles of cell contraction. Almost all known human dystrophin mutations that cause (DMD) typically result in the loss or degradation of the dystrophin protein at the sarcolemmal membrane

Currently, there are no effective long-term therapies for treating DMD, the most common form of the disease affecting approximately 1 in every 3,500 males born in the United States. Currently, the steroid prednisone is the only treatment option available for muscular dystrophy patients in the United States. It does not treat the underlying cause of the disease and has significant side effects.

### SUMMARY OF THE INVENTION

Aspects of the disclosure relate in part to the identification of Jagged1 as a gene that is capable of modulating the phenotype of DMD. In a study of Golden retriever muscular dystrophy (GRMD) dogs, two related dogs (referred to herein as ‘escapers’) were identified as having a milder phenotype than other GRMD dogs, even though the escapers had no expression dystrophin in their muscles, no utrophin upregulation, and raised serum creatine kinase (CK) levels. The mild symptoms of the escaper GRMD dogs suggested that a normal life span was possible even in the absence of dystrophin. Using genome-wide association (GWA) mapping, a chromosomal region associated with the escaper phenotype was identified. The gene Jagged1 was found within this region and was shown to have altered expression in the muscles of escaper dogs compared to other GRMD dogs. A mutation present only in escaper GRMD dogs was then identified that creates a new myogenin binding site in the Jagged1 promoter. It was then determined that overexpression of Jagged1 rescued the dystrophic phenotype in a sapje DMD zebrafish model.

Accordingly, aspects of the disclosure relate to various compositions and compounds for treating muscular dystrophy (MD), such as DMD, and/or restoring a muscle function or phenotype in a subject. Other aspects relate to use of Jagged1 in methods of prognosing MD, evaluating responsiveness to treatment for MD, and identifying compounds for the treatment of MD.

Aspects of the disclosure relate to a method of treating muscular dystrophy (MD), the method comprising administering to a subject having or suspected of having MD an effective amount of a composition that increases the expression of JAG1. Other aspects of the disclosure relate to a method, comprising administering to a subject an effective amount of a composition that increases the expression of JAG1 to restore a muscle function or phenotype.

In some embodiments, the composition comprises a vector for recombinant expression of JAG1. In some embodiments, the vector comprises regulatory elements for the overexpression of JAG1. In some embodiments, the composition comprises a vector comprising alternative regulatory element(s) for JAG1, wherein the alternative regulatory element(s) replace the endogenous regulatory element(s) of JAG1 and increase the expression of endogenous JAG1.

In some embodiments, the composition comprises a transcription factor or a vector for recombinant expression of the transcription factor, wherein the transcription factor increases the expression of JAG1. In some embodiments, the transcription factor is selected from the group consisting of myogenin, MyoD, Myf5, MRF4, and Rbp-J.

In some embodiments, the composition comprises a compound that increases the expression of JAG1.

In some embodiments, the composition increases the expression of JAG1 in one or more cells of the subject selected from the group consisting of muscle cells, satellite cells, myoblasts, muscle side population cells, fibroblast cells, smooth muscle cells, blood cells, blood vessel cells, stem cells, mesenchymal stem cells, and neurons. In some embodiments, the MD is Duchenne muscular dystrophy (DMD).

Other aspects of the disclosure relate to a method of treating muscular dystrophy (MD), the method comprising administering to a subject having or suspected of having MD an effective amount of a composition comprising a JAG1 agonist. In some embodiments, the JAG1 agonist is a JAG1 polypeptide or fragment thereof, a nucleic acid encoding a JAG1 polypeptide or fragment thereof, or a compound that promotes JAG1 signaling. In some embodiments, the MD is Duchenne muscular dystrophy (DMD).

Other aspects of the disclosure relate to a method of treating muscular dystrophy (MD), the method comprising administering to a subject having or suspected of having MD an effective amount of a composition that promotes JAG1 signaling. In some embodiments, the composition increases the expression of JAG1. In some embodiments, the composition comprises a compound.

In some embodiments, the composition comprises a transcription factor or a vector for recombinant expression of the transcription factor, wherein the transcription factor increases the expression of JAG1. In some embodiments, the transcription factor is selected from the group consisting of myogenin, MyoD, Myf5, MRF4, and Rbp-J.

In some embodiments, the composition comprises a JAG1 agonist selected from the group consisting of JAG1 polypeptide or fragment thereof, a nucleic acid encoding a JAG1 polypeptide or fragment thereof, or a compound that promotes JAG1 signaling.

Yet other aspects of the disclosure relate to a method of treating muscular dystrophy (MD), the method comprising administering to a subject having or suspected of having MD an effective amount of a composition comprising a compound provided in Table 2. In some embodiments, the MD is Duchenne muscular dystrophy (DMD).

Other aspects of the disclosure relate to a method of increasing the proliferation of a muscle cell or a muscle progenitor cell, the method comprising increasing the expression of JAG1 in a muscle cell or a muscle progenitor cell, wherein increasing the expression of JAG1 increases the proliferation of the cell.

Yet other aspects of the disclosure relate to a method of increasing and/or enhancing myofiber structure in a muscle cell or muscle progenitor cell, the method comprising increasing the expression of JAG1 in a muscle cell or a muscle progenitor cell, wherein increasing the expression of JAG1 increases and/or enhances the myofiber structure of the cell. In some embodiments, the method is performed in vitro or ex vivo. In some embodiments, the muscle cell or muscle progenitor cell is in a subject. In some embodiments, the subject has or is suspected of having muscular dystrophy (MD). In some embodiments, the MD is Duchenne muscular dystrophy (DMD).

Other aspects of the disclosure relate to a method of prognosing muscular dystrophy (MD), the method comprising selecting a subject having or suspected of having MD; measuring an expression level of JAG1 in a sample obtained from the subject; and identifying the subject as having a more favorable prognosis when the expression level of JAG1 is higher than a control level. In some embodiments, the sample comprises muscle cells or muscle progenitor cells. In some embodiments, the JAG1 mRNA or protein expression level is measured. In some embodiments, the control level is the level of JAG1 expression in a subject not having MD. In some embodiments, the control level is the level of JAG1 expression in a subject having a MD disease course.

Other aspects of the disclosure relate to a method of prognosing muscular dystrophy (MD), the method comprising selecting a subject having or suspected of having MD; detecting a variant in a JAG1 gene, a JAG1 promoter, or a JAG1 regulatory element in a sample obtained from the subject; and identifying the subject as having a more favorable prognosis when the variant in the JAG1 gene, JAG1 promoter, or JAG1 regulatory element is detected. In some embodiments, the variant comprises one or more of a mutation, a SNP, or a haplotype in the JAG1 gene, JAG1 promoter, or JAG1 regulatory element.

Yet other aspects of the disclosure relate to a method of evaluating responsiveness to treatment for muscular dystrophy (MD), the method comprising measuring an expression level of JAG1 in a sample obtained from a subject having MD prior to the subject receiving a treatment for MD; measuring an expression level of JAG1 in a sample obtained from the subject after the subject has received a treatment for MD; and comparing the expression levels of JAG1 measured prior to and after treatment; wherein an increase in JAG1 expression after the subject has received the treatment identifies the subject as responsive to treatment; or wherein a decrease or no change in JAG1 expression after the subject has received the treatment identifies the subject as unresponsive to treatment. In some embodiments, the sample comprises muscle cells, muscle progenitor cells, or blood vessels obtained from muscle tissue. In some embodiments, the JAG1 mRNA or protein expression level is measured. In some embodiments, the MD is Duchenne muscular dystrophy (DMD).

Other aspects of the disclosure relate to a method for identifying a compound for the treatment of muscular dystrophy (MD), the method comprising contacting a cell with a candidate compound; measuring an expression level of JAG1 in the cell; and identifying the candidate compound as a compound for the treatment of MD if the expression level of JAG1 is higher than a control level. In some embodiments, the control level is a level of expression in the cell in the absence of the candidate compound. In some embodiments, the cell is selected from the group consisting of fibroblasts, blood cells, mesenchymal stem cells, muscle progenitor cells, muscle satellite cells, smooth muscle cells, muscle side population cells, myoblasts, iPS cells, and embryonic stem cells.

Other aspects of the disclosure relate to a method for identifying a JAG1-modulating compound for the treatment of muscular dystrophy (MD), the method comprising contacting a zebrafish with a candidate compound; assessing the muscle phenotype of the zebrafish; and identifying the candidate compound as a JAG1-modulating compound for the treatment of MD if the muscle phenotype of the zebrafish is improved compared to a control muscle phenotype. In some embodiments, the zebrafish is a sapje zebrafish, sapje-like zebrafish, LAMA2 zebrafish, or dag1 zebrafish. In some embodiments, the muscle phenotype is assessed using a muscle birefringence assay. In some embodiments, the method further comprises determining the expression of JAG1 in the zebrafish. In some embodiments, the method further comprises testing the candidate compound in a mammalian model of MD, the method further comprising administering the candidate compound to a mammal; and identifying the candidate compound as a therapeutic compound for the treatment of MD if the phenotype of the mammal is improved as compared to a control. In some embodiments, the mammal is a mouse, dog, cat, or pig model of MD. In some embodiments, the improvement in phenotype is assessed by muscle tissue biopsy, determining muscle strength, or assessing blood levels of creatine kinase. In some embodiments, the control is the phenotype of a mammal in the absence of being administered the candidate compound. In some embodiments, the MD is Duchenne muscular dystrophy (DMD).

The details of one or more embodiments of the disclosure are set forth in the description below. Other features or advantages of the present disclosure will be apparent from the following drawings and detailed description of several embodiments, and also from the appending claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. The specification includes drawings in color.

FIGS. 1A-C are a series of graphs showing that combining GWAS and haplotype analysis identifies a 30 Mb candidate region on chromosome 24. (FIG. 1A) A QQ plot of 129,908 SNPs tested for association finds 27 SNPs exceed the 95% confidence intervals (dashed lines) and minimal stratification relative to the expected distribution (red line), suggesting the mixed model approach corrected for close relatedness among the 2 escapers and 31 controls. (FIG. 1B) Only the association on chromosome 24 also falls in a region where the 2 cases (sire and offspring) share one haplotype likely to be identical by descent (LOD=64.7, red). Other peaks on chromosomes 24, 33 and 37 show no evidence of IBD (LOD=0, grey) and are most likely false positives due to the small GWA sample size. (FIG. 1C) The region of IBD extends 27 Mb from the start of chromosome 24 and includes the putative driver gene JAG1 (blue line) identified through gene expression profiling (FIG. 2).

FIGS. 2A-D are a series of diagrams and graphs showing altered Jagged1 expression in escaper GRMD dogs. (FIG. 2A) mRNA microarray result comparing the muscle gene expression of escaper GRMD dogs with related severely affected and normal littermates. (FIG. 2B) mRNA expression of escaper dogs confirming the expression array findings. Relative Jagged1 gene expression in muscle samples of escaper GRMD (E) dogs as compared to related severely affected (S) and normal GRMD dogs (N); bars indicate standard deviation from the mean. (FIGS. 3C and D). Jagged1 protein levels in the muscle of escaper GRMD (E) dogs as compared to severely affected (S) and normal control dog muscle (N); Beta-actin was loading control.

FIGS. 3A-F are a series of diagrams and a photograph of a Western blot showing a variant at the Jagged1 promoter in escaper GRMD dogs. (FIG. 3A) Dog and Human Jagged1 locus. Arrow: variant at dog chr24:11644709. SEQ ID NOs: 13, 14, 15, 16, 17, 18 and 19 appear from top to bottom, respectively. (FIG. 3B) Conservation of the variant position. SEQ ID NOs: 19 and 20 appear from top to bottom, respectively. (FIG. 3C) Predicted transcription factor binding site at the region with the base pair change. (FIG. 3D) Consensus sequence of Myogenin binding site, note the T as an important base for binding. (FIG. 3E) Electromobility shift assay (EMSA) showing Myogenin binding to mutated probe (T) and not to the wild type probe (G). Luciferase reporter assay showing activity of wild-type (Wt) and escaper (E) genotype vectors in muscle cells (C2C12) and embryonic kidney cells (293T) with Myogenin or MyoD overexpression, as compared to empty vectors controls (V). Error bars indicate SEM (n=3 replicates) (FIG. 3F).

FIGS. 4A-F are a series of graphs and photographs showing the functional analysis of jagged1 expression. (FIG. 4A) Percent affected sapje fish as determined by birefringence assay at 4 dpf. Note fewer affected fish in the jagged1 injected sapje cohort. Four separate injection experiments were performed. (FIG. 4B) Genotype of sapje injected fish with jagged1a and jagged1b as compared to non-injected sapje fish. In red dystrophin null fish with normal phenotype, recovered by jagged1 overexpression. (FIG. 4C) Immunofluorescence of jagged1a and jagged1b overexpression in the sapje fish. Normal, phenotypically affected homozygous fish for the dystrophin mutation and jagged1 a and jagged1b injected with normal birefringence (recovered) stained for myosin heavy chain (MCH) antibodies. Note the organization of the muscle fibers in the recovered fish muscle comparable to the normal fish (n=10). (FIG. 4D) Jagged1 protein levels in the muscle of cardiotoxin injured mice 1,4 and 7 days after injury. (FIG. 4E) Jagged1 protein levels in muscle cells during in vitro muscle differentiation. (FIG. 4F) Muscle cell proliferation rate, as measured my MTTA, of normal, escaper and affected GRMD dogs.

FIG. 5 is a diagram showing GRMD dogs pedigree and genotype. All dogs were genotyped for the GRMD mutation and for the jagged1 variant; the presence or absence of the mutation is indicated by a symbol located in the key legend. The first escaper dog, the proband, is indicated by a black arrow.

### DETAILED DESCRIPTION OF THE INVENTION

Duchenne muscular dystrophy (DMD) is an X-linked skeletal myopathy that affects 1 in 3500 boys and is caused by protein disrupting mutations in the dystrophin gene [refs. 1-4]. Absence of functional dystrophin causes myofiber degeneration [ref. 5,6], but additional factors involved in the pathogenesis of DMD remain poorly understood and represent an unexplored territory for therapy. Animal models of dystrophin-deficiency [refs. 7-9] show marked differences in the degree of disease severity [refs. 10-12]. The golden retriever muscular dystrophy (GRMD) dog shows the greatest phenotypic variability among all the animal models for DMD [ref. 16,17], which suggests the presence of modifying mechanisms. As described herein, two related escaper GRMD dogs having a mild and molecularly uncharacterized phenotype were identified. The escaper dogs were clearly clinically distinguishable from other affected GRMD dogs despite the absence of muscle dystrophin, the lack of utrophin upregulation and the raised serum creatine kinase (CK) level in the escaper dogs [ref. 12]. The majority of the DMD therapeutic approaches aim to rescue dystrophin expression in the muscle [ref. 13]. However, the phenotype of the escaper GRMD dogs suggested that it is possible to have a normal lifespan even in the absence of dystrophin [ref. 12]. Using genome-wide association (GWA) mapping, a chromosomal region was identified that was associated with the escaper phenotype. Only one gene within this region, Jagged1, showed altered expression when comparing muscle tissue from escaper and affected dogs. By whole genome sequencing, a candidate mutation was identified that was present only in escaper GRMD dogs in a myogenin binding site in the Jagged1 promoter. It was then determined in the sapje DMD zebrafish model that overexpression of Jagged1 rescued the dystrophic phenotype of the fish. These results suggested that increasing the expression and/or activity of Jagged1 (JAG1) protein is useful for treating MD and/or restoring muscle function or phenotypes in subjects in need thereof.

Accordingly, aspects of the disclosure relate to compositions and compounds for increasing expression of JAG1 and/or that act as a JAG1 agonist for use in methods of treatment. Other aspects of the disclosure relate to method of prognosing MD, evaluating responsiveness to a treatment for MD and identifying compounds for the treatment of MD and/or modulating JAG1 expression.

### Treatment

Aspects of the disclosure relate to methods and pharmaceutical compositions for the treatment of MD, such as DMD. To “treat” a disease described herein, e.g., MD or DMD, means to reduce or eliminate a sign or symptom of the disease, to stabilize the disease, and/or to reduce or slow further progression of the disease. For example, treatment of MD, such as DMD, may result in e.g., a slowing of muscle degeneration, decreased fatigue, increased muscle strength, reduced blood levels of creatine kinase (CK), decreased difficulty with motor skills, decreased muscle fiber deformities, decreased inflammation or fibrotic tissue infiltration in the muscle, stabilization of the progression of the disease (e.g., by halting progressive muscle weakness) etc.

In some embodiments, a method of treating muscular dystrophy (MD) is provided, the method comprising administering to a subject having or suspected of having MD an effective amount of a composition that increases the expression of JAG1. In other embodiments, a method is provided, comprising administering to a subject an effective amount of a composition that increases the expression of JAG1 to restore a muscle function or phenotype. Muscle function or phenotype includes, e.g., muscle regeneration, muscle strength or/and stabilization or improvement of the progression of a disease such as MD. Muscle function or phenotype can be measured, e.g., by treadmill, rota-road, grip, or by the standard Motor Function Measure for Neuromuscular Diseases used for humans.

A composition that increases the expression of JAG1 is a composition that increases the expression of JAG1 protein, e.g., by increasing transcription, translation, mRNA stability, protein stability, etc., compared to a control level. The increase in expression may be, e.g., at least 5%, at least 10%, at least 20%, at least 30%, at least 40%, at least 50%, at least 60%, at least 70%, at least 80%, at least 90%, at least 100%, at least 150%, at least 200%, at least 250%, at least 300% or more than a control expression level. The control expression level may be a level of JAG1 expression in a control cell, control tissue, or control subject. In some embodiments, the control level is a level of JAG1 expression in the subject, e.g., in a muscle cell of the subject, prior to the administration of the composition. In some embodiments, the control level is a level of JAG1 expression in a population of subjects having MD, e.g., a population of subjects having DMD. The expression level may be measured using an assay known in the art or as described herein, such as western blot, qPCR, Rt-PCR, ELISA, RNA sequencing, etc. (see, e.g., Purow et al. Expression of Notch-1 and Its Ligands, Delta-Like-1 and Jagged-1, Is Critical for Glioma Cell Survival and Proliferation. Cancer Res 2005; 65:2353-2363; or Current Protocols in Molecular Biology, Wiley Online Library, Online ISBN: 9780471142720). In some embodiments, the composition increases the expression of JAG1 in one or more cells of the subject selected from the group consisting of muscle cells, satellite cells, myoblasts, muscle side population cells, fibroblast cells, smooth muscle cells, blood cells, blood vessel cells, stem cells, mesenchymal stem cells, and neurons.

In some embodiments, the composition comprises a vector for recombinant expression of JAG1. Any vector known in the art for recombinant expression is contemplated for use herein. The vector may be a DNA vector or an RNA vector. The vector may comprise one or more synthetic nucleotides (e.g., locked nucleic acids, peptide nucleic acids, etc.) or nucleoside linkages (e.g., phosphorothioate linkages). The vector may be single-stranded, double-stranded, or contain regions of both single-strandedness and double-strandedness. Exemplary vectors include, but are not limited to a plasmid, a retrovirus, a lentivirus, an adenovirus, an adeno-associated virus, a herpes simplex virus, poxvirus, and baculovirus. In some embodiments, the vector comprises a nucleic acid sequence that encodes a JAG1 polypeptide or fragment thereof. In some embodiments, the vector comprises a nucleic sequence that encodes a Jagged1 mRNA. In some embodiments, the vector comprises a Jagged1 gene nucleic acid sequence, e.g., including the Jagged1 promoter, or a fragment thereof. Exemplary JAG1 polypeptide, mRNA and Jagged1 gene sequences are provided below.

Homo sapiens jagged 1, gene
(SEQ ID NO: 1)
CTGCCCGGCGTGCTGGGTAGAGGTGGCCAGCCCCGGCCGCTGCTGCCAGACGGGCTCTCCGGGTCCTTCT
CCGAGAGCCGGGCGGGCACGCGTCATTGTGTTACCTGCGGCCGGCCCGCGAGCTAGGCTGGTTTTTTTTT
TTCTCCCCTCCCTCCCCCCTTTTTCCATGCAGCTGATCTAAAAGGGAATAAAAGGCTGCGCATAATCATA
ATAATAAAAGAAGGGGAGCGCGAGAGAAGGAAAGAAAGCCGGGAGGTGGAAGAGGAGGGGGAGCGTCTCA
AAGAAGCGATCAGAATAATAAAAGGAGGCCGGGCTCTTTGCCTTCTGGAACGGGCCGCTCTTGAAAGGGC
TTTTGAAAAGTGGTGTTGTTTTCCAGTCGTGCATGCTCCAATCGGCGGAGTATATTAGAGCCGGGACGCG
GCGGCCGCAGGGGCAGCGGCGACGGCAGCACCGGCGGCAGCACCAGCGCGAACAGCAGCGGCGGCGTCCC
GAGTGCCCGCGGCGCGCGGCGCAGCGATGCGTTCCCCACGGACGCGCGGCCGGTCCGGGCGCCCCCTAAG
CCTCCTGCTCGCCCTGCTCTGTGCCCTGCGAGCCAAGGTAGGAGCCCTTCTCCGGGCCTCCCTCCCAGCC
GTCCTCTCCCTCCCCTCGCGGCGCCCTGGGCGGTTGAGCCCCGCGAGCAGGCTCGGCAGCCCTCGAGCGC
CCGACTCCTCGCCCCTGCGCCCGAGCGGCTGCACCTGGGCTGAGCGCGCCAACTCCCCCACGGGAGCCCC
TGCCTCCTGAGGGTGACCCCTCCAGGCCGGACAGGGGCGCGTTCGCTCCGCTGTTGCCCCCCGGGAAGTC
GTCGGATTTCTTCCGCACTCGTGCATTTTTTTTTTTTTTTGCCCCGGAATCATGTTGAGTCCTTGAACGT
AGTTTCGCAAAATTATTTTCACTCGGCGAGGAGGGCTCGTCGGGGCGGGGAGTGGGAGGGAGTCGCCACC
TCTATACTCGAAGAAAACAGCTTTCTCCCGCGCTGACCTACCTCCTTCCCTCGCCGGCAGGTGTGTGGGG
CCTCGGGTCAGTTCGAGTTGGAGATCCTGTCCATGCAGAACGTGAACGGGGAGCTGCAGAACGGGAACTG
CTGCGGCGGCGCCCGGAACCCGGGAGACCGCAAGTGCACCCGCGACGAGTGTGACACATACTTCAAAGTG
TGCCTCAAGGAGTATCAGTCCCGCGTCACGGCCGGGGGGCCCTGCAGCTTCGGCTCAGGGTCCACGCCTG
TCATCGGGGGCAACACCTTCAACCTCAAGGCCAGCCGCGGCAACGACCGCAACCGCATCGTGCTGCCTTT
CAGTTTCGCCTGGCCGGTGAGTGACTACTCGGGAAGGAGGCCGGGCGGAGCCTCACACCCGCGCCTGGCC
GAGCCCTGTTATCCCTTGCGAGAGGGATTTAAAGGGCTTTGGCTTGAAACTAGGCGCTAACTGGGGAAAT
CTCTAGGGGTGGCGGGTGAGGGAGCCGGGACACTATAGTTAGATCTCGGCTGGGGTAGTCGAGGTGGGTC
CCATTCCTGCTGCTATTTGCTTAGTGCACAAAAGTGGGGATCCCAGAGGGTTTATGGGACCAGGCTCCTA
GGAAGGCCGGTGTAAACTTGCAACTTGAAGTTCCCAGAGTAACTCTCCCGGCCCCAAGCCAAGAGATCGT
TGTAGCTGATCTCTTTGCTTAACTAATTAAACCTGGTGCGCCGTGGAAGGCGACTCGGCGCAGTTCTGGC
CTGCGAACGCCGTCCAAATTGGCAACCTCCCCTTTCCCGTGAACGGCCGGGAGGGCGGGTGTGTCCTTCC
TGGAGGGTGTGGGGGAGTCCGTTTCCCGCTGCTCCCCGGGAGATTTTAGTGTGTGCGGGGGCGCTCTCCA
GGTTCGGCTTAAGGGCCGGGGGCGGCCGCCGCAGGAGGAGGTTCTTGGGGCCCTCACAGGAGCTGTAGAC
CTGGGAACCCCCGCCCGCTGCTGGCCCCTTCCTGCCCCCGCCCCCGAGTGGTGTAGCTCTTGTGGCCTCA
CTTCCAGCGGTTCCCGGGCTCTGGAACAGCTGTGTTTGCAGACTTCCCCGGGAAGGGCGGGTGCACGGCC
CCCGCAAGCCTTCGGGGGCCGTGCCAAGGCTTGGAAGGGGTGGTCTTCTGGAGGTAGTGCCTGAGGGTGT
AAGTGATAGGCCCTCATCCCCCCGCCCCATTCCGCCCCCAAAGGAGCTCGAGGCCCGGGCCCAGCTGGCA
GATCCTGCAGCGAGGCCCTTGCAACATCCCAAAATGGGTGGAAGGAAGATGGGTGCGATTGCAGAAGGGG
AGGAAAGGGAAATGTTGGGGGTGTGCCCTCTGCCTGGCTCCTGCTGTCCAGGAGGCATGACTCATTGCCA
AGACTGGGGCTTGGCCTAGCCTGTGCTGGGAGGGTGGGTGGTGAGAGGGGGAGCTCGGTGCCCCAGAGCA
TTGCTTTCCACCACGTGCTCTAATGGGGTGCACTCTGCTGCCTCCTGTCCGCCGGCTTAGGTTGTGATTC
TGGGAGGAGACAGAGGGGAGACTATCTGGCTGTGTCTGTGTATGAAGCTTGGGGTGGCTCTTAAAATCCA
CGATTACAGGAGCTGGCATTATAAAGTGCCCTCTGCTGTGGGTGAGATAAGACAGTTATCAAAAACTGCC
ATGAAAAGGGGTTTATAAAAAGAAAAATTAAAACCAACGCTGTCTGTGAAGCTCTCTTGAGGTCCAAGGG
CTGTTTCTGTTGTGGGATTTCATTTTTACCAAGAGTATTTATCTTGTTTGTTAATCCCCTATTAGGGAGA
ACTTTAAGCTAAAGAGTCTGGCTGCGTTTAAATCTGGCTGGCCCAAGCTGACTGTGAATACTCAAACTGC
CCTTGAACGTGTGTTTGGAGTTTAGGGGGAAGGGGCCTGCAGGTGGTTCCACCCTCCTGCCGGAGGATCT
GTCTGGAGCCCCTCGCTTTTCACAAGACCATAAAAGGAATCTTATTAGTGATGTGGAAAAGCAGCAGCCT
GGCTGAGGCTCATTTTGGAGAGTAGGGTCAGAGGGATGGCTAACATGAAAACAAGGAGGCTTGCTCCCTC
GCTGGCCAAGGAAAAGTGACCTAGAAAAGTGCGTCTGTCCGCTTTTGACACTTGGTCAGAGTCCTCAATG
GAAAACGAGCGGAAATAGTGTGGGAGGGTAGGAGAGGCGATTGATTTGCATTTGCTGGTGCTCTTCTGTG
GTTTCAGGCCCCACGGGTCACCAACCATGAAATAGACTCTCGGCTTTTCTCGAATGGGGGAAGACCGATG
GGGCACCACTGAAAATGTGGTTCTTTTGAAGTTGGGACAAACGCATGTATCACTCATAAACAAGTCTCCA
CCCTGATGCCCCAGAAAGGCCTGCTGTGAAACATAAGATTTAATTCTCAAGAGAAATTGGAGTTTAGGTG
TATTTGTTTTTCTGTATGTGGTTGGAGAAACGGCACAGTCCGTAACTTTCAAGAATGATTCCAGGCTGAG
TAGAAACGTAACTGAAACACTTAAGTAGCTGAGCGAGTATTTAACTAGAGGGCACAGCTTCATTTAGTTT
GGCAATTAACTATGTCTTTTAAAACTGATTTCCTTGAATGGGAAATAAATGAACTTAGTACAGAATTCAA
GAGATAGCAAGAAAAGGATATATGGTTAAAAACAAAACAAAACTCCAAACCACCTTGGGCTCCCAACCCC
TAGCAGGCACAATTACCATTTGCATGAGTCCTCCCAAATTTGCTGACGCTGGGTATCAGCAAAGGGTGCA
GGGGTAGGTCAGTGTAGAAGGTCATGTAGTTCATGCTTTTTGTTTGGAATTATGACCCCTATCCAAGCAA
CTGTTCATGTTCTAGAGAGGACGAGGAGGAAACGATTTGGTCAGATATGAAATACAGTCCATGTTCTGGT
GTGCATGTGAAAGGCTTTGTAGAGGTGGCCCTTCCTGTCATATATCATATCTGAGTATGTCCCACGTGGT
GTCCCCTGCCCCGGCTCTGGTCTTGGGGTAGTTTTTCACCACTGGCTTCTAATTCTCATACTGTTGCTGC
TCTAAGGGGCTTAACTTGCAGCAACTATCTGGATTAATGATTCTCAAATTGGGGGACCAAGTGATAAGAA
CCATTATTGTGGGAAGTAATTGGGCTTAACAGGAAATAATTTAGGAATTACCACTTATAAATCACTGAAA
CGGTTAATCTGTACCTCCCAAAATTAGTGCTAAATCCCAGTAGTGCCTGCTTATTAAAAATACATTAGTG
TAATTACCACTACTCATCTCCAAAAGCTTTTTGGGAACTTCAGAACCCCAGTTAGCTGTATTGCTGCATC
GGTGGCCCCAGAGTGAAATGAATCTAGAGAAACGTTAGGGGAAGAAATTGGGGTATCTGGCTTATTAGCC
TCATATTTAAATTCCATTGATCCTATTAAAAGACTTGCTGAGATCAACTTTTTTTTTTTTTTTTTTTTTT
TTTTTTTTTTTTTTTTTTTGGAAAATCCAATGAAACGTGATTCTTCTGTGTGAGGGATGCCAGCTTTGGC
CCCCTTCATGCTTCAGGCCTGAATGACCAATTGCCACGATGCTAATCACAGAATGTAATTGACAGGCCCT
TACTTTTATGCCTTTTGCTGTCTTTTCCAGTCCCTGAGCTTCTGTAGATATGGTGTGGGCACATGGAGGG
CTTAAGGATGGGGTTCTGATTTGACCTGCCTCCTTTCTGCAGCCAGGATTTGCATCCTGCCCGTGTAGTC
AGCATGTATACAAAGAAAGAGCATGTGTGTACGTATGTGCTCAGAGCAAGGGACTTTCTGCATGTGGAGA
AAACGCCTTTTGCCCTAAGAAGTCTACCTCAAACCAAAGGGATGAAGTGATGTATCTGTGTTGCAACACT
GGGCTTGGGGGAAAGTGAAGCTGTGCTTGCTTTGAAAGAGCTGTGCTAAGTATCTGCAGAATAGCTGCTC
CAAAAATGGAATCTTTATTCCAGGTACTTGGTTTCTAATTTTTTTTTCTCTTGCAGGTGTAACCTTCTGT
GTTTTCTTTCTCCTTGTTGTCTTGTTTCTTAGCAACTTATTCAAACCTCCAGTACTTATAGGAATTTGCA
ACCCACCAGTTCCACACCCGTAATTCTACAGGGACTTAAGATGTTTTTAGAAAACGGTGAAACTTTGGCG
TGGAGGCTCTTGGGGGAGTTCAGTGGGTTAAAAGGAGAGTCAGCAGCAACTTACAGGCATGCAACCTCCC
AGAAAATCAGCAATCTAAGTGCTGCTCAAAATCTTCACACCAAATAGTGGTGCCTTTGGGGACCTTTTAG
ATAGAAGACGTGGAAGTTTAGCATGGAGAAGTTACTTAAAAAAAAAAAAAAAAAACAGTGTCAAGGTAAC
AAGGAGCCTTCACAGTTCCTGATCTGAAAACAGGAATGTGAACCAGAGGCCTTGGCTGGATTCATATCAC
CCACTGGCCCCATTGTAGAAGGGCCAGAAGTTGGTCCCAATGTCCGGCTTTCAAGGTTAGTGACAATGTA
AGAATGCATGATCTCTGGATGTGCATACAATTAGCGTTGTAATCCATAAATGCAAATACTCCTGCAAATT
CTTCCATGGTAAACTTCTGTAAGGCATGTTGCTCTTCCACCTACCCCCATCCTTCAAGCCTAGGATCTAG
GCTGCTGAGAGAAGTGACTCTTGGCTTCAGCTTTTAAATATATATATATCTCTCAAAACTTAATCTTTTC
TTCCAACTGTTGGAAGATACATTGGGATGAATGGAAAGTGGTGATTCTAAAATGGTGGTGGAAATGGAGG
AGGGTCCTGGGTTCAGAAAAATCCCACCTTTACTTTGGTGCATGGCACTGGGTGGCTGTGTTGTGACTTC
TTTGCTGATTTCTGATTACCGCATGGACCACAGCAGCCCTGTGACTGCTGTTTTTTTTTTTTTTTTTTTT
TTTTATGGTGCCAGTGACATCTACCATTGGTGCAGTGCGTGTGCTGGGAAGAAATTCCGCAAGGTGGCTC
CCAGTAGTTAAAGGAGTACATAAATAGTCAAACATGCAGAGTCCTCTACCAGCTCAACCCAGTTTTTATG
CACTTTTGGTAAATAAACAGAAAGGCTTCTTGTTCAGGCTTGCTCTGTGTGAACCAGACCGTTGTGCTTG
GCTGGCTTCTTGGTATTCTATCTATTAACCTCGCCACCCTTTTGTAACTGGTGCATTTAATTATCAGTGG
GATGCTTTTCAAACAGGTAAGGGTAATGATGGGAAAGGGGGTGGGGATGTCTCATTGAGAGTCACAAATC
TGGATATGAGCTTTGGGGCCCCTTGGTGAGCCTTTGAGTGGGCTTTGATGAGAAAATCACCCTTTGTAAC
CAGATCGGACTCTATTGACATGTCAAGCCCTACAGACTTGATTGCTTTCTGTGCCTGAAGTCTTAGCCTA
GATGCTATAGGATAGTTGTCCTTTCTTATCTCACTTTCCCAAAACCCACTCGCATTGTGTGCATGAGTAC
GTGTGCATGCGCCACACATGGACTCCCTGCAACCTTTTCTGCTTAATATGTTTTGTGACTGGAATATTTT
ATACTCTGGCCACCCAGACACAGGCAGGACAATAGAGTTGGCCGAATTGACTGAAATGAGGATTATGGAG
AATAATAGGGCCTCTCCAAAAATAATGCTAACACAGGACAAGCGACAGGAAGCAACAGTTGAGTCAGCAG
AGCTCAGGGCCCCTGACGAACCCTGGGAGCCGACTGACTGTGCACAATGGCCGCGTTGGCACGTCGGGCA
TTCCCAGGATGTGCGTGCCGCCGGCGGGCCTGCCTGTCGCAGCAGCCTGGGGTTTTGGACGCCGCACGCG
TGCCGTTTGGATGGCGGTTTATTTGTGTCCGTCAGCTTGGCAGGAGGGGAGGCAGCCGCTTCTCAGGGCT
TGGTTTGGCTGTCACTGTTGCCAGGAAAAGGAAGCTCCCGGCTGCCAACACAATTACCTTTGTTTCTTAC
ATCCCCCCTTCTTAATGTGGCAATTAAGTGCTTACCGCTCTGCCCAGTGCCAAGGTAACCGCCATGGAGT
GGGACAGGGCAGAGAACACAACCTTGGCACAGCAGCCTAAGTGTGGAGGGCGGGAGGAGGTGCAGGGGCC
CCAGCCTTGCTGGGAACCTGGTGGCCCTTGGATGCCTTGGGGCTTTGATGCCAGGACTCTCCTCTAGGTA
GAATTCCACAGAAATCTTCCAAAAAGCAATTCCGAAGGCTGACCATTCCTCTTTGATGCCACACCAGAAT
GGGAACCAGCTGCCAGGTTTCCAGATTTCCTTGGGCTTGGAGCTGGGAAGGGGCTGAAGTGACTCACTTT
GCAAATCCAGACTAAAGATCTGTTTGCTACCCCTCCTTCCTGTGTGGCCTTCACAGTACTGTTCTGGAAA
ACATAAGGAAAGATGAAAGCCAGGTGACTCTTCGGCAGAGTCTTAGTTCTGTAGTCTGAAATCAAGAAAA
ACCCAGGTATTTGCTTTTGTTGTTGTTGTTTTGCTGTATAAACTTAAGTTTGAACACTTGCCTTTCCAAG
CCCTCCTGGGATGGAGGACTCCGCCTCTACTGGGGGTCAGAGTTGCAGTGGCTGCTTGTATGTGACTTCG
GCATAGTGGATTTGCTGCTCTGTGGCCTGGTCCCTGCTTTGATGTGGGGTGCATGATCTGGGATCTCAGT
GCCTGGGTGTATGTTCAGGGATGTACAGAGGTCAAGTGTGGGCCTGGGAAAAATTAAAGTTCTCTAGCAA
CTAATAATCATTGTTAAATTATCTTGATTGCTGATCTATAGTGTGGGGATGGAGTGGGAGGTTGAGTGCC
TCCTAATAGGAACCTTGGAGGGAATCCTTTTTCTCTGTAGGGCTGGGCAGGAGGAATCCTCCTGCCCCAG
GAGACCCTAATCCCCTCCCTGAAACAGACAGGAAAAAAAGTCCAGGCTTTGTTGCCGTCACTGTAGCTCC
CTGCCCTCACCCTGTCTCTGCCTCTGAGAAAGAAGGGCAGTTCATGGAAAATCACAGAGCCAGGCACGTA
GGGCTGTGTGCAATTCTTTTCTCTCCAGGTGGGGTGATGTCGGTCAGCAACTTCTGAGGTTTTCCCAGTT
CTGGCGAGGATGAGCCTGGTAGACTGCTTGGTCGTCCAAGTTCTTGTGCAAGACCTAAGGATGCTTTCCT
CTTTGAGTATTAAACTGAGTTAGTACTGCCATGCCAGCTCAGTCCCGGCACCTTCCCTTGCAGTGGGGTT
TGTGGGTATTATGTGTTTAGCGCTTGTGCAAGTCTGTCAAAACTCCTGGTATCAATGCATTTAATCAGAG
GATTAATTACAACAATACTGTTGAAGTGGAGGACGTGGGCCTCAACGTGACCAGCAAATGAAGGGCTTAA
TTATACAGAATCAGCCACACATCTGGCCTCATTTTGACTGTGCTCTTACCTCAAATCAGTAGAGAAATAT
AATTTCTTGCTGCTCTTAGGGAAGAGTCCTGTAATTTGCAGGAAGTCCTAAGAAACTTAAAGAGGCAGTA
AATAAGGTATAACTTTCGCTAATAAAATAGTAACAACAGCAATGAATTTGGACCTGTGGCCACTAACAGG
AGAGGGCTGGGTATGTGCCCTGGCTTGCCCGGTTTTGCTTGCTTTTCTGAGCATTTTCCCACAGTTATCA
GTGAACGAAATGGAATCCTACTCTGCTTTGAGTATGAAACATATTTCGGCTGAATTCCAAGGCAGGGTCT
ATCACAAGATATTTTTATATTCAGAGATGACCTGCTGTGTCACTCTGAGAGCGCTAAGCTGGCCTTGAGT
TATGAATGAAACTCCCTTTTACCCTCCCTTTTTCTCTTTTTCCTTTATCTTATAGGTGCTTTCTCTTCTC
TTGGCAGCAGTGTGCTTCTGGTGGCACAGCACGGTATTATTTAATCTATACACGCTTCTATTTCCTATGG
CTTGGTTTCTGACACAGCAAAGCAGGGATTCATAACCTAGTTCATGTTCCTTGTCTACCCCACCTGCCTT
GAGTACATTTCTTCAGTGCCAAGATAGCAGAAGCTTCATTTAAACGCTGGTGTTTTGTGAGACTGACCTT
ACTGTAAATTACGAAGGAAAATTTTAGAAAAGGAGGGAATTTGCTAATAGCTTAGGTTTGGCTGGGGGTG
GGGAGGTGGAATTCTGAGATAAGCTTGGAAGATAAAAGGTGCCTTCAGTTTAACAGCTGGCATCTTGCCA
GTATCCAGGGAATTTGTTTAGTTAGTAGACTCTGATTATTCTTGGAATGTTAGATCTTATTAACAGATAA
GTTCATCAATAAATATAACCCATGGGTGTGCGGTTGTTTCTCCCTTTGTACTTTCTTCCCTGGGATCTGC
CCCCAACTCCACCGCCCAAGTGAGGTTGACTTCTTTGTCTTGCAGAGTCACTTTGCATCTACCCCCTCCT
GTTTCCTAGAGTTACCCGATACTTGTGGGAGCCAGGATGGTCTCACATAGACAGAGTTTTCAAAGGGGTT
CTAAAAAGGAAAAGGGGGCCTCCCTAGAGACTGCCTCACCAGGGCCCAAAGTGTAACCTTAGCAATACCC
CCTCGGTACAAATACCTGGTTAGGTTAGCTTAGAATAAATTCTTTGTGTGTGTGTGTGTGTGTGTGTGTG
TGTGTGTGTGTGTTTCCTCATGCACTAGGGTCAGGGTCTGGAAAGTTTTGGGAAACTATATGCTAAATAC
TCTCATATCATATAGTGAGGTGGCATTTCCTGATCTCAACTTTGAGCTGCCTGCATGCATGTTGACTCCA
GTGCGTCCCTGCCAGGTGAAAGGAGTCATTTGTCAACCTGGGAAGAGTGCTGACAAAGGAAGGGGAGTTG
GTTTGGATTTAAACATCCAGGAACTCAATGCAACAACCAACAGAATTTTTATTTTGGCATTATGGTTTTG
TTTGGTGGGGTATTCTGGGAGGCCAAGAATGTACGAGATTTTGGAAAGTCGACCCTGTTTCTGAATTGCT
AAATTGTTTGTTTGTCTTGCAGAGGTCCTATACGTTGCTTGTGGAGGCGTGGGATTCCAGTAATGACACC
GTTCGTAAGTATCGCTTCTGTGGACTTTTCTCTAAACACACGTGGAAGTGTGGGGCTTGGCCAACCAGTT
GGAGCTGGATCTGTCTCCTTAGTACTTAGCATTTCTGCCACCTCTTGCCTTCTGTTCTCCCCTGTACCCC
AGGGTGAACCTTCAGTGGAATGTATTCCTTAACTTTTCTAGGATTTTTGCTTGGAATATTAAGTGGCCAG
AACAGAATCCCAGTTAACTCATTCCCTGATTATGAAACCTAGATTATCTTCGGGATAGAGATCTTAATGG
TTTTACAACTTTAAAACCTTGTAGTCTGCTAAAGGATCAAAGTAGTGACTTTTTAAAAGTCCATTTAAAC
TTTAAGTGAGTGTTTAATCTCTCATCAGAAAGATAAAGCCATAAAGGTGGTGCTGGGCTCTAGAGACCGA
CCTTGAATTTCACTTGGTGGGCAGGTGAGCTTCTAGTCTCTCCGGGTTTCCCACACTGCTCCACAAACCT
TTCATTGAAGCGCAATTCCAGAGCTTCACAGCCATTACCTGCCTTGTCCCTTTCTGAAAGATGGCTCTGT
TTTCAGTGCAGGTTGCCTTTCAGGACTGAGGGGTTGGTTTTGGTGGATAAGCCATGGAGGATGTGATTGA
AATGACATAGTCTGGCTTACAAAGTAGTCTTGCTCTTTAATTGTAACTTTACAGTGTTTTCGCTCACGAG
CCGAAACTTCAAGTTCCCTTTTTGCCAGCAGAGTTTGGGTTGCGGTCGCTGGAAAACTTGATGCTCACTG
AAATCTCTTGGTCTGTGCATGAGGAAGAGTGGATCCAGCTTCTTGTCTTGGCTCTCTGTTCTCTGAATGC
CTTGTACTTTTTGCTTTTCTATCTTACCATTTTTTTTTGTTTGCTTTCACTGTGAATAATATATTTTCAT
CTTTCCTTGCCTTGCTTTTTTTTGCTGACAGAAAATGGTGAATGCACACCGTGGGTGCAGTGAGGGCTTG
GTGCCTGCTACGTGGGCAGCTTCCACCTGTGGCTGGCTTATTGGGCTACTTGGAGCCACAGAATCATTGC
TGAGCTCCGATGTGCCAGAGAGTCCCAGGCCATTTGGGTCACTCTTCTAGCTTGGGAATCTTAAGTCCAG
CTTTTTGGATGTCATCCTCCCTGGAGGGGGAGCTCATGAAAGTCCAAGGTTCGAGTCTTCCTCCCCAGAG
GGCTACCCTGTAATGCCGTGGGGGGGTGTGTGTGGGGGTGGCAGGGAAGCTCTGTCAGCCTTGGCTATGG
CTGATGCCAGTCAGGGTCATAGACACCTGTTTGTTCTCCCCTCCCTGCCACAAACGTTGAGGACTGTGAA
CATTATGTCACTGTGACCTGCTTACAATGGTAACTAAGCGTTAGGCAGAAGGGGTTGAGGAGGGGGGAGA
CATAGGCTCTTCTGTAATAGTATGGTGTCGACATGGTGTTCTTTCCCCTCCCTTTTAGCTATTGAAACAA
TCCACCCGGTAGAGTGAACAGCTTGAGGTTGATCCTGGCCTGTGATAAGAGCCAGGTCAGTATACTTGTT
AGGGACATGTGAGAGACCTCCCCGCCACACTGCAGCATTCACAGCACCTTTCCTGGACCTCCTGTCACCT
CGCAGGTCAGGGAGTTATGTCCCCTGGGAACCCAGGGCTTGTCCAGAGCTACCCACAGTTGTCTGCATTT
CCAGGTAGCCCGATTTTGGGGTTCTAGGATGTTTCATCTCCTGGGGGGGTAATCAAATCCCTGGAAGAGG
ACAAGAAAGACTGAGGTAGGAATAAAGTTCTTTTAAACCTCAAGGGTGCCCATTGCAGGTTATCAAAACT
CACTTGGTGAACCCTGAAGAGGGAGATGGGTTTGGTGGAAATGGTTTCTCCACTTGCCTATTTGGCCTCT
TTTATGATGCTTCCAAGGAATCTTGAATTCAGCACAGTCAAAACCAAACTCAGGATCTTCTCTTGCCAAA
AGCAGTCCCTTCCCAGGGCCCCTTCTGGGTGTCTGGCACCGATATTTGAACACTCATTGCCATAAGCCAG
AAATATAGAAGGCGATCTGTGTACCCCCTCTCTGTTAGCATCCATACCTAGTCCACCTCCAAGTTCTGAA
AAATCTCTCCAGCTCTTCACTTGAATCTGCCTGCCTTCTGCCACTGCCCTGGTCCAAGTCACCATCTGCT
CATGACTGAACTTAGAGTAGCCTTCGTCTGGTCCACCTTTATCTGCTCTGACTCACCTCTAGTGCTTTCT
CCATTCTTCTGCCATCCTGGTCTTTTCAGAGCCATAACATTTGCTTGTCACCCTTATTATGAAAAACCAA
CCACTTGGTGGCCTTCTGTTGCTCTTGGGACAGATCTTCATGTGGCCCACAAGGCTCTGTGCAGCCCAGC
TTTGTCTCCCTGCCAGCCTTGTCTTACACACCGTGTTGTCCATCGTATCTGTAGCCTGGCCATACCCTAG
CAGTTGCCTTCCTATCCTCAACTACATCACATGCTTTCTGGCCTCAGGACCTTTGCACATGCTGTTTATG
CTACTTGGAGTGTTTTGTTCCTTTCTTCACCCTCAGCCACTCCCTCTGGACTGCACTCCACAGGGCAGAA
GCTGAGGATGTTCAGAAGCCCAAACGGAGTTGGCTGCCCTGTGGGACCCAAGCGAGTTGTAAACATTCTG
GCTTCAGATGTAAATCAAAGGCAGAGCCCTGAGTTTTAGGGCAGAGAATTCCTTCTATCAGCTCTGCAGT
GAGCCCTCACAGGCAGACTCGGGCCCAAATATAGCCTAGGTGCTGTTTATGTATTTGAAAGTATTTAAGG
CTGGTCCTTCTGTCATCGGTCCTCCAAAGTCTTTTATTACATTTTGGGACTGTGGTATATAGAGTTCCAA
ATTTCTTTCTCCCCTAGAGCAAATGGTTTCAGTTTACTGTAATGCATAATAAACATGTAAACATAAATAG
GCACACTTCAGACCAGGTTTTCCCTGTAGCTTAGCTTTCTCGGCTAAGGCCCCCTTCCAGGTTTTTGGGT
CGGTGTGGTCCCAGGTTATGCTCAGACTCGCCCTTCATCACCTCCTCCTTGGCCTGCGAGGCGGTCATGG
CTTCTTCGTGACTCATCTTCGTGGCACTGGGGATTGCAGGGAGGCATGGTGATGTCCTTTTCCAGTCACA
AGGCTGGACTGCCAAACGAACTGCCACAGATTCCTTTCCAGTGCCCCAAGCTGAAGGAAAGCGTGATCAG
GAAGCAGGCAGCAGACGTATTTGAACCCAGACATGCTCGACCCCCTCCCTGAGCAGGTGTGAAATTATGT
ATGCAGCTCCATAGCTCCACTGAGGATTCTGAAGTGATCCTCTGCACGACACTTCCCGGAAATAAGTGGA
AAGCTTACTGCATGACTGAAAAGTACATATCAGTCCTGCACCCCTAGGATTGCCCTGGACTCTTGTCTAA
ACTGTTTGTTGTTGATACCAGCCTCAGAAGCTGGATGCCTTTAAGCCATGGCTAGTGTGTTTAACCGATC
CCTTTTATGAAGATCTTGTAAGCGCGTGGTAATAGCCCACTATGCTTTTTAACTGAACCAGCAGAGCAAA
CATAGTTAATGGAGAGACATTTTTGTCATTCCTTGGCCTCTGTTTATTTGCAAAAACGAGTAAAGTGTTA
CCTGCCTGCAAGTGGTTCAGTGCGTGCCGGGGTGTCAGGCTGCAGGTATGTGAGCTTGTTCAAGGCTTGT
CTTGCCCACGCAGCAGTTTGAGAGCCCTAGAGGGCGAACGCTGGGGCCCTGTTGGAAGCGGTGTGTGGAT
GAAGCCACCCAGGAGTCCCTTCTCTCCTGGTCACTTATAGGGCTTGCATCACTTTTTAAAGGAGCACTGG
CCCGAGGCCTAGAAAAACAGCCATGTGGGTGGGTAAGAATCACAAAGACATTGCAAGTCAGTCCTAACTG
TCTCTCCAAGATGGTCTTGAATTTGACAAGGTGATGAAGAGTTGGTGCTGGCAGGTTTAAGAAAACAAAC
ACAAAACCAGGTTGGGGTGCTGATTTAGTGCCTTGCTGCCTTTTCAGATCCCTCCCCTGAACTGCTGGCA
CCTGATGTTTGAGCTATTTTTGTACCTGTCTCTTCTCAAACTAGATGATAAGTGGCCTCAGGGCAGGGAC
TACATACTCCTGAGAGCTGCTTGAGCTCGAGGTGTTTATTATCCTCAGTAAGCATTTTTTTTCAGGAGCA
TGACTTGGCTCAAAAAAACAAAAAGAAAAAAGAAACCCTCCCCTCAATTTACCTGTGTCTAAACTTTGGT
GAATTATTCTCCCATCTGCCTTCTGGGCAGAGGAGAAAGGTGGAATGCATCAAGTTCAAGGTCTTGGTAT
TTAAGAGCTGGCTTTAAAGGTTGCCATGTTAACAATTGTAAAAAAAAAAAAAAAAAAAAAAAAAAAAGTA
ACTTAATCGTCTCCTTGGCTTCCAGTTAAGAGTTTTGAAATGGACAAAAAGCTTTTCTGAGCAAGACATA
AAACCCTAAAACCAGAAATGATTGCTGACCTTTATCTGGAATAATGAAATTTCTTCAAGAGCAACATTCC
ATAGGGAGCTACTGTCTCTCATATGTAAAGAGCACTTGCAAGTCAGTATGGCAATGTAATAGAAAAGTTG
GCAGAGAAGAGAAGCTGTTGCTGTTCACAGGAACAAATATAAATGGTTTTGTAACTACCAGAAAGGGCTC
TTGCCATCACTTAGTTAAATGCTAATTAAATGAGATACCTCTTTACCTATCAGGCATAGATTAGAAGGTT
TGATAATGTACAACGTTGGCAGTGTTATTGGGAAACACAGCTTCCTGGGTGGTGGGAGGACAGACTGGTA
GTTTGAGACTGTGGACAGAGGTTGATTTGTTGTGCACGGACTCTGATTCAGCACTTCCATTTCTAGAGTA
TCTCATTGTGGGAACCATACACAGGCAGATACGTTCAAGGAATGCTCACTGTGGCTTTGTTTAATAGTAA
GAAGCCCAAACGACTCACATGCACACATGTCAGGGACTAGGTAAATTATTTATTGGTGTTTCCAGACAAT
GAAATACTGTGTACCCATTAAAGAAGGTAAACAAGTGGTAACAGGTTGTCTCTGGGTAGGGGCATAAACT
TTTCATTTCTTTGCCTTTTTTACAACAGATAGAATTTTGTAGCAGGTGCATATGTTTTTATTTTAAAAAG
CCAAAATGGTCAGGGAAGAAGGCTGCAATGTGAATATTAACATGATTCATCACTTTGTCTCTAGAACCTG
ACAGTATTATTGAAAAGGCTTCTCACTCGGGCATGATCAACCCCAGCCGGCAGTGGCAGACGCTGAAGCA
GAACACGGGCGTTGCCCACTTTGAGTATCAGATCCGCGTGACCTGTGATGACTACTACTATGGCTTTGGC
TGCAATAAGTTCTGCCGCCCCAGAGATGACTTCTTTGGACACTATGCCTGTGACCAGAATGGCAACAAAA
CTTGCATGGAAGGCTGGATGGGCCCCGAATGTAACAGAGGTATGTGTGTGTGTGTGCATGTGCACGTGTG
CCTGTTGCTTTTAGTGTCCATTTATCACCCCACCAGCAGGCAGGTGGCATGCGGCTATCTCAGGTGGGGT
GGGATTTTGGCTGTTATTCTGGGCCTGCAGTATGAAGATGCGGAGTGTAGAAGATGCTGTTGGTGCCCCA
TCCAGATCCCCTTTACTGGCATCCCCATCCCCCAGGTGCTGTGTTGGCTGCCAACAGCTCACAGCTGCTC
CTTCTCAGGAGACTCGTCCTTGGCCAAACCAGTGCATGCCCCATTCCTCCAGCTCACAGTCAGTGATTGA
CAGACATAGGGCTACAAAGCCCAGCTTCCTTTGCCTTAAAACAGTAGTACAATTTGCACTCTAGAGCTTA
CCCTGGGATTAGGCTGAAATCAGACTCCAGTTGAGACCATGTTCTTGCTCAGCTCTTTCCCCTACCCCAT
CTGCTTCCCTCAATTCCTTCTACCTATGTCCAGATCCCCTTCTCAGACTCTGCTTCTAAGGAACTTGACC
CAAAATAGATCCAGACAGGACTAGGTCAGCCCAGTTGTTGTCTGAACAGGCTATCCTATAGGAAATGATA
GCCCCTGCATTCAGGGTAGCCTTGCAGCAAGTTTTCACTAGGTGTCTTTGGGGGCATGGCTTTGATGGGG
AGAGGTATGTGGTTAGCTCAAGCTTTCCTATTGGTGTAGGAGTGGGCCCACTTTTAGTCTCTGTGCTGTC
TACTGTCCCAGTCCCTTTATAGAATTCAGTGATTCCAATTCATAGCAACTTGATGGGTAATTCTGTTAAC
TGTATAATATCCCCTCATGAGCAATAATGTACTCTGCTTATTTTACATGATAATTAGGTAAATTGTTTTG
ATAGACTCAATTTCAAAGTGAGTTTTTCAACCATGAGAGAGGGGTGGGTCATGGGGGAAGGAATTTACAG
CTCTCCCGGGTAGTACTACTGCTTTCTTAAATAATTGGAGAGAAGAGTTTAAAAAGGATGGCATTTCTGG
AATTCAAATGAAAAGGGGAATTCATTAACATATTTTAGTGTGTTTTAAAAATTAAACGACCTAACACTTA
GTGTGGGTTTTTTGTTTGGTGTTTTTTTTTTTTTTTAAGATAAGGTGCTCACTTTGTCACCCATGATGGC
ACAATCATGGCTCACTGGAGCCTTGAACTCCTGGGTTCAAGTGATCCTCCTGCCTCAGCCTCCCAAGTAG
CTGGGACTATAGGCACATGCCACCACACCCAGCTTTTTTATTTTGTGTAGACATAGTCTTGCTATGTTGT
TGAGGCTGGTCTCGAACTCCTGGCCTCAAGCTATCCTTCTGCCTCAATCTCCCAAAGTGTTGGGATTATA
GGCATGGGCCACTGTTGCCTGGTCTTAGAAACTGACATTTTTTAGAAGTTACTTTTTTCTTTTTTTTAAA
CGAAGTAGAGAATTGCTCTGCAAGGATACTTTATAAAAGTCTCACCAGATCTGGCCACCAGATTAGCCTG
CCCAATGAGTCCAGCCTGGCTGGCTCTGTTAGTCCCATGGAATTTTCAAACCAGGCATTTGGTATGGGTT
GTTTTTTTTTTTTTTTTTTTTTTTAAAGGCTGAGGCTCATCTGAATGTGTCCTGGTGGGATGGGGAAGGA
TTATCTATACCTTTTTTTCTTCTAATGAAAAGGGTGTGGGCAGAATGGAAACTGACCAAGCTCTGCTCAA
GGAGCCAGGGTAGCTGTGGTTTAGAGAGGCTGGACGAAGGTCCCCCTTCCCTCGTCAACTTGTCTGCATT
TTGTCCCCAAACAATATCATGTTTTCAGAATCTGCTAACAAGGCCTTGAAGTTTAAAGCTGGTTTTGTTA
TTTGGGCCCCTCCCATCTTCCTCTCCTTACAAGGGACTTGATATGAGACCTGTTAAAATCGTGGAATTTG
CAGACTTTAATGCAATGGGCCATTCTCATGACCGGGGCGATGCAAGGAACAGGCAGTGTGCTGACACGCC
CTTCTCTGTTTTTTACAGCTATTTGCCGACAAGGCTGCAGTCCTAAGCATGGGTCTTGCAAACTCCCAGG
TGACTGCAGGTAAATCAACTGGTCTTTTGTGAGATTTCTTTTAATTTTCCTTTATGTAAGAGAGGAACTG
ACTTTATTGTGACTATGCCTCTTTTTTCCTTGGAGAACAAGAAGCTTTTTTAAAAAAAAAAATTCAGATA
GACACATCCGAAGCTTATTAAATTATACCAGCTGTCTGGTGGAATGCACACACCACTTCTCTCTTGGAAT
AAAAAGAAACTCTTTGGGCATAGCCAGTGACTTCATGTATTAACTGTTCTGTAAATTGATAACACATTTT
TATAATCTTCCCTTATAGTGCTGATGGGCTAGATGGAGGGATGTGGGCTTGTTTCTTTGCGTCTCTCTCC
TCTTCCACCAAGGCTTTCTTCTCCCAGCCCCCTCCCCGACTTTTCCCATCAATAGGCGAAAGTCCAGCAT
TCTTTAGAATGGGATTAGTCCAATCTCCTGGCCCCTTTGCAGGCGGGAGCTGGGTTGGTGCTGAGAGGTT
AATGGCTGCCCTGGAATCTTGTCAGTTGAGCCTGATGAATGTAGACTTTTGCTGCAGTCCTTTGAAGTCT
GTTCCTTTATGGTCCAGGAACAATGCGGAAGCCAAAGGGAGGCCTGCCCTGACCACTTCACGATGGCGGG
TTTTGGAACTTCTTGAAATTTATATTACGCCCGTTTGGAAAAGGCTTATTTTGGGAAATATTGTTGGTGT
CTGTGGTGTTTTTGGCAGTCTTAATGCTTTGCTCTCCCTCCTCCCAAATTTTAAGGAGTAACGCTAAATG
GATCCTTTGAGCCTGTCACTTTTGCCCCCAAAGCTACTCTGACATGAGTAAAGGGAATCTTATGAAACAT
AGTCTAAACCAGTATGTCCCAAACTAATGTTATTTGGGGCTCTTATTAAAACGCAGGTCCTGATTCTATA
GGTCTGGGCTGAGCCCAGGTTTCTGCATTTCTAACAAGCTCCCAAGCGATGCTGCTGATGGAACCATCAG
CAATCACTGCCCCCAGACCTCAGTACTCAAAGTGTAACTTCCAGAATTAACAAGGGAGCTGTTAAAATGC
AGGTTCGTGGGCTCCGTCCCACATCCATAGAATCCTAGTCCTTGGTTTGGCCCAGGAGTCTGCATTTGAC
AAGCTGGCCACATGACTTTGCTGCTTAGTAAATTTTGAGGCTACTCCTTTAAAGAGAACTATCTGTGGCC
TTTACCTTTATGATTCCACCGCTCCGTCTGTTCCCCTCCCATGCAAGGGGGAACATGGAACTAAATTAAG
AACTGGTTGTCCCGAATTGTCTCTTCCTACACTTCAGAAAGATAGTTTATTTCCTGGAAGGCTGAGGGGC
TTCTCCTGCTGTCTCCCATTGGTGGCGATGGACTGTAGTATTTAACAGCATTGCTGTCCAATGCGGAGCA
GGAATCCACCTACCTTAACTCTTCCTAATTAAGGGTGTTCTGGAGAAAGTCCCAATGGCAGTGCTATTAG
TGATTCCAAGCCTGCTGCCTGCTCAGATGGATATCAGCGGAGGCCGACTGTGATTTGTGCTGCTGTCATC
AGAACACACATACAGCTATACAGGGGAGGGCCCTCCCAGCCAGAACAACAGGCTTTGTTTCCAGAATTTA
GCCCTTTCTCTTTCGAAGTCCCACCTCCCTCTCCCTGCCCAGACACGCACTGATGGCCTTATTTACTTAA
GGTGCGACTTTCCCACACATCATTTGCATAGTCTCCCAGCCCAGTTTTCCCTCCCTACCTTGAAAGAAAG
TCAAAACAGAACCTTCACGCTGAGGAAGTTCAGGTTGTGTCCTGCTGCAGAAATAGTTGAATGGCAAGTT
ACCTGCAAGTTGTCTTTTGGATTATGTATTACACAGGTTATTTTTTTTTTCACCCCCACATGAGAAAATT
TACTGGTTTTTGTTGTTCCCAAGTAGACTGAGTCCCTTTCCCTGGGTATACATGCTTGCTTATTGCAAGC
CTGCCAATAATATCAAACTTAATTATGAATCATCATCTGAGGAAAGTCACTTCAATTTCTCATGAGAATC
TTAATCATATATTATTAAACTGGGATAACTTGGTTGGTGCCTCTTTTAGGGTCAGTATTTTTATTTGTTT
TCTTTTGAAGTCATCCATGGCCTCTTGTGGTTGCTTTTTTATGCTTGGGGATTGAGGTCTCTTTAATTAG
GTGAGGTTGGGTCCTCAGCTGCCTCTCCTTGCCTTTATGAGGCAACTTGAAGAGGGCTAGTGATCCTGAA
TTTGGAAGAGAATAAGGGAAGACGGTGAGAATATTGCCATTTCGTAATCAGTTCCCTCTTACTATCTTTC
AGGCTAAGAATATCAGCCTTGACAGAATGGCTACACCCTTACCTCCCACTAACGTGGGTGTCCGCTTTTG
AGAAATTATTTAGATTGTAATTTTGTTCTTATCTTACTCCTTTAGTCACTTGTCACGTTGACATTCCATT
TTACATAAACCAGAATCCAGTGTCTAACTTTAAACCACACATTCTAGTGATTTCTTTGCATGTGGGTGTG
CATAGCTTGGTTTCAGAAGTTAGGAAGTGGTTAGTCAGTATTCTCAGGGGTTAAGTTGACTCTCAGCCGT
GGGTGATTAGCTATTAGCTAGATCCCTATAATTTTCCCATGACAAGGATTTGCTAGCCTGCCTCCCGCTC
TTGGGAAACCGAAAGCCTTTCCTTCTGGGGAGCTAGAGGGGGCGGGGGTTGTTTTCCAGAGTTACAGCTG
AGCAGCTTAGGGGGTTGGCGCTGATCGTGGCTTTTCCCAGGTCCCGTTCTGGAAACATGTTGCCCATCCC
ACCCGCACCCCACCCTCCACCCTCCACCCTCCACGCTCCACCGGGAGGCTAGTTGATGTTTCTCCCTGCC
TTCCCCCACCCTTGTTAAAGCTTGGCTCTTGAGAAGGCAATTAAAGTTTCAGAGGGCTTGAAAATTCAAG
GGAGAGTGGTTTGAGACGCTGCCCCCTCTCCCACCCCAGATGAAAGTTGTTGTATTTGTTTTTGTTTTTG
CTTGGGGATCAGAGGCACTATTTGGCCCCAAAAGAGTATGATTAATCAAGGTGCTTATATAAAAGAACCT
CAACTCCGCCAATACTAGATAAATTAAGCAGACTGCTTTCTTTTTTTTTTCTTTTCTTGGCAAAGCAAGA
AATCACTATAAATTAGCACACAGTAATGGGACACAAATTATGGAGTTGGTTCCAAGTGTTGTCATTTCCT
TCTCCTTCCCTATAGAAGTGGGTTTAGAGAGCCTACTCTGGCTTTGGCCAAGGGAGGGTGCCCCTTTCTC
TCCTTGTTTTTTTAAATAAAGCGTCATCGTGAGGCAGTTTCCTGCTGCACTTGGTGGCTTGTTGCATTTC
TGTGCACTGGGGTCATGACTTTCTGTGCAGACAAAGAGCAGAGTGGCAAGTGCAGCGTCTGCAGAGACTT
TGGCATAGCTCTCTTGTCACCGAGAGAGTCCTTTCCTCTATAGTCTGTCGCAACTAGGTGCACGGGTTGA
TTTATTCCATGCCTTCACCTTTGGCTCACTCGTGTGAGCCTGAGAAGAGAGTGGCTTTGTGAAGGGCATC
CCCATGCTGGCCTCTTCCCAAACAGCCCTCCTTTTGTCAGGAGTCGGCTGGGAGGGGACCTCAGCTCAAC
TGTGAAAATGTTTGTTTATACGGAGCCCCTGATGAGCCTTGGGAGTCTACCTTCCTGTCCCCAGAAACAA
AACAGAAAAAGCCTGGACTTTGCAGTCTTCCATTTGAATTGTACAGGTGTCTCTTAACGTTCAAAAGGCT
AACCTGGAGGTGTGCTGTGTGTCTCCAGGTGCCAGTACGGCTGGCAAGGCCTGTACTGTGATAAGTGCAT
CCCACACCCGGGATGCGTCCACGGCATCTGTAATGAGCCCTGGCAGTGCCTCTGTGAGACCAACTGGGGC
GGCCAGCTCTGTGACAAAGGTATGGCCCTTAGGGATGAGACCCAGGGTGGGATCTGGGGGTGGGATGGCA
CTAATTTGGTTTCTAATTTTATTGGCAGGGGCTTTTATTCTGAATTCTCCTTGAATGCTGTGTGGGTTTT
CTGGTTTTTCTTTTTAAAAGACGACTGGGGCTAGAAACATGGTGGGTTCGCCATCTTCACAGGCGTCTCT
TAGTGACTCCAGGATCTTTTTGGCAGATCTCAATTACTGTGGGACTCATCAGCCGTGTCTCAACGGGGGA
ACTTGTAGCAACACAGGCCCTGACAAATATCAGTGTTCCTGCCCTGAGGGGTATTCAGGACCCAACTGTG
AAATTGGTAAGTGGTCCAAGATGAATGAGAGCCCTTTGTCTAATTTTTCTGTACTCAGCTTTTAAAAAAT
ATACTTAAGGACTTGTTTAAGGAAAAAAAAGTGAAAATAGCTTCCAAAGATAGGCCTTATCCCAACCATT
CTATAAGCCTTTGTAACTTTCAAGTCTCTAAAGTTTTTTGGGTGATGCTGATTCGTATAGGAGGCAGCGG
GAGATGAAATGACCCCAGCTCTGAGATGTCTTGGAATGTGGTTAGAGACAGCTCAGCGAGAAAGATGACT
TCCTTTCTTTCCTAGACACAGCAGGGTTCTATCACACGCTTGCTTTTGGTTTTTGAAATATGCTTGCACT
AGTGGCTGTGGTGTGGGATTCGGTTGGAGGGGGGTGTGGGGAGGTAACGGTGTGCAGGCATCCCTCTCTG
ACTGCCATCCTGGTTTTTGCAGCTGAGCACGCCTGCCTCTCTGATCCCTGTCACAACAGAGGCAGCTGTA
AGGAGACCTCCCTGGGCTTTGAGTGTGAGTGTTCCCCAGGCTGGACCGGCCCCACATGCTCTACAAGTAA
GTCCAATTTTCAGCCTGTGTCCATTCTAAGGATCCTTGTGCCAAGATACCACGCTGGGGAGAGGTACCTA
GGCTTGTCCAGTGTGAATGTCTCGGTGAGAGAGGGGCGGGTGCAGGCCTAAGTCTGGTAAGCAGTCTGGT
GGCTTGTGTGTTTGGAAGTCTCCCAGCACCCCCATCCTGTCCCACTAAGGTAGCGCATGTATCTGTTCTT
GGAAGGAACGTCCTCGTGGAATCGCTTGTTCCTTTGCATCTGCTTCCTGGAGCTGGCCACCTTCCAATTG
TGGTTATTCTTCTTGCAGCCATGGAGAGGCTGCCTTCAGGGGAGGTTGTCACTTACTACTGGATTCTGTC
ATAGGGCCAGGGGGCCCCAGAGAAGAACCTGGCCTGCAGAGTTCCTTTGGCTGTGAGACAGGGCTGTATA
TCAGAATCACCTGGGCTGCTTTACAGTGTAAATCTCTGGGTCCCCCAGATTCCCTCCAGAATCGGGGAGT
CCATGGTCTTTGATAAACTTCTGGAATTCTGCAGTGCAGCCAGGTCTGAGAACCGCTGTCATCAGCACCC
CAGGTGTGGTTATGGATGTGGATTTCTGGGGTCAGATTGCCTGTGCTGGTCCTAGGCCCCCACCTGTCAC
TTGCGGAACTCCAGCAGGCCACAGAAGCTCCCCGGGCCCTCTTTAAATGGCACAGTAGCAATTCCCATCT
CCGAGGTTGTCATAAAGACACAAAAGTGAATACTTCATAAATTGTTAGGAGTTGTCTCTGGTCAGAGCAG
AAGTGGAGAATGCAGCCTCTAGGTGTGCTATTTTGGGCAAACTCTGTAAGCAAATGAGAGGAATAAGGTT
TTCCACCTGTAAAGAACTATTTTTCCTGACTTCTTAGCTACTTCTGAGCCATGATGAAACTTTTCTTTAA
AAATAAAGATTAGGCAGATAGAGATGTCCTGTGTTCCTCCTGGGCGGATGCTCCGTCAGTATCCAGCTGT
CTTTCTCCCCCACTGCTTAGGTTGGGGATTACATGAACTGGGGCAGTGGGGGATTTAAAGCTAAAATCAA
GGCTTCGGCCTTATCGTCACCGTCCCACTTGTCCTGCTGTAGCTTTCCTTGTAGCAGGTGTCTGGCTCTT
CAATGACAGCCCCTCATGTAAGAGGTGATGTTAAAGGATCTAATTGTCAGATGGATTGAATTAAATTGTC
AACCCCCTCCTTTTCTTGTTGACCAGACATTGATGACTGTTCTCCTAATAACTGTTCCCACGGGGGCACC
TGCCAGGACCTGGTTAACGGATTTAAGTGTGTGTGCCCCCCACAGTGGACTGGGAAAACGTGCCAGTTAG
GTAAGAACATCCCTGTGACTGTAATTTTTATACCAAGGTTGGCTTTGATTGTCAAAGCCATTACAGAAGA
CGAGCGTGTGGCCGGCAGTGTGAGTTCACACTCCATCATGCTAAGAAGTAATTTATTATCAAGAGTCTAG
CAGGCCTGGGAGAAATAACTTGAATGCAAATGGACTAGCTCTTTTAGGCCCTGTGGGAAAGTCGTGGGAT
TCCTTGAACCATGTCAATCGTCTGTCCGAGGAGTGTTCAGCTGGGTTCCAAAAGGGGATGATCTCATGAG
GAATGAGCTGTCTGCATTCTTTTTGCCACATTTAAATCCCAGCAACTAGGCTGCCTGGGTTTTGCACAGA
AATGCCTGGTGGCTTTCTGCTGTTTTGTTGGGGGCTGGTTAACTAGCAGCTGGAAAGTTATCAATTAGGG
TTCTTATTGGATAATAGCTAGGGAGAGGGATCAGATGCTTAAAATAGAGGGAACCTCTAGTATCTTTTAT
TTAGCTAGCTTATTAAAATGTCTCATGTCTCAGACAAACTCTGGCCTGTTCTTTTTTTTTCCAAGGTGGG
GGAGATGGGGGTTGAACTTCATTTCTCATGCTCATCCCCATCTCCTTTCAGATGCAAATGAATGTGAGGC
CAAACCTTGTGTAAACGCCAAATCCTGTAAGAATCTCATTGCCAGCTACTACTGCGACTGTCTTCCCGGC
TGGATGGGTCAGAATTGTGACATAAGTGAGTGACTTTGTTTCCATTTTGATTTTCATGAAACTTGGGTAG
CCGACTTGCTGGTCCGTACTGGGGCTCCAGTCCTAGGACTTGAGCAGTAGAAGGAAAACCTTAGAGTGGG
AATGGTTTTTTGAGAGGGAGTGGTCAGGGCAGCAGTGAAAACATACACTTTATCAGGCTCATTAGCTGCG
AAAACCAGCCATATTCTGATAATGTGGCTGAAACAATGATGTGAAGTTTCACTCCCTCATTCTGGAAGAA
TTAGGGAGGGTCCCCACCCAGCGTAATAACCTTATCTCACGGAAAGCACACAGGAAATCTGATTTGAAAC
TTATCTATCCTGAGCTGAGAAGGCTTTTCAATTAAGCCCAATTTCACTGTAAATTACCTCTTTAAAATGA
TGACTTATTTATTTTTTAGATATTAATGACTGCCTTGGCCAGTGTCAGAATGACGCCTCCTGTCGGGTAT
GTAAATCTTTGCTTAAATCAAAACTTTGACAATTGACAACAAATCTTTAGATTTGTGCGACACTAGGAGA
GCTCTGTCCCTGTGAGATTTAAAAATTTGGGTGAAAAAAGAGTCTCTTGGAAGTTTCGCTAGGGCTTATG
TTACCCCAGCCTCGTGGGAGTCATTTTGAAGGCATGCACATAGAAATTCCTCTAACCACTTTCAGAGGTT
TAGCATTTGGCTATTTCAGTGAACCAGCTAAACCGCAACAGTCATGCTAAACCGCTGAAGCCCTGTGTTT
GTGGAATACATAGAAAAAAAGCATGTGTCTCTCTCTCCCCTTCTCCTCTTCTAGGATTTGGTTAATGGTT
ATCGCTGTATCTGTCCACCTGGCTATGCAGGCGATCACTGTGAGAGAGACATCGATGAATGTGCCAGCAA
CCCCTGTTTGAATGGGGGTCACTGTCAGAATGAAATCAACAGATTCCAGTGTCTGTGTCCCACTGGTTTC
TCTGGAAACCTCTGTCAGGTGAGTGGTGGCAAAACCTCAGATGGCCAAGTTTTTAGAGCAGAGCCGCTGC
CTCCTCCGCTTCCCTTTACTTTTCCCTCAGCTCTTGTGTCTGGAATTTCCTAACATCTATTAACATCCTA
TTGATGAGAGAGTCTCTGCTAGACAAATGCTGGTGGAAGGAGATACTGACGAATTATTTTCTTCTTAGTG
TCTTCGTCTTTGTGTTCTTTTTGGTTCCCTCCACCCCCTCTTGTGCTCCCTCTCCCACTCTGCCCCGTTG
TTTCAAATACTGGACATTGGCAGTCCCTACAGCATAGAGATGTATCTTACAGGGTGTCTCCTAATAGAAA
CCGAAGGAACTGTCAGATTCCAGTGCCTGCTGCCCCAGAGAAGTTATCGTGACACCTTTGTTTTACTCTG
ATCCCTCCCCCTTTTCGCTGTTCCTGATATTTGCAGCTGGACATCGATTATTGTGAGCCTAATCCCTGCC
AGAACGGTGCCCAGTGCTACAACCGTGCCAGTGACTATTTCTGCAAGTGCCCCGAGGACTATGAGGGCAA
GAACTGCTCACACCTGAAAGACCACTGCCGCACGACCCCCTGTGAAGGTACCTCCCTTCTCCCGGGACCT
GGCTGTCTCCAGACTTGCTCCTTTTGTCCCCACTTACTACCACTGCCCCTTGTTACACTTGAGAAGTAAT
GTATTGGTGAGGAGGACCTCTATAGTGAAATGATCCTGCCTGTGTAGCGGGCCGCAACACGTGGAAACAA
TCTATTCCGGCCTGCCAGTGGGAGAGCCGTGTCTACAAGTAGCCCAGGGGAGAGAATGTGAAATCCATTT
AGCATTCCTGTTCTGGGGTGGGCTGATGTGCAACGCTGAGGCAAATGCCTGAGAACAACACTGTCAACAG
AACTTCCTGCAACGGCAGACAGAGGGGGATGTTCTGTATCTGCACGGTCAGGTATGGGAGCCCCAGGCCA
TGTGGACTGTGACTAGTGTAACTAGAAACTGATTTTATTTCATTTTAGTTCATTTAAATGTAAATAGCCA
CATGTGGCCGGTGGCTGCTGTATTAGGCAGCACAACATTTAAAGCTTCTATAAGATTGTCCTTGAGCCTT
GCTTAAGATCAAAAAGGAAGTAAATAAAGCAGGTCCTTTTGCCTTAGCAAGTGTTTACCAAATTGGGTAG
ATGTCAAAGGTAGGATGAAGGGGATCCACAAGCATGTGGGTTTTCCTGGTTGTGTGGAGGGAGAAAATGC
TTTAAAACTCTTTGCCTGGAGTCTCAACTTTGCCTGTCATAATGTGATGTGGCACGTTGCCAGTATCTTG
GCTTCCCTGGGGTGTGTCTCATTAGCCAGAGGGTCCCTGTCTCCAGGAAAGTGGGCTTGGCCCAGGCCCT
TGGCTTAGGAATGCCGCATCTGTGGGTGCATGGCTGTGATCTGATCCCAAGCCTATCTTCTAGTGATTGA
CAGCTGCACAGTGGCCATGGCTTCCAACGACACACCTGAAGGGGTGCGGTATATTTCCTCCAACGTCTGT
GGTCCTCACGGGAAGTGCAAGAGTCAGTCGGGAGGCAAATTCACCTGTGACTGTAACAAAGGCTTCACGG
GAACATACTGCCATGAAAGTAAGACTCCCTCATCCGGGAATGGACCAGTGCTCCCCAGCCTGCCCCTGCC
CCTGCCTCTGCCCCTGGCCCACCCTGGGATCATTGGTGTTGAAAGTTTTTTTTTTTTTTTTGAGACCGAA
TTTCGCTGTTGTTGCCCAGGCTAGAGTGCAGTGGCACGATCTCGGCTCACCACAACCTCGGCCTCCCAGG
TTCAAGCGATTCTCCTGCCTGCCTCAGCCTTCCTGAGTAGCTGGGATTACAGGCATGCGCACCACGCCCA
CCACGCCCAGCTAATTTTGTATTTTTAGTAGAGATGGGGTTTCCCCATGTTGGTCAGGCTGGTCTCGAAC
TCCCAGCCTCAGATGATCCACCCCCCGACCCCCAACTTGGCCTCCCAAAGTGCTGGGATTACAGGTGTGA
GCCACCATGCCCAGCAGTGTTGAAATTGTTTTAATCAGAAACAGTAAGGCTGGGGAGACTGGTTGAGACT
TATCCTTACATCTGGGATCTAAGACTGAGGTCGTGTTCAATTTACCAGATCACTCTGCCTCGGAGCCTCC
ATATCTATGTTGGAACATTCTGGAATGTTGCTAGGTTGGGTGATTCCTCTTTTCTGTAGGAGTTTCTTCC
CCACTTTCACCTAGTACCGGTGACTAAATGCCCATGTGAGGGCATTAAGTGGGTAAGGCACTCTCTGGAA
GCTTCTGCTTGATAAGCCGCCTGTCTAATTTGAGAAACAGCCCTGATGGAGGGTAGCAGGTGGAGGTGCC
AGGTGCATTGTGTCAGGAGGGAGCCATGAAAACTGCTGAACAGTGCTTGTCTATGGCTTCACAAGTCCTT
CAGTTCTTTTTTTGCTTTACTTTGTTTCATAGATATTAATGACTGTGAGAGCAACCCTTGTAGAAACGGT
GGCACTTGCATCGATGGTGTCAACTCCTACAAGTGCATCTGTAGTGACGGCTGGGAGGGGGCCTACTGTG
AAACCAGTGAGTCTGCCACTCTTTGGGGAGCTCTGGGATGTATGGGTCATGTTGGAGGGATCCCACTTCA
ACGTGGGAAAACTTTCTTTGTACTGGAAATCTCTGGAAGCAAGACCGCAGTGATTTCTGCACTGGGACAG
TATCACTTGTGTCTCATTTGCATTTATGAGTCCTGATGATAGCTTATGGGGAGGAGCTTCTGCTGAGGAC
CTATAGAATTAGACTGTGTAATAAATATTCCTGCTTGCAAGGAAAGTCATATTTCTACTTTCATTTCCAT
GGGACTTCCAGAGGCCCACCCCACATCTTGCCTAACTTGTGGCATTCAAAGTAACCCAGTTGGGAGCTCC
TTTATTCCTGGAAAAGCCTGATTGTGTTAAAGTACTACGACTGTGGCTGTTCTCAGAATAGGATGTTGGA
TGTAAGCAGTAGGAAAGGCTTGGCTCTCGCCTGTCGTGAATGGTCCTGGATCTCGTCTTCAGCCGATTGT
CTCCCCTTTGATTCTAGATATTAATGACTGCAGCCAGAACCCCTGCCACAATGGGGGCACGTGTCGCGAC
CTGGTCAATGACTTCTACTGTGACTGTAAAAATGGGTGGAAAGGAAAGACCTGCCACTCACGTAAGTGGT
AGTTGGCCTTGGATGCTATCTCTGGGACCCTTCCCTCTGTCTTCTGTGGGAGGGGGCCACTGGGACTCAC
ACTGAAATATTTTCCTCCAGGTGACAGTCAGTGTGATGAGGCCACGTGCAACAACGGTGGCACCTGCTAT
GATGAGGGGGATGCTTTTAAGTGCATGTGTCCTGGCGGCTGGGAAGGAACAACCTGTAACATAGGTAACT
TTATGCCCCACGTGGGATCTGCAGTGGGCATGGCCACCTGGGCGTGTCTGCTGGTTGGCCCCTGGAGCTT
ATGATATTTCTCTGGGCCTGGTTCTTGCCCCTCCAGACTGTTTCTGGATACTCAGGGAGGGTGGTCGTGT
CTGCTCTTCTTTTATAGCCCGAAACAGTAGCTGCCTGCCCAACCCCTGCCATAATGGGGGCACATGTGTG
GTCAACGGCGAGTCCTTTACGTGCGTCTGCAAGGAAGGCTGGGAGGGGCCCATCTGTGCTCAGAGTGAGT
GTCCTCCCCCCTTGAACTCTCCCAGGGCTGTCGGAGCAGATCCTGACACCCTTGGGGACTTGCAATGGAA
ACCAGAAGCAGAGCTGTTTGATGTCTGCTTTTGTATCACCTGGGTCTGCATTAATGACTTGATGGGATAC
AGCCCTGACCTGGGCATGAGAAAGTCGCCAATAAGATAAGTGGAGACAGATGGCGTCTCAGTTCCCAGCT
CTGGGTCTGACCAGCAGCCTGTCTCCTTCCATTCTCACCCCCACCCCTATCCCCTACCAGGGCCTCTTCC
TGTGTACATGTGTCCCACAGCAAATGAAACTAAGTGTGGTGCGGGCCTGGTTCCAATTTAGCAAAGGTAT
TTTGCTGGTGTTAACTACGACTTTATCATTTGGGTATTAGGAAAATAAGTGGTGGTTTTACTTAGGGCTA
AGACCGCTTTCCCTGTTGAAGTCCTCACTATTAAGTTGTCTTTTCTTTGCAGATACCAATGACTGCAGCC
CTCATCCCTGGTAAGTGTGACATCCTTTTAAGCCAGCACTCATCCACTATCGTGTGTGTGTGTGTGTGTG
TGTGTGTGTGTGTGTGTGTGAGAATCACCCAGGGATTGTGTTTAAAATGCAAGTTCTGAGTCAGGAGGTC
TGGGGTGGGGCCTCAGACTGCTTTTCTAACAAGCTCCCAGGTGAAGCGATCCTGCTGCCCCATGGACCAC
ACTTTAATTAGCCAGGTTGTGTCCTCATTTAGAGCAGTGATTTTTCAACCTTGGCTGCACACCAGAATCA
CCTGGGAGCTTTTAAAAATCCCGCTGCCTGGGCTGCAACCAAGACCTGTGAAATTAGACTTTCTGGGGGT
GGGACCCAGGCATCAGTATTCTTTTAAAATTCCCCAGGCAACGGCCATGGGGTGAGAGCCACTGACTGTA
ACTGGTGTGGTGACATGTGAGTGATTGGCAGCCAGAGTAATGGACTGGGAGGTTGGTAACCAAGGTGGTC
TTTGCTTTCTTAGTTACAACAGCGGCACCTGTGTGGATGGAGACAACTGGTACCGGTGCGAATGTGCCCC
GGGTTTTGCTGGGCCCGACTGCAGAATAAGTAAGGACTGTCTCCGTCTGGTTTTCCCAATGGCTTTACCG
CTTCATTCCATGCCTCACCCCAAGACTTTCATCTTTAAAAACAAAGCCACCATTCCCTTTAAGTAGCTCT
CATAGAGCAAAGGATCTCTCTCCTCCAGCTGGGATTAAGATATGCCAGGCTGGATCCTCCCAGGTACCTG
CTGGTAGTTCCCTGGGTGGTGTAGCCCTCCTTGTTCTTGGGGAGAGAGTGCCAGTCACAGAATCTGATGA
TTAATCTACAGTGGGGTCCTGAAGAAAGTATGTGCATTTCCCCATGACTAACATGAAATTATGGGCAAGC
AGATGTTAACCACAGCTACACTGGTATAGGATAAAGCTAATACAAACGGAAGGGAGGACGACTGTGGCTT
CCAGAGGCTTGTCTGTGCTGGGTCTGATCAGGCCCTGGAGAAATGACTAAAACAGGAAAATAGGGGCAGG
CAACGCAGTTTTTCAAGAGGCTTAAAGCATATTATTTTGAGCCTTTTCTCTACTTTTTTACAAGCCCTTT
CTCTTACAGAACAAAAAAGGTGCTAGAGTAGTTATGGTTTGATTCTTGTACCCAAATTGTCTGAAATTTT
ACTGGTGCTTGAAATCCAAGTCTGGGATTAACTAGTCTTCAGCACTAGATGGGGGCAGGTTGTATCTGTT
TCATGTTTCTAAATAAACAGTTGGCAAACTGTGGCCCAGGGGCCGTATCCAGCCCACTGTCTGTTTTTAT
AAATACAGTTTTCTTGGCACACAGCCACACATTCATTTATGTATTGTCTATGGCTACTTTCATGATACCA
CAAGAGTTGGATAGTTGTGACAGAGACTATATACCCCACAAAGCCTAAGATGTTTACTGTCTGACCCCTT
CCAGGAAACTTTGCCAGGCTTGCTGTAAATTGCACTGTCAAAGGAATCAAATTAAAAATTGCTGCCTCCC
AAAATGGTTCTCCACTGTTAGAACTCAGGATGGTGGTGGTCTTTGGGAGCCAGTGATTGGAGGTTTTTGC
AATAGATCTGTTTCTTCTTTTGGATGCTGCTTACCTGAGTGAAAATTCATCAAGCTTATGATGATATGCT
TTTCTTGTATGTTATGTCAATAAAAACAATCTCAAAACATTGCCACACACCATCAGTCCCTAAACTTGAA
CTCCATTTCTCCTAGACATCAATGAATGCCAGTCTTCACCTTGTGCCTTTGGAGCGACCTGTGTGGATGA
GATCAATGGCTACCGGTGTGTCTGCCCTCCAGGGCACAGTGGTGCCAAGTGCCAGGAAGGTATGTGTGCC
AGGCTTCAGCTGCCCATGGGTCTTCTGGGGTGAGCGAGCTTTGCAGTCACCATTTGACTTTTCACAAATA
AGCCACTGTGCTCAGGGAAGGGAACACACAGTGGGACTACAAGGGAAAGGGAACGTGAAGAGTCTTCCCC
AAAACAGGTAGCTGTTGTTTTTGGTCAGCTGTTGCCACAAAACTGTGTACCAAACCATCCCAAACTGCTG
TGGCTTGCAGCAAGCATTATTTTTATTGCTCATGATATGCAGCATGGGCAGAGAGATTGCGCTGTGGGTT
GGGTTCAGTTCGACCAGTGGCTGTGTCTGGGGCATATTCCTTTCATCGCCAGTGGGTAGGTGTGCAAGAG
GCTAGTCAAAGGCTCTGCTCACATCACATCTGCCAATGTTACATTGGCCAGAGCAAGTCTTGTGGCCAAG
CGCAGAGCCAGCAAAGGCAGGAAGTACACATGCTGTTACTGTGGGAAGAATGATCTTAACATGGCAAAGG
GCACAGGCATAACCATTTCATAACAGGGACCAAAGAATTGAACCCCGATCCGGTTCACAGAAAAATAAAC
TGCTCACCCGTCTCCACCTGTCAGTTTCAGGGAGACCTTGCATCACCATGGGGAGTGTGATACCAGATGG
GGCCAAATGGGATGATGACTGTAATACCTGCCAGTGCCTGAATGGACGGATCGCCTGCTCAAAGGTAGGA
CATGATGGCTGCCGCAGTTCACCTGTGTTCTGGAATCAGGGATGAGCATGCCTGCTAAGCTGCCAGCTTC
TGTTTTTCTCCAGGTCTGGTGTGGCCCTCGACCTTGCCTGCTCCACAAAGGGCACAGCGAGTGCCCCAGC
GGGCAGAGCTGCATCCCCATCCTGGACGACCAGTGCTTCGTCCACCCCTGCACTGGTGTGGGCGAGTGTC
GGTCTTCCAGTCTCCAGCCGGTGAAGACAAAGTGCACCTCTGACTCCTATTACCAGGATAACTGTGCGAA
CATCACATTTACCTTTAACAAGGAGATGATGTCACCAGTATGTAACAACCTTTGTTTTTTTTTTGAATGG
TGGATGTCTGCTTGCTTGCTTTAAAGGAGGAATGCCATGAGCTAGGATCTACTTCTCCCTAGCTTACATT
CCTGTTTTATAATAAAGTAAGCTTGAACTTAGCCAGCCTCAAAGAGAACATCTCAGCCTTTTGTTCTTCC
TCTCAATCTTACACGTGTGTGGGTTTTTAAAAATCGTTTTAGGGTCTTACTACGGAGCACATTTGCAGTG
AATTGAGGAATTTGAATATTTTGAAGAATGTTTCCGCTGAATATTCAATCTACATCGCTTGCGAGCCTTC
CCCTTCAGCGAACAATGAAATACATGTGGCCATTGTAAGTATAAGACCCATTCACACCTCATTATTCGAT
GGCAAGGCAGTTCGGTTAACCAGTGTCTGAATGGAGCAAATTCACTGACAAAAAGCTTTGCAGACACAGA
TTGTCGAGTAATTTTGAAGAAAGGCTGCTTTGAGTATTCCTCTGACTCTCAAGTCTGACAATTGTTTTTC
CAGTCTGCTGAAGATATACGGGATGATGGGAACCCGATCAAGGAAATCACTGACAAAATAATCGATCTTG
TTAGTAAACGTGATGGAAACAGCTCGCTGATTGCTGCCGTTGCAGAAGTAAGAGTTCAGAGGCGGCCTCT
GAAGAACAGAACAGGTAGGTGTCAAGTGGGACTAGTTTGTGTGATGATAGTAGATGATCTCATTATACTA
TTCAATAAAGCCATCAGGTCGAGGGATTATCTGGCAGTGCCCTACCCTCGCAGGAGCTCCCAATTTAACT
GAGAACTGAGGGCAGCCTACTTTACTAAGGTCACATGGCCCATGGGAACAGAGGTCTGCCTCATATTTTC
CTTCCCAACACACTATGCAACGTTCCCTGCAGAACAGGTATTGTGCTAGATCCGTGACAAAGCCCTAGGA
CACTGCTTGTCCTTTGTCCTTGAGTTTGAAGCTGTACAAAGCCTGACTTGGCATTTTGGTGTCCTGGGAC
AGCCTAGCCACTGCTGAGCTCTGTGAGGCTCGATGGCCCCGCAGGGGTGTCCAGGCTTCCCTCCTGAGGA
TCTCGAGCACCGTTTCAGCCAAGGGAGAGATTGGGCATGGAGCCATGGCACGTTTGAGCTGTCTGTGCTA
ATTTGAATCGGATTTCTAGTTCTGAAAACCAACTCCTGTACTGAGCTGCCTCAGTAGCCAGACTTAACAA
GACACTGCTCTCCCCATGCGAAGTCAAAGAGCCTGTAGGAGCTGCCGCTCCACCCAGTCTCTCCAGGGAT
AAGAGTATCAAGGTCTACAGGGGCCTGATCCCAGCATTTGCTTATCAAGTTGTGAAGTGACCATATGCAA
TGATGAAAAGGGACTTGTCCCTGTGGCCCCCCTCTTAGGGATAAAGGGCAGGAGAACCACTGTTGGTATT
CTTTTGTTCCTGCGATGATGCTTTTTTCTTTCTTTCTTGGAGAGTTAATTGGTTTTGTGCCTGCCTTACA
GATTTCCTTGTTCCCTTGCTGAGCTCTGTCTTAACTGTGGCTTGGATCTGTTGCTTGGTGACGGCCTTCT
ACTGGTGCCTGCGGAAGCGGCGGAAGCCGGGCAGCCACACACACTCAGCCTCTGAGGACAACACCACCAA
CAACGTGCGGGAGCAGCTGAACCAGATCAAAAACCCCATTGAGAAACATGGGGCCAACACGGTCCCCATC
AAGGATTATGAGAACAAGAACTCCAAAATGTCTAAAATAAGGACACACAATTCTGAAGTAGAAGAGGACG
ACATGGACAAACACCAGCAGAAAGCCCGGTTTGCCAAGCAGCCGGCGTACACGCTGGTAGACAGAGAAGA
GAAGCCCCCCAACGGCACGCCGACAAAACACCCAAACTGGACAAACAAACAGGACAACAGAGACTTGGAA
AGTGCCCAGAGCTTAAACCGAATGGAGTACATCGTATAGCAGACCGCGGGCACTGCCGCCGCTAGGTAGA
GTCTGAGGGCTTGTAGTTCTTTAAACTGTCGTGTCATACTCGAGTCTGAGGCCGTTGCTGACTTAGAATC
CCTGTGTTAATTTAAGTTTTGACAAGCTGGCTTACACTGGCAATGGTAGTTTCTGTGGTTGGCTGGGAAA
TCGAGTGCCGCATCTCACAGCTATGCAAAAAGCTAGTCAACAGTACCCTGGTTGTGTGTCCCCTTGCAGC
CGACACGGTCTCGGATCAGGCTCCCAGGAGCCTGCCCAGCCCCCTGGTCTTTGAGCTCCCACTTCTGCCA
GATGTCCTAATGGTGATGCAGTCTTAGATCATAGTTTTATTTATATTTATTGACTCTTGAGTTGTTTTTG
TATATTGGTTTTATGATGACGTACAAGTAGTTCTGTATTTGAAAGTGCCTTTGCAGCTCAGAACCACAGC
AACGATCACAAATGACTTTATTATTTATTTTTTTTAATTGTATTTTTGTTGTTGGGGGAGGGGAGACTTT
GATGTCAGCAGTTGCTGGTAAAATGAAGAATTTAAAGAAAAAAATGTCAAAAGTAGAACTTTGTATAGTT
ATGTAAATAATTCTTTTTTATTAATCACTGTGTATATTTGATTTATTAACTTAATAATCAAGAGCCTTAA
AACATCATTCCTTTTTATTTATATGTATGTGTTTAGAATTGAAGGTTTTTGATAGCATTGTAAGCGTATG
GCTTTATTTTTTTGAACTCTTCTCATTACTTGTTGCCTATAAGCCAAAATTAAGGTGTTTGAAAATAGTT
TATTTTAAAACAATAGGATGGGCTTCTGTGCCCAGAATACTGATGGAATTTTTTTGTACGACGTCAGATG
TTTAAAACACCTTCTATAGCATCACTTAAAACACGTTTTAAGGACTGACTGAGGCAGTTTGAGGATTAGT
TTAGAACAGGTTTTTTTGTTTGTTTGTTTTTTGTTTTTCTGCTTTAGACTTGAAAAGAGACAGGCAGGTG
ATCTGCTGCAGAGCAGTAAGGGAACAAGTTGAGCTATGACTTAACATAGCCAAAATGTGAGTGGTTGAAT
ATGATTAAAAATATCAAATTAATTGTGTGAACTTGGAAGCACACCAATCTTACTTTGTAAATTCTGATTT
CTTTTCACCATTCGTACATAATACTGAACCACTTGTAGATTTGATTTTTTTTTTTAATCTACTGCATTTA
GGGAGTATTCTAATAAGCTAGTTGAATACTTGAACCATAAAATGTCCAGTAAGATCACTGTTTAGATTTG
CCATAGAGTACACTGCCTGCCTTAAGTGAGGAAATCAAAGTGCTATTACGAAGTTCAAGATCAAAAAGGC
TTATAAAACAGAGTAATCTTGTTGGTTCACCATTGAGACCGTGAAGATACTTTGTATTGTCCTATTAGTG
TTATATGAACATACAAATGCATCTTTGATGTGTTGTTCTTGGCAATAAATTTTGAAAAGTAATATTTATT
AAATTTTTTTGTATGAAAACATGGAACAGTGTGGCCTCTTCTGAGCTTACGTAGTTCTACCGGCTTTGCC
ATGTGCTTCTGCCACCCTGCTGAGTCTGTTCTGGTAATCGGGGTATAATAGGCTCTGCCTGACAGAGGGA
TGGAGGAAGAACTGAAAGGCTTTTCAACCACAAAACTCATCTGGAGTTCTCAAAGACCTGGGGCTGCTGT
GAAGCTGGAACTGCGGGAGCCCCATCTAGGGGAGCCTTGATTCCCTTGTTATTCAACAGCAAGTGTGAAT
ACTGCTTGAATAAACACCACTGGATTAATGGCC
Homo sapiens jagged 1 (JAG1), cDNA
(SEQ ID NO: 2)
GGGAGGGGCGTGCCCAGGGTGAGCACGCCCTCTCATGAATATTAATAAGCGCGCATGCGC
CCTGCCCGGCGTGCTGGGTAGAGGTGGCCAGCCCCGGCCGCTGCTGCCAGACGGGCTCTC
CGGGTCCTTCTCCGAGAGCCGGGCGGGCACGCGTCATTGTGTTACCTGCGGCCGGCCCGC
GAGCTAGGCTGGTTTTTTTTTTTCTCCCCTCCCTCCCCCCTTTTTCCATGCAGCTGATCT
AAAAGGGAATAAAAGGCTGCGCATAATCATAATAATAAAAGAAGGGGAGCGCGAGAGAAG
GAAAGAAAGCCGGGAGGTGGAAGAGGAGGGGGAGCGTCTCAAAGAAGCGATCAGAATAAT
AAAAGGAGGCCGGGCTCTTTGCCTTCTGGAACGGGCCGCTCTTGAAAGGGCTTTTGAAAA
GTGGTGTTGTTTTCCAGTCGTGCATGCTCCAATCGGCGGAGTATATTAGAGCCGGGACGC
GGCGGCCGCAGGGGCAGCGGCGACGGCAGCACCGGCGGCAGCACCAGCGCGAACAGCAGC
GGCGGCGTCCCGAGTGCCCGCGGCGCGCGGCGCAGCGATGCGTTCCCCACGGACGCGCGG
CCGGTCCGGGCGCCCCCTAAGCCTCCTGCTCGCCCTGCTCTGTGCCCTGCGAGCCAAGGT
GTGTGGGGCCTCGGGTCAGTTCGAGTTGGAGATCCTGTCCATGCAGAACGTGAACGGGGA
GCTGCAGAACGGGAACTGCTGCGGCGGCGCCCGGAACCCGGGAGACCGCAAGTGCACCCG
CGACGAGTGTGACACATACTTCAAAGTGTGCCTCAAGGAGTATCAGTCCCGCGTCACGGC
CGGGGGGCCCTGCAGCTTCGGCTCAGGGTCCACGCCTGTCATCGGGGGCAACACCTTCAA
CCTCAAGGCCAGCCGCGGCAACGACCGCAACCGCATCGTGCTGCCTTTCAGTTTCGCCTG
GCCGAGGTCCTATACGTTGCTTGTGGAGGCGTGGGATTCCAGTAATGACACCGTTCAACC
TGACAGTATTATTGAAAAGGCTTCTCACTCGGGCATGATCAACCCCAGCCGGCAGTGGCA
GACGCTGAAGCAGAACACGGGCGTTGCCCACTTTGAGTATCAGATCCGCGTGACCTGTGA
TGACTACTACTATGGCTTTGGCTGCAATAAGTTCTGCCGCCCCAGAGATGACTTCTTTGG
ACACTATGCCTGTGACCAGAATGGCAACAAAACTTGCATGGAAGGCTGGATGGGCCCCGA
ATGTAACAGAGCTATTTGCCGACAAGGCTGCAGTCCTAAGCATGGGTCTTGCAAACTCCC
AGGTGACTGCAGGTGCCAGTACGGCTGGCAAGGCCTGTACTGTGATAAGTGCATCCCACA
CCCGGGATGCGTCCACGGCATCTGTAATGAGCCCTGGCAGTGCCTCTGTGAGACCAACTG
GGGCGGCCAGCTCTGTGACAAAGATCTCAATTACTGTGGGACTCATCAGCCGTGTCTCAA
CGGGGGAACTTGTAGCAACACAGGCCCTGACAAATATCAGTGTTCCTGCCCTGAGGGGTA
TTCAGGACCCAACTGTGAAATTGCTGAGCACGCCTGCCTCTCTGATCCCTGTCACAACAG
AGGCAGCTGTAAGGAGACCTCCCTGGGCTTTGAGTGTGAGTGTTCCCCAGGCTGGACCGG
CCCCACATGCTCTACAAACATTGATGACTGTTCTCCTAATAACTGTTCCCACGGGGGCAC
CTGCCAGGACCTGGTTAACGGATTTAAGTGTGTGTGCCCCCCACAGTGGACTGGGAAAAC
GTGCCAGTTAGATGCAAATGAATGTGAGGCCAAACCTTGTGTAAACGCCAAATCCTGTAA
GAATCTCATTGCCAGCTACTACTGCGACTGTCTTCCCGGCTGGATGGGTCAGAATTGTGA
CATAAATATTAATGACTGCCTTGGCCAGTGTCAGAATGACGCCTCCTGTCGGGATTTGGT
TAATGGTTATCGCTGTATCTGTCCACCTGGCTATGCAGGCGATCACTGTGAGAGAGACAT
CGATGAATGTGCCAGCAACCCCTGTTTGAATGGGGGTCACTGTCAGAATGAAATCAACAG
ATTCCAGTGTCTGTGTCCCACTGGTTTCTCTGGAAACCTCTGTCAGCTGGACATCGATTA
TTGTGAGCCTAATCCCTGCCAGAACGGTGCCCAGTGCTACAACCGTGCCAGTGACTATTT
CTGCAAGTGCCCCGAGGACTATGAGGGCAAGAACTGCTCACACCTGAAAGACCACTGCCG
CACGACCCCCTGTGAAGTGATTGACAGCTGCACAGTGGCCATGGCTTCCAACGACACACC
TGAAGGGGTGCGGTATATTTCCTCCAACGTCTGTGGTCCTCACGGGAAGTGCAAGAGTCA
GTCGGGAGGCAAATTCACCTGTGACTGTAACAAAGGCTTCACGGGAACATACTGCCATGA
AAATATTAATGACTGTGAGAGCAACCCTTGTAGAAACGGTGGCACTTGCATCGATGGTGT
CAACTCCTACAAGTGCATCTGTAGTGACGGCTGGGAGGGGGCCTACTGTGAAACCAATAT
TAATGACTGCAGCCAGAACCCCTGCCACAATGGGGGCACGTGTCGCGACCTGGTCAATGA
CTTCTACTGTGACTGTAAAAATGGGTGGAAAGGAAAGACCTGCCACTCACGTGACAGTCA
GTGTGATGAGGCCACGTGCAACAACGGTGGCACCTGCTATGATGAGGGGGATGCTTTTAA
GTGCATGTGTCCTGGCGGCTGGGAAGGAACAACCTGTAACATAGCCCGAAACAGTAGCTG
CCTGCCCAACCCCTGCCATAATGGGGGCACATGTGTGGTCAACGGCGAGTCCTTTACGTG
CGTCTGCAAGGAAGGCTGGGAGGGGCCCATCTGTGCTCAGAATACCAATGACTGCAGCCC
TCATCCCTGTTACAACAGCGGCACCTGTGTGGATGGAGACAACTGGTACCGGTGCGAATG
TGCCCCGGGTTTTGCTGGGCCCGACTGCAGAATAAACATCAATGAATGCCAGTCTTCACC
TTGTGCCTTTGGAGCGACCTGTGTGGATGAGATCAATGGCTACCGGTGTGTCTGCCCTCC
AGGGCACAGTGGTGCCAAGTGCCAGGAAGTTTCAGGGAGACCTTGCATCACCATGGGGAG
TGTGATACCAGATGGGGCCAAATGGGATGATGACTGTAATACCTGCCAGTGCCTGAATGG
ACGGATCGCCTGCTCAAAGGTCTGGTGTGGCCCTCGACCTTGCCTGCTCCACAAAGGGCA
CAGCGAGTGCCCCAGCGGGCAGAGCTGCATCCCCATCCTGGACGACCAGTGCTTCGTCCA
CCCCTGCACTGGTGTGGGCGAGTGTCGGTCTTCCAGTCTCCAGCCGGTGAAGACAAAGTG
CACCTCTGACTCCTATTACCAGGATAACTGTGCGAACATCACATTTACCTTTAACAAGGA
GATGATGTCACCAGGTCTTACTACGGAGCACATTTGCAGTGAATTGAGGAATTTGAATAT
TTTGAAGAATGTTTCCGCTGAATATTCAATCTACATCGCTTGCGAGCCTTCCCCTTCAGC
GAACAATGAAATACATGTGGCCATTTCTGCTGAAGATATACGGGATGATGGGAACCCGAT
CAAGGAAATCACTGACAAAATAATCGATCTTGTTAGTAAACGTGATGGAAACAGCTCGCT
GATTGCTGCCGTTGCAGAAGTAAGAGTTCAGAGGCGGCCTCTGAAGAACAGAACAGATTT
CCTTGTTCCCTTGCTGAGCTCTGTCTTAACTGTGGCTTGGATCTGTTGCTTGGTGACGGC
CTTCTACTGGTGCCTGCGGAAGCGGCGGAAGCCGGGCAGCCACACACACTCAGCCTCTGA
GGACAACACCACCAACAACGTGCGGGAGCAGCTGAACCAGATCAAAAACCCCATTGAGAA
ACATGGGGCCAACACGGTCCCCATCAAGGATTATGAGAACAAGAACTCCAAAATGTCTAA
AATAAGGACACACAATTCTGAAGTAGAAGAGGACGACATGGACAAACACCAGCAGAAAGC
CCGGTTTGCCAAGCAGCCGGCGTACACGCTGGTAGACAGAGAAGAGAAGCCCCCCAACGG
CACGCCGACAAAACACCCAAACTGGACAAACAAACAGGACAACAGAGACTTGGAAAGTGC
CCAGAGCTTAAACCGAATGGAGTACATCGTATAGCAGACCGCGGGCACTGCCGCCGCTAG
GTAGAGTCTGAGGGCTTGTAGTTCTTTAAACTGTCGTGTCATACTCGAGTCTGAGGCCGT
TGCTGACTTAGAATCCCTGTGTTAATTTAAGTTTTGACAAGCTGGCTTACACTGGCAATG
GTAGTTTCTGTGGTTGGCTGGGAAATCGAGTGCCGCATCTCACAGCTATGCAAAAAGCTA
GTCAACAGTACCCTGGTTGTGTGTCCCCTTGCAGCCGACACGGTCTCGGATCAGGCTCCC
AGGAGCCTGCCCAGCCCCCTGGTCTTTGAGCTCCCACTTCTGCCAGATGTCCTAATGGTG
ATGCAGTCTTAGATCATAGTTTTATTTATATTTATTGACTCTTGAGTTGTTTTTGTATAT
TGGTTTTATGATGACGTACAAGTAGTTCTGTATTTGAAAGTGCCTTTGCAGCTCAGAACC
ACAGCAACGATCACAAATGACTTTATTATTTATTTTTTTTAATTGTATTTTTGTTGTTGG
GGGAGGGGAGACTTTGATGTCAGCAGTTGCTGGTAAAATGAAGAATTTAAAGAAAAAAAT
GTCAAAAGTAGAACTTTGTATAGTTATGTAAATAATTCTTTTTTATTAATCACTGTGTAT
ATTTGATTTATTAACTTAATAATCAAGAGCCTTAAAACATCATTCCTTTTTATTTATATG
TATGTGTTTAGAATTGAAGGTTTTTGATAGCATTGTAAGCGTATGGCTTTATTTTTTTGA
ACTCTTCTCATTACTTGTTGCCTATAAGCCAAAATTAAGGTGTTTGAAAATAGTTTATTT
TAAAACAATAGGATGGGCTTCTGTGCCCAGAATACTGATGGAATTTTTTTGTACGACGTC
AGATGTTTAAAACACCTTCTATAGCATCACTTAAAACACGTTTTAAGGACTGACTGAGGC
AGTTTGAGGATTAGTTTAGAACAGGTTTTTTTGTTTGTTTGTTTTTTGTTTTTCTGCTTT
AGACTTGAAAAGAGACAGGCAGGTGATCTGCTGCAGAGCAGTAAGGGAACAAGTTGAGCT
ATGACTTAACATAGCCAAAATGTGAGTGGTTGAATATGATTAAAAATATCAAATTAATTG
TGTGAACTTGGAAGCACACCAATCTTACTTTGTAAATTCTGATTTCTTTTCACCATTCGT
ACATAATACTGAACCACTTGTAGATTTGATTTTTTTTTTTAATCTACTGCATTTAGGGAG
TATTCTAATAAGCTAGTTGAATACTTGAACCATAAAATGTCCAGTAAGATCACTGTTTAG
ATTTGCCATAGAGTACACTGCCTGCCTTAAGTGAGGAAATCAAAGTGCTATTACGAAGTT
CAAGATCAAAAAGGCTTATAAAACAGAGTAATCTTGTTGGTTCACCATTGAGACCGTGAA
GATACTTTGTATTGTCCTATTAGTGTTATATGAACATACAAATGCATCTTTGATGTGTTG
TTCTTGGCAATAAATTTTGAAAAGTAATATTTATTAAATTTTTTTGTATGAAAACATGGA
ACAGTGTGGCCTCTTCTGAGCTTACGTAGTTCTACCGGCTTTGCCATGTGCTTCTGCCAC
CCTGCTGAGTCTGTTCTGGTAATCGGGGTATAATAGGCTCTGCCTGACAGAGGGATGGAG
GAAGAACTGAAAGGCTTTTCAACCACAAAACTCATCTGGAGTTCTCAAAGACCTGGGGCT
GCTGTGAAGCTGGAACTGCGGGAGCCCCATCTAGGGGAGCCTTGATTCCCTTGTTATTCA
ACAGCAAGTGTGAATACTGCTTGAATAAACACCACTGGATTAATGGCC
Homo sapiens jagged 1 (JAG1), mRNA
(SEQ ID NO: 3)
CTGCCCGGCGTGCTGGGTAGAGGTGGCCAGCCCCGGCCGCTGCTGCCAGACGGGCTCTCCGGGTCCTTCT
CCGAGAGCCGGGCGGGCACGCGTCATTGTGTTACCTGCGGCCGGCCCGCGAGCTAGGCTGGTTTTTTTTT
TTCTCCCCTCCCTCCCCCCTTTTTCCATGCAGCTGATCTAAAAGGGAATAAAAGGCTGCGCATAATCATA
ATAATAAAAGAAGGGGAGCGCGAGAGAAGGAAAGAAAGCCGGGAGGTGGAAGAGGAGGGGGAGCGTCTCA
AAGAAGCGATCAGAATAATAAAAGGAGGCCGGGCTCTTTGCCTTCTGGAACGGGCCGCTCTTGAAAGGGC
TTTTGAAAAGTGGTGTTGTTTTCCAGTCGTGCATGCTCCAATCGGCGGAGTATATTAGAGCCGGGACGCG
GCGGCCGCAGGGGCAGCGGCGACGGCAGCACCGGCGGCAGCACCAGCGCGAACAGCAGCGGCGGCGTCCC
GAGTGCCCGCGGCGCGCGGCGCAGCGATGCGTTCCCCACGGACGCGCGGCCGGTCCGGGCGCCCCCTAAG
CCTCCTGCTCGCCCTGCTCTGTGCCCTGCGAGCCAAGGTGTGTGGGGCCTCGGGTCAGTTCGAGTTGGAG
ATCCTGTCCATGCAGAACGTGAACGGGGAGCTGCAGAACGGGAACTGCTGCGGCGGCGCCCGGAACCCGG
GAGACCGCAAGTGCACCCGCGACGAGTGTGACACATACTTCAAAGTGTGCCTCAAGGAGTATCAGTCCCG
CGTCACGGCCGGGGGGCCCTGCAGCTTCGGCTCAGGGTCCACGCCTGTCATCGGGGGCAACACCTTCAAC
CTCAAGGCCAGCCGCGGCAACGACCGCAACCGCATCGTGCTGCCTTTCAGTTTCGCCTGGCCGAGGTCCT
ATACGTTGCTTGTGGAGGCGTGGGATTCCAGTAATGACACCGTTCAACCTGACAGTATTATTGAAAAGGC
TTCTCACTCGGGCATGATCAACCCCAGCCGGCAGTGGCAGACGCTGAAGCAGAACACGGGCGTTGCCCAC
TTTGAGTATCAGATCCGCGTGACCTGTGATGACTACTACTATGGCTTTGGCTGCAATAAGTTCTGCCGCC
CCAGAGATGACTTCTTTGGACACTATGCCTGTGACCAGAATGGCAACAAAACTTGCATGGAAGGCTGGAT
GGGCCCCGAATGTAACAGAGCTATTTGCCGACAAGGCTGCAGTCCTAAGCATGGGTCTTGCAAACTCCCA
GGTGACTGCAGGTGCCAGTACGGCTGGCAAGGCCTGTACTGTGATAAGTGCATCCCACACCCGGGATGCG
TCCACGGCATCTGTAATGAGCCCTGGCAGTGCCTCTGTGAGACCAACTGGGGCGGCCAGCTCTGTGACAA
AGATCTCAATTACTGTGGGACTCATCAGCCGTGTCTCAACGGGGGAACTTGTAGCAACACAGGCCCTGAC
AAATATCAGTGTTCCTGCCCTGAGGGGTATTCAGGACCCAACTGTGAAATTGCTGAGCACGCCTGCCTCT
CTGATCCCTGTCACAACAGAGGCAGCTGTAAGGAGACCTCCCTGGGCTTTGAGTGTGAGTGTTCCCCAGG
CTGGACCGGCCCCACATGCTCTACAAACATTGATGACTGTTCTCCTAATAACTGTTCCCACGGGGGCACC
TGCCAGGACCTGGTTAACGGATTTAAGTGTGTGTGCCCCCCACAGTGGACTGGGAAAACGTGCCAGTTAG
ATGCAAATGAATGTGAGGCCAAACCTTGTGTAAACGCCAAATCCTGTAAGAATCTCATTGCCAGCTACTA
CTGCGACTGTCTTCCCGGCTGGATGGGTCAGAATTGTGACATAAATATTAATGACTGCCTTGGCCAGTGT
CAGAATGACGCCTCCTGTCGGGATTTGGTTAATGGTTATCGCTGTATCTGTCCACCTGGCTATGCAGGCG
ATCACTGTGAGAGAGACATCGATGAATGTGCCAGCAACCCCTGTTTGAATGGGGGTCACTGTCAGAATGA
AATCAACAGATTCCAGTGTCTGTGTCCCACTGGTTTCTCTGGAAACCTCTGTCAGCTGGACATCGATTAT
TGTGAGCCTAATCCCTGCCAGAACGGTGCCCAGTGCTACAACCGTGCCAGTGACTATTTCTGCAAGTGCC
CCGAGGACTATGAGGGCAAGAACTGCTCACACCTGAAAGACCACTGCCGCACGACCCCCTGTGAAGTGAT
TGACAGCTGCACAGTGGCCATGGCTTCCAACGACACACCTGAAGGGGTGCGGTATATTTCCTCCAACGTC
TGTGGTCCTCACGGGAAGTGCAAGAGTCAGTCGGGAGGCAAATTCACCTGTGACTGTAACAAAGGCTTCA
CGGGAACATACTGCCATGAAAATATTAATGACTGTGAGAGCAACCCTTGTAGAAACGGTGGCACTTGCAT
CGATGGTGTCAACTCCTACAAGTGCATCTGTAGTGACGGCTGGGAGGGGGCCTACTGTGAAACCAATATT
AATGACTGCAGCCAGAACCCCTGCCACAATGGGGGCACGTGTCGCGACCTGGTCAATGACTTCTACTGTG
ACTGTAAAAATGGGTGGAAAGGAAAGACCTGCCACTCACGTGACAGTCAGTGTGATGAGGCCACGTGCAA
CAACGGTGGCACCTGCTATGATGAGGGGGATGCTTTTAAGTGCATGTGTCCTGGCGGCTGGGAAGGAACA
ACCTGTAACATAGCCCGAAACAGTAGCTGCCTGCCCAACCCCTGCCATAATGGGGGCACATGTGTGGTCA
ACGGCGAGTCCTTTACGTGCGTCTGCAAGGAAGGCTGGGAGGGGCCCATCTGTGCTCAGAATACCAATGA
CTGCAGCCCTCATCCCTGTTACAACAGCGGCACCTGTGTGGATGGAGACAACTGGTACCGGTGCGAATGT
GCCCCGGGTTTTGCTGGGCCCGACTGCAGAATAAACATCAATGAATGCCAGTCTTCACCTTGTGCCTTTG
GAGCGACCTGTGTGGATGAGATCAATGGCTACCGGTGTGTCTGCCCTCCAGGGCACAGTGGTGCCAAGTG
CCAGGAAGTTTCAGGGAGACCTTGCATCACCATGGGGAGTGTGATACCAGATGGGGCCAAATGGGATGAT
GACTGTAATACCTGCCAGTGCCTGAATGGACGGATCGCCTGCTCAAAGGTCTGGTGTGGCCCTCGACCTT
GCCTGCTCCACAAAGGGCACAGCGAGTGCCCCAGCGGGCAGAGCTGCATCCCCATCCTGGACGACCAGTG
CTTCGTCCACCCCTGCACTGGTGTGGGCGAGTGTCGGTCTTCCAGTCTCCAGCCGGTGAAGACAAAGTGC
ACCTCTGACTCCTATTACCAGGATAACTGTGCGAACATCACATTTACCTTTAACAAGGAGATGATGTCAC
CAGGTCTTACTACGGAGCACATTTGCAGTGAATTGAGGAATTTGAATATTTTGAAGAATGTTTCCGCTGA
ATATTCAATCTACATCGCTTGCGAGCCTTCCCCTTCAGCGAACAATGAAATACATGTGGCCATTTCTGCT
GAAGATATACGGGATGATGGGAACCCGATCAAGGAAATCACTGACAAAATAATCGATCTTGTTAGTAAAC
GTGATGGAAACAGCTCGCTGATTGCTGCCGTTGCAGAAGTAAGAGTTCAGAGGCGGCCTCTGAAGAACAG
AACAGATTTCCTTGTTCCCTTGCTGAGCTCTGTCTTAACTGTGGCTTGGATCTGTTGCTTGGTGACGGCC
TTCTACTGGTGCCTGCGGAAGCGGCGGAAGCCGGGCAGCCACACACACTCAGCCTCTGAGGACAACACCA
CCAACAACGTGCGGGAGCAGCTGAACCAGATCAAAAACCCCATTGAGAAACATGGGGCCAACACGGTCCC
CATCAAGGATTATGAGAACAAGAACTCCAAAATGTCTAAAATAAGGACACACAATTCTGAAGTAGAAGAG
GACGACATGGACAAACACCAGCAGAAAGCCCGGTTTGCCAAGCAGCCGGCGTACACGCTGGTAGACAGAG
AAGAGAAGCCCCCCAACGGCACGCCGACAAAACACCCAAACTGGACAAACAAACAGGACAACAGAGACTT
GGAAAGTGCCCAGAGCTTAAACCGAATGGAGTACATCGTATAGCAGACCGCGGGCACTGCCGCCGCTAGG
TAGAGTCTGAGGGCTTGTAGTTCTTTAAACTGTCGTGTCATACTCGAGTCTGAGGCCGTTGCTGACTTAG
AATCCCTGTGTTAATTTAAGTTTTGACAAGCTGGCTTACACTGGCAATGGTAGTTTCTGTGGTTGGCTGG
GAAATCGAGTGCCGCATCTCACAGCTATGCAAAAAGCTAGTCAACAGTACCCTGGTTGTGTGTCCCCTTG
CAGCCGACACGGTCTCGGATCAGGCTCCCAGGAGCCTGCCCAGCCCCCTGGTCTTTGAGCTCCCACTTCT
GCCAGATGTCCTAATGGTGATGCAGTCTTAGATCATAGTTTTATTTATATTTATTGACTCTTGAGTTGTT
TTTGTATATTGGTTTTATGATGACGTACAAGTAGTTCTGTATTTGAAAGTGCCTTTGCAGCTCAGAACCA
CAGCAACGATCACAAATGACTTTATTATTTATTTTTTTTAATTGTATTTTTGTTGTTGGGGGAGGGGAGA
CTTTGATGTCAGCAGTTGCTGGTAAAATGAAGAATTTAAAGAAAAAAATGTCAAAAGTAGAACTTTGTAT
AGTTATGTAAATAATTCTTTTTTATTAATCACTGTGTATATTTGATTTATTAACTTAATAATCAAGAGCC
TTAAAACATCATTCCTTTTTATTTATATGTATGTGTTTAGAATTGAAGGTTTTTGATAGCATTGTAAGCG
TATGGCTTTATTTTTTTGAACTCTTCTCATTACTTGTTGCCTATAAGCCAAAATTAAGGTGTTTGAAAAT
AGTTTATTTTAAAACAATAGGATGGGCTTCTGTGCCCAGAATACTGATGGAATTTTTTTGTACGACGTCA
GATGTTTAAAACACCTTCTATAGCATCACTTAAAACACGTTTTAAGGACTGACTGAGGCAGTTTGAGGAT
TAGTTTAGAACAGGTTTTTTTGTTTGTTTGTTTTTTGTTTTTCTGCTTTAGACTTGAAAAGAGACAGGCA
GGTGATCTGCTGCAGAGCAGTAAGGGAACAAGTTGAGCTATGACTTAACATAGCCAAAATGTGAGTGGTT
GAATATGATTAAAAATATCAAATTAATTGTGTGAACTTGGAAGCACACCAATCTTACTTTGTAAATTCTG
ATTTCTTTTCACCATTCGTACATAATACTGAACCACTTGTAGATTTGATTTTTTTTTTTAATCTACTGCA
TTTAGGGAGTATTCTAATAAGCTAGTTGAATACTTGAACCATAAAATGTCCAGTAAGATCACTGTTTAGA
TTTGCCATAGAGTACACTGCCTGCCTTAAGTGAGGAAATCAAAGTGCTATTACGAAGTTCAAGATCAAAA
AGGCTTATAAAACAGAGTAATCTTGTTGGTTCACCATTGAGACCGTGAAGATACTTTGTATTGTCCTATT
AGTGTTATATGAACATACAAATGCATCTTTGATGTGTTGTTCTTGGCAATAAATTTTGAAAAGTAATATT
TATTAAATTTTTTTGTATGAAAACATGGAACAGTGTGGCCTCTTCTGAGCTTACGTAGTTCTACCGGCTT
TGCCATGTGCTTCTGCCACCCTGCTGAGTCTGTTCTGGTAATCGGGGTATAATAGGCTCTGCCTGACAGA
GGGATGGAGGAAGAACTGAAAGGCTTTTCAACCACAAAACTCATCTGGAGTTCTCAAAGACCTGGGGCTG
CTGTGAAGCTGGAACTGCGGGAGCCCCATCTAGGGGAGCCTTGATTCCCTTGTTATTCAACAGCAAGTGT
GAATACTGCTTGAATAAACACCACTGGATTAATGGCCA
Human JAG1 precursor protein
(SEQ ID NO: 4)
MRSPRTRGRSGRPLSLLLALLCALRAKVCGASGQFELEILSMQNVNGELQNGNCCGGARNPGDRKCTRDE
CDTYFKVCLKEYQSRVTAGGPCSFGSGSTPVIGGNTFNLKASRGNDRNRIVLPFSFAWPRSYTLLVEAWD
SSNDTVQPDSIIEKASHSGMINPSRQWQTLKQNTGVAHFEYQIRVTCDDYYYGFGCNKFCRPRDDFFGHY
ACDQNGNKTCMEGWMGPECNRAICRQGCSPKHGSCKLPGDCRCQYGWQGLYCDKCIPHPGCVHGICNEPW
QCLCETNWGGQLCDKDLNYCGTHQPCLNGGTCSNTGPDKYQCSCPEGYSGPNCEIAEHACLSDPCHNRGS
CKETSLGFECECSPGWTGPTCSTNIDDCSPNNCSHGGTCQDLVNGFKCVCPPQWTGKTCQLDANECEAKP
CVNAKSCKNLIASYYCDCLPGWMGQNCDININDCLGQCQNDASCRDLVNGYRCICPPGYAGDHCERDIDE
CASNPCLNGGHCQNEINRFQCLCPTGFSGNLCQLDIDYCEPNPCQNGAQCYNRASDYFCKCPEDYEGKNC
SHLKDHCRTTPCEVIDSCTVAMASNDTPEGVRYISSNVCGPHGKCKSQSGGKFTCDCNKGFTGTYCHENI
NDCESNPCRNGGTCIDGVNSYKCICSDGWEGAYCETNINDCSQNPCHNGGTCRDLVNDFYCDCKNGWKGK
TCHSRDSQCDEATCNNGGTCYDEGDAFKCMCPGGWEGTTCNIARNSSCLPNPCHNGGTCVVNGESFTCVC
KEGWEGPICAQNTNDCSPHPCYNSGTCVDGDNWYRCECAPGFAGPDCRININECQSSPCAFGATCVDEIN
GYRCVCPPGHSGAKCQEVSGRPCITMGSVIPDGAKWDDDCNTCQCLNGRIACSKVWCGPRPCLLHKGHSE
CPSGQSCIPILDDQCFVHPCTGVGECRSSSLQPVKTKCTSDSYYQDNCANITFTFNKEMMSPGLTTEHIC
SELRNLNILKNVSAEYSIYIACEPSPSANNEIHVAISAEDIRDDGNPIKEITDKIIDLVSKRDGNSSLIA
AVAEVRVQRRPLKNRTDFLVPLLSSVLTVAWICCLVTAFYWCLRKRRKPGSHTHSASEDNTTNNVREQLN
QIKNPIEKHGANTVPIKDYENKNSKMSKIRTHNSEVEEDDMDKHQQKARFAKQPAYTLVDREEKPPNGTP
TKHPNWTNKQDNRDLESAQSLNRMEYIV
Human JAG1 mature protein
(SEQ ID NO: 5)
QFELEILSMQNVNGELQNGNCCGGARNPGDRKCTRDECDTYFKVCLKEYQSRVTAGGPCSFGSGSTPVIG
GNTFNLKASRGNDRNRIVLPFSFAWPRSYTLLVEAWDSSNDTVQPDSIIEKASHSGMINPSRQWQTLKQN
TGVAHFEYQIRVTCDDYYYGFGCNKFCRPRDDFFGHYACDQNGNKTCMEGWMGPECNRAICRQGCSPKHG
SCKLPGDCRCQYGWQGLYCDKCIPHPGCVHGICNEPWQCLCETNWGGQLCDKDLNYCGTHQPCLNGGTCS
NTGPDKYQCSCPEGYSGPNCEIAEHACLSDPCHNRGSCKETSLGFECECSPGWTGPTCSTNIDDCSPNNC
SHGGTCQDLVNGFKCVCPPQWTGKTCQLDANECEAKPCVNAKSCKNLIASYYCDCLPGWMGQNCDININD
CLGQCQNDASCRDLVNGYRCICPPGYAGDHCERDIDECASNPCLNGGHCQNEINRFQCLCPTGFSGNLCQ
LDIDYCEPNPCQNGAQCYNRASDYFCKCPEDYEGKNCSHLKDHCRTTPCEVIDSCTVAMASNDTPEGVRY
ISSNVCGPHGKCKSQSGGKFTCDCNKGFTGTYCHENINDCESNPCRNGGTCIDGVNSYKCICSDGWEGAY
CETNINDCSQNPCHNGGTCRDLVNDFYCDCKNGWKGKTCHSRDSQCDEATCNNGGTCYDEGDAFKCMCPG
GWEGTTCNIARNSSCLPNPCHNGGTCVVNGESFTCVCKEGWEGPICAQNTNDCSPHPCYNSGTCVDGDNW
YRCECAPGFAGPDCRININECQSSPCAFGATCVDEINGYRCVCPPGHSGAKCQEVSGRPCITMGSVIPDG
AKWDDDCNTCQCLNGRIACSKVWCGPRPCLLHKGHSECPSGQSCIPILDDQCFVHPCTGVGECRSSSLQP
VKTKCTSDSYYQDNCANITFTFNKEMMSPGLTTEHICSELRNLNILKNVSAEYSIYIACEPSPSANNEIH
VAISAEDIRDDGNPIKEITDKIIDLVSKRDGNSSLIAAVAEVRVQRRPLKNRTDFLVPLLSSVLTVAWIC
CLVTAFYWCLRKRRKPGSHTHSASEDNTTNNVREQLNQIKNPIEKHGANTVPIKDYENKNSKMSKIRTHN
SEVEEDDMDKHQQKARFAKQPAYTLVDREEKPPNGTPTKHPNWTNKQDNRDLESAQSLNRMEYIV
Canis familiaris jagged 1, gene
(SEQ ID NO: 6)
ATGCGGTCCCCACGGACGCGCGACCGGCCCGGGCGCCCCCTGAGCCTCCTGCTCGCCCTG
CTCTGCGCCCTGCGAGCCAAGGTAGGAGCCTGCCGGGCCTCCCTCCCGGCCCGCCCTCTC
CTTTCCCTCGCAGCTCCCTGGACGCCTGCGGCTGAGCGCCTCGAGCGGGCGCGGGAGCCC
CCGGGCGCCCCGCTCCCCGGCTGGGTCCCCCGCGCCCGGGGGGCTGCACCTGGGCCGGGA
AGTCCCGGACCTCCTACGTGTCCCCTCCACCCTCCGGGCGGGGCCGGGGGCTGTGTTCTC
TCCGCTCCTCGCCCCCGGGAGCACTCGTCGGATTTCTCCGGCACTCGTGCATTTTGTGCT
CGGGAATCACGTTGAGTCCTTGCACCCAGTTTTGCAAAATCCTTTTCACTCGGCGCCGGG
GGCCCGTCGGGGCGGGCGGGGAGGGAGTCGCCGCTTCCACCCTCGGAGCAAACCCCTCTC
CCCCGCGCTGACCTCCCTCCCCCCTCGCCGGCAGGTGTGCGGCGCCTCGGGCCAGTTCGA
GCTGGAGATCCTGTCCATGCAGAACGTGAACGGGGAGCTGCAGAACGGGCACTGCTGCGG
CGGCGTCCGGAGCCCGGGGGACCGCAAGTGCTCTCACGACGAGTGTGACACGTACTTCAA
AGTGTGCCTCAAGGAGTACCAGTTCCGCGTCACGGCCGGGGGGCCCTGTAGCTTCGGGTC
GGGCTCCACGCCAGTCCTCGGGGGCAACACCTTCAATCTCAAGGCCGGCCGTGGCAGCGA
ACGCAACCGCATCGTGCTGCCTTTCAGTTTCGCCTGGCCGGTGAGTGCGCCACGCGGGGA
GGGCGGCCCGCCGGGGTCCCAGCCCGCGCCGGCGGAGCCCCGCGGCCTCGCCAGAGGGAC
GGCGGGTCTGGGCTGGAGCCTGCGCGCCGGCTGGCAAAGCCTTGCCGGGTGCGGCGTGAG
GCGGCTGCGACTCCGGTTACGGTCTCCGCGGCCTCTTGCCTAGCGCGCGACAGTGGGGAG
CCCGCGGGGGCTCGCGGGGGCTCGCGGGGCAAAGCTCCCAGGGAGGCGGGCTTATTAAAC
CTGCATCTAGAAGGCCCCAGAGTGACCCTACCCCCCCCCCCCCCCAGCTGGGCGTCTTTA
TGGACGATCTCTCTTTGCTTAACGAATTGAACCTGATGCGCCGTGGAAGGCGACGCGCAG
TTCTGGCCTTCGAAGCCGTCCAAATGGTCACTCCCCCCTTTCTCGTGAGCTGCCGCGAGG
GCGGGTGTGCCCTTCCTGGAGGGCGTGGGGGAGCCAGTTTCCCGCCGCTGCCCGGGAGAC
TTTGGGGCGTGCGGGGACGCGCTCCGGGCTGGACGGGAGGGTGCGGGGGCGGCCGCCGCC
TGGGAGGTGGGTGGGGGTTGACTCTGGGCCGAGGCTGGAGCCGGGAGCCCGAGAGCCTCC
GCCCCCTGCCGGCCCCTTCCTGCCCTCGCCCCCGCGCGGTGTAGCCCTTGTGGCCTCACT
TCCAGCGGTTCCCGGGCTCTGGAACCAGCTGTGTTTGCAAACTTCCCCGGGAAGGGCGGG
GGTGCGCCCTCGTCCGGCGGGCTCCGGCCCCCGCTAGCCTCCGGGGGCGTGTCAAGGCCT
GGAGGGGGCGGCCCGCTGGAGGCGGCTGCGGAGAAAGTGCCAGGGCTCGGACCCCTGCCC
CGGGCGCCGCTCGAGGCCCCGGGCCCGGCTGGCCCGCCCTGCAACCAGGCCCTTGCAACA
TCCCAAACTGGGTAGAAGGAAGATGTGTGTGATTGCAGAAGCGGAGGAAAGGGGAAAACG
CTGGTGCCCTCTGCTCCGCTCCTGCCGTCCAGGAGGCGTGGCTCATCGCCAGGACTGGGG
CTTGGCCCGGCCTGTGCGGCGAGGGTGGGCGGTGAGGGGGGGAGGGGAAAGCTCCGTGCC
CGGGGGAATACTCTCCGCCACGTGCTCTGATGGGGTGCGCTCTGCTGCCTCCTATCCGCG
GGCTGGGGCTGTGATTCGCAGGGCCGGGAGGAGAGGGAGGGGGGAGAGTGCCTCGGTGTG
GGCGTGGGGGGGGGGCACGTGTGATTGTGAGTGTGTGTGTGTGTGTGTGTGTGTGTGTAT
CTTGGGGTGGCTCTCAAGCCGCACATTGACTGGAGCTGGCGTTATAAAGTGCCCTCTGCC
ATGGGTGAGATAAGACAGTTATCGGAAACATGCCATTAAAAGGGACTTATAAAAACCCAT
GGTATGTAGAGTTCTGAGAATGGAGGGCTGCGTCTGTTGTGAGATTTCAATTTTACAAAG
ACTGTTTATCTTGTTTGTTAATCCCCTACTAGGGAGAACTTTAAAGAGGCTGGCCACTTT
GTAATCTGCCCGTGCCCAAGTTCACTGTGAATACTGCCCGCCCTTGAACTTGTGTTTAGA
GTTTAGGGGGGAAGGGGCCTGCAGGTGGTTCCACCCTCCTGAGGATCAGTCCGGAGCCCC
TTGCTTTTCACAAGACCATAAAAGGAATCTTATTAGTGATGTGGAAAAGCTGCAGGCCTG
GCTGAGGCTCACTTTGGAGAGTAGGGTCAGAGGATGGCTTGGATGAGAACAGGGAGCCTT
GCTCACCAGACTGGCCCCGGAAAAGTGACCCAGAAGGTGTGTCTGTCCCTATCTAGGACT
CTGCCAAATGTCAAATGGGGAATGAGCAGAAGCGGTGTGTGGGGCAGGGAGACGATTGAT
TTGCTGGTGCTCTTCTGTGGTTTCAGGGCCCACGGGTCACCAACCATGAAATAGACTCTC
GGCTTTTCTAGAATAGGGAGACTGCTGGGGCTACACAGAGTGTAGTTCTTTTGAAGTTGG
GCCAAATGCATGTATCACTCATAAATGAGTCTCCACCCTGCCCCCCCTAAAAGACCTGCT
GTGAAACATAAGCTTTGCTTCCCAAGAGAAAGTGAAGTTTAGGTGTATTGTTTTAGTTTA
TTTTCCGAGTGTGGTTAGAGAAACACCACAGTCTGTAACTTTTAAAGAATGATTCCAGGG
TGAATGTTAACCTGAAGTGAAAGCATTTAGGTATCGAAGAACATAGAACAGTCTCATTCA
GTTTGGCAATAAAATATTAATAAGCACGTCTATTTAAGACTGACTTTCTTAAATAGGTAA
TAACTGAACTTGGTACAGAATTCAAGATGGAAGGTGATACAATTTTTTTTTCTTTTTTTT
TTAATCAAACCACTTGGGACTTCCATTGGAGGCCCTGGCCCTTAAGCAGTCACAATTACC
CTTAGCATGGGTCTTCCCAAATGTGCTGACAGTGGAAGAGCTGTGGTTCAGAGGGCACGG
TAGAAGGTCAGTTTAGAAGGTGGTGTAATTCATGTCTTTTGTTTGGAATTATGACTGTAG
TCAAACCAACTGTTCGTGTTATAGAGAGGAAGAAGAGGAAATGAGTGGTCACATTTGAAA
TCTAGTCCACATTCCAGTGTCCCTGTGGGGAGCTCTGTAGAGGTGGGTCTGCCTGTTGGA
TTGTATCTGATCAATGTGCCACTGGTTGTCTGCTGCCCCTGTTCCAGGCTTGTGGTTCTC
ATTGGTGGCTTCTAATCCCCAAACCAGGGCTGTTCACAGGGTCTTAACTGGAAGTAACTG
GCCTGATTGATGACCCTCCAATCAGACGCACCCAGTGATAAGAACATATGTTATGGAAGG
TAATTGAGCTCAACGGGAAGTGATTTGAGAATAACCATTTGTAAATCATTGAAATGGTTA
AGTTGTACCTCTCAGAATTAGTGCTAACTCCCCATAGTACCTGCTTATTGAAAATCCATT
CATGCAGTTACCACTACCAAGCCCCCAGAGCTTTTTAGGAACTCCAGGACCCCAGTTCCT
GAACTGATGCTTTGCGGACCCTGCAGTGAAATGAATCTAGAGAAACTTGGGGTGGGGTGG
AGGTAGTTGACTATCAGGTCTTTTGGCGTCCTATTTAAAGGCCATTGACAGTGATAAAAG
GCTTGCTGGGAGCAGCTGATTTTTTTTTTTTTTTTCAGGAAAATCCAATGAAGTGTCAGA
GATTACAGAGGGATGCCAGCTTTGGCCCCTTTCACACTTAGGGCCTGATTGAGCAACTGC
GGGGGCGGGGAGGGGGGGTTTGAGGAAATCCGATGCCACTAGGTTCATCACAGAATATAA
TTGATATGTCCTTGCCTTTATGCCCTCTGCTGTGTTTCTGGGTCCCTGTGCTTCTGTTAG
GTGCGGTGTGGACACATGGATGGGGTTCTCCTCTGAACTGGCTCCTTTCTGCAGCCAGTA
TGTACAGCCCATGTGGTCATCATGCATGCAGACAACCGCACACAGGAGTTCAAGTGCTTT
GAGCACAGGCCTTTTCTCATGTGGAGAAAACCTCTTCTGTTCTCAGACCCCTGTGTCAAA
TGAAAGTGATGAAATGACACATCTGCGTTGCAAAACCAGGCTTGGGGGGAAAGGGGACCT
GTGCTTACTTGGTGCTAAGTGTCTGTAGGATGGCTGCTCCCCACATGAAATCCCTGTTCA
GATTACTTGGTTTCTAATTTTTTCTTCTCCTCAAGGTGTAAACCTCTGTGTTTTCTTCCT
CCTTGTCTTGTTTCTTAGCAACTTAAACCTCCAGTACTTACATGAGTTTGCAACCCACCA
ACTCCACACCCATAATTCTTCAGCGACTTCAGACATTTGTAGACAACGGTGAAGCTTTTG
GCATCGAGGCAGTAGGGGTAAGGAGGAGTGGCAGCCACTTAAACAGGCATGTCACTTCCC
TGAAAACCAATGATCTGAATACTGCCTACAACCTTTACATCAAGGAGCAGGAAGGGGTCC
CTTGGTAGAAGGCATGTGGAAAGTTTCGCATGGATGAAAAATGTTGTTCCTGAAAAACAT
GCAGCCAAGGCAACAAAGGAGTCTTCATGTTCCTGATCTGAAAACAGGAAAGCAAGCAGA
GGTCTTGGCTGGATTCATAGCAGCCACCAGTCCATTCTAAAGGCCCAGAACTTGGTTCCA
GTGTCAGGCTTGCCATGTAGGAATGCGTCCTCTCTGGATGTGCACACAGTTAGCATGGTA
GTCCTTAAGGCAAATATTCCCAAGAGTTCCCCCTTGGTAAACTTCTATAAGTGTGTTCCT
TCTGAGAGAAGTGGCACATGGCTCGAGCTTTTAAATACACTTAAAAAAATAAAATAAAAT
AAACATAATCCACTTTTCAACTTCAGGAAGATGTATTAGGATGCATGAGAAGTGATGACT
CTTGGGTTTCCTAGAACGGTGGCAGAGGTAGAACCGGGTTCTGCATTCAGAAGTGTTCTA
CCTTTATCTTGGCTCGTGGTGCTGGGTGGCCGGCCGTGTGGTGATTTCCTTGCTGGTTTC
TGGTCACTGCGTGGACTGCCCTGCCCTGTGGTGACGTTTGTCACTGGTGCAGTGCATGTT
CAGGGAAGAAATTCTGCGATGTGGCTCCCAGTGGCTAAAGTGGTACATAAATAGTCAAAC
ATTCAGAGTCCTCCACCAGCTAAGCCCAGTTTTTCTACACTTTTGGTAAATAAGTAGAAA
GGCTTCTTGTTCAGGCTTGCTCTGTGTGAACCGGACGGTTGTGCTTGGCTGGCTTCTCGG
TATTCTATCTATTAACCTCGCCACCCTTTTGTAACTGGTGCATTTAATTATCAGTGGGAT
GCTTTTCAAACAGGTAAGGGTAATGATGGGAAAGGCGGTGGGGGCATCTCATTGAGAGTC
TCCATTCTGGATATGAGCTTTGGGGCCCACTTGGTGAGTCTTTGAGGTGGGCTTTCATGA
GAAAATCACCCTTTGTAACCAGATCGGACTCTATTGAAATGTCAAGCCCTGCAGACTAAA
TTGCAGTCCACGGCCGAAGTCATGTCAGCTCGGATGCTGTGGGGCACTTGTCCTTTCTTC
AGGCCTCCCCTGGCCACGACCTGGTGACACGTGAATTGTTCGTGCGAGCGTGCGTGTGCC
CACACATGCGATCGCTGAAGCCTTTAATATGTTTGTGACTGGAATATTTTATACCGTGGC
CACCCAGACGCAGGCAGGACAATAGTGTGGCCGAATTGACTGAAATGAGGATTACAGAGA
ATAATGCCGCCTCTCCAAAAATACTGCTAATACAGGGCCCGCGACAGGAAGCGCCAGTTG
AGTCAGCAGAGCACAGGGCCCCAGGCAAACCCCGGGCCGACCGCCTGGCACAGTGGCCCC
CCGGGCACGTCGGGCATCCCGGGGTCTGTGCTCCTCGGCCGCCCCGTCGCAGCGGCCTGG
GGTTTTGGACGCCGCACGCGTGCCGCTCGGACGGCGGTTTGTTGCGTCCGTCAGCTTGGC
AGGAATGGAAGCAGCCTCTTCTCGGGCTTGGTTTGGCCGCCACTGTTGCCAGGAAAAGGA
AGCTCCCAGCTGCCAACACAATTACCTTTGTTTCTTACATCCCCCTTCTTAATGTGGCAA
TTAAGTGCTTACCACTCTGCGCGGTGCCAAGGCAACCACCGTGGAGTCGCCCGCAGCCTC
CGTGCCCCGGGTGCTGGGAGGCCCCGGGTGCCTCCGGGGGCCTTGGGGTTTGGAGGCCAG
ACCCTCCTCCCCAGAGGTCACTCTGAGAGCTGGCCATTCTTCTTTCCTGCTGCCTGGGAA
TGGGGACTAGCTGCTGTGGCCAGATTTCCCTGGGCCTGGAGGTGGGAAGGGAGGCACAAA
CCCACACTGAAGTAGCTTCCCATCTACCCATCCATCCTGCTCTGCCCCCAGGCTCTCATT
CTGGGAGAGATTTAAGAAAGAAAAAAAAAAAAAAAAAGCAAGGTGACTCTTCTTCTAGTC
TTACTTTTGTAGTCTGAGGCCAAGAAAGATCCAGGTATTTGCTGGTTCTGTTTCTCGTGG
GTGGTATTTTTGTATGAACTTGTTAAGTTGGACACTTGCCCATCATCTGGCCTGGCACCC
CCTTTCCAAGCCCTTAAGAGGGTGCCCAGGGACTTGGCCTCCGCTGGGGGACAGATTGGC
AGTGACAAATTGGGGGTGACGTGATCAGAGCAGATTGCGTCCAATTCTTGGCCTGATACT
GGCCTAACATGATGACACAAAGTGTGCGGTCTGGACCTCCGAGTATGGATATATATTCTG
GGATGTACAGAGGTCAAGCGTGGGCCTGGGAAAAATTAAAGTTCCTAGCAACTAATAATC
ATTACCAAATTATCTTGATTGTTGATCTATAGTGTGGGGATGGAGTGGGAGGGAAGAGGG
CCTCCTAATAGGGGCCTTGGAGGGAATCCTTTCTTCTGAAGGCTGGGTGGGAGGAATCCT
CCTGCCCCAGGAGGTGAGGCCCAGAACCATCAGCACTTCTCAGAAACCAGGGACAGAGAC
GATCCAGGGCTTTTTATTTCTTTTGTTTATTTTTTTTGACAAGGCCTTGTTGTCAGCACT
GTAGCCCCAGCCCTCACCCCGTGTCCGTCTCTGAGAAGGCAGTTCACGGAAGATTAGGAC
ACTTGGCGCCCCGAGCTCAGTGCTGCCGGGTCGCTCTTTTCCCTCGAGGTTGGGCAGTGT
CGGTCAGCACCTCCTGAGGTCTTCCCGGCAATGGTGGAGATGAACCTGGTACACAGCTTG
CTCTTCCTGGTTCTTTTGCAAGAACTGAGGGATGCCACCTTCTTTGAGCATCAAGCAGAG
TTAATACTGTCATGCCAGCTCAGTCCAGGCACGCCTTCTCCTCCTGTGGGGTTTGTGGAT
ATTACATGTTTAGTGCTTGTGCAAGTCTGTCAGAACCGCGGGTATCAATGCATTTAATCA
GAGGATTAATTACAATACGGGAATGTATAGAACGTGGTCTCGACATGGCCAGCCAGCGAT
GGGCTTAATTATACAGAATCAGCCCCACATCGGGCCTCATTTGGACTTGGTTCTCACCTC
CAATCAGTAGAGAAATGTAATTTCTTGCTGCTCTCAGGGAAGAGTCCTGTAATTTGTTGT
TAAGTCCCGGGAAATTTAGTGGCTGTAAACAAGGTGTAACTTTTACTAATAAAATATTAA
CGATGACAACAATGAATTGGGACCCATGGCCACCAAACAGGAGAGGGCTGGGTATGTGCC
CTGGCTTGCCTGGTTTTGCTTGCTTTTCTGAGCATTTTCCCACAGTTATCAGTGAACGAA
ATGGAATCCTACTCTGCTTTGAGTATGAAACATATTTCGGCTGAATTCCAAGGCAGGGTC
TATCACAAGATATTTTTATATTCAGAGACAACATGCTGTGTCTCTGTGCGAGCCATAAGC
TTGCCTGGAGTTATGAATGAAGCTCCTTTTTTCTTTCTTTTGCTCCATGTTTGCCTTCAC
CTTGTAGGTCTGCCTCTTCTTGGTGACTGTGCTTCTGGCCACACGGTGCTCTTTGAGCTC
CGTGCGCCACTGTGTCCAGTGCGTTGGTATCTGACAGTGACCATGCAGGGATACAGAGGA
GGAAGGCCTCATGGGCCTTTTCTCCCCACCCCCCTCAGATATATTTCTCCAGCGCCAAGA
TAGCAGGTTTATTTAAACTGTGGTGTTTTGTGAGACTGACCTCATTCTAAATTACAAGTG
AAGATTTGAGTAAAAGCGGGAATTTGCTAATGGTTTGGATTTGGGGGGGGGGGGGAGGTG
TGGAATTCTGAGATAAGCCTGGAAGATAAAAAGGTGCCTTCAGTATAATAGCTGGCATCC
TGCCAGTGTCCAGGGAATTTGTTTAGTTAGTGGACTCTGATTATTCCTGGAATGTTAGAT
CTTATTGACAGATAAGTCCATCAATAGCTATAACCCGTGGGGGTGTGGTGGTTTCTCCCT
TTTTGTGCTTTCACCCCCAGAGTCTGGCCCCCAAGCACCCCAAGTTAGGTCGCCGCCTTT
GTCCTGGTCTCTTTGCACCTACCCCCCCCCCCTGCTTCCTAGAGTCACCTGATACTTGTG
GGAGCTGAGATGGTCTCACAGACAGGGCTTTCAAAGGGGTTCTGGAAAGGAAAGGGGGCC
TCCCCAGGGGCTGCCCCACCAGGGCCCAAAGTGTAACCTTAGCAATACTCAGCCCTCCTC
ACTGGATGCCCTCCCCCCGCCCCCATGCAAATACCTGGTTAGGTTAGCTTAGAAGAAATT
CTTTGTGTGTGTTTCTCATGCACTAGAGTTAAGGTCTGGAAAGTTTTGGAAAACTGTACT
GTGTTCACATATAGGGAGGTGGTACTTCCTGATGATGACTTTGGGCAGCTTGCACAGCTG
ACAGTGACCAATCGTGGTGGGCCCCTGCCAGGCGGAAGGAGTCAACCTGGGGATGGGTAC
CAAGGAGGGGCGTTGGGGGATCTCTGCTCCACAGCAAACAAGAGTTTTCATTTTGGTTTT
ATGGTTCTGTTCGGTGGAGTATGTGAGGGAGCGCGAGCAAGTGTGACATGTGGGAGCTGA
CTCTTATTTCTGAATACTAAGTGGTGGTTTTTCTCTGTTGCAGAGGTCCTACACTTTGCT
GGTGGAAGCGTGGGATTCCAGTAATGACACTCTCCGTAAGTATCACAGCAACGGGCTTTA
CTCCAGGTGTGCCTTTCTGGAAGTGCGGTGCTTGGGGCACAGTAGTGTTGGGCTGGATCT
GTCTCCCGAGTGCTGCAGAGGGCTTGTCCTACACTCCCTGCTGTCTGTCCCCATCCTGTG
CCCACCCCACCATCCCCAGGGTGAACTCCCTGGAGTCTTTTCCATAACTTCCCCTAGAAT
GTGTCCACTTGGAACATTAAGTGGCCAGGACAGATTCCCAGTTACCTCAGTCCCTGAATA
TGAGACCTAGATTATCTTTGGGATAGAGATCTTAATGGTTTTACAACTTTATTTTTTTTT
TAAAGATTTTATTTATTTATGAGAGAGAGAGAGAGAGAGAGAGAGAGGCAGAGACATAGG
CAGAGAGAGAAGCAGGCTGCATGCAGGAGCCCGACGTGGGACTCGATCCCAGATCCCGGG
ATCATGCCCTGAGCTGAAGGCAGAACCGCTGAGCCACCCAGGCATCCCGGTTTTACAACT
TTAAAACTGTGCAGACTGCTAAAGGATCAAAGTAGTGACTTTTTAAAAGTCCATTTAAAC
TTTAAGTTGAGCGTTTAATCTCTCATCAGAAAGATAAAGCCTGCAAAGGTGGCGCTGAGC
TCTAAATCTGAAGACCTTGAATTTCACTCGGTGGGCAGGTGAGCCTCTAGCGGCTCAGGG
TTTCTTTCCCACACTGTGCATGACAGACCTTTCATTGAAGTGGAATCCCAGAGCCTCACA
GGCACCGCAGCTGCCTGTCCCCTTCTGAAAGATGACGGTTTCAGGGGCAGGTTGCTTTTC
AGAGGTGCGAGGTGGGGAAGTCACAGGGATTGTGTCTGGAAATGGCAGCAGCCTGGCTTC
TATGGTGTAGTCTTTGCTCTTTAACTTAAAAATGTTTTCCCTCCCTCGCTGAAACTCTGC
ACTTCCTTTTTTGCTAGCAAGATCTGGAAGGTTGTCCTGGGAAAACTTGATTCATGCTGA
AATCCCTTAGTCTGCATAGGGGGAGGGTGATCCAACTCTCTTTGCCCTCCACCCTCTGAA
TGTCTCCTCCCTTCTTCCGTACCTCACTGACCCTTTCTGGTTTTGGCTACTGATAACATC
TCCCCTCATCTCCTCACCATGCCATTTTCTTTTAAGCACATCTGAGGATGCTGAATGCCC
AGAGTGGATGTGGTGAGGGCTTGGCACTTGTGAGGAAGGCTACTTTGGGCTGTGTCTGGC
TCATCGGCTCTGCCCTCCAGCCCCCCACCTCCCTACCCTCCGCCCGGGGCCGCATAATTA
CTGCTAAGCCCTGTAGTGTTGAAGGTTCCAGTCAATTTGGGTCACTCCTCTTGCTCTGAT
ATCCGAAGCCCAGATCTGTGGACTCTCTGAGGGCCACCCCGTCCTGCTGTGGTGTGGTGG
CAGAGGAGGGTGGTAACCAGCCTCAGTTGTGGCCCATGCCTGTCAGGGTCATTAGACACC
TTTTCGTTCTCCTCCCTCATGGGCCACAAACATTGGGGATTGTGTGAGAGTTGTGTCACT
GTGAACTGGTTACCATGGTAACTAAGCTTTGGGAAGATGGGGAGGGGGCAGTTAGACACT
TCCGATATTCTGGTGCAGATGTGATCTTCTCCTTCCCTTCCCTGTTAGATGTTAAAACAA
TCTGGTAGAGTGAAGATCCTCGAGATTGATTCTGACTCATGGCAGGAGCTGGTTCAGGAC
ACCTGTTAGGGTGTGCAGCACAGTTAGTGTCTGTAGGGCCCTTCTTGGGCCTTCCGTCAC
CCCACAGGTCAGCTAGCTGCGCCCCTGGGAAGCTGTTGACCATCCAGAGCTGCCTGCGGT
TGTTTCCATTTCCGAGTAGCCCTGTTTTGGGGTTCTGAGGCCCCTCCTCACTGAGGAGGG
GGTAAAGAAATCCCCAGAAGGGCACAAGGAAGACTTGAGGTAGGGAGAAAGAGCTTTCAG
ACCTCTACTCTTGAAGACCGGAGTTGCCACAAAGCCCTGAAATGCCTACTGCAGGTCGCT
GAATCTCACCGTGTGAGCCCCAAAGAGGAGGAAGTGGTTTATACACTTGTTTATTCAGCC
TCTCTCTTTACAGCACTTCCAAGGGATCTTGAATTCACGTTCAGAACCATATTTGGCATC
TTCTCTCTCCAAATTAGATCCTGCCTAGGGCCCTTCATGGATGACTGGCACCAGCATTTC
AACACCTAGTACCATGAGCCAGAAATCTGGAAGACACCCTGTAGTACTTCCCTTTCTCTT
CCCATCCATATCTAATCCACCTTCAGGTTCTGAAAATCTTATCTCTAATGCCTCACTGGA
ATCCTGCCACCTCCCACATGTCCCCTGCTGGTCCAAGTCACCACCTGCTAACACCTGAAC
CCCTATGGTAGTGGTTGAATTGGCCCATCCATATCTACTCTGGTCCACCACCAGTGCTTT
TTCCATCCTCTTGCCATCCTGGTCTTTCCAGAGCCTTTCCCTTTTTTTCATTTAAAATGA
TTGACCTACAATATCCTCTGTTTCTGGTTTGCATCTTGATTCGATTTGATTGTCCTCTTC
ACTAAGAGCCACTTGGTGGCTTCCTGTTGCTCTTGGGACAGCTCTGTGTGTGGCCCCAAA
GGCCTTGTACAGCTGTTTTGTTTCCTTGCTAGCTCCTAACTCCATCTTACATGCTGCATT
GTCTTCTACCATATCCAAATCCTCATCCTGTTCCACCAGTCCCCATTCTGTCCTCAAATG
CATCATCACCTTCTGTCCTGCCGCAGGGCCCTTGCACATGCTGTTTGCACTGCCTGGAAT
GTTTTGCTTATTACTCTTTACCCTCTGTCCCTCCCATGAGGCAGTACTCCATGAGGGCAA
AGCCTAAGGATGTGTAGCAACCCAGATGGAGTTGGCTGCTCTTTGGGGGCCAAACAAGTT
GCCAAAATTCTGGTTTCATATGAGAGGCAGAACCCTGAGTTTTAGGACAGAGGATTCCTT
CTCACCTAGGCTCTGTAGAGAGATTGGCCCCGACGAAGCCCAGGTGCTGTTTGAGTGTTA
GACTGTTGCCTAAAGCCTGGCCCTCCTGGCATTAGTCCTCTTGAGACCATTTCTCTTCTC
TTTCAGAACTATATAAGACCCACATTTTTCTTTTCTCCCCCAGTGCAAACGGTTTCAGTT
TATGGCAGTGCTCAGTAGATCAAATAAGTGTTAGTCCAGGTTTTCCTTCTAGCTTAGGCA
CCTTGACTAAGCTCCTGTCCTAGGTTTGTGGGTTGGCGTGGCCTCAAGGTTATGCTCAGA
CTCGCCTTATTACCTCTGCCTTGGCCTGCGAGGCTGTTGTGGGGGCTACGTGCTCCATCT
TCACACAGCTGAAGATCACAGGAGGGTCTGCTAACATCCTCTTCCGGTCATAGAGCTGGA
CCCCCAGGTGAAATACCCCCTGGATACACCTCTATGCCCCAAGATGAGAGGACGTGCAAT
TCAGGTGGCGGCAGCTTGGAAGAAGCAGGTAGCAGAGTTGATGTCTACAACCAGATACCA
GAAAGTCTGTCCTGCACCCATGACTCCCCCCCCCACCCCCCCCACCACCACCTCCAGCTG
CTGGGAGGAACCTGCCACACAGGTGTGAGGCTGGTTCTATGGATCTGTTAGCCATGGTGA
GGATTCCAGAATAATAGTTTTCACATTTTCTAGACCTAGGTGGAAAGCCAACTGAACTGT
TGAAAATGACCTGTCAGCCTGTGTCTTTAGGATTACCCTAGACTCTTGTCTGAACTGTTT
GTTGTTACCTGCCCAAGAGATGCTGGAGGCCTTTGAGCCGTGTGTTTAAATAATTCCCGT
ATTAAGATCTCATAATCACATGGTAATAGCTCGCTACAGTTTTTAATTGAATCACGGAGC
AAACACACAGTTAATGGAGGGACATTTTTGTCATTCCTTGACCTGTGTTATTTGTAAGAC
TTCATGTTAGTAAAGCGTTATCTGCAGGTGGTTCTGTTTGTGCTGGGGTGTAAGTCTACA
GGTATGTGGGCTTGTTCGACGCTTGTCCTGCCCACACGACAGTTAAACCCAGAGGGTGAA
TGCTGGGGCGAAGCTGGAACTCGTGTGGATGGAGCCCCTCAGGAGCTCCTCCTCCCCTGG
TCACTTGTTGGCTTTGCATAATCTTGTAGATGAGCACAGGCGCTGGTCCTAGAAAAACAG
GGGTGTGGGTGGGTAAGAATCACAGACATTGTGAGTAGGTCCTAACACAGTCTCCCGAAG
GTGGCCTTGCCCTTGACAAGGCAGTGAAGGCTTGGTGCTGGCAGGTTTAAGAAAACAAAC
ACCACATCAGGCTGTGGTGCTGACTTACTGCCTTGTTTCCCTTTCCCGTCCCTCCCCTGG
ACTCCTGGCACCTGGCGGTTGGATTTATTTTTGAACCTGTCTCTTCTCTGACTAGATTAT
AAGTGACCTCAGGGCAGGGACCACTTCTTACTCCTCTCGAGTGAGAGCTGGCTGCTTGAG
CTCTGGGCGTGTTCATTTTCAATAGGCACCTTTTAAAGAGCATGACTTGGCTCTGAAAAA
CCCCTAGTCTACCTGTCCCTAAACTGGGGTGTGTTACTTTCCCCTCTGCCTTCTGGGCAA
AGGTGAAAGGTGAAGCACATCAAGTTCGTGGTACAAGTGCCAGCTGAAAAGGTGGCAAAG
TTTATAAATTTTTAAAGACCTAAAACTCTACCCTTGGCTTCCAGGGAGGAGTTTAGAAGT
AGACCAAGTCTTTCTAGAGCAAGACCCAAAACTAAAACCAGAAGAGGAGGATTGCTAACC
TTTACCTGGAATAAGGTGCTTTCTCTAATATGTAAAAGGCTTTTGCAAATTGGTGTGGCC
AGCAACGCACTAGAGCACTTGGCAAAGGAGAGAAACAGGTTACAGTTCACAGGAACAGAT
GCAGATGGCTTTGTAACCACGTGGAAGGGTGCTCACCATCACTTAATTAAATGCTAATTA
AAGTAAGAGATCCGTCTTTACCTATCAGGCAAAGATCAGAAAGTTTGGTAACCTATACTG
TGCTGGCATCGCCGTCGGGAAAGCCAGTTTCCTGTGCGGGTGGCAGGACAGGCTGACAGG
CCTTTGTTCGTGGTCGTCTGTCGATGGATGTGGGTTGTGTGCACTGTCCTCTGACCCAGC
GCTTCCCTTTCTAGGAGGTCTCTTACACTAGAACCACGCACATGGGCAGTCGGTTCAAGG
ATGTTCGTTATGGCTTTGCTCACTGGTAAAAGGTCAAAATAGCCTAAGTGCACATATGTC
AGGAACTAGGTGAACGCGTTTCACTGTATTCCCAGACAGGGAAATACTACACAGCCGTTT
AAAAGGGTAAATAGGGCAGCCCCCGGTGGCTCAGCGGTTTAGCGCCTCCTGCAGCCCAGG
GCCTGATCCTGGAGACCCGGGATCGAGTCCCACGTCGGGTTCCCCGCGTGGAGCCTGCTT
CTCCCTCTGCCTGTGTCTCTGCCTCTCTCTCCCTCTCTGTGCCTCTCATGAATGAATAAA
TAAAACCTTTAAAAAAATAAAAGGGTAAATAAACGTAAAGGTGGCTGCCTCTGGGGAGAG
GGACAGACTTTTCACTTTTCTGCCTGTTTGTAGTGTTTGAATTTTGTACTATGTGCGTTT
GTTTTCATTGTTAAAAATAGGCAAAATGGTCAGGGGAGAAGCTTCTGGTGCAAATGTTAA
TACAAATCAATACTTTTTTTTTTCTTTTTTTGTTTTGTCTCTAGAGCCCGACAGCATTAT
TGAAAAGGCCTCTCACTCAGGCATGATCAACCCCAGCAGGCAGTGGCAGACACTGAAGCA
GAACACAGGGGTTGCCCACTTTGAGTATCAGATCCGTGTCACCTGTGATGACTATTACTA
TGGCTTTGGCTGCAACAAGTTCTGCCGACCCAGAGATGACTTCTTTGGACACTATGCCTG
TGACCAGAATGGCAACAAAACTTGCATGGAGGGCTGGATGGGCCCAGAATGTAACAAAGG
TGTGTGGGCAGGCGTGTGCTGGGTGGGTTAACCAGCTCTGAGTGGCTGGCACTGTTGGTG
TCAGGTTCTCTCCCCACCCCCACCCAGTCAAGTGGCTATCTCACATGGGGGGATATTTGG
GCTGTTTTCTAGGCCTGAAGCACAAAGATGCTGAAGATGCTGTTGGTACTCCCCTTTCCC
CCAGCTGCTGAGTGTTGGCTGCTAACGGCTCACAGCTGTTCCTTCTCTAGAGCCTTGTCC
TTGGCCAAACAGCCACTCCATGCCTCTTCCCTCCAGCTCACAGTCTGCGAATGACTGGAT
GGGGGTACAAAGGCCAGCCACCACCCCCACCACTCCCCACCCCCCGTTGGCCTTGCACTC
TGTGCTACAGTTCACACGCCAGGGCTTCCCCTGGGATCACGTTAAAGCCAGATTCCAGCT
GAGACCTCTGCTTGGCTCCTTCCCCACCCCATCTGCTGCCTTCAACTCCTTTCTCCTGTA
CCCAAACCCCTGTCTCAGGCTCTGCTTCTAAGGAACCTGACCTAAAGCAGATGCTAATTG
GGACAGGGTTAGCTCAGTGCCTGAATATGTGATCCTGCAGCAAATGACAGCCCCTAGTTC
ACGGCCATCTGCTGGCAAGCTTTTACGAGGTGTTTTCGGGGGCTGTGGTGTTGAGGGGGA
TGGGGTGAGTGGTGAACCAGAGCTTTAACACTTGCATAGAGTCGAACCCATCACCGCAGC
ATAAGCTCTCAGATGTGCTGTCTGTTGTCCCAGCCTTCTCCTGGGATTCAGGGAGTTAAC
TGCATAGTCACAGATGTTCACAGAATTATCTGTGCTTTGTACCTTTACAAAGCTTCTAAT
TCATAGCAAGTGGGTAATTCTAGTTGAACAGTGTAGGCCCTCACGAACATTAGTGTACTC
TGCTTATTTCACCTAATTTGGTAAATTCTTTCAAGATACACTCAAATTTAGAAAGGGAGG
TTTTGAATTGACTGAAGGGCAAGACACAGAGGAATTTGCAGGTCTCCTGAGCTTTTCTTA
AATCACTAGAGTTTTAAAGGGATGGCATTTCCGGTATCCAAATGAAAGAATTAGCAAACG
TACCTAAAGTAGTTGGTATTCTGAAAATAAGTAATGATTACTGGGATACGATGTTGAGTA
TTTTTTTTTTATCTGTTTTTTTTTTTTTTTTTTGTTTTGTTTTTGTTTTTTTTTTTTTTA
ATGAAAGGAAAAAGAATTCCTTTGCAAGGATACTTTGCAGAAGTACCACCAGATCTGGCC
TCTGGATTAGCCTGCCAAATGAGGCCTGCCTCATTGGCTTTGTTAGTCCCATGGGATTTT
TGAGACATTGGGCATTCTAGGGGGTGGGGATGAGGGGGTTAAGGATGCTGGGGCTCATCT
TGAATGTGCCCTCATGGGACAGGGTGGTGAGATGAGGAAGGATCACAGCTCCTGTTCTTT
TGGAGAGGGTGGGAATCAGGGAGCTCGCTAGCTATTGTGGAGAGCCTCAGAATGAAGATC
CCCCTCCCCCATTTAGTCCCTGAGTAGTATCATGTTTTGAAGAGCTGCTAGCATGGCCTT
GAAGTTTAAAGCTGGATTTGCTATTTGGGCACCTCCCATCTTCCTTCCCTTACAATGGAC
TTGACATGAGATCTCTTAAAGTCATGGATTTTGAGGCTTAATGTACTGGGCCATTCTCGT
GGTGGTGGTGGTGGTGGTGGTGGTGGTGGTGGTGGTGCAAGAAACCATGTGCTTGACTCA
TCTTTGTGTGTGTTTTGTTGTTGTTTTTATAGCTATTTGCCGACAGGGCTGTAGTCCCAA
GCATGGATCTTGCAAACTCCCAGGTGACTGCAGGTAAATGACTAAACTTTTTTTTTTTTT
TTTAATCCTTTAATGCAAGAGGGGGACTGAATTTATTGTGAACATGCCTCTTTTCCTTTG
GAGGACAAGAAGCTTAAAAAAAAAAAAAAAAAACCAATAGGCACATCCGAAGCTTATTAA
ATTATACCAGCTGTCTGGTGGAATGTGCACACTTCTCTCTTGGAAGGAAAAAAAAAAAAC
TCTTATGGGCATAGTCGGTGACTTCATGTATTAACTGTTCTGTAAATTGATAACACGTTT
TTATAATCTTCCCCTTAAAGTGCTAATGGATGAGATGGAGGGATGTGGGTTTCTTTGAGT
CTGTCTCCTCCCCCAACCTGGTTTTCGGTCTCCCAACCCTCCCCCACTTTTCCCATCACT
AGGGGACAGCCGAGCATTCTTTAGAATGGGATTAGTACAATCTCCTGGCCCCTTTGCAGG
CGGGAGCCAGGCCGGTGCTGAGAGGTTAATGGCTGCGCTGGAATCTTGTCAGTTGAGCCT
GATGAATGTAGACTTTTGCTGCTGGCCTTTGAAGTCTGTTCTTTTATGGTCCCGGACAAT
GCAGAAGCCAAACAGAGACCCACCCCAACTGCTTCATGATTACAGTTTTTGGAACCTTCT
TGAAATGTACATTATGCCAATTTGGGAAAGACTTGCTTCAGGAAATGGTGTTGGTGCCTG
TGACATTCTGACACAGTCTCACTTGCCTCCCCCTCCTCTCAGATCTGAAGGATTAGCAGA
TTTAGTGACACTGAGAGGATCCTTGGGGCCCAGCAGTTTTCCCCCAGGAGCTACCCTGGT
TGAGCCCAGGGAGGCAAACGAAACACACTAAACCAGCACATCTCAAGTGGACTCTCTTCC
ACCGCTCTGGTTACCATGCAGGGTCTGATCCCATAGGTCTGGGGGGACCTGAATGTGCAT
TTCCAATAAGTGGCCCGTCCCCCCGCCCGGTGATGCCCATGTCGCTGGCAGGACCATACT
TTGAGAATCACTGCCCCAAACCTCAGTGTTCACGTCCGTAGTTCCCTGAATGAACTGGGG
ACTTGGACATGCAGATTCCCGGGTGCTGCACCGCACCCACTGAATCCCAGGCCAGGGTTC
GGCCCAGGAATCTGCATCTTGACGAAGCCCACCGAGCGATCTTGCTGTTCTCTGTGTTTG
GGGGACTGCCACTTTGCAGGGAATTCTCTGGTCTTTACTTTGCGGCCCCGCCGGCGCACG
TCTCTTCCCCTCCTCTGCAGCGGGGAGCACTGCTCTGAGGCTTGATGCCAAAGTGAGAAC
TGAGGCAGCTGGAAGCAAACTCAAACCAGGTTGACCGGGCATTGTCTCTTCCTCCATGTC
AGAGAGGTTTATTTTCTGTTGGGCCGTGGGTGGTGGCATGCTAAGCCCCATTGGTGGCAG
CTGAGCATTGGAGTTGACAGCATTGCTGTCAAAGTGTGAGCAGGAATCCACCTACCTTCG
CCCCTCTTAAGGGTGTTCTAGAGAAAGTCAGATGAGCAGCGCTGTTCGCCACTCTGGGCC
TGCTCTGCTCAGATGGGTGTCGGGAGGCCGGCCGCCGCGATTTCGGATTTCTGCTACTGT
CATCAGAACGGCTGCGCAGGGAGGGCCCTCCCAGCCAGAACAACAGGCTTTGTTTCCAGA
ACGGAGCCCTTTCTCTTTCGAAGTCTCCCCTCCCACTCCCTGCCCAGACATGCACCGGTG
CCTTTATTTACTTAAGGTGTGACTTTCCCACACGTCATTTGCATAGTCTCCCAGCCTGTC
CTGTTCTGCCTACCTTGAAGGAAAATCAAAACAGAACCTTCTAGCTGAGGAAGCTCAGGT
TGTGTCCTGCTCCAGAAATAGTTGAATGGCCAGTTACCTGTAAGTTGTCCTATGGATTAT
GTATTACAGAGGTGGTGTTTTTTTGTTGTTTGGTTTGTTTTTGCTGCCTTTTTTTTTTTT
TTTTTTAATCCCCGCATGAGAAAACTTCACTGGTTTTGTTGTTCCCAAGTAGACTCAGAT
TCCTTTTCCTGGGTGCACATGCTTGCTTTATTGCAAGCCTGCCAATAATATCAAACTTAA
TGATGAATCATTGTAGGAGGAAAGTCACTTCAAATTCTCATGAGAACCTTAATCATGTAT
TATTAAACTGGGATAACTTTGTAGATGCCTCTTTTAGGGTCAGTATTTTTATTTGTTTTC
TTTTGAAGTCATCCATGGCCTCTTGTGGTTTCTTCTTTATGCTTGGGGATCCAGCTCTCG
TTAATTAAGAGGTGAGGTTGGGTCCTCAGCTGCCTCTTCTTTTATTAGGCAAAGTGAAGA
GGTGTCGCCATCCATATTTTGGAAGGAAGAGAAGACAGTAAGGATACTGCTATTTCCGAA
TCAGTTTCCTCTTAATAGCTCTCAAGCTAAGATGATCTTTTCCTGAATGAAACCCCCTCC
CCCAACTCCCACTAACTTGAGTGTCCACTTTAAAAAAAAAAAAAAAATTTCTACCAGATT
TTTTTTTTTTTTTCTTACTGCTCTGAATGGTCACGGTCATTTAGACTGCTGTTCTGTTAT
ACACGAAGCCAAACCCTCTGTCTGATTAATTTTAAACCACGCATTCTGGTGGTTCTTTGA
ATTGGGGTGTGGATGGCTTGTTTGTGGAAGTTAGGAAGTGGTTAGTCAGTGTTCTCAGGG
GTTAAGTTGACTCTTGGCCGTGGGTGATTAGCTATTAGCTGGATCCCTATAATTTTCCCA
TGATAAGGATTTGCTAGCCTGCCTCCGGCTCTCTGGAAACGGAAAGCCTTTCCTTCTTGG
GGGTGCTTGTTTTCCAGAGTTACAGCTGAGTGGCTGGGGGTGGGGGAGGGCGCTGGTCGT
TCTTACTGTGGCTTTTCCCAGGTCCAGTTCCATGCTCGCCCCACCTTCTCTGGGCTTTCT
CCTGTTTACTAAAGCTTGAGTTTTAAGAGGGCAGTTAAAGTTTCTGAGGGCTTAAAACTT
GAGGGGAGAGTGAGTGAGACCCTGCCCTCTCTCCCACCCCAGGTGAAGGTTTATGTTTGT
GTTTCTGTTTGGGGATCAAAGGCACCGTTGGCCTCTAAAGAGTCCGATTAATGAGACGCT
TATGAAGGAAACTTGACTCTGTTTCCCAATACTAGATAAACCGAGTTGACCTCTTCCTCC
CCCCCCCCACCCACCCCGAGTGAGAAATCACTATAAAATGAATTAGCTTAAGCAGCCCCG
GTAGTGGGCCATAAATTATCTGTGGGATTGGTTCCAAGTGCTGCGGCTCCGCTACCTGTC
CTGCAGAAGTGGGTTCCGAGATCTTTGCCTCCTCTTGGCCAGAGGAGGGTGCCCCCCCCC
TTTGATTTCCTAAAGCCTCCCAGGGAGGTGGCTTCCTGCCTCACTGGGTGTCTTGTTGCG
TTTCTGTGCACTGGTGACTTGCTGTTTAGACAAAGAACCAGGCCTCCGTGGCAAAGTGGC
CGACGGTGCCGTGGTTGCGACGACTTTCACTGGCTCTTGTCACCCGAGAGTCCTTCCTGT
GCCTCCGCAGCCGCTCGCCGGGGCCCTGGACTGGTTTATTCCACGCCCCCACCGTCGCGC
AGCCCTGTGAGCCTGAGAAGAGAGTGGCTTTGTGAGGTCAGCCCCCGCCGGCCTCTCCCC
AGACAGCCCTGCTTTTGTCGGCAGCCGCCTGGGAGGGGACCTCGGCCCAAGGACCGAAAA
TGTTTGTTTAGCCGGAGCCCCCGCCGGAGCCCCCGCACAAGCCTTCCTTGCGAGTCCACT
GTCCTGTCACCAGAAATGAAACAAGGCAGGAAAGGCCTGGACTTCTTAGCCTTGCACTTA
AATGGTGCAGATCGCCCCGAATGTTCTAAGGACTAAAGGGGGGGTGCGTGCTGTCCGTCC
CCAGGTGCCAGTACGGCTGGCAAGGCCTGTACTGCGACAAGTGCATCCCGCACCCCGGGT
GTGTCCACGGCACCTGTAACGAGCCCTGGCAGTGCCTCTGCGAGACCAACTGGGGCGGCC
AGCTCTGCGACAAAGGTAGGCTCCTGGGGGTGGGACCCTGGGCGGGACGGGGCCTCGGGG
TTCACGGGAGAGTTAAGGAGTGCGGCCGCTGTCGTGGGGCTTTCACTTTTATTTTATTTT
TATTTCTAGGGACTTAATTCTGAATCTGTTCGAATTTGCCTTTAAGGCCGTGGCTTGGGG
TCTTTTTTTTCTGGTTTTAGTCTTTAAAGAGGAACGGTGGGGGTTGGGGCCCCCCCGCTA
GAAACGTGGCAGCCGAGCAGCCTGTGCCGCCTGCGAGCCACTGACCCCGCGGTCTCTCTG
GCAGATCTCAACTACTGCGGGACTCGGCAGCCCTGCCTCAACGGGGGCACCTGCAGCAAC
ACGGGCCCTGACAAGTACCAGTGCTCCTGCCCCGAGGGCTACTCAGGCCCCAACTGTGAG
ATCGGTGAGCGGTCCGGGGGGACCGAGAGCCCTCGGCTTAATTTCTTTTTTTTTTTTTTT
CTTTTGGTACTCTGAGTTTACAAAAAAATAACAATACCCTTAAGGCCTCATAAAAGGACG
AAAATATCCTCCAAAGTGAAGACCTGTTACCCCAGCGGTTCTGTGGGTCCCCTAAACTTC
CTTCAGGGTGATGCCGCCCACGCGGGGGGGCGTGACCCCGGCTCTGAGGCTCTGAGCCGC
CTTGGAATGTGGCTGGGGACACTTAGGCCGAAAGCCGGCTTCCTGCCTTCCCTGGACACA
GCGGGCCTATAGCCCGGGCTCGCTCCCGCTTGTCCGAGGCGAGCTGGAGCTGCGGCCCAG
GCGTGGGACTGGCTGCGGGGAGAGAGGACGTAGGTCGAAACACGCGGGTGCCCCTTGCTC
ACTGCCCTCCGGGTTTCGCAGCGGAGCACGCCTGCCTGTCCGACCCCTGCCACAACAGAG
GCAGCTGCAGGGAGACCTCGCTGGGCTTCGAGTGTGAGTGCTCTCCGGGCTGGACGGGCC
CCACGTGTTCCACAAGTAAGTCTGGGGTTGGGCCCGTGTCTTTTCCAGAGGATCACTGCA
TTGAGGTGGGGCTTGGGGGCAGGTCTCAGTGAGGAGTCTTGTCTCGGTGGGAGGGGGGAG
GTGCCATCAGCAGTCTGATGGGGAGTCTGGCTTGGGCTTCCAGCCCTTGCACCCGAAGTC
TCTGGGCACCCTCCTCCTACCAAGCCAAGTTTGCATGGTGTGGCTGCTACCAGAAGGGAC
TTCTCCGCGGGATACCCCCGTCCTCTGTGAGTGCTCCCTGGAGCTCTGCTGCCTCCAAAT
CAAGTCCTAGCTCTTCTCCTTGGAGCCATGGAGAGGCTGCCGGCAAGGGAGGTAGCCACT
CCTCAATTTGGTCATGGGTGGAGGGTGGCCCCAAAAGAGAGACCCAGGCCACAGAGTGGT
CCTGGCTCTGAGGCCTGGCTGCGCATCAGAACCCCCAAAGTGCTGGACAGTGCAGATGAG
GGGGTCCTCACAATTCCCCTCAGGACTGGGGAGTCCACATTCTTTCACGAGCACCTGGGC
CTCTCAGGTGCAACCTGGCCTAAGAACCACTGTCCTCAGCACCCTGAAAGTAGAGCATGC
CTGGTTAAGAATGTGGGTTCTGGGGTTGGGTCACAGGGGCTGGCACCCTGGCCCCACCTG
CCTCTTGTAGGACCACTCGTTACCTCTCTGTGCCCTGTTGAACGGGACAGTGGCAATGCC
CGTCTCTTAGGGTTGTGTAGAATCAATGAGCAGATCACAAGAGAGCCTGGTACTTCCTAA
GTGGGCAGCAGAGGAAGAGGCTACTAGAGACAAGTGCAGAAGTGGGATGTGGGGCCCCCA
GCTATGTGGTTTTGGGAAAACTCTGAGAAAATTAGAAGAATGAAGTTTTCTACCAGAAAA
GAGTCAGTTTTCCTTACCTCTTCTTAGCTACCTCTGAGCCACGCTGAAACTTCGCTTTAA
AAATAAAGAGTAGGTAGGGTGCCCAGGTGGCTCAGTCAGCCTTTGGCTCGGGTCATGATC
CTGGAGATCCAGGATCAGCTGCATCGGGCTCCCTGCTCTGCGGGGAGTCTGCTTCTCCCT
CTACTCCTAAACACCCTCCTCCCATGCACTTTCTCTCTCAAATAAATAAAATCTTTAAAA
ATAGAGAGTAGACAGAGCTAGAGGTCCCATGTTAGCAGTGTGGTGATGCTCTTGGGGATC
CAGCTGGCTTTTCCTGCTGCTTAGATGGGGGATTAAATGAACTGGGGCAGTGGAGGATTT
AAAGCTAAAATCAGGCCTTATCGTCATAGCAGTCACTTAGAGCAGGTGTCTGGCTCTTAA
GTGAAGGCCCCTCATGTGAGGGTTGGCATTCAATTATCTAATTGTCAAACGGACTGAGAT
TAAATTGCCAACCCTCTTCCCTTTTGTCCAACAGACATTGATGACTGTTCTCCAAATAAC
TGTTCCCACGGGGGCACCTGCCAGGACTTGGTTAATGGATTCAAGTGCGTGTGCCCACCC
CAGTGGACTGGCAAAACGTGCCAGTTAGGTAAGGAAGTTCGGGCACCCAGGTTGGCCCTT
ATGGATCCGAGTTGTTACTGGAGATGAGCCTGTGGCTAGTGGTGTGAGTTTGCACACCAT
CATACTAAAAAGTAATTTATTGTCAGGAGTCCAGCGGGCCTGGGAGAGGGTACTTGAATG
CTAATGGCCTAGCTCTTTAGGCCCTGTGGGAAAGTCGTGGGATTCCTTGAACCATGTCAA
TCGTCTGTCCGAGGAGTGTTCAGCTGGGTTCCACGGGGATGGTCTCATGAGGAATGAGCT
GTCTGCATTCTTCCTGCCACATTTAAATCCCAGCAACTAGGCTGCGCCGCGCTCGCACAG
CAATGCCTGGTGGCTTTTTGCTCTCCTGTTGGGGGCCAGTTGTTAACCACCAGCCAGAAA
GTTATCAATTAGCATTCCTTATTGGGTAATGGCTAGGGAGAGGGATCAGCTGTTCCAAAT
AGTGGGAACCTAGTCTCTGATTTAGCGAGGTTATTAAAACGTCTCCTAGTTCAGACTATT
GATAGGGTGTGTTTGGGCGGGGGTGGGGGAGTGGGGGGTAAATCTCCCATTTCTCAATGC
CCCTCCACATTTTCTTTCAGATGCAAATGAATGTGAGGCCAAACCTTGTGTAAATGCCAA
GTCCTGTAAGAACCTGATTGCGAGCTACTACTGTGATTGCCTCCCTGGCTGGACGGGTCA
GAATTGTGACATAAGTGAGTCCCTTTGTTTCGATTTTGATTTTTGTTGAAACTTGGGTAT
TAAAGGCTTTCCTCAGCCTCTATGTCAGCTCCAGCTCTAAGACTTGAGCGGTAAAAGGAA
AACCTCAGAGAGGGAATGTTTTTGGGGAGGGAGCGGTCAGTGCAGAAATGAAAACATGCA
CTTTATCAGCTCATTAGCCGGTAAAACAGCCGTATGCTGATAAGGTGGCTAAAACAATGA
TGTGAAGTTTCACTCGCTCAGTCTGGAGGAATGAGGGAGGGTTCCCACCCAGTGTAATAA
CCGCATGTCAAGAGAAGCACACAGGACATCTGATTTGAGGCTTTTATCTGTCCTGAGCGG
AGAACGCTTTTCAATTAAGCCCAATTTCAATGTAAATTGCCTCTTTAATACGACAACCTG
TTTATTTTTCAGATATTAATGACTGCCTTGGCCAGTGTCAGAATGATGCCTCCTGTCGGG
TATGTGAATCTTTGCTTAAGTGAAAACCTTACTGATTGACACAAATCTGTTAAGATACAT
GTGACCCTGGGAGAGCTTGATCCCTCTGAGATTCTAAAAATGCAGGTGAAAAGTGATTCT
CCGCAAGTTTCAGTTGGGCTCCTGTTACCTTCCCAGCCCCCTGGGAGCCATCTCGAAGGC
ATGCCTATAGAAATTCCTCTCTCACGTTGCGAGAGGCTGGGCATTTAGCGATGTCAGGGA
TTCAGTGGAAGGGCAGCGGGTGTGCTAGACTGCCAAGGCCCCATGTTTGTGGAATACAGA
GGAAAAAGCATGTGTTTCTCCCCCCCTCCTTCTCCTCTTCCAGGATTTGGTTAATGGTTA
TCGCTGTATCTGTCCACCTGGCTATGCAGGCGATCACTGTGAGAGAGACATCGATGAATG
CGCCAGCAACCCCTGTTTGAATGGGGGTCACTGTCAGAATGAAATCAACAGATTCCAGTG
TCTGTGTCCCACTGGTTTCTCTGGAAACCTCTGTCAGGTGAGTGGGGCTAGAACCTCCAA
AGGCCAAGTTTCTACAGCAGAGCAGCTCCCCACCCCTGCCCAGTTCCCTCCTGATCTCTG
GACTTGCCATGAAATCTGTTACCATCCAGTGACCAGGGACTTCTGCTCGGTGAACACAGG
TGGAAGGACACCAAGGAGTGGGTTTTGTTCACAGTGTCGGCAGCCTGTCCATTTGTGCCC
CCAACTCCGTGCTCTCCCACTCTGCCCTGTTTTGCCGTGCATCCCTGGACAGTCTCTAGG
ACAAAACTGTCTATTATGTAGGGTGTTCTCCAAATGGAAGGAGGTGAAGGGGTCAGATTC
CAGCAACTGCTGCCCCAGAGCACTTCTAATCACACCTCTGTTTCCTCTCCCCCTTCCCTG
CTCCTGGCAGCTGGACATAGATTATTGTGAGCCCAATCCCTGCCAGAACGGTGCCCAGTG
CTACAACCGTGCCAGCGACTATTTCTGCAAGTGTCCCGAGGACTACGAAGGCAAGAACTG
CTCCCACCTCAAAGACCATTGCCGCACGACTCCCTGCGAAGGTACCTGCCCCACCTGTGG
TCCTGGCTGGCCTCCGAGGCTCACTCCTCTACCCCTATTTGTCAGTACCCCCCAATATTC
TCAGCAGCTGGCTGGGTGAGACGGTAACGCCCTAACCTGCCTCGCTAGGGGAGCCAGAAC
ACGTGGAAACCCTCTGTCCGGGCCTGCCAGTGGGAGAACCTTGTCCACACGTAGCATTCC
TGTTCTGGGGTGGGCTGATTTGCAACCCTGAGACCTTCCCCAAAGCAGTGCTGCCGGAAG
AACTGTCTGCAGCACTGGAGATGTGTGCTGTCTGCACTGTCGGGTGTGCTAGCCACAGGA
GCATTTAGTGTGACCACTACGACTGGAAACTTCCTTGACTCCAGTTCATTCAAATCTAAA
TAACCCCACGTGTCTCATGGTCGTTGTATTAGGTGGCCTGGCTCTGCGGGTTCTCTGTCC
TTCCTGCTTGCTTAGGTAGGAAGTAAAGCTCCTCCTCCCTGTAGCAAGCTTCTGCCCGAC
TGAGGAGATGTCAGAGGTAGGAGGAGAGGAGCCCACAGACCCAGAGGCTTCTTAGCCACG
TAGGGCGAAATGCCTTAAAAACAAAAACAAAAAAAAAAAACAGAGCTTCTGTCTGTCTGG
AGTCTTGGCTTTGCCTGTCACAGTGTGACGTGGCACATCGCCAGAGTCTTGGCCCCGGGG
TGCCTCTTGATTTCTTACCCTCATTAGCCAAAGGGTGCCTGTTTCCAGGAAAGCGAGCTT
GGGCGGAGGTCCTCAGCTTAGGAATGCCATCCATGCCTTATGGGTCTGTGGCCCTGATCT
GACCCCGAGTCTGCCTTCTAGTGATTGACAGCTGCACAGTGGCCATGGCTTCCAACGACA
CACCTGAGGGGGTGCGGTACATCTCCTCGAACGTCTGTGGTCCCCATGGGAAGTGCAAGA
GTCAGTCGGGAGGCAAATTCACCTGTGACTGTAACAAAGGCTTCACTGGAACGTACTGCC
ATGAAAGTAAGAGCCCTCTTCGGGGAATGGGGTGTGGTGCCCCCCACCCCTCCTCTGCCC
AGTTCAAGGGAGCATTTGAGCTTGATTCGTTTCAGTCGAGACCAGTGACGGTTTACCATT
ACATCTAAGATCTAAGACCGAGTTGTGTTTAATTTATTGAGATCACAGACACCTCTGACC
CCTTCAGCACGTTTATGTTCTGGAATCTTACTAGTTTGGGTGGTCCTGCTTTCTGTAGGC
TTTTCTTCCCTACTCCATCCGAGTAGCTTAGCCTCCAAGGCATTAAATAAAACAGACAAC
GTATCCTCTGGAGGCTTCTGCCTGGTAAGCTGCTTGCTGAAGTCGGAAAAACCTTCCTGA
TGGAGTTGAGGATGTTGAGGTGCCTGGCTGCCCATTTTCTCAGGAGGTGGCCTTTGGGCC
AGTGCCTGGCTATGGCATCGGTTGTCCTTTACTTACCAGCTTTTTTGTCTTCATTTCACA
GATATTAATGACTGCGAGAGCAATCCCTGTAAAAACGGCGGCACTTGCATCGATGGGGTC
AACTCCTACAAATGCATCTGCAGTGACGGCTGGGAGGGGGCCTACTGTGAAACCAGTGAG
TGTGTGGTCTCCCTGGGCCAGACAGCTCTGGGATGCTGGGGTAGGAGGTATCATCAGGGA
AGCTTGCCTTTTCAATGGGAATTAGATAGTAAGACCCCAACCGTTCCTGTCCTGGGACAG
CATTGCCTGTGTCCCGTCTGTATTTGTGAATGCTGAGGACGGCCAAAGGGGAGGAACTTC
TGCTGAGGAGCTGTGGAAACAGACTGTCATCAGGCTAAATATCCCTGCTTGCAGAGAAAG
TAGTCTTCTGTTTTAATAGTAATTTGTGCCTGTCCTGATTTTTAACTTCCATGGGAGTGA
CAGAGGCCTGCCCTTGTCTTGCCACCTTGTGGGGTTTAACCCAGCTGAGGAGAAGAGAAG
GTGTTTAAGTCCCAGGATGGATGGCTCAGCTGGTCTCGGACCTTAGAGTGGAAAGCTGTG
GGTGGAAGTGGCCATAGGAAGGGTGTGGCTCTGACACGCCGTGGATGGCCCTAGAAGCTT
CTGGAGCTGGGTGTCTCCTCTTTGATTCTAGACATTAACGACTGCAGCCAGAATCCCTGC
CACAATGGTGGCTCATGTCGTGACCTCGTCAACGACTTCTACTGTGACTGTAAAAACGGG
TGGAAAGGAAAGACCTGCCATTCGCGTAAGTGGTAGCTGGCTGGGGCCCCCTCTCCGCAT
CCCCTTCCCACCACCCTGTCTTCTGTGAAGGGACCTCACATGGAGATCCCCCTCCTCCAG
GTGACAGCCAGTGTGATGAGGCCACGTGCAACAACGGTGGCACCTGCTATGACGAAGGGG
ATGCTTTCAAGTGCATGTGTCCTGGAGGCTGGGAAGGGACGACCTGCAACATAGGTAGCT
TTCTGCCCCACATGGGGTCGGCAGTGGGTGTGGCCATCTCAACACCTAGGACCGCTTTCC
TGGCGGTCTGCTGCTCGGCTCTCCTACTCTCTTTGGATCCTCAGGGAGGGTGGTCCTGTC
TTGTCTTGTCTTACAGCCCGAAATAGTAGCTGCCTACCCAACCCTTGCCACAACGGGGGC
ACCTGTGTGGTCAACGGGGACTCCTTCACGTGTGTCTGCAAAGAAGGCTGGGAGGGGCCG
ATCTGCGCTCAGAGTGAGTGTCCCCTGCCCCACCCGCCTTGAACTCCACAGAGGGCTGCA
GGAGTTCATCCTGACACCCTCAGGGACTCGCGATGGAAACCAGAAGCAGGGCTGTTTGAT
GTCTGCTTCTGCATCACCTGGGTCTGTTCTAATGCACTTGATGGACCGCAGCCCTGACCT
GGGCATGAGAAAGTCGCCGATGAGATAAGTGGAGACAGATGGTGTCTCTGTTCCCAGCGT
GGGTCTGACCAGCAGCCTGTCTCCTTCCATTCTCACAGCCCCCTTCCCGCCCTGAGCGGG
GCCTCTTCCTGTGTAATGTCCCACGGCACACAGAACAAAGTGTGGTGCAGGCCTGGTTCC
AATTGAGCAGAGGTCTTAAGCGCATTTTGCCGGTGTAAACTACAGTGTGATCATTGGGGT
ATTAATGGAAATAAGTGGTGCTTCTACTTAGGGTTAAAACTGCTTTGCCCGGTGAAGGCC
TCGCTGTTAAGTCGTCTTTTTACTTTGCAGATACCAATGACTGCAGTCCTCATCCTTGGT
AAGCATGAGCGCCTTGGAAGCCAGCCCTTGCTGGCTACGATGTGCATGCGCATCACCTGG
GGGTCCTGTCTAAGATGCAGGTTCTGAGGCAGGAGGTCTGGGTGGGGCTGCAGACCCTTC
CTGTCTAACAAGCTCCCAGGTCCTGCTGGTCCGTGGACCACACTCTGATTAGCCAGGTCA
TGTCTTGGTTGGGGGCTGTGATTATTCTCAACGATTCCCAACCTTGGCTGTGCATAAGAA
TCACCTGGGGAGCTTTTAGGAGTCCCAATGCCTGGATTGCAGCACAGCCCCATGAAATGA
GACTCTCTGGGGTTGGGACCCAGGCACCATTATTTTGAAATCTCCCCAGGCTACAGGGGT
GTGTGGCCAGGGGTGAGAGCTGCTGAATGGAACTGGCGTGGCCGCCTGTGGTCAACTGGC
TGGTGGAGTCGTGGATCAGGAGGTCGGTAACTGAGGCCGTCTTTGCTTTCTTCTTAGTTA
CAACAGTGGCACGTGCGTGGATGGAGACAACTGGTACCGGTGTGAGTGTGCCCCTGGTTT
TGCTGGACCCGACTGCAGAATAAGTAAGGACCAGCCCTGGTTGGTTTCCCCAGTGGCCTT
CTTCCCCCTGATTCAGCCCTCAGCCTGAGACTTTTCATCATGAAAAGTTAAGGTTACTGC
TCTCTGTAGCTCTGAGAGGCACAGAACCTCCCACCTCCAGCTGGGATTAGGATAGCCTGG
CTGGAATGTCCCAGGTCCTTGCTGTCCCTTCCCCAGGGGACACAGCCCTCTTCCAAGGAA
GAGAGGAGCAGCCACAGGATCTGATGATTAATCTGTTCTGAGGCCCCAAGGAAAGGGTGC
TTTTCCTCATGACTACCTGAAATTAGGGGCGAGTGGACATGTGCCACAGAGGACCGAGCT
AAGGACAGTGGCCAGGACTAGGGTTGTGGTTCCCAAAGGATTGATCCGACCCTAGTGAAA
TTAAAATGGGAGACAGGCAGGGTGCCTAAAGTCTATTTTGAGCCTTTTCCTACTTTCTTA
TATGCCTTTTAGTTTGGGAGGAGGGAGTGATACTTGTATTTTTTGTTATTCTCATCCCCA
AATTATTTGGAATTTTACTAGTGTTTGAGATCCCCCAAGTCTGGGGCTGACTGGTGTTCA
CCAAAATGGGGTACATTTGGTCTATCCCATGTTTCTAAATTGTGTTGGCAAACTGTAGCC
TTGGCCCACTGCCTGTTTTTGTAAATAAAGTTTTATTGGCACACAGCTACTCTTTTATAT
ACTATCTAAAGCTGCTTTCATGCTATAACAAGGGTAGTCATGACAAAGCCTACAAGGCCT
GCAAGGGTTAAAATATTTGACCTTTCCCAAGAACTTTACCCTTGCTCTTAAGTTGTAGTA
TCTGATGAAAAGGAACTTACTTAATTAAAAAGTGCCGCCTCCTGAAATGATTCTCAGCAG
CTTGAAGTCAGGCTAATGATGATCTTTGGGAGGAAGGGACTAGTGGAAATTTGGGGGCTA
CTGATCGGTTTATTTGGGTGTTGGTTACAGAATTCATCAAGCTTATACCTAGGGTTTACA
CGCTCTTCTATCTGTATGTTAGATTAGTAAAAACTTAGAAGAATGTCATTGCTTCCCATT
GGAGCCTAAAGTTGGTCTCCATTTCTCCTAGACATCAATGAATGCCAGTCCTCACCGTGT
GCCTTTGGGGCAACCTGTGTGGATGAGATTAATGGCTACCGGTGCGTCTGCCCCCCAGGG
CACAGCGGTGCCAAGTGCCAGGAAGGTATGTGTGGGTCAGGCTGCAGCTGCCCATGTGTC
TTGTGGGGGCAGTGGGCACTGCAGTCACCACGGTTACCCTTTGACTGTTCCTGAGTAGGC
AAAGGAGAACATGGCGGGGATATGCACAGGGGGCCACAGGGTCGGGGAAACTTAGGGGGT
CCCCTACAAACAGGATAGCTGTTACTAGTCAACTCCCGAAGTTTTGCCGTGTGACAAACC
ACCCCCAAACCTGCGGCTTACAGCAGGCTTTTATTTGCATTGCTCATGGGCACGCAGGCT
GGGCTCAGTTCTGTTCTGTGTGCTTCGTCACCCTCCTTGGGCCAGCGGCCATATCTCAAC
TGCGAGAGGCTGCCCACATCACATCCCCTAGTGCTGCACAGGTCCCACAGGCCCCACAGG
TCCTGAGGCCACGTGCAAAGGCAGCAGGTGAGGGAAGAACACCTATTGCCCCGTGGCACA
GGGCAAAGGTACAGCCCTTCCCTGACAGGGAGGGAGTGAAGAATAGAACGGTCAATAGAA
AAGGAAATCTGTTCCACTCATGCGCTCTACCTTTCAGTTTCAGGGAAACCTTGCATCACT
ATGGGAAGTGTGATCCCAGATGGGGCCAAGTGGGACGATGACTGCAATACCTGCCAGTGC
CTGAACGGAAGGATCGCCTGCTCCAAGGTACAGCATGAGGGCCCTCTGCCTCTCACCTGG
GTTCTAGATTGCTGCTGGGTGTTTTCTCCTGAGCTGCCCGCTCCATCTCCTCTCCCCCTG
CTCCCCAGGTCTGGTGCGGCCCTCGACCTTGCCTGCTCCACAAAGGGCACAGCGAGTGCC
CCAGCGGGCAGAGCTGCATCCCTATCCTGGAGGACCAGTGTTTCGTCCGCCCCTGCACGG
GCGTGGGCGAGTGTCGGTCTTCCAGTCTCCAGCCGGTGAAGACCAAGTGTACCTCTGACC
CCTACTACCAGGATAACTGTGCCAACATCACATTCACCTTCAACAAAGAGATGATGTCAC
CGGTACGAATAACTTCTCATTCAGTTTGGGCTGGCGGTGTGTGTCTCCTGGTTCCAAAGC
AGGAGCATTGCAAGCTAGGATCTTCCCCTTAGCTTAAATTCCTAACTTGCAAGGAAACGT
CAGCCTTTTTTATTTTTTTCCTGTTCCTCGCAATATCTTACATGTGTGGTTTTTACATGT
GTTTCTAGGGTCTAACCACAGAGCATATCTGCAGCGAATTGAGGAATCTGAATATCTTGA
AGAACGTTTCTGCTGAATATTCAATCTACATAGCTTGTGAGCCTTCCCCTTCTGCGAACA
ACGAAATACATGTGGCCATTGTAAGTATGAGACCCAGTCACGCCTCATGACTCGACTGCA
GGGTAGTTCTCAAAGTCCAGTTAGCCGGTGTCCAGACAGAGCAAGGACACTGAAACAGCT
TGGCACACGGGCTAAGTCACTTTCAGTGATGGCTGCTGTGTAGTGTTGCCCTGATCCCAC
ATCTGGCCCTTCTTTTTCCAGTCTGCTGAAGACATACGGGACGATGGAAACCCTATCAAG
GAAATCACTGACAAAATCATAGACCTCGTTAGTAAACGTGACGGGAATAGCTCGCTGATC
GCTGCTGTGGCGGAAGTGCGAGTACAGAGGCGGCCTCTGAAGAACAGAACAGGTAGGTGG
CAGGTGGGGACAGTCCTTTCGCGTGAGGACTGTCCGACGGATGGCTCCTCGGCTCACCGT
TCAGGGAGGGCATTAGCTGACAGCTTCTCTCAAGGAAACTCCCCACCCCCCCCACCCCCA
TTTAACTGGCGGAAAAACTGGGACCAGCTGCCATGACCAAGGTCCCGTGGTGATGGGCAG
AAGTACCGTTTTTTCCCTGCTGCCAGGCTGTGCTCTGGGAAGCAGGCACCCCTGCGGAGC
ATGCATTGTTTGCAGACCCCCTGCAAAGCCCTAGGGCAGAGCATTGCTGCGCCTCGAGCT
GGTGGCATCCTGGTGTCGTGGGATCACCCGGCCTAAGCACTGAGAGACCCGGGTTTGATA
GGCAGCTGTATGGCCCTGCAGGGGAGCCACACCTTTAGACCACCATTATTCTAGCTAGGT
GAGAACCGGGGTGCCTGGGAGCACGGCCCATCGGAGCTATGCACGCTCAGATCGGGTTTC
TGGTAATGCTGAAAACCAGCTCCTCTAATGAGCTGCCGCCTGAGTAGCCAGACTTCCCTT
GCGTGAACCAGATGCTCCTCTCCACACGGGAGTCAGGAGCCTGCAGGAGCTGCTGCTGTG
CCCGGTCCCTGGAGACCAGCACATCACGGTCCCCACGGGCCCTGGTCCTGTGCTTATCAA
GCTGTGAAGTGACAATATGTGGTTGTGAAAAGGGACTTGTCCCCATGGCACCCCTTTCAG
GAATTAAGGACAGGAGCGCCACTGTTGGTATTCTTTTGTTCGTGTGATGGCTTTTTTTTT
TCCCCTTCTTCTTTGAGAGTTAATTGGTTTTGTGCCCGCCTTACAGATTTCCTGGTCCCT
CTGCTGAGCTCTGTCTTAACGGTGGCTTGGATCTGTTGCCTGGTGACGGCCTTCTACTGG
TGCGTGCGGAAGCGGCGGAAGCCCAGCAGCCACGCGCGCTCGGCCTCCGAGGACAACACC
ACCAACAACGTGCGGGAGCAGCTGAACCAGATCAAGAACCCCATCGAGAAGCATGGGGCC
AACACGGTCCCCGTCAAGGACTACGAGAACAAAAACTCCAAAATGTCAAAAATAAGGACA
CACAACTCAGAGGTGGAGGAGGACGACATGGATAAGCACCAGCAGAAAGCCCGGTTTGCC
AAGCAGCCCGCATACACGCTGGTAGACCGAGAGGAGAAGCCTCCCAACGGCACGCCAGCC
AAACACCCAAACTGGACAAACAAACAGGACAACAGAGACCTGGAAAGTGCCCAGAGCCTA
AACCGGATGGAGTACATCGTATAGCAGACAGCAGGCGCTGCCGCTAGGTAGAGTCGGAGG
GTGCTGGGGGCTTGTAGTTCAGTTCTTCAGCTGTCCTGTCCTCTTCCAGTCTGAGGCTGT
CGTTGACTTAGAATCCTGTGTTAATTTTTGTTTTTGACAAGCTGGCTTACACTGGCAATG
GTAGTTTCTGTGGTTGGCTGGGAAATCCAATGCTGCAGCTCACAGCTATGCAAAAACCAG
TCCAAAGTGCCGCCCCCCCCCCCCCCCACCGCCGCCCCTGCCCCACAGCTGACACCTCTT
GGACCAGGCTCCCAGGAGAATGCCCGGCCCCGGGGCCTTGAGCTTCCACCTCTGCCAGAT
GTCCCGATGGTGATGCAGTCTTAGGATCATAGTTTTTATTTATATTTATTGACTCTTGAG
TTGTTTTTGTATATTGGTTTTATGATGACGTACAAGTAGTTCTGTATTTGAAAGTGCCTT
TTGCAGCTCAGAACCACAGCAACTATCACAAATGAGTTTATTATTTATTTTTTTTATTGT
ATTTTGTTGTTGGGGGAGGGGGGACCTTGATGTCAGCAGTTGCTGGTAAAAATGAAGAAT
TTAAAGAGGAAAAATGTGTCAAAAGTAGAATTTTGTATAGTTATGTAAATAATTCTTTTT
TATTAATCACTGTGTATATTTGATTTATTAACTTAATAATCAAGAGCCTTAAAACATCAT
TCCTTTTTATTTATATGTACGTGTTTAGAATTGAAGGTTTTTGATAGCATTGTTAAGTGT
ATGGCTTTATTTTTTTGAACTTATTTTCTTATTACGTGTTGCCTATAAGCCAAAACTAAG
GTGTTTGAAAATAGTTTAAAACAATAGGATGGGCTTCAGTGCCTGGAATACTGGTGGAAT
TTTTTTTTTTTTTTTTTTGTACGACGTCAGATGTTTAAAACACCTTCTATAGCATCACTT
TAAAACACGTTTTAAGGACTGAGGCAGTTTGAAAATTAGTCTAGAAAAGCAGGTGGTTTT
TTTGTTTTTTTATGCTTTAGACTTGAAAAGAGACAGGCAGGTGATCGGCTACACAGCAGT
TTAAGAGAACATGTTGAGCTTCAACTTAACGTAGCCAAAATGTGAGTGGTTGAATATCAT
TAAAAATATCAAATTGTGTGAAGTTGGAAGCACACCAATCTTATTTTGTAAATTCTGATT
TCTTTTCACCATTCGTATGTAATACTGAACCACTTGTAGATTTTTTTTGTTTGTTTGTTA
TCTACTGCATTTAGGGAGTATTCTTATAAGCTAGTTGAATACTTGAACCTTAAAATGTCC
AGTAAGATCACTGTTTAGATTTGCCATAGAATACACTGCCTGCCTTAAGTGAGGAAATAA
AAATGCTATTTACAAAGTTGAAGATCAAAACGGCTTCTAAAACAGTTCACGTCGTCGGCT
CACTACGGAGACCGTGAAGATACTCTGTATTGTCCTATTAGTGTTATGAACATAAAAATG
CATCTTCGATGTGTTGTTCCTGGCAATAAATTTTGAAAAGTACTATTTATTAAATTTTTT
GTATGAAACCTGGAACAGTGTGGCCTCTTCTGAGCTTCTGTAGTTTTGCTTGGCTCTACC
TTGTGCCTTTGCCACCCCACCGAGGCCGTTCTGGTAATCAGGGTGTGCTAAACAGGCTGT
GCTTGACAGGGGTGCTGAGGAAGACTGAAAGCCTTTTCAGCCACAAAATTTCAGCTGAAG
TTCTCAAAAAACCATGGGCTGCTGTGAAGCCAGAGCTGTGAGAGCCCCAGCTCGGGGGTC
CTGGATTCCCTTGTTATTCAACAGCAAGTGTGAATACTGCTTGAATAAACACCACTGGAT
TAATGGCCTGTAGTGTGGAGGTGAATTGTTTGTGAGCACTCACGGGAAATGCCCACCGCA
CTAAAAGGGGTTTTAAAAAGCTTGGAATTAGTGTTGGGGGAGGAAGAGGGAGATTTGGTG
CTTTCGGTCTTTTTTTTTTTTTTTTTCCCCTGGCTGCTTGGATTAATATTAATGTCCTGC
CGTCATTTTAGACCACACCTTCGTGGCTTCTGTGCACGCTTACGCCTCCAGGGCGGTTTC
CTCGCTGCACAGGAGGTGGTAGTTACAAGCTTCTGGGGTTAGAGCCATGTACTCATCACC
TTGTTCCAAGGGAATCCGTGCGGCTGTGGGGTGCCAGGTATCTGGCCTTCCTGGAGGTTC
CAGAGGCCAACCATCGCCACCTGTTCAGGACAGGTGAACTGTGGTTCTCCTTGTGGCTGC
TGGTGCCAGAGGCCAGAATGGCCCTGCCCGCACATCACGGGTGGGGCTGGGAAAGCTTTG
CCTCTGGCACCTCAGTAGTCAGAGATGAGCCAGAGTGACGGGCACATGGATGGCTTCCAG
CAGCTTCATGGAGTTGCCAGGAGTTCCCTGCTTCTGATGAGTGAGCCAAAGCCTAACCTC
CCTGGAGCTGTCCCCTAGCGCTGCACATTCAGGCCATCCGTCCACCTCTGATTTTCATAG
TATGCTTTTACTCTTTCTTGGGAAAATTCTCTTCTACTGGACTAGGAACTGTGTTCTCTG
ATGGGCATCTGCTTAAGAATGGGCAATGGGCTTTAAAAATATCCCAAGTTTCCTGTGCAT
GAGCAGAGTACTGAGGAGTCAGAATCCCTAACTATGCGTTCGGGCTTGCTGGGAAAGCAG
CCCGAAGGACCTGGAATCCCGTGACGACTCCCTGCCCTGGTGTAGCAATACTTTCTGTCC
CTAATCTCTGCTGTGGGACAGCTGGTTTCTGCAAGGTGGGGTCACAGGGAGAAGTGCTCT
GGTACAGACCCAGGGAGCCAGGACACAGTCCGATTTAGCAGCACTCAAACACCATCCCGA
GGCAATATCTTGCTCCGGTGGTTTGTGAACTTGGATTCTTCAGGACCCCAGAATTCCTTG
GGTAGTTGCCAAGTGCTTGCTTTTGTGCCAAGAGGAAAAAAAAAAAAAAGTTTAACGAGC
CTCTGCAATTCTTGATTCCAGGGGGGAAGTGATCTTGAGATTCACATTTGTTGTTTTAAG
AAGCCATCTCAGTAGTGAGAAGGCAGAGATCAGGCTCCTCTGGGCATGGCTCAAAGCCAG
GACCTTAATCATAATGGGCAGTTCCCCTCTTTTCCAGGTAGAGGGAATCCGGCAAGGGAG
CCCTGGTTATCGTGGGAACAAGCTGGAAAAGAAGGGACTCCATACCTTTCCCATTCCCCA
GCAATGTTCTGAGCCCTCGTTTAAGCAGACCCT
Canis familiaris jagged 1 (JAG1), cDNA
(SEQ ID NO: 7)
ATGCGGTCCCCACGGACGCGCGACCGGCCCGGGCGCCCCCTGAGCCTCCTGCTCGCCCTG
CTCTGCGCCCTGCGAGCCAAGGTGTGCGGCGCCTCGGGCCAGTTCGAGCTGGAGATCCTG
TCCATGCAGAACGTGAACGGGGAGCTGCAGAACGGGCACTGCTGCGGCGGCGTCCGGAGC
CCGGGGGACCGCAAGTGCTCTCACGACGAGTGTGACACGTACTTCAAAGTGTGCCTCAAG
GAGTACCAGTTCCGCGTCACGGCCGGGGGGCCCTGTAGCTTCGGGTCGGGCTCCACGCCA
GTCCTCGGGGGCAACACCTTCAATCTCAAGGCCGGCCGTGGCAGCGAACGCAACCGCATC
GTGCTGCCTTTCAGTTTCGCCTGGCCGAGGTCCTACACTTTGCTGGTGGAAGCGTGGGAT
TCCAGTAATGACACTCTCCAGCCCGACAGCATTATTGAAAAGGCCTCTCACTCAGGCATG
ATCAACCCCAGCAGGCAGTGGCAGACACTGAAGCAGAACACAGGGGTTGCCCACTTTGAG
TATCAGATCCGTGTCACCTGTGATGACTATTACTATGGCTTTGGCTGCAACAAGTTCTGC
CGACCCAGAGATGACTTCTTTGGACACTATGCCTGTGACCAGAATGGCAACAAAACTTGC
ATGGAGGGCTGGATGGGCCCAGAATGTAACAAAGCTATTTGCCGACAGGGCTGTAGTCCC
AAGCATGGATCTTGCAAACTCCCAGGTGACTGCAGGTGCCAGTACGGCTGGCAAGGCCTG
TACTGCGACAAGTGCATCCCGCACCCCGGGTGTGTCCACGGCACCTGTAACGAGCCCTGG
CAGTGCCTCTGCGAGACCAACTGGGGCGGCCAGCTCTGCGACAAAGATCTCAACTACTGC
GGGACTCGGCAGCCCTGCCTCAACGGGGGCACCTGCAGCAACACGGGCCCTGACAAGTAC
CAGTGCTCCTGCCCCGAGGGCTACTCAGGCCCCAACTGTGAGATCGCGGAGCACGCCTGC
CTGTCCGACCCCTGCCACAACAGAGGCAGCTGCAGGGAGACCTCGCTGGGCTTCGAGTGT
GAGTGCTCTCCGGGCTGGACGGGCCCCACGTGTTCCACAAACATTGATGACTGTTCTCCA
AATAACTGTTCCCACGGGGGCACCTGCCAGGACTTGGTTAATGGATTCAAGTGCGTGTGC
CCACCCCAGTGGACTGGCAAAACGTGCCAGTTAGATGCAAATGAATGTGAGGCCAAACCT
TGTGTAAATGCCAAGTCCTGTAAGAACCTGATTGCGAGCTACTACTGTGATTGCCTCCCT
GGCTGGACGGGTCAGAATTGTGACATAAATATTAATGACTGCCTTGGCCAGTGTCAGAAT
GATGCCTCCTGTCGGGATTTGGTTAATGGTTATCGCTGTATCTGTCCACCTGGCTATGCA
GGCGATCACTGTGAGAGAGACATCGATGAATGCGCCAGCAACCCCTGTTTGAATGGGGGT
CACTGTCAGAATGAAATCAACAGATTCCAGTGTCTGTGTCCCACTGGTTTCTCTGGAAAC
CTCTGTCAGCTGGACATAGATTATTGTGAGCCCAATCCCTGCCAGAACGGTGCCCAGTGC
TACAACCGTGCCAGCGACTATTTCTGCAAGTGTCCCGAGGACTACGAAGGCAAGAACTGC
TCCCACCTCAAAGACCATTGCCGCACGACTCCCTGCGAAGTGATTGACAGCTGCACAGTG
GCCATGGCTTCCAACGACACACCTGAGGGGGTGCGGTACATCTCCTCGAACGTCTGTGGT
CCCCATGGGAAGTGCAAGAGTCAGTCGGGAGGCAAATTCACCTGTGACTGTAACAAAGGC
TTCACTGGAACGTACTGCCATGAAAATATTAATGACTGCGAGAGCAATCCCTGTAAAAAC
GGCGGCACTTGCATCGATGGGGTCAACTCCTACAAATGCATCTGCAGTGACGGCTGGGAG
GGGGCCTACTGTGAAACCAACATTAACGACTGCAGCCAGAATCCCTGCCACAATGGTGGC
TCATGTCGTGACCTCGTCAACGACTTCTACTGTGACTGTAAAAACGGGTGGAAAGGAAAG
ACCTGCCATTCGCGTGACAGCCAGTGTGATGAGGCCACGTGCAACAACGGTGGCACCTGC
TATGACGAAGGGGATGCTTTCAAGTGCATGTGTCCTGGAGGCTGGGAAGGGACGACCTGC
AACATAGCCCGAAATAGTAGCTGCCTACCCAACCCTTGCCACAACGGGGGCACCTGTGTG
GTCAACGGGGACTCCTTCACGTGTGTCTGCAAAGAAGGCTGGGAGGGGCCGATCTGCGCT
CAGAATACCAATGACTGCAGTCCTCATCCTTGTTACAACAGTGGCACGTGCGTGGATGGA
GACAACTGGTACCGGTGTGAGTGTGCCCCTGGTTTTGCTGGACCCGACTGCAGAATAAAC
ATCAATGAATGCCAGTCCTCACCGTGTGCCTTTGGGGCAACCTGTGTGGATGAGATTAAT
GGCTACCGGTGCGTCTGCCCCCCAGGGCACAGCGGTGCCAAGTGCCAGGAAGTTTCAGGG
AAACCTTGCATCACTATGGGAAGTGTGATCCCAGATGGGGCCAAGTGGGACGATGACTGC
AATACCTGCCAGTGCCTGAACGGAAGGATCGCCTGCTCCAAGGTCTGGTGCGGCCCTCGA
CCTTGCCTGCTCCACAAAGGGCACAGCGAGTGCCCCAGCGGGCAGAGCTGCATCCCTATC
CTGGAGGACCAGTGTTTCGTCCGCCCCTGCACGGGCGTGGGCGAGTGTCGGTCTTCCAGT
CTCCAGCCGGTGAAGACCAAGTGTACCTCTGACCCCTACTACCAGGATAACTGTGCCAAC
ATCACATTCACCTTCAACAAAGAGATGATGTCACCGGGTCTAACCACAGAGCATATCTGC
AGCGAATTGAGGAATCTGAATATCTTGAAGAACGTTTCTGCTGAATATTCAATCTACATA
GCTTGTGAGCCTTCCCCTTCTGCGAACAACGAAATACATGTGGCCATTTCTGCTGAAGAC
ATACGGGACGATGGAAACCCTATCAAGGAAATCACTGACAAAATCATAGACCTCGTTAGT
AAACGTGACGGGAATAGCTCGCTGATCGCTGCTGTGGCGGAAGTGCGAGTACAGAGGCGG
CCTCTGAAGAACAGAACAGATTTCCTGGTCCCTCTGCTGAGCTCTGTCTTAACGGTGGCT
TGGATCTGTTGCCTGGTGACGGCCTTCTACTGGTGCGTGCGGAAGCGGCGGAAGCCCAGC
AGCCACGCGCGCTCGGCCTCCGAGGACAACACCACCAACAACGTGCGGGAGCAGCTGAAC
CAGATCAAGAACCCCATCGAGAAGCATGGGGCCAACACGGTCCCCGTCAAGGACTACGAG
AACAAAAACTCCAAAATGTCAAAAATAAGGACACACAACTCAGAGGTGGAGGAGGACGAC
ATGGATAAGCACCAGCAGAAAGCCCGGTTTGCCAAGCAGCCCGCATACACGCTGGTAGAC
CGAGAGGAGAAGCCTCCCAACGGCACGCCAGCCAAACACCCAAACTGGACAAACAAACAG
GACAACAGAGACCTGGAAAGTGCCCAGAGCCTAAACCGGATGGAGTACATCGTATAGCAG
ACAGCAGGCGCTGCCGCTAGGTAGAGTCGGAGGGTGCTGGGGGCTTGTAGTTCAGTTCTT
CAGCTGTCCTGTCCTCTTCCAGTCTGAGGCTGTCGTTGACTTAGAATCCTGTGTTAATTT
TTGTTTTTGACAAGCTGGCTTACACTGGCAATGGTAGTTTCTGTGGTTGGCTGGGAAATC
CAATGCTGCAGCTCACAGCTATGCAAAAACCAGTCCAAAGTGCCGCCCCCCCCCCCCCCC
ACCGCCGCCCCTGCCCCACAGCTGACACCTCTTGGACCAGGCTCCCAGGAGAATGCCCGG
CCCCGGGGCCTTGAGCTTCCACCTCTGCCAGATGTCCCGATGGTGATGCAGTCTTAGGAT
CATAGTTTTTATTTATATTTATTGACTCTTGAGTTGTTTTTGTATATTGGTTTTATGATG
ACGTACAAGTAGTTCTGTATTTGAAAGTGCCTTTTGCAGCTCAGAACCACAGCAACTATC
ACAAATGAGTTTATTATTTATTTTTTTTATTGTATTTTGTTGTTGGGGGAGGGGGGACCT
TGATGTCAGCAGTTGCTGGTAAAAATGAAGAATTTAAAGAGGAAAAATGTGTCAAAAGTA
GAATTTTGTATAGTTATGTAAATAATTCTTTTTTATTAATCACTGTGTATATTTGATTTA
TTAACTTAATAATCAAGAGCCTTAAAACATCATTCCTTTTTATTTATATGTACGTGTTTA
GAATTGAAGGTTTTTGATAGCATTGTTAAGTGTATGGCTTTATTTTTTTGAACTTATTTT
CTTATTACGTGTTGCCTATAAGCCAAAACTAAGGTGTTTGAAAATAGTTTAAAACAATAG
GATGGGCTTCAGTGCCTGGAATACTGGTGGAATTTTTTTTTTTTTTTTTTTGTACGACGT
CAGATGTTTAAAACACCTTCTATAGCATCACTTTAAAACACGTTTTAAGGACTGAGGCAG
TTTGAAAATTAGTCTAGAAAAGCAGGTGGTTTTTTTGTTTTTTTATGCTTTAGACTTGAA
AAGAGACAGGCAGGTGATCGGCTACACAGCAGTTTAAGAGAACATGTTGAGCTTCAACTT
AACGTAGCCAAAATGTGAGTGGTTGAATATCATTAAAAATATCAAATTGTGTGAAGTTGG
AAGCACACCAATCTTATTTTGTAAATTCTGATTTCTTTTCACCATTCGTATGTAATACTG
AACCACTTGTAGATTTTTTTTGTTTGTTTGTTATCTACTGCATTTAGGGAGTATTCTTAT
AAGCTAGTTGAATACTTGAACCTTAAAATGTCCAGTAAGATCACTGTTTAGATTTGCCAT
AGAATACACTGCCTGCCTTAAGTGAGGAAATAAAAATGCTATTTACAAAGTTGAAGATCA
AAACGGCTTCTAAAACAGTTCACGTCGTCGGCTCACTACGGAGACCGTGAAGATACTCTG
TATTGTCCTATTAGTGTTATGAACATAAAAATGCATCTTCGATGTGTTGTTCCTGGCAAT
AAATTTTGAAAAGTACTATTTATTAAATTTTTTGTATGAAACCTGGAACAGTGTGGCCTC
TTCTGAGCTTCTGTAGTTTTGCTTGGCTCTACCTTGTGCCTTTGCCACCCCACCGAGGCC
GTTCTGGTAATCAGGGTGTGCTAAACAGGCTGTGCTTGACAGGGGTGCTGAGGAAGACTG
AAAGCCTTTTCAGCCACAAAATTTCAGCTGAAGTTCTCAAAAAACCATGGGCTGCTGTGA
AGCCAGAGCTGTGAGAGCCCCAGCTCGGGGGTCCTGGATTCCCTTGTTATTCAACAGCAA
GTGTGAATACTGCTTGAATAAACACCACTGGATTAATGGCCTGTAGTGTGGAGGTGAATT
GTTTGTGAGCACTCACGGGAAATGCCCACCGCACTAAAAGGGGTTTTAAAAAGCTTGGAA
TTAGTGTTGGGGGAGGAAGAGGGAGATTTGGTGCTTTCGGTCTTTTTTTTTTTTTTTTTC
CCCTGGCTGCTTGGATTAATATTAATGTCCTGCCGTCATTTTAGACCACACCTTCGTGGC
TTCTGTGCACGCTTACGCCTCCAGGGCGGTTTCCTCGCTGCACAGGAGGTGGTAGTTACA
AGCTTCTGGGGTTAGAGCCATGTACTCATCACCTTGTTCCAAGGGAATCCGTGCGGCTGT
GGGGTGCCAGGTATCTGGCCTTCCTGGAGGTTCCAGAGGCCAACCATCGCCACCTGTTCA
GGACAGGTGAACTGTGGTTCTCCTTGTGGCTGCTGGTGCCAGAGGCCAGAATGGCCCTGC
CCGCACATCACGGGTGGGGCTGGGAAAGCTTTGCCTCTGGCACCTCAGTAGTCAGAGATG
AGCCAGAGTGACGGGCACATGGATGGCTTCCAGCAGCTTCATGGAGTTGCCAGGAGTTCC
CTGCTTCTGATGAGTGAGCCAAAGCCTAACCTCCCTGGAGCTGTCCCCTAGCGCTGCACA
TTCAGGCCATCCGTCCACCTCTGATTTTCATAGTATGCTTTTACTCTTTCTTGGGAAAAT
TCTCTTCTACTGGACTAGGAACTGTGTTCTCTGATGGGCATCTGCTTAAGAATGGGCAAT
GGGCTTTAAAAATATCCCAAGTTTCCTGTGCATGAGCAGAGTACTGAGGAGTCAGAATCC
CTAACTATGCGTTCGGGCTTGCTGGGAAAGCAGCCCGAAGGACCTGGAATCCCGTGACGA
CTCCCTGCCCTGGTGTAGCAATACTTTCTGTCCCTAATCTCTGCTGTGGGACAGCTGGTT
TCTGCAAGGTGGGGTCACAGGGAGAAGTGCTCTGGTACAGACCCAGGGAGCCAGGACACA
GTCCGATTTAGCAGCACTCAAACACCATCCCGAGGCAATATCTTGCTCCGGTGGTTTGTG
AACTTGGATTCTTCAGGACCCCAGAATTCCTTGGGTAGTTGCCAAGTGCTTGCTTTTGTG
CCAAGAGGAAAAAAAAAAAAAAGTTTAACGAGCCTCTGCAATTCTTGATTCCAGGGGGGA
AGTGATCTTGAGATTCACATTTGTTGTTTTAAGAAGCCATCTCAGTAGTGAGAAGGCAGA
GATCAGGCTCCTCTGGGCATGGCTCAAAGCCAGGACCTTAATCATAATGGGCAGTTCCCC
TCTTTTCCAGGTAGAGGGAATCCGGCAAGGGAGCCCTGGTTATCGTGGGAACAAGCTGGA
AAAGAAGGGACTCCATACCTTTCCCATTCCCCAGCAATGTTCTGAGCCCTCGTTTAAGCA
GACCCT
Canis familiaris jagged 1 (JAG1), protein
(SEQ ID NO: 8)
MRSPRTRDRPGRPLSLLLALLCALRAKVCGASGQFELEILSMQNVNGELQNGHCCGGVRS
PGDRKCSHDECDTYFKVCLKEYQFRVTAGGPCSFGSGSTPVLGGNTFNLKAGRGSERNRI
VLPFSFAWPRSYTLLVEAWDSSNDTLQPDSIIEKASHSGMINPSRQWQTLKQNTGVAHFE
YQIRVTCDDYYYGFGCNKFCRPRDDFFGHYACDQNGNKTCMEGWMGPECNKAICRQGCSP
KHGSCKLPGDCRCQYGWQGLYCDKCIPHPGCVHGTCNEPWQCLCETNWGGQLCDKDLNYC
GTRQPCLNGGTCSNTGPDKYQCSCPEGYSGPNCEIAEHACLSDPCHNRGSCRETSLGFEC
ECSPGWTGPTCSTNIDDCSPNNCSHGGTCQDLVNGFKCVCPPQWTGKTCQLDANECEAKP
CVNAKSCKNLIASYYCDCLPGWTGQNCDININDCLGQCQNDASCRDLVNGYRCICPPGYA
GDHCERDIDECASNPCLNGGHCQNEINRFQCLCPTGFSGNLCQLDIDYCEPNPCQNGAQC
YNRASDYFCKCPEDYEGKNCSHLKDHCRTTPCEVIDSCTVAMASNDTPEGVRYISSNVCG
PHGKCKSQSGGKFTCDCNKGFTGTYCHENINDCESNPCKNGGTCIDGVNSYKCICSDGWE
GAYCETNINDCSQNPCHNGGSCRDLVNDFYCDCKNGWKGKTCHSRDSQCDEATCNNGGTC
YDEGDAFKCMCPGGWEGTTCNIARNSSCLPNPCHNGGTCVVNGDSFTCVCKEGWEGPICA
QNTNDCSPHPCYNSGTCVDGDNWYRCECAPGFAGPDCRININECQSSPCAFGATCVDEIN
GYRCVCPPGHSGAKCQEVSGKPCITMGSVIPDGAKWDDDCNTCQCLNGRIACSKVWCGPR
PCLLHKGHSECPSGQSCIPILEDQCFVRPCTGVGECRSSSLQPVKTKCTSDPYYQDNCAN
ITFTFNKEMMSPGLTTEHICSELRNLNILKNVSAEYSIYIACEPSPSANNEIHVAISAED
IRDDGNPIKEITDKIIDLVSKRDGNSSLIAAVAEVRVQRRPLKNRTDFLVPLLSSVLTVA
WICCLVTAFYWCVRKRRKPSSHARSASEDNTTNNVREQLNQIKNPIEKHGANTVPVKDYE
NKNSKMSKIRTHNSEVEEDDMDKHQQKARFAKQPAYTLVDREEKPPNGTPAKHPNWTNKQ
DNRDLESAQSLNRMEYIV

In some embodiments, the vector comprises regulatory elements for the overexpression of JAG1, e.g., one or more promoters and/or enhancers. In some embodiments, the promoter(s) and/or enhancer(s) comprise the promoter(s) and/or enhancer(s) present in a Jagged1 gene, such as a human Jagged1 gene. In some embodiments, the promoter(s) and/or enhancer(s) are heterologous promoter(s) and/or enhancer(s) (i.e., not a native Jagged1 promoter and/or enhancer found in a Jagged1 gene). Exemplary promoters and/or enhancers include, but are not limited to, constitutive promoters, tissue-specific promoters, inducible promoters, and synthetic promoters. Exemplary constitute promoters include, but are not limited to, Cytomegalovirus virus promoter (CMV), human ubiquitin C promoter (UBC), Human elongation factor 1α-subunit promoter (EF1-1α), Simian virus 40 promoter (SV40), Murine Phosphoglycerate Kinase-1 promoter (Pgk1), and promoter derived from beta actin (CBA or ACTB). Exemplary tissue-specific promoters include, but are not limited to, human skeletal actin (HSA) and Muscle creatine kinase (MCK) promoters. In some embodiments, the composition comprises a vector comprising alternative regulatory element(s) for JAG1, wherein the alternative regulatory element(s) replace the endogenous regulatory element(s) of JAG1 and increase the expression of endogenous JAG1.

In some embodiments, the alternative regulatory element increases promoter activity. In some embodiments, the alternative regulatory element comprises a Myogenin binding site. In some embodiments, the alternative regulatory element comprises the nucleic acid sequence CAGXTG, where X can be any nucleic acid and is preferably a C. In some embodiments, the vector further comprises other elements suitable for replication, selection or integration (e.g., origins of replication, nucleic acids that encode selectable markers such as antibiotic resistance and/or fluorescent proteins, restriction enzyme sites, barcode sequences, inverted terminal repeats, long terminal repeats, etc.).

In some embodiments, the composition comprises a transcription factor or a vector for recombinant expression of the transcription factor, wherein the transcription factor increases the expression of JAG1. Vectors are described herein. In some embodiments, the transcription factor is selected from the group consisting of myogenin, MyoD, Myf5, MRF4, and Rbp-J.

In some embodiments, the composition comprises a compound that increases the expression of JAG1. The compound may be e.g., a small molecule. In some embodiments, the compound is a compound in Table 2. Table 2 provides compounds from the that have been shown to increase expression of JAG1 (Nextbio).

TABLE 2
Exemplary compounds

Idoxuridine
Lithium Carbonate
Quinacrine
halofuginone
N-(2-aminophenyl)-4-(N-(pyridin-3-
ylmethoxycarbonyl)aminomethyl)benzamide
N-(2-cyclohexyloxy-4-nitrophenyl)methanesulfonamide
phosphonoacetamide
cyanoginosin LR
Nordihydroguaiaretic Acid
Trapidil
vorinostat
Novobiocin
Econazole
Diazinon
Ethacrynic Acid
Diclofenac
nabumetone
Saquinavir
Coumaphos
6-bromoindirubin-3′-oxime
trichostatin A
Acetazolamide
Gentamicins
4-methyl-N-(3-(4-methylimidazol-1-yl)-5-(trifluoromethyl)phenyl)-3-((4-
pyridin-3-ylpyrimidin-2-yl)amino)benzamide
Metyrapone
lapatinib
decitabine
zardaverine
geldanamycin
Histidinol
fragment C, human serum albumin
Emetine
monorden
Cyclosporine
Dichlorvos
Dicumarol
Thapsigargin
Mycophenolic Acid
Caffeine
benzyloxycarbonylleucyl-leucyl-leucine aldehyde
17-(allylamino)-17-demethoxygeldanamycin
cephaelin
Anisomycin
Dapsone
3-Iodobenzylguanidine
Stavudine
Digoxin
BW B70C
Vigabatrin
rottlerin
lycorine
Inosine Monophosphate
Phenelzine
9-(2-hydroxy-3-nonyl)adenine
Pyrimethamine
Etodolac
8-Bromo Cyclic Adenosine Monophosphate
Dyphylline
piperlonguminine
Pargyline
Sulindac
Y 27632
ozagrel
Lamivudine
HC toxin
Cycloheximide
Oxolinic Acid
Ketorolac
Puromycin
4-amino-6-hydrazino-7-beta-D-ribofuranosyl-7H-pyrrolo(2,3-d)-
pyrimidine-5-carboxamide
Isoflurophate
Dichlorphenamide
velnacrine
bafilomycin A
4-hydroxy-2-nonenal
Mitomycin
celecoxib
cilostazol
Ritonavir
Methionine Sulfoximine
Atovaquone
dorzolamide
Azacitidine
Rifampin
Methotrexate
ellipticine
naringin
Clarithromycin
3-deazaneplanocin
Alpha-Amanitin
Digitoxin
valdecoxib
amprenavir
fluvastatin
versipelostatin
Erythromycin
1-Methyl-3-isobutylxanthine
Selegiline
4-(4-fluorophenyl)-2-(4-hydroxyphenyl)-5-(4-pyridyl)imidazole
Tranylcypromine
3,3′,4′,5-tetrachlorosalicylanilide
4-(5-benzo(1,3)dioxol-5-yl-4-pyridin-2-yl-1H-imidazol-2-yl)benzamide
apicidin
bortezomib
Galantamine
meloxicam
Miconazole
Netilmicin
Piroxicam
resveratrol
Sarin
3-nitropropionic acid
insulin-like growth factor I (57-70)
Epirizole
enterotoxin I, staphylococcal
Prilocaine
Cefoxitin
Chromium
Mannitol
Paraquat
betulin
Heptaminol
Phorbol Esters
Diazoxide
Hycanthone
bergenin
Clopamide
bacterial lysate
titanium dioxide
pyrvinium
nickel chloride
Diethylhexyl Phthalate
chloroxylenol
Amphotericin B
Nefopam
Tolbutamide
acemetacin
2,2-bis(bromomethyl)-1,3-propanediol
Lindane
homatropine
nifuroxazide
bromperidol
Salicylic Acid
Chenodeoxycholic Acid
Metformin
Eugenol
4-vinyl-1-cyclohexene dioxide
Dihydrotestosterone
Medroxyprogesterone
arsenic acid
Dimethylnitrosamine
fazarabine
17-(dimethylaminoethylamino)-17-demethoxygeldanamycin
quintozene
5-fluorouridine
R 848
Chlorhexidine
8-(3-Chlorostyryl)-1,3,7-trimethylxanthine
epidermal growth factor (1-45)
KCB-1 protein, recombinant
beta-1,3-glucan
enrofloxacin
sapphyrin
adiphenine
rescinnamine
oxfendazole
Estrone
CPG-oligonucleotide
Fluocinonide
cetraxate
Platelet Activating Factor
Dinoprostone
norflurane
Piperonyl Butoxide
Benzocaine
Gossypol
Clomiphene
Prednisone
testosterone 17 beta-cypionate
Deoxycholic Acid
Thiamphenicol
cidofovir
DDT
Hydrocortisone
Pentamidine
Ivermectin
15-deoxy-delta(12,14)-prostaglandin J2
Mimosine
pioglitazone
sodium chlorate
tribenoside
Buformin
Cytochalasin B
Primidone
Cefixime
Ultraviolet Rays
Cyproterone Acetate
Sulfamethazine
Methocarbamol
epitiostanol
Daunorubicin
Ethinyl Estradiol
Androsterone
Oxymetholone
ciclopirox
cyclobenzaprine
Ethisterone
Prostaglandins E
alphaxalone
Doxorubicin
Phenol
Acrolein
cinchonine
Immunoglobulin M
Tetradecanoylphorbol Acetate
sulforafan
norethindrone acetate
Cytokines
Megestrol
hydroquinidine
Fenretinide
Dydrogesterone
kavain
Doxycycline
Acetylmuramyl-Alanyl-Isoglutamine
Lithocholic Acid
Hemin
acidocin CH5, Lactobacillus acidophilus
Diethylstilbestrol
Pemoline
Probenecid
Escin
eburnamonine
bis(tri-n-butyltin)oxide
Minocycline
Ondansetron
Diphenhydramine
Acebutolol
Reserpine
Betazole
Norepinephrine
tropisetron
Diazinon
Coumaphos
neuropeptide Y (18-36)
Chlorprothixene
methantheline
Isoproterenol
Domperidone
Amphetamine
Dimaprit
bamipine
Biperiden
Droperidol
Dichlorvos
Chlorpromazine
Caffeine
Loxapine
Doxylamine
ergocryptine
Trifluoperazine
Pindolol
Promazine
Apomorphine
pralidoxime
Vigabatrin
Dicyclomine
Doxazosin
ifenprodil
Pancuronium
1-Methyl-4-phenyl-1,2,3,6-tetrahydropyridine
salsolinol
chlorcyclizine
Nadolol
dironyl
Prochlorperazine
Clonidine
Methapyrilene
discretamine
ozagrel
thioperamide
Tropicamide
Bretylium Tosylate
Isoflurophate
Guanabenz
Amoxapine
Imipramine
velnacrine
Terfenadine
bromopride
Ritodrine
cyanopindolol
Metaproterenol
Dizocilpine Maleate
Phenoxybenzamine
Dobutamine
Fluspirilene
Ticlopidine
Chlorpheniramine
Dexfenfluramine
Timolol
Zimeldine
Dimenhydrinate
Famotidine
Galantamine
Levodopa
Oxymetazoline
Paroxetine
Perphenazine
Sarin
Sertraline
vanoxerine
Nicardipine
Hydroflumethiazide
Felodipine
Nifedipine
Calcitriol
Pinacidil
ochratoxin A
Furosemide
eperisone
1-ethyl-2-benzimidazolinone
Nitrendipine
Bepridil
Nimodipine
tripterine
Phytohemagglutinins
chelidonine
Mebendazole
Paclitaxel
Nocodazole
Podophyllotoxin
Nordihydroguaiaretic Acid
procyanidin
flavanone
6-hydroxy-2,5,7,8-tetramethylchroman-2-carboxylic acid
Vitamin E
Selenomethionine
alpha-Tocopherol
Catechin
naringin
Acetylcysteine
gamma-Tocopherol
resveratrol
Heparin
Vitamin K 3
ozagrel
Aminocaproic Acids
cilostazol
Ticlopidine
Azaguanine
Deoxyglucose
Metyrapone
decitabine
Azathioprine
Stavudine
Cytarabine
Isoniazid
Fluorouracil
Ethionine
Trifluridine
Puromycin
Chitosan
benfluorex
Azacitidine
Methotrexate
fluvastatin
Propylthiouracil
tetrafluoroethylene
Ethyl Methanesulfonate
Methylnitrosourea
temozolomide
Lomustine
Busulfan
Methyl Methanesulfonate
Chlorambucil
Mitomycin
Cyclophosphamide
Carmustine
Ethylnitrosourea
Ifosfamide
2,2′-(hydroxynitrosohydrazono)bis-ethanamine
Molsidomine

In some embodiments, a method of treating muscular dystrophy (MD) is provided, the method comprising administering to a subject having or suspected of having MD an effective amount of a composition comprising a JAG1 agonist.

In some embodiments, the JAG1 agonist is a JAG1 polypeptide or fragment thereof, a nucleic acid encoding a JAG1 polypeptide or fragment thereof, or a compound that promotes JAG1 signaling (e.g., a compound provided in Table 2). Jagged1 (JAG1) is a Notch ligand. Without wishing to be bound by theory, the notch signaling pathway represents a central regulator of gene expression and is critical for cellular proliferation, differentiation and apoptotic signaling during all stages of embryonic muscle development. Notch signaling in mammalian cells is initiated by the ligation of the extracellular ligands (Delta and Jagged) to the Notch family of transmembrane receptors. It results in the cleavage of Notch intracellular domain (NICD) and subsequent activation of gene expression in the nucleus. Transcription factors known to be activated by Notch include the Hairy and Enhancer of Split (Hes) and Hes-related (Hey) families that are sequestered to the nucleus. The regulation of Hes and Hey by Notch in skeletal muscle is still not clear, however it is known that they play a role in satellite cell activation and differentiation during muscle regeneration, and the upregulation of Hes and Hey via activation of Notch1 promotes satellite cell proliferation. The Notch pathway also plays an important role in regeneration after myotoxic injury and overexpression of Notch improves muscle regeneration in aged mice. Exemplary sequences of JAG1 polypeptides and nucleic acids encoding a JAG1 polypeptides are provided herein. In some embodiments, a fragment of a JAG1 polypeptide is a fragment that has substantially the same activity as a full-length JAG1 polypeptide, e.g., activation of Notch signaling. Activation of Notch signaling may be measured using any method known in the art such as Western blot, qPCR, Rt-PCR, ELISA, or RNA sequencing. (e.g., by measuring cleavage of Notch or levels of one or more of downstream targets of Notch signaling, such as those provided in Table 3). Commercial kits for assessing Notch signaling are also available (see. e.g., TaqMan® Array, Human Notch Signaling, Fast 96-well from Life technologies and Human Notch-1 ELISA Kit from Sigma).

In some embodiments, a method of treating muscular dystrophy (MD) is provided, the method comprising administering to a subject having or suspected of having MD an effective amount of a composition that promotes JAG1 signaling. In some embodiments, the composition promotes Notch signaling via JAG1 activation of Notch. Exemplary components of the Notch signaling pathway are shown in Table 3. Sequences can be obtained by entering the below Ensembl IDs into the Ensembl database (Build 77).

TABLE 3
Exemplary components of the Notch pathway

| Gene | Ensembl gene ID | Ensembl transcript ID | Ensembl protein ID |
|---|---|---|---|
| ADAM17 | ENSG00000151694 | ENST00000310823, | ENSP00000309968, |
| | | ENST00000497134, | ENSP00000418728, |
| | | ENST00000538558, | ENSP00000439780 |
| | | ENST00000478059 | |
| AKT1 | ENSG00000142208 | ENST00000349310, | ENSP00000270202, |
| | | ENST00000407796, | ENSP00000384293, |
| | | ENST00000402615, | ENSP00000385326, |
| | | ENST00000554848, | ENSP00000451166, |
| | | ENST00000554581, | ENSP00000451828, |
| | | ENST00000555528, | ENSP00000450688, |
| | | ENST00000544168, | ENSP00000443897, |
| | | ENST00000554192, | ENSP00000450681, |
| | | ENST00000555380, | ENSP00000451290, |
| | | ENST00000555926, | ENSP00000451824, |
| | | ENST00000555458 | ENSP00000451470 |
| APH1A | ENSG00000117362 | ENST00000360244, | ENSP00000353380, |
| | | ENST00000369109, | ENSP00000358105, |
| | | ENST00000236017, | ENSP00000236017, |
| | | ENST00000414276 | ENSP00000397473 |
| APH1B | ENSG00000138613 | ENST00000261879, | ENSP00000261879, |
| | | ENST00000380343, | ENSP00000369700, |
| | | ENST00000560353, | ENSP00000453327, |
| | | ENST00000560890, | ENSP00000453002, |
| | | ENST00000380340, | ENSP00000369697, |
| | | ENST00000559971 | ENSP00000453516 |
| CDKN1A | ENSG00000124762 | ENST00000244741, | ENSP00000244741, |
| | | ENST00000373711, | ENSP00000362815, |
| | | ENST00000405375, | ENSP00000384849, |
| | | ENST00000448526 | ENSP00000409259 |
| CIR1 | ENSG00000138433 | ENST00000342016, | ENSP00000339723, |
| | | ENST00000377973, | ENSP00000367211, |
| | | ENST00000414336, | ENSP00000395036, |
| | | ENST00000362053, | ENSP00000355034, |
| | | ENST00000425101 | ENSP00000405693 |
| CUL1 | ENSG00000055130 | ENST00000409469, | ENSP00000387160, |
| | | ENST00000325222, | ENSP00000326804, |
| | | ENST00000433865, | ENSP00000396011, |
| | | ENST00000543583 | ENSP00000441340 |
| DLL1 | ENSG00000198719 | ENST00000366756 | ENSP00000355718 |
| DLL3 | ENSG00000090932 | ENST00000205143, | ENSP00000205143, |
| | | ENST00000356433 | ENSP00000348810 |
| DLL4 | ENSG00000128917 | ENST00000249749 | ENSP00000249749 |
| DTX1 | ENSG00000135144 | ENST00000257600 | ENSP00000257600 |
| EP300 | ENSG00000100393 | ENST00000263253 | ENSP00000263253 |
| FBXW7 | ENSG00000109670 | ENST00000263981, | ENSP00000263981, |
| | | ENST00000281708, | ENSP00000281708, |
| | | ENST00000296555, | ENSP00000296555, |
| | | ENST00000393956 | ENSP00000377528 |
| FHL1 | ENSG00000022267 | ENST00000370690, | ENSP00000359724, |
| | | ENST00000345434, | ENSP00000071281, |
| | | ENST00000370676, | ENSP00000359710, |
| | | ENST00000370683, | ENSP00000359717, |
| | | ENST00000420362, | ENSP00000391779, |
| | | ENST00000370674, | ENSP00000359708, |
| | | ENST00000452016, | ENSP00000408038, |
| | | ENST00000434885, | ENSP00000413798, |
| | | ENST00000458357, | ENSP00000389920, |
| | | ENST00000456445, | ENSP00000412642, |
| | | ENST00000449474, | ENSP00000414604, |
| | | ENST00000394153, | ENSP00000377709, |
| | | ENST00000394155, | ENSP00000377710, |
| | | ENST00000456218, | ENSP00000392813, |
| | | ENST00000535737, | ENSP00000444815, |
| | | ENST00000536581, | ENSP00000445335, |
| | | ENST00000539015, | ENSP00000437673, |
| | | ENST00000542704, | ENSP00000446441, |
| | | ENST00000543669 | ENSP00000443333 |
| GATA3 | ENSG00000107485 | ENST00000379328, | ENSP00000368632, |
| | | ENST00000346208, | ENSP00000341619, |
| | | ENST00000544011 | ENSP00000439641 |
| GSK3B | ENSG00000082701 | ENST00000264235, | ENSP00000264235, |
| | | ENST00000316626, | ENSP00000324806, |
| | | ENST00000539838 | ENSP00000437981 |
| HAT1 | ENSG00000128708 | ENST00000264108, | ENSP00000264108, |
| | | ENST00000392584, | ENSP00000376363, |
| | | ENST00000412731, | ENSP00000407921, |
| | | ENST00000457761 | ENSP00000403466 |
| HDAC1 | ENSG00000116478 | ENST00000373548, | ENSP00000362649, |
| | | ENST00000428704, | ENSP00000407859, |
| | | ENST00000373541 | ENSP00000362642 |
| HDAC10 | ENSG00000100429 | ENST00000216271, | ENSP00000216271, |
| | | ENST00000448072, | ENSP00000397542, |
| | | ENST00000349505, | ENSP00000343540, |
| | | ENST00000415993, | ENSP00000397517, |
| | | ENST00000429374, | ENSP00000407640, |
| | | ENST00000454936 | ENSP00000406150 |
| HDAC11 | ENSG00000163517 | ENST00000295757, | ENSP00000295757, |
| | | ENST00000433119, | ENSP00000412514, |
| | | ENST00000402259, | ENSP00000384706, |
| | | ENST00000402271, | ENSP00000384123, |
| | | ENST00000404040, | ENSP00000385475, |
| | | ENST00000404548, | ENSP00000385528, |
| | | ENST00000405025, | ENSP00000384019, |
| | | ENST00000405478, | ENSP00000385252, |
| | | ENST00000458642, | ENSP00000405403, |
| | | ENST00000418189, | ENSP00000411792, |
| | | ENST00000434848, | ENSP00000398651, |
| | | ENST00000416248, | ENSP00000402298, |
| | | ENST00000455904, | ENSP00000396122, |
| | | ENST00000437379, | ENSP00000395188, |
| | | ENST00000522202, | ENSP00000429794, |
| | | ENST00000446613, | ENSP00000401487, |
| | | ENST00000425430 | ENSP00000399792 |
| HDAC2 | ENSG00000196591 | ENST00000519065, | ENSP00000430432, |
| | | ENST00000425835, | ENSP00000417026, |
| | | ENST00000368632, | ENSP00000357621, |
| | | ENST00000519108, | ENSP00000430008, |
| | | ENST00000518690, | ENSP00000428653, |
| | | ENST00000523240, | ENSP00000429236, |
| | | ENST00000521610, | ENSP00000429901, |
| | | ENST00000524334, | ENSP00000428989, |
| | | ENST00000520895, | ENSP00000428861, |
| | | ENST00000523628, | ENSP00000427861, |
| | | ENST00000522371, | ENSP00000428599, |
| | | ENST00000521163, | ENSP00000428024, |
| | | ENST00000398283 | ENSP00000381331 |
| HDAC3 | ENSG00000171720 | ENST00000305264, | ENSP00000302967, |
| | | ENST00000523088, | ENSP00000429099, |
| | | ENST00000523353, | ENSP00000430667, |
| | | ENST00000519474 | ENSP00000430782 |
| HDAC4 | ENSG00000068024 | ENST00000345617, | ENSP00000264606, |
| | | ENST00000446876, | ENSP00000392912, |
| | | ENST00000454542, | ENSP00000405226, |
| | | ENST00000445704, | ENSP00000391226, |
| | | ENST00000430200, | ENSP00000410551, |
| | | ENST00000544989, | ENSP00000438111, |
| | | ENST00000393621, | ENSP00000377243, |
| | | ENST00000456922, | ENSP00000406618, |
| | | ENST00000541256, | ENSP00000443057, |
| | | ENST00000543185 | ENSP00000440481 |
| HDAC5 | ENSG00000108840 | ENST00000225983, | ENSP00000225983, |
| | | ENST00000336057, | ENSP00000337290, |
| | | ENST00000393622 | ENSP00000377244 |
| HDAC6 | ENSG00000094631 | ENST00000334136, | ENSP00000334061, |
| | | ENST00000376643, | ENSP00000365831, |
| | | ENST00000426196, | ENSP00000402189, |
| | | ENST00000430858, | ENSP00000397697, |
| | | ENST00000376619, | ENSP00000365804, |
| | | ENST00000423941, | ENSP00000392815, |
| | | ENST00000438518, | ENSP00000403370, |
| | | ENST00000376610, | ENSP00000365795, |
| | | ENST00000441703, | ENSP00000393916, |
| | | ENST00000443563, | ENSP00000402751, |
| | | ENST00000440653, | ENSP00000394377, |
| | | ENST00000413163, | ENSP00000398801, |
| | | ENST00000436813, | ENSP00000405449, |
| | | ENST00000444343 | ENSP00000398566 |
| HDAC7 | ENSG00000061273 | ENST00000080059, | ENSP00000080059, |
| | | ENST00000354334, | ENSP00000351326, |
| | | ENST00000417107, | ENSP00000387792, |
| | | ENST00000450805, | ENSP00000397236, |
| | | ENST00000433685, | ENSP00000403149, |
| | | ENST00000447463, | ENSP00000389501, |
| | | ENST00000427332, | ENSP00000404394, |
| | | ENST00000434070, | ENSP00000388561, |
| | | ENST00000445237, | ENSP00000390415, |
| | | ENST00000421231, | ENSP00000412155, |
| | | ENST00000417902, | ENSP00000400811, |
| | | ENST00000430670, | ENSP00000396159, |
| | | ENST00000440293, | ENSP00000411058, |
| | | ENST00000422254, | ENSP00000410068, |
| | | ENST00000552960, | ENSP00000448532, |
| | | ENST00000380610, | ENSP00000369984, |
| | | ENST00000548080, | ENSP00000446538, |
| | | ENST00000548938, | ENSP00000448305, |
| | | ENST00000547259, | ENSP00000447191, |
| | | ENST00000425451, | ENSP00000401872, |
| | | ENST00000485796, | ENSP00000448448, |
| | | ENST00000551602, | ENSP00000449193, |
| | | ENST00000477203 | ENSP00000449171 |
| HDAC8 | ENSG00000147099 | ENST00000373573, | ENSP00000362674, |
| | | ENST00000439122, | ENSP00000414486, |
| | | ENST00000373556, | ENSP00000362657, |
| | | ENST00000373571, | ENSP00000362672, |
| | | ENST00000373554, | ENSP00000362655, |
| | | ENST00000373568, | ENSP00000362669, |
| | | ENST00000373560, | ENSP00000362661, |
| | | ENST00000373559, | ENSP00000362660, |
| | | ENST00000421523, | ENSP00000398997, |
| | | ENST00000373583, | ENSP00000362685, |
| | | ENST00000415409, | ENSP00000396424, |
| | | ENST00000373561, | ENSP00000362662, |
| | | ENST00000373589, | ENSP00000362691, |
| | | ENST00000429103, | ENSP00000388459, |
| | | ENST00000412342, | ENSP00000400180, |
| | | ENST00000444609, | ENSP00000409778, |
| | | ENST00000436675 | ENSP00000416489 |
| HDAC9 | ENSG00000048052 | ENST00000406451, | ENSP00000384657, |
| | | ENST00000405010, | ENSP00000384382, |
| | | ENST00000406072, | ENSP00000384017, |
| | | ENST00000417496, | ENSP00000401669, |
| | | ENST00000433709, | ENSP00000409003, |
| | | ENST00000413509, | ENSP00000412497, |
| | | ENST00000430454, | ENSP00000411422, |
| | | ENST00000413380, | ENSP00000392564, |
| | | ENST00000441986, | ENSP00000404763, |
| | | ENST00000456174, | ENSP00000388568, |
| | | ENST00000401921, | ENSP00000383912, |
| | | ENST00000441542, | ENSP00000408617, |
| | | ENST00000524023, | ENSP00000430036, |
| | | ENST00000432645, | ENSP00000410337, |
| | | ENST00000428307, | ENSP00000395655, |
| | | ENST00000262069, | ENSP00000262069, |
| | | ENST00000341009, | ENSP00000339165, |
| | | ENST00000446646 | ENSP00000415095 |
| HES1 | ENSG00000114315 | ENST00000232424 | ENSP00000232424 |
| HES5 | ENSG00000197921 | ENST00000378453 | ENSP00000367714 |
| HES6 | ENSG00000144485 | ENST00000272937, | ENSP00000272937, |
| | | ENST00000409002, | ENSP00000387155, |
| | | ENST00000409160, | ENSP00000387215, |
| | | ENST00000436051, | ENSP00000392596, |
| | | ENST00000409574, | ENSP00000387008, |
| | | ENST00000409182, | ENSP00000387343, |
| | | ENST00000409356, | ENSP00000387107, |
| | | ENST00000450098, | ENSP00000390870, |
| | | ENST00000417803 | ENSP00000401797 |
| HEY1 | ENSG00000164683 | ENST00000354724, | ENSP00000346761, |
| | | ENST00000523976, | ENSP00000429792, |
| | | ENST00000518733, | ENSP00000429705, |
| | | ENST00000337919, | ENSP00000338272, |
| | | ENST00000542205 | ENSP00000445025 |
| HEY2 | ENSG00000135547 | ENST00000368364, | ENSP00000357348, |
| | | ENST00000368365 | ENSP00000357349 |
| HIF1A | ENSG00000100644 | ENST00000337138, | ENSP00000338018, |
| | | ENST00000323441, | ENSP00000323326, |
| | | ENST00000394997, | ENSP00000378446, |
| | | ENST00000557538, | ENSP00000451696, |
| | | ENST00000394988, | ENSP00000378439, |
| | | ENST00000539097, | ENSP00000437955, |
| | | ENST00000539494 | ENSP00000446436 |
| ITCH | ENSG00000078747 | ENST00000374864, | ENSP00000363998, |
| | | ENST00000262650, | ENSP00000262650, |
| | | ENST00000535650 | ENSP00000445608 |
| JAG1 | ENSG00000101384 | ENST00000254958, | ENSP00000254958, |
| | | ENST00000423891 | ENSP00000389519 |
| JAG2 | ENSG00000184916 | ENST00000331782, | ENSP00000328169, |
| | | ENST00000347004 | ENSP00000328566 |
| JAK2 | ENSG00000096968 | ENST00000381652, | ENSP00000371067, |
| | | ENST00000539801, | ENSP00000440387, |
| | | ENST00000544510 | ENSP00000443103 |
| LCK | ENSG00000182866 | ENST00000336890, | ENSP00000337825, |
| | | ENST00000482949, | ENSP00000431517, |
| | | ENST00000333070, | ENSP00000328213, |
| | | ENST00000495610, | ENSP00000435605, |
| | | ENST00000373557, | ENSP00000362658, |
| | | ENST00000477031, | ENSP00000436554, |
| | | ENST00000461712, | ENSP00000434525, |
| | | ENST00000373562, | ENSP00000362663, |
| | | ENST00000373564, | ENSP00000362665, |
| | | ENST00000398345, | ENSP00000381387, |
| | | ENST00000436824 | ENSP00000400092 |
| LFNG | ENSG00000106003 | ENST00000222725, | ENSP00000222725, |
| | | ENST00000359574, | ENSP00000352579, |
| | | ENST00000402506, | ENSP00000385764, |
| | | ENST00000402045, | ENSP00000384786, |
| | | ENST00000338732 | ENSP00000343095 |
| MAGEA1 | ENSG00000198681 | ENST00000356661 | ENSP00000349085 |
| MAML1 | ENSG00000161021 | ENST00000292599, | ENSP00000292599, |
| | | ENST00000376951 | ENSP00000366150 |
| MAML2 | ENSG00000184384 | ENST00000524717, | ENSP00000434552, |
| | | ENST00000440572 | ENSP00000412394 |
| MAML3 | ENSG00000196782 | ENST00000509479, | ENSP00000421180, |
| | | ENST00000502696, | ENSP00000422783, |
| | | ENST00000327122, | ENSP00000313316, |
| | | ENST00000398940, | ENSP00000381913, |
| | | ENST00000538400 | ENSP00000444397 |
| MFNG | ENSG00000100060 | ENST00000356998, | ENSP00000349490, |
| | | ENST00000442496, | ENSP00000389274, |
| | | ENST00000436341, | ENSP00000394081, |
| | | ENST00000424765, | ENSP00000407110, |
| | | ENST00000454291, | ENSP00000407094, |
| | | ENST00000416983, | ENSP00000413855, |
| | | ENST00000450946, | ENSP00000396605, |
| | | ENST00000430411, | ENSP00000414342, |
| | | ENST00000438891 | ENSP00000414222 |
| MTOR | ENSG00000198793 | ENST00000361445, | ENSP00000354558, |
| | | ENST00000376838, | ENSP00000366034, |
| | | ENST00000455339, | ENSP00000398745, |
| | | ENST00000539766 | ENSP00000440730 |
| MYC | ENSG00000136997 | ENST00000377970, | ENSP00000367207, |
| | | ENST00000259523, | ENSP00000259523, |
| | | ENST00000517291, | ENSP00000429441, |
| | | ENST00000524013, | ENSP00000430235, |
| | | ENST00000520751, | ENSP00000430226, |
| | | ENST00000454617 | ENSP00000405312 |
| NCOR1 | ENSG00000141027 | ENST00000268712, | ENSP00000268712, |
| | | ENST00000436828, | ENSP00000387727, |
| | | ENST00000395851, | ENSP00000379192, |
| | | ENST00000395849, | ENSP00000379190, |
| | | ENST00000436068, | ENSP00000389839, |
| | | ENST00000395848, | ENSP00000379189, |
| | | ENST00000411510, | ENSP00000407998, |
| | | ENST00000430577, | ENSP00000410784, |
| | | ENST00000395857, | ENSP00000379198, |
| | | ENST00000458113 | ENSP00000395091 |
| NCOR2 | ENSG00000196498 | ENST00000429285, | ENSP00000400281, |
| | | ENST00000404621, | ENSP00000384202, |
| | | ENST00000458234, | ENSP00000402808, |
| | | ENST00000420698, | ENSP00000405367, |
| | | ENST00000405201, | ENSP00000384018, |
| | | ENST00000448614, | ENSP00000408247, |
| | | ENST00000453428, | ENSP00000400687, |
| | | ENST00000440187, | ENSP00000396044, |
| | | ENST00000440337, | ENSP00000398963, |
| | | ENST00000418829, | ENSP00000391389, |
| | | ENST00000413172, | ENSP00000407357, |
| | | ENST00000448008, | ENSP00000403034, |
| | | ENST00000443451, | ENSP00000405246, |
| | | ENST00000542927, | ENSP00000443689, |
| | | ENST00000356219, | ENSP00000348551, |
| | | ENST00000397355, | ENSP00000380513, |
| | | ENST00000404121, | ENSP00000385618, |
| | | ENST00000447011, | ENSP00000396746, |
| | | ENST00000447675 | ENSP00000401058 |
| NCSTN | ENSG00000162736 | ENST00000294785, | ENSP00000294785, |
| | | ENST00000368063, | ENSP00000357042, |
| | | ENST00000438008, | ENSP00000389370, |
| | | ENST00000421914, | ENSP00000390409, |
| | | ENST00000437169, | ENSP00000415442, |
| | | ENST00000424645, | ENSP00000388118, |
| | | ENST00000435149, | ENSP00000407849, |
| | | ENST00000424754, | ENSP00000410124, |
| | | ENST00000368065, | ENSP00000357044, |
| | | ENST00000368067, | ENSP00000357046, |
| | | ENST00000392212, | ENSP00000376047, |
| | | ENST00000535857 | ENSP00000442605 |
| NFKB1 | ENSG00000109320 | ENST00000226574, | ENSP00000226574, |
| | | ENST00000394820, | ENSP00000378297, |
| | | ENST00000505458, | ENSP00000424790, |
| | | ENST00000507079, | ENSP00000426147, |
| | | ENST00000511926, | ENSP00000420904, |
| | | ENST00000509165, | ENSP00000423877, |
| | | ENST00000508584 | ENSP00000424815 |
| NOTCH1 | ENSG00000148400 | ENST00000277541 | ENSP00000277541 |
| NOTCH2 | ENSG00000134250 | ENST00000256646, | ENSP00000256646, |
| | | ENST00000369342, | ENSP00000358348, |
| | | ENST00000401649, | ENSP00000384752, |
| | | ENST00000538680, | ENSP00000439516, |
| | | ENST00000539617 | ENSP00000438937 |
| NOTCH3 | ENSG00000074181 | ENST00000263388, | ENSP00000263388, |
| | | ENST00000539383 | ENSP00000446150 |
| NOTCH4 | ENSG00000204301 | ENST00000375023, | ENSP00000364163, |
| | | ENST00000443903 | ENSP00000398123 |
| NUMB | ENSG00000133961 | ENST00000554546, | ENSP00000452416, |
| | | ENST00000555394, | ENSP00000451625, |
| | | ENST00000557597, | ENSP00000451117, |
| | | ENST00000555238, | ENSP00000451300, |
| | | ENST00000356296, | ENSP00000348644, |
| | | ENST00000556772, | ENSP00000451513, |
| | | ENST00000559312, | ENSP00000452888, |
| | | ENST00000554521, | ENSP00000450817, |
| | | ENST00000560335, | ENSP00000453209, |
| | | ENST00000555738, | ENSP00000452069, |
| | | ENST00000554818, | ENSP00000451959, |
| | | ENST00000555307, | ENSP00000452357, |
| | | ENST00000555987, | ENSP00000451559, |
| | | ENST00000555859, | ENSP00000451326, |
| | | ENST00000554394, | ENSP00000451374, |
| | | ENST00000326018, | ENSP00000315193, |
| | | ENST00000355058, | ENSP00000347169, |
| | | ENST00000359560, | ENSP00000352563, |
| | | ENST00000454166, | ENSP00000394025, |
| | | ENST00000535282, | ENSP00000441258, |
| | | ENST00000544991 | ENSP00000446001 |
| NUMBL | ENSG00000105245 | ENST00000252891, | ENSP00000252891, |
| | | ENST00000540131 | ENSP00000442759 |
| PSENEN | ENSG00000205155 | ENST00000222266 | ENSP00000222266 |
| PSEN1 | ENSG00000080815 | ENST00000324501, | ENSP00000326366, |
| | | ENST00000357710, | ENSP00000350342, |
| | | ENST00000394164, | ENSP00000377719, |
| | | ENST00000394157, | ENSP00000377712, |
| | | ENST00000406768, | ENSP00000385948, |
| | | ENST00000556864, | ENSP00000451588, |
| | | ENST00000557037, | ENSP00000451347, |
| | | ENST00000556533, | ENSP00000452128, |
| | | ENST00000556066, | ENSP00000452267, |
| | | ENST00000553599, | ENSP00000452477, |
| | | ENST00000557356, | ENSP00000451498, |
| | | ENST00000556951, | ENSP00000450551, |
| | | ENST00000557293, | ENSP00000451880, |
| | | ENST00000553719, | ENSP00000451674, |
| | | ENST00000554131, | ENSP00000451915, |
| | | ENST00000555254, | ENSP00000450652, |
| | | ENST00000556011, | ENSP00000451662, |
| | | ENST00000557511, | ENSP00000451429, |
| | | ENST00000560005, | ENSP00000453466, |
| | | ENST00000261970, | ENSP00000261970, |
| | | ENST00000344094, | ENSP00000339523, |
| | | ENST00000555386, | ENSP00000450845, |
| | | ENST00000553855, | ENSP00000452242, |
| | | ENST00000559361 | ENSP00000454156 |
| PSEN2 | ENSG00000143801 | ENST00000366783, | ENSP00000355747, |
| | | ENST00000366782, | ENSP00000355746, |
| | | ENST00000495488, | ENSP00000429682, |
| | | ENST00000460775, | ENSP00000427912, |
| | | ENST00000472139, | ENSP00000427806, |
| | | ENST00000422240, | ENSP00000403737, |
| | | ENST00000524196, | ENSP00000429036, |
| | | ENST00000340188, | ENSP00000339860, |
| | | ENST00000391872, | ENSP00000375745, |
| | | ENST00000496965 | ENSP00000430647 |
| PTCRA | ENSG00000171611 | ENST00000304672, | ENSP00000304447, |
| | | ENST00000418903, | ENSP00000407061, |
| | | ENST00000441198, | ENSP00000409550, |
| | | ENST00000446507 | ENSP00000392288 |
| RBPJ | ENSG00000168214 | ENST00000345843, | ENSP00000305815, |
| | | ENST00000361572, | ENSP00000354528, |
| | | ENST00000342320, | ENSP00000340124, |
| | | ENST00000512351, | ENSP00000424789, |
| | | ENST00000512671, | ENSP00000423644, |
| | | ENST00000505958, | ENSP00000426872, |
| | | ENST00000507561, | ENSP00000423907, |
| | | ENST00000504907, | ENSP00000423703, |
| | | ENST00000506956, | ENSP00000425750, |
| | | ENST00000514807, | ENSP00000424989, |
| | | ENST00000509158, | ENSP00000424804, |
| | | ENST00000514730, | ENSP00000425061, |
| | | ENST00000507574, | ENSP00000422617, |
| | | ENST00000514675, | ENSP00000423575, |
| | | ENST00000515573, | ENSP00000423406, |
| | | ENST00000511546, | ENSP00000422838, |
| | | ENST00000504938, | ENSP00000424459, |
| | | ENST00000504423, | ENSP00000421804, |
| | | ENST00000510778, | ENSP00000427170, |
| | | ENST00000348160, | ENSP00000339699, |
| | | ENST00000342295, | ENSP00000345206, |
| | | ENST00000355476, | ENSP00000347659, |
| | | ENST00000513182,, | ENSP00000427344,, |
| | | ENST00000510778, | ENSP00000427170, |
| | | ENST00000348160, | ENSP00000339699, |
| | | ENST00000342295, | ENSP00000345206, |
| | | ENST00000355476, | ENSP00000347659, |
| | | ENST00000513182 | ENSP00000427344 |
| RFNG | ENSG00000169733 | ENST00000310496, | ENSP00000307971, |
| | | ENST00000429557 | ENSP00000402931 |
| RING1 | ENSG00000204227 | ENST00000374656 | ENSP00000363787 |
| SKP1 | ENSG00000113558 | ENST00000353411, | ENSP00000231487, |
| | | ENST00000522552, | ENSP00000429472, |
| | | ENST00000519321, | ENSP00000429415, |
| | | ENST00000517625, | ENSP00000429961, |
| | | ENST00000522855, | ENSP00000429686, |
| | | ENST00000520417, | ENSP00000429996, |
| | | ENST00000523359, | ENSP00000428962, |
| | | ENST00000328392, | ENSP00000331708, |
| | | ENST00000521216, | ENSP00000431067, |
| | | ENST00000519718, | ENSP00000430774, |
| | | ENST00000523966, | ENSP00000429995, |
| | | ENST00000519054 | ENSP00000430885 |
| SNW1 | ENSG00000100603 | ENST00000261531, | ENSP00000261531, |
| | | ENST00000555761, | ENSP00000451129, |
| | | ENST00000554775, | ENSP00000452059, |
| | | ENST00000554324, | ENSP00000452473, |
| | | ENST00000416259, | ENSP00000387847, |
| | | ENST00000556428 | ENSP00000451741 |
| STAT3 | ENSG00000168610 | ENST00000264657, | ENSP00000264657, |
| | | ENST00000404395, | ENSP00000384943, |
| | | ENST00000389272 | ENSP00000373923 |
| TLE1 | ENSG00000196781 | ENST00000376499, | ENSP00000365682, |
| | | ENST00000418319, | ENSP00000391347, |
| | | ENST00000376484, | ENSP00000365667, |
| | | ENST00000376463, | ENSP00000365646, |
| | | ENST00000355002, | ENSP00000347102, |
| | | ENST00000376472 | ENSP00000365655 |

In some embodiments, the composition increases the expression of JAG1. In some embodiments, the composition comprises a compound, such as a compound provided in Table 2. In some embodiments, the composition comprises a transcription factor as described herein or a vector for recombinant expression of the transcription factor as described herein, wherein the transcription factor increases the expression of JAG1. In some embodiments, the composition comprises a JAG1 agonist selected from the group consisting of a JAG1 polypeptide or fragment thereof as described herein, a nucleic acid encoding a JAG1 polypeptide or fragment thereof as described herein, or a compound that promotes JAG1 signaling, such as a compound in Table 2.

In some embodiments, a method of treating muscular dystrophy (MD) is provided, the method comprising administering to a subject having or suspected of having MD an effective amount of a composition comprising a (e.g., at least one) compound provided in Table 2.

Any compound or composition described herein may be formulated for administration, e.g., a pharmaceutical composition. Formulations are further described herein.

The term “administering” or “administration” means providing a compound or composition as described herein (e.g., as a pharmaceutical composition) to a subject in a manner that is pharmacologically useful. Compositions and compounds as described herein may be administered by a variety of routes of administration, including but not limited to subcutaneous, intramuscular, intradermal, oral, intranasal, transmucosal, intramucosal, intravenous, sublingual, rectal, ophthalmic, pulmonary, transdermal, transcutaneous or by a combination of these routes.

An “effective amount,” or an “amount effective,” as used herein, refers to an amount of a compound and/or composition described herein that is effective in producing the desired molecular, therapeutic, ameliorative, inhibitory or preventative effect, and/or results in a desired clinical effect. For example, an effective amount of a composition or compound described herein when administered to a patient results in e.g., increased muscle strength, increased motility, restoration of muscle function or phenotype, decreased fatigue, decreased difficulty with motor skills, etc. In some aspects, the desired therapeutic or clinical effect resulting from administration of an effective amount of a composition or compound described herein, may be monitored e.g. by monitoring the creatine kinase (CK) levels in a patient's blood, by electromyography, and/or by histological examination of a muscle biopsy. In the combination therapies of the present invention, an effective amount can refer to each individual agent or compound in a composition or to the combination as a whole, wherein the amounts of all agents administered are together effective, but wherein the component agent of the combination may not be present individually in an effective amount.

### Formulations

Compositions and compounds described herein may be formulated for administration to a subject. Exemplary formulations for exemplary routes of administration are described below.

### 1. Parenteral Formulations

The compounds and compositions described herein can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intraocular, intravesicularly, intrapericardially, intraumbilically, by injection, and by infusion.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds or compositions as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds or compositions in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

i. Controlled Release Formulations

The parenteral formulations described herein can be formulated for controlled release including immediate release, delayed release, extended release, pulsatile release, and combinations thereof.

Nano- and Microparticles

For parenteral administration, the one or more compounds or compositions, and optional one or more additional active agents, can be incorporated into microparticles, nanoparticles, or combinations thereof that provide controlled release of the compounds and/or one or more additional active agents. In embodiments wherein the formulations contains two or more compounds or compositions, the compounds or compositions can be formulated for the same type of controlled release (e.g., delayed, extended, immediate, or pulsatile) or the compounds or compositions can be independently formulated for different types of release (e.g., immediate and delayed, immediate and extended, delayed and extended, delayed and pulsatile, etc.).

For example, the compounds or compositions and/or one or more additional active agents can be incorporated into polymeric microparticles which provide controlled release of the compounds or compositions. Release of the compounds or compositions is controlled by diffusion of the compounds or compositions out of the microparticles and/or degradation of the polymeric particles by hydrolysis and/or enzymatic degradation. Suitable polymers include ethylcellulose and other natural or synthetic cellulose derivatives.

Polymers which are slowly soluble and form a gel in an aqueous environment, such as hydroxypropyl methylcellulose or polyethylene oxide may also be suitable as materials for composition or compound containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the compound or composition can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins which are water insoluble, such as zein, can also be used as materials for the formation of compound or composition containing microparticles. Additionally, proteins, polysaccharides and combinations thereof which are water soluble can be formulated with composition or compound into microparticles and subsequently cross-linked to form an insoluble network. For example, cyclodextrins can be complexed with individual compounds and subsequently cross-linked.

Encapsulation or incorporation of compound or composition into carrier materials to produce compound or composition containing microparticles can be achieved through known pharmaceutical formulation techniques. In the case of formulation in fats, waxes or wax-like materials, the carrier material is typically heated above its melting temperature and the compound or composition is added to form a mixture comprising compound or composition particles suspended in the carrier material, compound or composition dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, compound or composition is added, and the molten wax-composition mixture is congealed under constant stirring as the mixture cools. Alternatively, the molten wax-composition mixture can be extruded and spheronized to form pellets or beads. These processes are known in the art.

For some carrier materials it may be desirable to use a solvent evaporation technique to produce compound or composition containing microparticles. In this case compound or composition and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, compound or composition in a particulate form is homogeneously dispersed in a water-insoluble or slowly water soluble material. To minimize the size of the compound or composition particles, the compound or composition powder itself may be milled to generate fine particles prior to formulation. The process of jet milling, known in the pharmaceutical art, can be used for this purpose. In some embodiments compound or composition in a particulate form is homogeneously dispersed in a wax or wax like substance by heating the wax or wax like substance above its melting point and adding the compound or composition particles while stirring the mixture. In this case a pharmaceutically acceptable surfactant may be added to the mixture to facilitate the dispersion of the compound or composition particles.

The particles can also be coated with one or more modified release coatings. Solid esters of fatty acids, which are hydrolyzed by lipases, can be spray coated onto microparticles or particles. Zein is an example of a naturally water-insoluble protein. It can be coated onto microparticles or particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding compound or composition containing microparticles or compound or composition particles, a water soluble protein can be spray coated onto the microparticles and subsequently cross-linked by the one of the methods described above. Alternatively, compound or composition containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

In certain embodiments, it may be desirable to provide continuous delivery of one or more compound or composition to a patient in need thereof. For intravenous or intraarterial routes, this can be accomplished using drip systems, such as by intravenous administration. For topical applications, repeated application can be done or a patch can be used to provide continuous administration of the compounds over an extended period of time.

ii. Injectable/Implantable Solid Implants

The compounds and compositions described herein can be incorporated into injectable/implantable solid or semi-solid implants, such as polymeric implants. In one embodiment, the compounds or compositions are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the compound or composition dissolved or dispersed in the polymer solution and the solvent is then evaporated. Solvent processes require that the polymer be soluble in organic solvents. Another method is compression molding of a mixed powder of the polymer and the compound or composition.

Alternatively, the compounds or compositions can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods. The release of the one or more compounds or compositions from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

### B. Enteral Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art. Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof. Carrier also includes all components of the coating composition which may include plasticizers, pigments, colorants, stabilizing agents, and glidants. Delayed release dosage formulations may be prepared as described in standard references. These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard compound or composition decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

i. Controlled Release Formulations

Oral dosage forms, such as capsules, tablets, solutions, and suspensions, can for formulated for controlled release. For example, the one or more compounds or compositions and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the compound or composition and a controlled release polymer or matrix. Alternatively, the composition or compound particles can be coated with one or more controlled release coatings prior to incorporation in to the finished dosage form.

In another embodiment, the one or more compounds or compositions, and optionally one or more additional active agents, are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds or compositions, and optionally one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the compound or composition with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In some embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In some embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In some embodiments, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm Pharma under the tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Eudragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS may be mixed together in any desired ratio in order to ultimately obtain a sustained-release formulation having a desirable dissolution profile. Desirable sustained-release multiparticulate systems may be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired compound or composition release profile can be achieved by combining low permeable and high permeable coating materials in suitable proportion.

The devices with different compound or composition release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the compound or composition is mixed with a wax material and either spray-congealed or congealed and screened and processed.

### Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a composition with a selected coating material. The composition may be, e.g., a tablet for incorporation into a capsule, a tablet for use as an inner core in a “coated core” dosage form, or a plurality of compound or composition-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### 3. Topical Formulations

Suitable dosage forms for topical administration include creams, ointments, salves, sprays, gels, lotions, emulsions, and transdermal patches. The formulation may be formulated for transmucosal, transepithelial, transendothelial, or transdermal administration. The compositions may further contain one or more chemical penetration enhancers, membrane permeability agents, membrane transport agents, emollients, surfactants, stabilizers, and combination thereof.

“Emollients” are an externally applied agent that softens or soothes skin and are generally known in the art and listed in compendia, such as the “Handbook of Pharmaceutical Excipients”, 4th Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

i. Lotions, Creams, Gels, Ointments, Emulsions, and Foams

“Hydrophilic” as used herein refers to substances that have strongly polar groups that readily interact with water.

“Lipophilic” refers to compounds having an affinity for lipids.

“Amphiphilic” refers to a molecule combining hydrophilic and lipophilic (hydrophobic) properties

“Hydrophobic” as used herein refers to substances that lack an affinity for water; tending to repel and not absorb water as well as not dissolve in or mix with water.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic compounds or compositions will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These compound or composition delivery systems are typically capsules (hard shell or soft shell) comprised of the compound or composition dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove. The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the compound or composition. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams may consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers may be used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a some embodiments, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, it may be desirable to provide continuous delivery of one or more compounds or compositions to a patient in need thereof. For topical applications, repeated application can be done or a patch can be used to provide continuous administration of the compounds over an extended period of time.

### 4. Pulmonary Formulations

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where compound or composition absorption occurs. The alveoli are covered by a thin epithelium without cilia or a mucus blanket and secrete surfactant phospholipids.

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung. The deep lung, or alveoli, are the primary target of inhaled therapeutic aerosols for systemic compound or composition delivery.

Pulmonary administration of therapeutic compositions comprised of low molecular weight compounds has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for compound or composition absorption due to the coverage of the epithelial surface by numerous microvilli, the subepithelial layer is highly vascularized, the venous blood from the nose passes directly into the systemic circulation and therefore avoids the loss of compound or composition by first-pass metabolism in the liver, it offers lower doses, more rapid attainment of therapeutic blood levels, quicker onset of pharmacological activity, fewer side effects, high total blood flow per cm3, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solutions is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, Calif.).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no compound or composition) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different EGS may be administered to target different regions of the lung in one administration.

Formulations for pulmonary delivery include unilamellar phospholipid vesicles, liposomes, or lipoprotein particles. Formulations and methods of making such formulations containing nucleic acid are well known to one of ordinary skill in the art. Liposomes are formed from commercially available phospholipids supplied by a variety of vendors including Avanti Polar Lipids, Inc. (Birmingham, Ala.). In one embodiment, the liposome can include a ligand molecule specific for a receptor on the surface of the target cell to direct the liposome to the target cell.

### Prognosis and Evaluation of Responsiveness to Treatment

Other aspects of the disclosure relate to methods of prognosis or treatment evaluation, such as MD treatment evaluation.

In some embodiments, a method of prognosing muscular dystrophy (MD) is provided, the method comprising (a) selecting a subject having or suspected of having MD; (b) measuring an expression level of JAG1 in a sample obtained from the subject; and (c) identifying the subject as having a more favorable prognosis when the expression level of JAG1 is higher than a control level. A more favorable prognosis may include, e.g., amelioration or stagnation of muscle strength or not showing a phenotype, such as muscle weakening, if the subject treated before the start of the symptoms, such as in a child.

In some embodiments, the sample comprises muscle cells or muscle progenitor cells. The sample may be obtained from the subject, e.g., by muscle biopsy.

In some embodiments, a JAG1 mRNA or protein expression level is measured. Assays for measuring mRNA and protein levels are described herein.

In some embodiments, the subject is identified as having a more favorable prognosis when the expression level of JAG1 is at least 5%, at least 10%, at least 20%, at least 30%, at least 40%, at least 50%, at least 60%, at least 70%, at least 80%, at least 90%, at least 100%, at least 150%, at least 200%, at least 250%, or at least 300% higher or more than a control level. In some embodiments, the control level is the level of JAG1 expression in a subject not having MD. In some embodiments, the control level is the level of JAG1 expression in a subject having a MD disease course.

In other embodiments, a method of prognosing muscular dystrophy (MD) is provided, the method comprising (a) selecting a subject having or suspected of having MD; (b) detecting a variant in a JAG1 gene, a JAG1 promoter, or a JAG1 regulatory element (e.g., a variant that upregulates JAG1 expression) in a sample obtained from the subject; and (c) identifying the subject as having a more favorable prognosis when the variant in the JAG1 gene, JAG1 promoter, or JAG1 regulatory element is detected. In some embodiments, the variant comprises one or more of a mutation, a single nucleotide polymorphism (SNP), or a haplotype in the JAG1 gene, JAG1 promoter, or JAG1 regulatory element. As used herein, a mutation is one or more changes in the nucleotide sequence of the genome of the subject. As used herein, mutations include, but are not limited to, point mutations, insertions, deletions, rearrangements, inversions and duplications. Mutations also include, but are not limited to, silent mutations, missense mutations, and nonsense mutations. A SNP is a mutation that occurs at a single nucleotide location on a chromosome. A haplotype is a chromosomal region containing at least one mutation that correlates with the more favorable prognosis. Variants, such as mutations, may be measured using any method known in the art, such as sequencing-based methods or probe-based methods.

In other embodiments, a method of evaluating responsiveness to treatment for muscular dystrophy (MD) is provided, the method comprising (a) measuring an expression level of JAG1 (e.g., a JAG1 mRNA or protein level) in a sample obtained from a subject having MD prior to the subject receiving a treatment for MD; (b) measuring an expression level of JAG1 in a sample obtained from the subject after the subject has received a treatment for MD; and (c) comparing the expression levels of JAG1 measured prior to and after treatment; wherein an increase (e.g., at least 5%, at least 10%, at least 20%, at least 30%, at least 40%, at least 50%, at least 60%, at least 70%, at least 80%, at least 90%, at least 100%, at least 150%, at least 200%, at least 250%, or at least 300% higher or more) in JAG1 expression after the subject has received the treatment identifies the subject as responsive to treatment; or wherein a decrease or no change in JAG1 expression after the subject has received the treatment identifies the subject as unresponsive to treatment. The treatment for MD may be a composition or compound as described herein.

In some embodiments, the sample comprises muscle cells, muscle progenitor cells, or blood vessels obtained from muscle tissue. The sample can be obtained from the subject, e.g., by muscle biopsy.

### Screening

Other aspects of the disclosure relate to methods for screening compounds, e.g., to identify compounds that increase or modulate JAG1 expression and/or treat MD.

In some embodiments, a method for identifying a compound for the treatment of MD is provided, the method comprising (a) contacting a cell with a candidate compound; (b) measuring an expression level of JAG1 in the cell; and (c) identifying the candidate compound as a compound for the treatment of MD if the expression level of JAG1 is higher than a control level. The candidate compound may be present in a library, e.g., a small molecule library. Accordingly, a method of screening may be a high-throughput screen. In general, a molecular library contains from two to 10¹² molecules, and any integer number therebetween. Methods for preparing libraries of molecules and screening such molecules are well known in the art.

In some embodiments, the control level is a level of expression in the cell in the absence of the candidate compound, such as a cell that has not been contacted with the candidate compound or contacted with a composition not containing the compound (e.g., media or PBS only).

The cell may be a single cell or a population of cells. The cell may be contained within a well, e.g., in a multiwell plate. In some embodiments, the cell is selected from the group consisting of fibroblasts, blood cells, mesenchymal stem cells, muscle progenitor cells, muscle satellite cells, smooth muscle cells, muscle side population cells, myoblasts, iPS cells, and embryonic stem cells. The cell may also be a DMD cell line, such as SC604A/B-MD (available from Systembio), GM07691 (available from the Coriell Institute), or RCDMD (see Caviedes et al. Ion channels in a skeletal muscle cell line from a Duchenne muscular dystrophy patient. Muscle Nerve. 1994 September; 17(9):1021-8).

In other embodiments, a method for identifying a JAG1-modulating compound for the treatment of MD is provided, the method comprising (a) contacting an animal model, such as a zebrafish, with a candidate compound; (b) assessing the muscle phenotype of the animal model; and (c) identifying the candidate compound as a JAG1-modulating compound for the treatment of MD if the muscle phenotype of the zebrafish is improved compared to a control muscle phenotype. The muscle phenotype can be measured using any method known in the art, such as a muscle birefringence assay.

In some embodiments, the animal model is a zebrafish, such as a sapje zebrafish, sapje-like zebrafish, lama2 zebrafish, dag 1 zebrafish, cav3 zebrafish, co16a1/co16a3 zebrafish, desma zebrafish, dmd zebrafish, dnajb6b zebrafish, dysf zebrafish, fkrp zebrafish, ilk zebrafish, ispd zebrafish, itgalpha7 zebrafish, lama4 zebrafish, lamb2 zebrafish, lmna zebrafish, pomt1/pomt2 zebrafish, ryr1b zebrafish, sepn1 zebrafish, sgcd zebrafish, tcap zebrafish, tnnt2a/tnnt2c zebrafish, tnni2a.4 zebrafish, tnnt3a/tnnt3b zebrafish, ttn zebrafish, or vcl zebrafish (see, e.g., Berger and Currie. Zebrafish models flex their muscles to shed light on muscular dystrophies. Disease Models & Mechanisms 5, 726-732 (2012)). In some embodiments, the zebrafish is a sapje zebrafish, sapje-like zebrafish, lama2 zebrafish, or dag1 zebrafish.

In some embodiments, the method further comprises determining the expression of JAG1 in the animal model such as a zebrafish. Assays for determining expression are described herein.

In some embodiments, the method further comprises testing the candidate compound in a mammalian model of MD, by administering the candidate compound to a mammal; and identifying the candidate compound as a therapeutic compound for the treatment of MD if the phenotype of the mammal is improved as compared to a control. The mammalian model of MD may be any model known in the art or described herein. In some embodiments, the mammal is a mouse, dog, cat, or pig model of MD (see, e.g., Gaschen et al. Dystrophin deficiency causes lethal muscle hypertrophy in cats. Journal of the Neurological Sciences, 110 (1992): 149-159; Hollinger et al. Dystrophin insufficiency causes selective muscle histopathology and loss of dystrophin-glycoprotein complex assembly in pig skeletal muscle. FASEB J. 28, 1600-1609 (2014); Allamand et al. Animal models for muscular dystrophy: valuable tools for the development of therapies. Hum. Mol. Genet. (2000) 9(16): 2459-2467; and Ng et al. Animal models of muscular dystrophy. Prog Mol Biol Transl Sci. 2012; 105:83-111).

The improvement in phenotype in the mammal can be measured using any method known in the art or described herein. In some embodiments, the improvement in phenotype is assessed by muscle tissue biopsy, determining muscle strength (e.g., by treadmill, rota-road, or grip), or assessing blood levels of creatine kinase.

In some embodiments, the control is the phenotype of a mammal in the absence of being administered the candidate compound.

### Subjects

The term “subject,” as used herein, refers to an individual organism. In some embodiments, a subject is a mammal, for example, a human, a non-human primate, a mouse, a rat, a cat, a dog, a cattle, a goat, a pig, or a sheep. In some embodiments, the subject is a subject, such as a human, having or suspected of having MD. In some embodiments, the subject is a subject, such as a human, having or suspected of having DMD. In some embodiments, the subject has a mutation in the dystrophin gene, such as a mutation that results in decreased or no expression of Dystrophin protein in the muscle of the subject. Exemplary mutations include a frame-shift mutation or exon 45 deletion.

Muscular dystrophies (MDs) are a group of diseases characterized by weakening of the musculoskeletal system, often resulting the decreased locomotion and early death. Exemplary MDs include, but are not limited to, Duchenne muscular dystrophy (DMD), Becker muscular dystrophy (BMD), X-linked dilated cardiomyopathy (XLDC), Limb-Girdle muscular dystrophy (LGMD), Distal muscular dystrophy, Facioscapulohumeral muscular dystrophy, congenital muscular dystrophy (CMD), Emery-Dreifuss muscular dystrophy (EDMD), Myotonic muscular dystrophy, and Oculopharyngeal muscular dystrophy.

Duchenne muscular dystrophy (DMD) is an X-linked form of muscular dystrophy caused by mutations in the dystrophin gene. Symptoms of DMD include muscle weakness, usually associated with wasting of the muscles that begins with voluntary muscles, fatigue, muscle contractures, muscle fiber deformities, pseudohypertrophy, and skeletal deformities. Symptoms usually appear by the age of 6 and may be present in infancy.

A subject having MD can be identified using any method known in the art or described herein. Exemplary diagnostic assays for identifying subjects having MD, such as DMD, include physical examination, muscle biopsy, creatine kinase levels, electromyography, electrocardiography, and DNA analysis (e.g., genotyping).

### Other Methods

Other aspects of the disclosure provide a method of increasing the proliferation of a muscle cell or a muscle progenitor cell, the method comprising increasing the expression of JAG1 in a muscle cell or a muscle progenitor cell, wherein increasing the expression of JAG1 increases the proliferation of the cell. Methods for identifying muscle cells or muscle progenitor cells are known in the art, e.g., by cell surface markers. In some embodiments, an increase in muscle cells or muscle progenitor cells is measured by flow cytometry or Fluorescence-activated cell sorting (FACS). The expression of JAG1 may be increased, e.g., by contacting a composition or compound as described herein to the muscle cell or muscle progenitor cell. The increase in expression may be, e.g., at least 5%, at least 10%, at least 20%, at least 30%, at least 40%, at least 50%, at least 60%, at least 70%, at least 80%, at least 90%, at least 100%, at least 150%, at least 200%, at least 250%, at least 300% or more than a control expression level. The control expression level may be a level of JAG1 expression in a control cell, such as a cell comprising a mutation in the dystrophin gene and/or a cell that has not been contacted with a composition or compound as described herein. The expression level may be measured using an assay known in the art or as described herein. In some embodiments, the method is performed in vitro or ex vivo. In some embodiments, the muscle cell or muscle progenitor cell is in a subject, such as a subject having or suspected of having MD, such as DMD.

Other aspects of the disclosure provide a method of increasing and/or enhancing myofiber structure in a muscle cell or muscle progenitor cell, the method comprising increasing the expression of JAG1 in a muscle cell or a muscle progenitor cell, wherein increasing the expression of JAG1 increases and/or enhances the myofiber structure (e.g., by increasing the number of myofibers and/or increases the size of myofibers) of the cell. An increase and/or enhancement of myofiber structure of the cell may be measuring using any method known in the art or described herein. Exemplary methods for measuring an increase and/or enhancement of myofiber structure include, but are not limited to, a birefringence assay, muscle histopatological analysis, immunofluorescence for muscle proteins, and fiber type assays.

In some embodiments, the method is performed in vitro or ex vivo. In some embodiments, the muscle cell or muscle progenitor cell is in a subject, such as a subject having or suspected of having MD, such as DMD.

### Expression Level Analysis

Aspects of the disclosure relate to methods that include or measure an expression level of JAG1, such as an mRNA level or protein level of JAG1. Any method for expression level analysis known in the art is contemplated for use herein. Exemplary assays are described below.

mRNA Assays

The art is familiar with various methods for analyzing mRNA levels. Examples of mRNA-based assays include but are not limited to oligonucleotide microarray assays, quantitative RT-PCR, Northern analysis, and multiplex bead-based assays. Other mRNA detection and quantitation methods include multiplex detection assays known in the art, e.g., xMAP® bead capture and detection (Luminex Corp., Austin, Tex.).

An exemplary method is a quantitative RT-PCR assay which may be carried out as follows: mRNA is extracted from cells in a biological sample (e.g., muscle cells) using the RNeasy kit (Qiagen). Total mRNA is used for subsequent reverse transcription using the SuperScript III First-Strand Synthesis SuperMix (Invitrogen) or the SuperScript VILO cDNA synthesis kit (Invitrogen). 5 μl of the RT reaction is used for quantitative PCR using SYBR Green PCR Master Mix and gene-specific primers, in triplicate, using an ABI 7300 Real Time PCR System.

mRNA detection binding partners include oligonucleotide or modified oligonucleotide (e.g. locked nucleic acid) probes that hybridize to a target mRNA. mRNA-specific binding partners can be generated using the sequences provided herein or known in the art. Methods for designing and producing oligonucleotide probes are well known in the art (see, e.g., U.S. Pat. No. 8,036,835; Rimour et al. GoArrays: highly dynamic and efficient microarray probe design. Bioinformatics (2005) 21 (7): 1094-1103; and Wernersson et al. Probe selection for DNA microarrays using OligoWiz. Nat Protoc. 2007; 2(11):2677-91).

### Protein Assays

The art is familiar with various methods for measuring protein levels. Protein levels may be measured using protein-based assays such as but not limited to immunoassays (e.g., Western blots, enzyme-linked immunosorbent assay (ELISA), or immunofluroscence or colorimetric cell staining), multiplex bead-based assays, and assays involving aptamers (such as SOMAmer™ technology) and related affinity agents.

A brief description of an exemplary immunoassay, an ELISA, is provided here. A biological sample is applied to a substrate having bound to its surface protein-specific binding partners (i.e., immobilized protein-specific binding partners). The protein-specific binding partner (which may be referred to as a “capture ligand” because it functions to capture and immobilize the protein on the substrate) may be an antibody or an antigen-binding antibody fragment such as Fab, F(ab)2, Fv, single chain antibody, Fab and sFab fragment, F(ab′)₂, Fd fragments, scFv, and dAb fragments, although it is not so limited. Other binding partners are described herein. Protein present in the biological sample bind to the capture ligands, and the substrate is washed to remove unbound material. The substrate is then exposed to soluble protein-specific binding partners (which may be identical to the binding partners used to immobilize the protein). The soluble protein-specific binding partners are allowed to bind to their respective proteins immobilized on the substrate, and then unbound material is washed away. The substrate is then exposed to a detectable binding partner of the soluble protein-specific binding partner. In one embodiment, the soluble protein-specific binding partner is an antibody having some or all of its Fc domain. Its detectable binding partner may be an anti-Fc domain antibody. As will be appreciated by those in the art, if more than one protein is being detected, the assay may be configured so that the soluble protein-specific binding partners are all antibodies of the same isotype. In this way, a single detectable binding partner, such as an antibody specific for the common isotype, may be used to bind to all of the soluble protein-specific binding partners bound to the substrate.

Other examples of protein detection and quantitation methods include multiplexed immunoassays as described for example in U.S. Pat. Nos. 6,939,720 and 8,148,171, and published US Patent Application No. 2008/0255766, and protein microarrays as described for example in published US Patent Application No. 2009/0088329.

Protein detection binding partners include protein-specific binding partners. Protein-specific binding partners can be generated using the sequences provided herein or known in the art. In some embodiments, binding partners may be antibodies. As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and dAb fragments) as well as complete antibodies. Methods for making antibodies and antigen-binding fragments are well known in the art (see, e.g. Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609).

Binding partners also include non-antibody proteins or peptides that bind to or interact with a target protein, e.g., through non-covalent bonding. For example, if the protein is a ligand, a binding partner may be a receptor for that ligand. In another example, if the protein is a receptor, a binding partner may be a ligand for that receptor. In yet another example, a binding partner may be a protein or peptide known to interact with a protein. Methods for producing proteins are well known in the art (see, e.g. Sambrook et al, “Molecular Cloning: A Laboratory Manual” (2nd Ed.), Cold Spring Harbor Laboratory Press (1989) and Lewin, “Genes IV”, Oxford University Press, New York, (1990)) and can be used to produce binding partners such as ligands or receptors.

Binding partners also include aptamers and other related affinity agents. Aptamers include oligonucleic acid or peptide molecules that bind to a specific target. Methods for producing aptamers to a target are known in the art (see, e.g., published US Patent Application No. 2009/0075834, U.S. Pat. Nos. 7,435,542, 7,807,351, and 7,239,742). Other examples of affinity agents include SOMAmer™ (Slow Off-rate Modified Aptamer, SomaLogic, Boulder, Colo.) modified nucleic acid-based protein binding reagents.

Binding partners also include any molecule capable of demonstrating selective binding to any one of the target proteins disclosed herein, e.g., peptoids (see, e.g., Reyna J Simon et al., “Peptoids: a modular approach to drug discovery” Proceedings of the National Academy of Sciences USA, (1992), 89(20), 9367-9371; U.S. Pat. No. 5,811,387; and M. Muralidhar Reddy et al., Identification of candidate IgG biomarkers for Alzheimer's disease via combinatorial library screening. Cell 144, 132-142, Jan. 7, 2011).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### Jag1: A Genetic Modifier of Duchenne Muscular Dystrophy Phenotype

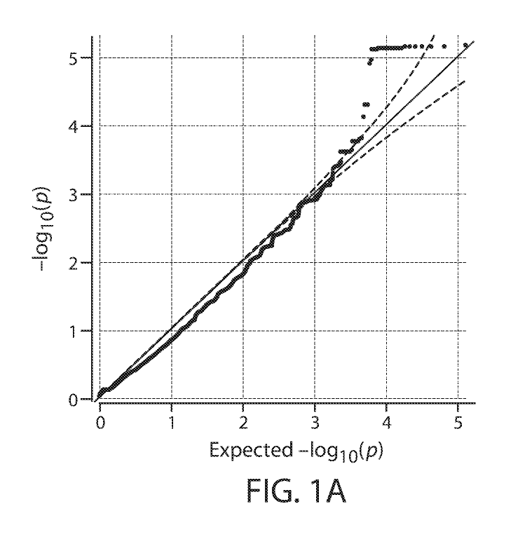

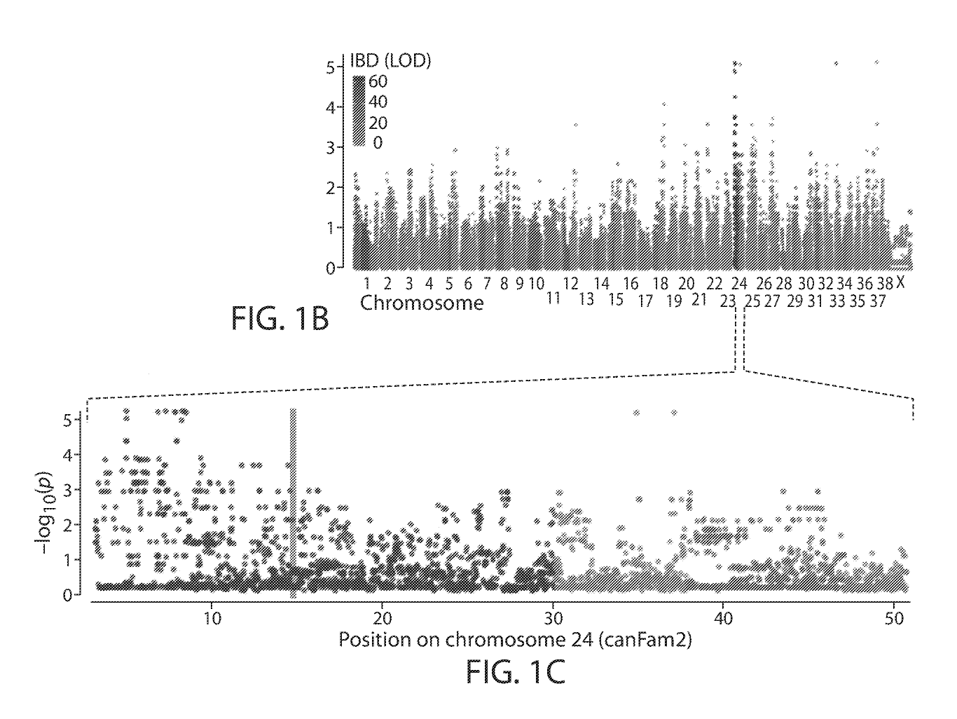

Aiming to understand the genetic basis behind the escaper phenotype in golden retriever muscular dystrophy (GRMD) dogs, a GWA was performed, comparing the 2 available escaper GRMD dogs (a male dog and his sire) and 32 severely affected GRMD dogs from a pedigree. Animals were classified based on functional analysis that evaluated the ambulation capacity and posture of each dog as compared to a normal dog [ref. 14]. The candidate region for the mutation was identified by comparing the 2 escapers to 31 severe affected GRMD dogs. All dogs were genotyped using the Illumina CanineHD 170K SNP array. The mixed linear model approach was used and implemented in EMMAX [ref. 15] to control for relatedness (FIG. 1A) and identified strongly associated SNPs (p<1×10⁻⁵) on chromosomes 24, 33 and 37 (FIG. 1B). The identity by descent (IBD) was measured across the genome between the two cases using Beagle [ref. 16]. Only associated SNPs on chromosome 24 overlapped a segment of IBD in the two escapers (LOD=64.7), consistent with a single origin of the causative mutation (FIG. 1B). The 27 Mb IBD segment associated with the escaper phenotype (chr24:3,073,196-30,066,497) contained approximately 350 protein coding genes.

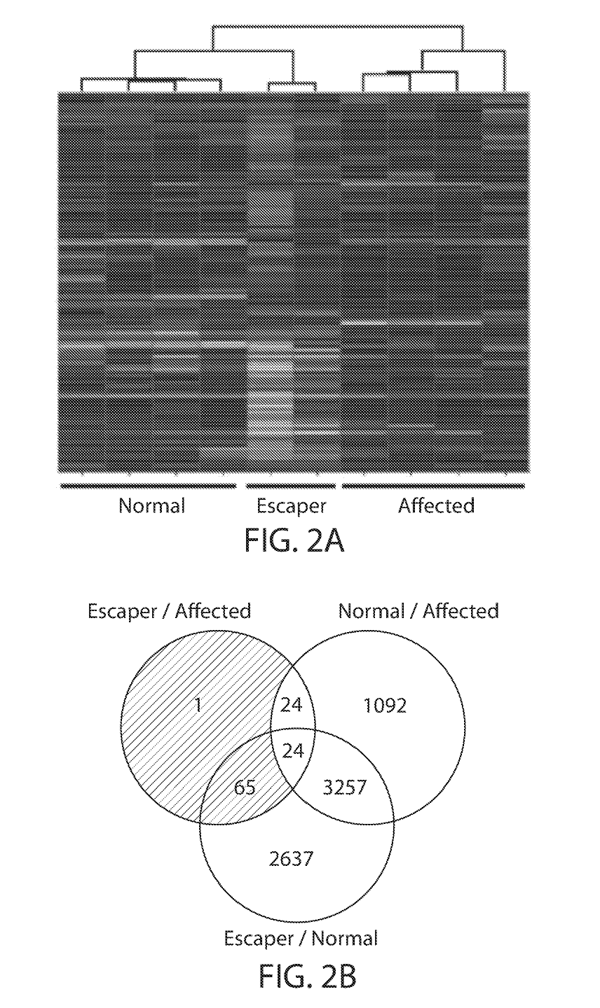

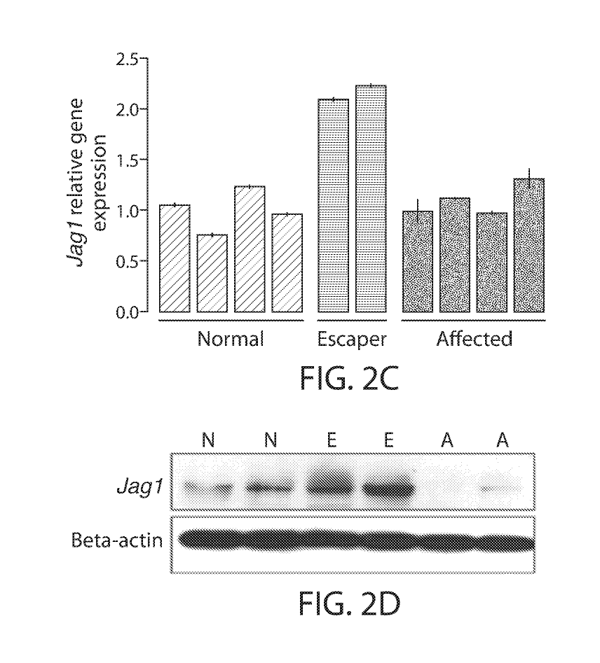

To search for the gene causing the escaper phenotype, the muscle gene expression of the two escapers, four affected and four normal littermates was compared by Agilent mRNA SurePrint Canine arrays, all at age 2 years. A total of 114 genes were identified as being differentially expressed between escapers and affected GRMD dogs as seen in the unsupervised clustering of all 10 samples (FIG. 2A). The analysis revealed very similar muscle gene expression patterns in the escaper GRMD dogs, which was more similar to the normal dogs than the expression patterns of affected dogs. 65 genes were differentially expressed between the escapers and both severe affected and normal dogs (FIG. 2B) pointing to a possible compensatory mechanism. Of the 65 genes differentially (Table 1) expressed between the escapers and severely affected GRMD dogs, only one gene, jagged1, was located on the associated haplotype on chr24. Jagged1 mRNA levels were two times higher in the escapers when compared to both normal and severely affected dogs (FIG. 2C). Protein level analysis confirmed the mRNA findings (FIG. 2D).

TABLE 1
Gene transcripts differentially expressed in Escapers vs Affected

| | | | Fold Change | | | q-value (<5%) | | |
|---|---|---|---|---|---|---|---|---|
| | | | Affected | Escaper | Escaper | Affected | Escaper | Escaper |
| | | | vs | vs | vs | vs | vs | vs |
| Name | Location | Description | Normal | Normal | Affected | Normal | Normal | Affected |
| A_11_P0000033563 | cf\|chr7: 062688468- | Unknown | 1.0 | 4.1 | 4.0 | 46.1 | 0.1 | 4.1 |
| | 062688527 | | | | | | | |
| LOC608476 | cf\|chr29: 31653690- | gb\|PREDICTED: | 0.9 | 3.0 | 3.4 | 34.7 | 0.3 | 2.8 |
| | 31653631 | Canis familiaris | | | | | | |
| | | similar to | | | | | | |
| | | Fatty acid- | | | | | | |
| | | binding protein, | | | | | | |
| | | adipocyte (AFABP) | | | | | | |
| | | (Adipocyte | | | | | | |
| | | lipid-binding | | | | | | |
| | | protein) | | | | | | |
| | | (ALBP) (A-FABP) | | | | | | |
| | | (P2 adipocyte | | | | | | |
| | | protein) | | | | | | |
| | | (Myelin P2 | | | | | | |
| | | protein homolog) | | | | | | |
| | | (3T3-L1 lipid | | | | | | |
| | | binding protein) | | | | | | |
| | | (422 protein) | | | | | | |
| | | (P15), transcript v. | | | | | | |
| CF410483 | unmapped | gb\|CH3#067_D03MF | 1.0 | 3.4 | 3.3 | 45.5 | 0.0 | 0.0 |
| | | Canine heart | | | | | | |
| | | normalized | | | | | | |
| | | cDNA Library | | | | | | |
| | | in pBluescript | | | | | | |
| | | Canis familiaris | | | | | | |
| | | cDNA clone | | | | | | |
| | | CH3#067_D0 | | | | | | |
| | | 3 5′, mRNA | | | | | | |
| | | sequence | | | | | | |
| | | [CF410483] | | | | | | |
| ENSCAFT00000024881 | cf\|chr26: 41769211- | ens\|Cardiac actin | 1.1 | 3.4 | 3.2 | 40.8 | 0.0 | 0.0 |
| | 41769152 | Fragment | | | | | | |
| | | [Source: UniProtKB/ | | | | | | |
| | | TrEMBL; | | | | | | |
| | | Acc: Q9GKL5] | | | | | | |
| | | [ENSCAFT00000024881] | | | | | | |
| TC73969 | cf\|chr6: 31185946- | tc\|D85924 myosin | 0.8 | 2.6 | 3.1 | 14.6 | 0.0 | 0.0 |
| | 31186005 | {Mus musculus} | | | | | | |
| | | (exp = −1; | | | | | | |
| | | wgp = 0; | | | | | | |
| | | cg = 0), partial | | | | | | |
| | | (7%) | | | | | | |
| | | [TC73969] | | | | | | |
| STOM | cf\|chr11: 77297989- | ref\|Canis lupus | 0.8 | 2.3 | 2.8 | 8.7 | 0.4 | 0.0 |
| | 77297930 | familiaris | | | | | | |
| | | stomatin | | | | | | |
| | | (STOM), | | | | | | |
| | | mRNA | | | | | | |
| | | [NM_001142670] | | | | | | |
| GRIP2 | cf\|chr20: 7626747- | ens\|Glutamate | 0.7 | 2.1 | 2.8 | 8.7 | 0.1 | 0.0 |
| | 7626688 | receptor-interacting | | | | | | |
| | | protein 2 (GRIP2 | | | | | | |
| | | protein) | | | | | | |
| | | [Source: UniProtKB/ | | | | | | |
| | | Swiss-Prot; | | | | | | |
| | | Acc: Q9C0E4] | | | | | | |
| | | [ENSCAFT00000007240] | | | | | | |
| HNMT | cf\|chr19: 43720863- | ens\|Histamine N- | 1.1 | 2.9 | 2.6 | 45.5 | 0.0 | 3.1 |
| | 43720921 | methyltransferase | | | | | | |
| | | (HMT)(EC 2.1.1.8) | | | | | | |
| | | [Source: UniProtKB/ | | | | | | |
| | | Swiss-Prot; | | | | | | |
| | | Acc: P50135] | | | | | | |
| | | [ENSCAFT00000008347] | | | | | | |
| Jag1 | cf\|chr24: 014690227- | Unknown | 0.9 | 2.4 | 2.6 | 28.6 | 0.2 | 3.1 |
| | 014690169 | | | | | | | |
| DN876671 | cf\|chr4: 62169188- | gb\|nae07h11.y1 | 1.0 | 2.5 | 2.6 | 44.7 | 0.3 | 4.1 |
| | 62169247 | Dog eye | | | | | | |
| | | eye minus | | | | | | |
| | | lens and cornea. | | | | | | |
| | | Unnormalized (nae) | | | | | | |
| | | Canis familiaris | | | | | | |
| | | cDNA clone | | | | | | |
| | | nae07h11 5′, | | | | | | |
| | | mRNA sequence | | | | | | |
| | | [DN876671] | | | | | | |
| STOX2 | cf\|chr16: 49394858- | ens\|Storkhead-box | 0.8 | 1.9 | 2.5 | 10.4 | 2.3 | 4.7 |
| | 49394799 | protein 2 | | | | | | |
| | | [Source: UniProtKB/ | | | | | | |
| | | Swiss-Prot; | | | | | | |
| | | Acc: Q9P2F5] | | | | | | |
| | | [ENSCAFT00000012467] | | | | | | |
| TC70027 | cf\|chr23: 6378395- | Unknown | 1.1 | 2.8 | 2.5 | 32.7 | 0.1 | 3.1 |
| | 6378454 | | | | | | | |
| ENSCAFT00000022262 | cf\|chr2: 9203729- | gb\|PREDICTED: | 1.0 | 2.4 | 2.4 | 43.9 | 0.6 | 2.8 |
| | 9188870 | Canis familiaris | | | | | | |
| | | similar to ankyrin | | | | | | |
| | | repeat domain 26 | | | | | | |
| | | (LOC480753), | | | | | | |
| | | mRNA | | | | | | |
| | | [XM_537873] | | | | | | |
| TC69710 | cf\|chrX: 46476130- | tc\|BC058581 | 0.8 | 1.8 | 2.3 | 24.1 | 0.9 | 3.1 |
| | 46476071 | Maged2 protein | | | | | | |
| | | {Mus musculus} | | | | | | |
| | | (exp = −1; | | | | | | |
| | | wgp = 0; | | | | | | |
| | | cg = 0), partial | | | | | | |
| | | (22%) | | | | | | |
| | | [TC69710] | | | | | | |
| DN873043 | cf\|chrX: 56231420- | gb\|nad24a11.y2 | 1.1 | 2.7 | 2.3 | 28.6 | 0.0 | 2.8 |
| | 56231479 | Dog eye cornea. | | | | | | |
| | | Unnormalized (nad) | | | | | | |
| | | Canis familiaris | | | | | | |
| | | cDNA clone | | | | | | |
| | | nad24a11 5′, | | | | | | |
| | | mRNA sequence | | | | | | |
| | | [DN873043] | | | | | | |
| TC59827 | cf\|chr15: 9458422- | tc\|Q9D7C0_MOUSE | 0.7 | 1.7 | 2.3 | 6.9 | 0.6 | 3.1 |
| | 9458481 | (Q9D7C0) | | | | | | |
| | | Adult male tongue | | | | | | |
| | | cDNA, RIKEN | | | | | | |
| | | full-length enriched | | | | | | |
| | | library, | | | | | | |
| | | clone: 2310015N14 | | | | | | |
| | | product: gene | | | | | | |
| | | trap locus | | | | | | |
| | | F3a, full insert | | | | | | |
| | | sequence | | | | | | |
| | | (Gene trap | | | | | | |
| | | locus F3a), | | | | | | |
| | | partial (11%) | | | | | | |
| | | [TC59827] | | | | | | |
| SLC27A3 | cf\|chr7: 46296100- | ens\|Long-chain fatty | 1.0 | 2.2 | 2.2 | 45.5 | 0.1 | 3.1 |
| | 46296041 | acid transport | | | | | | |
| | | protein 3 | | | | | | |
| | | (Fatty acid | | | | | | |
| | | transport protein | | | | | | |
| | | 3)(FATP-3) | | | | | | |
| | | (EC 6.2.1.—) | | | | | | |
| | | (Very long- | | | | | | |
| | | chain acyl-CoA | | | | | | |
| | | synthetase homolog | | | | | | |
| | | 3)(VLCS-3)(Solute | | | | | | |
| | | carrier family | | | | | | |
| | | 27 member 3) | | | | | | |
| | | [Source: UniProtKB/ | | | | | | |
| | | Swiss-Prot; | | | | | | |
| | | Acc: Q5K4L6] | | | | | | |
| | | [ENSCAFT00000027642] | | | | | | |
| DN412165 | cf\|chr28: 5904377- | gb\|LIB4215- | 0.9 | 1.9 | 2.2 | 24.1 | 0.1 | 3.1 |
| | 5904436 | 038-R1-K1-H6 | | | | | | |
| | | LIB4215 | | | | | | |
| | | Canis familiaris | | | | | | |
| | | cDNA clone | | | | | | |
| | | CLN10730368, | | | | | | |
| | | mRNA sequence | | | | | | |
| | | [DN412165] | | | | | | |
| TC48849 | cf\|chrX: 41759656- | tc\|HSU28686 | 1.2 | 2.6 | 2.2 | 30.7 | 0.0 | 3.1 |
| | 41759715 | RNPL {Homo sapiens} | | | | | | |
| | | (exp = −1; | | | | | | |
| | | wgp = 0; | | | | | | |
| | | cg = 0), | | | | | | |
| | | complete | | | | | | |
| | | [TC48849] | | | | | | |
| MAOA | cf\|chrX: 37692616- | ref\|Canis lupus | 1.1 | 2.3 | 2.1 | 26.4 | 0.1 | 0.0 |
| | 37692673 | familiaris | | | | | | |
| | | monoamine oxidase A | | | | | | |
| | | (MAOA), | | | | | | |
| | | nuclear gene | | | | | | |
| | | encoding | | | | | | |
| | | mitochondrial | | | | | | |
| | | protein, mRNA | | | | | | |
| | | [NM_001002969] | | | | | | |
| A_11_P000006238 | cf\|chr21: 027864292- | Unknown | 1.0 | 2.1 | 2.0 | 43.9 | 0.2 | 3.1 |
| | 027864233 | | | | | | | |
| DN748546 | cf\|chr27: 4736701- | gb\|GL-Cf-5338 | 1.3 | 2.6 | 2.0 | 5.4 | 0.0 | 2.8 |
| | 4736760 | GLGC-LIB0001-cf | | | | | | |
| | | Canis familiaris | | | | | | |
| | | Normalized | | | | | | |
| | | Mixed Tissue | | | | | | |
| | | cDNA Library | | | | | | |
| | | Canis familiaris | | | | | | |
| | | cDNA, mRNA | | | | | | |
| | | sequence | | | | | | |
| | | [DN748546] | | | | | | |
| PROCA1 | cf\|chr9: 46222817- | gb\|PREDICTED: | 1.3 | 2.4 | 1.9 | 10.4 | 0.1 | 0.0 |
| | 46222758 | Canis familiaris | | | | | | |
| | | similar to | | | | | | |
| | | proline-rich | | | | | | |
| | | cyclin A1- | | | | | | |
| | | interacting | | | | | | |
| | | protein, | | | | | | |
| | | transcript | | | | | | |
| | | variant 1 | | | | | | |
| | | (LOC610597), | | | | | | |
| | | mRNA | | | | | | |
| | | [XM_849043] | | | | | | |
| A_11_P0000032579 | cf\|chr5: 017926320- | Unknown | 1.3 | 2.4 | 1.8 | 6.9 | 0.0 | 4.1 |
| | 017926379 | | | | | | | |
| SULT1A1 | cf\|chr6: 21191775- | ref\|Canis lupus | 1.0 | 1.8 | 1.8 | 40.8 | 0.1 | 2.8 |
| | 21191834 | familiaris | | | | | | |
| | | sulfotransferase | | | | | | |
| | | family, | | | | | | |
| | | cytosolic, 1A, | | | | | | |
| | | phenol-preferring, | | | | | | |
| | | member 1 | | | | | | |
| | | (SULT1A1), mRNA | | | | | | |
| | | [NM_001003223] | | | | | | |
| TC76672 | cf\|chr31: 34373870- | Unknown | 0.9 | 1.5 | 1.8 | 28.6 | 2.3 | 2.8 |
| | 34373929 | | | | | | | |
| FBXO18 | cf\|chr2: 32904044- | ens\|F-box | 0.8 | 1.4 | 1.7 | 12.3 | 3.1 | 0.0 |
| | 32892958 | only protein | | | | | | |
| | | 18 (EC 3.6.1.—) | | | | | | |
| | | (F-box DNA | | | | | | |
| | | helicase 1) | | | | | | |
| | | [Source: UniProtKB/ | | | | | | |
| | | Swiss-Prot; | | | | | | |
| | | Acc: Q8NFZ0] | | | | | | |
| | | [ENSCAFT00000008418] | | | | | | |
| TC53319 | cf\|chrX: 125184886- | tc\|Q6IRF8_RAT | 1.1 | 1.7 | 1.6 | 32.7 | 0.1 | 0.0 |
| | 125184945 | (Q6IRF8) | | | | | | |
| | | ATPase, H+ | | | | | | |
| | | transporting, | | | | | | |
| | | lysosomal | | | | | | |
| | | (Vacuolar proton | | | | | | |
| | | pump), subunit 1, | | | | | | |
| | | partial (15%) | | | | | | |
| | | [TC53319] | | | | | | |
| RBBP4 | cf\|chr2: 71531092- | gb\|PREDICTED: | 1.2 | 1.9 | 1.6 | 19.5 | 0.2 | 3.1 |
| | 71531033 | Canis familiaris | | | | | | |
| | | similar to | | | | | | |
| | | retinoblastoma | | | | | | |
| | | binding protein 4, | | | | | | |
| | | transcript | | | | | | |
| | | variant 2 | | | | | | |
| | | (LOC478149), mRNA | | | | | | |
| | | [XM_846636] | | | | | | |
| A_11_P000002485 | cf\|chrX: 108772909- | Unknown | 1.2 | 1.9 | 1.6 | 12.3 | 0.0 | 2.8 |
| | 108772850 | | | | | | | |
| ENSCAFT00000025497 | cf\|chrX: 44409160- | ens\|Melanoma- | 1.0 | 1.5 | 1.5 | 40.8 | 0.2 | 4.1 |
| | 44409219 | associated | | | | | | |
| | | antigen D1 | | | | | | |
| | | (MAGE-D1 | | | | | | |
| | | antigen) | | | | | | |
| | | (Neurotrophin | | | | | | |
| | | receptor- | | | | | | |
| | | interacting MAGE | | | | | | |
| | | homolog)(MAGE | | | | | | |
| | | tumor antigen CCF) | | | | | | |
| | | [Source: UniProtKB/ | | | | | | |
| | | Swiss-Prot; | | | | | | |
| | | Acc: Q9Y5V3] | | | | | | |
| | | [ENSCAFT00000025497] | | | | | | |
| LOC480007 | cf\|chr7: 5860170- | gb\|PREDICTED: | 1.1 | 1.6 | 1.5 | 19.5 | 0.1 | 3.1 |
| | 5860113 | Canis familiaris | | | | | | |
| | | hypothetical | | | | | | |
| | | LOC480007 | | | | | | |
| | | (LOC480007), | | | | | | |
| | | mRNA | | | | | | |
| | | [XM_549690] | | | | | | |
| TC69589 | cf\|chr18: 54380910- | tc\|MMU64690WF-3 | 1.1 | 1.5 | 1.4 | 28.6 | 0.1 | 3.1 |
| | 54380969 | {Mus musculus} | | | | | | |
| | | (exp = −1; | | | | | | |
| | | wgp = 0; | | | | | | |
| | | cg = 0), partial | | | | | | |
| | | (23%) | | | | | | |
| | | [TC69589] | | | | | | |
| TC62893 | cf\|chr20: 58905268- | tc\|Q71RA9_HUMAN | 1.0 | 1.5 | 1.4 | 42.0 | 0.2 | 2.8 |
| | 58905209 | (Q71RA9) PP7706, | | | | | | |
| | | partial (12%) | | | | | | |
| | | [TC62893] | | | | | | |
| SSR4 | cf\|chrX: 124657159- | ens\|Translocon- | 1.0 | 1.4 | 1.4 | 43.9 | 0.1 | 3.1 |
| | 124657218 | associated protein | | | | | | |
| | | subunit delta | | | | | | |
| | | Precursor (TRAP- | | | | | | |
| | | delta)(Signal | | | | | | |
| | | sequence receptor | | | | | | |
| | | subunit | | | | | | |
| | | delta)(SSR-delta) | | | | | | |
| | | [Source: UniProtKB/ | | | | | | |
| | | Swiss-Prot; | | | | | | |
| | | Acc: P51571] | | | | | | |
| | | [ENSCAFT00000030625] | | | | | | |
| ENSCAFT00000026083 | cf\|chrX: 46271044- | ens\|Guanine | 1.1 | 1.5 | 1.3 | 17.1 | 0.1 | 3.1 |
| | 46271103 | nucleotide-binding | | | | | | |
| | | protein-like | | | | | | |
| | | 3-like protein | | | | | | |
| | | [Source: UniProtKB/ | | | | | | |
| | | Swiss-Prot; | | | | | | |
| | | Acc: Q9NVN8] | | | | | | |
| | | [ENSCAFT00000026083] | | | | | | |
| ENSCAFT00000024029 | cf\|chr27: 41526908- | ens\|39S ribosomal | 0.9 | 0.7 | 0.8 | 24.1 | 0.2 | 4.1 |
| | 41526967 | protein L51, | | | | | | |
| | | mitochondrial | | | | | | |
| | | Precursor | | | | | | |
| | | (L51mt)(MRP-L51) | | | | | | |
| | | (bMRP-64)(bMRP64) | | | | | | |
| | | [Source: UniProtKB/ | | | | | | |
| | | Swiss-Prot; | | | | | | |
| | | Acc: Q4U2R6] | | | | | | |
| | | [ENSCAFT00000024029] | | | | | | |
| ERGIC2 | cf\|chr27: 21667255- | ens\|Endoplasmic | 1.1 | 0.8 | 0.8 | 14.6 | 0.4 | 2.6 |
| | 21667314 | reticulum-Golgi | | | | | | |
| | | intermediate | | | | | | |
| | | compartment | | | | | | |
| | | protein 2 | | | | | | |
| | | [Source: UniProtKB/ | | | | | | |
| | | Swiss-Prot; | | | | | | |
| | | Acc: Q96RQ1] | | | | | | |
| | | [ENSCAFT00000017305] | | | | | | |
| ENSCAFT00000010962 | cf\|chr18: 34706803- | ens\|TNF receptor- | 0.9 | 0.7 | 0.7 | 28.6 | 0.2 | 4.7 |
| | 34706862 | associated factor 6 | | | | | | |
| | | (Interleukin-1 | | | | | | |
| | | signal | | | | | | |
| | | transducer)(RING | | | | | | |
| | | finger protein 85) | | | | | | |
| | | [Source: UniProtKB/ | | | | | | |
| | | Swiss-Prot; | | | | | | |
| | | Acc: Q9Y4K3] | | | | | | |
| | | [ENSCAFT00000010962] | | | | | | |
| CHCHD1 | cf\|chr4: 27429292- | ens\|Coiled- | 1.0 | 0.7 | 0.7 | 36.5 | 1.7 | 0.0 |
| | 27429351 | coil-helix- | | | | | | |
| | | coiled-coil- | | | | | | |
| | | helix domain- | | | | | | |
| | | containing | | | | | | |
| | | protein 1 (C2360) | | | | | | |
| | | [Source: UniProtKB/ | | | | | | |
| | | Swiss-Prot; | | | | | | |
| | | Acc: Q96BP2] | | | | | | |
| | | [ENSCAFT00000023617] | | | | | | |
| HUS1 | cf\|chr16: 3106555- | ens\|Checkpoint | 1.2 | 0.7 | 0.6 | 12.3 | 0.3 | 4.1 |
| | 3106614 | protein HUS1 | | | | | | |
| | | (hHUS1) | | | | | | |
| | | [Source: UniProtKB/ | | | | | | |
| | | Swiss-Prot; | | | | | | |
| | | Acc: O60921] | | | | | | |
| | | [ENSCAFT00000039863] | | | | | | |
| ENSCAFT00000028877 | cf\|chr9: 41164788- | ens\|TATA-binding | 1.2 | 0.7 | 0.6 | 19.5 | 1.7 | 2.6 |
| | 41164729 | protein-associated | | | | | | |
| | | factor 2N | | | | | | |
| | | (RNA-binding | | | | | | |
| | | protein 56)(TAFII68) | | | | | | |
| | | (TAF(II)68) | | | | | | |
| | | [Source: UniProtKB/ | | | | | | |
| | | Swiss-Prot; | | | | | | |
| | | Acc: Q92804] | | | | | | |
| | | [ENSCAFT00000028877] | | | | | | |
| GFM2 | cf\|chr2: 60126677- | ens\|Elongation | 1.1 | 0.6 | 0.6 | 36.5 | 1.7 | 4.7 |
| | 60126618 | factor G 2, | | | | | | |
| | | mitochondrial | | | | | | |
| | | Precursor (mEF-G | | | | | | |
| | | 2)(Elongation | | | | | | |
| | | factor G2) | | | | | | |
| | | [Source: UniProtKB/ | | | | | | |
| | | Swiss-Prot; | | | | | | |
| | | Acc: Q969S9] | | | | | | |
| | | [ENSCAFT00000013184] | | | | | | |
| MRPL13 | cf\|chr13: 22316208- | ens\|39S ribosomal | 0.9 | 0.5 | 0.6 | 24.1 | 0.1 | 2.6 |
| | 22316149 | protein L13, | | | | | | |
| | | mitochondrial | | | | | | |
| | | (L13mt)(MRP-L13) | | | | | | |
| | | [Source: UniProtKB/ | | | | | | |
| | | Swiss-Prot; | | | | | | |
| | | Acc: Q9BYD1] | | | | | | |
| | | [ENSCAFT00000001460] | | | | | | |
| ENSCAFT00000014803 | cf\|chr33: 9946512- | ens\|Mitochondrial | 1.0 | 0.6 | 0.6 | 42.0 | 0.1 | 4.7 |
| | 9946453 | import receptor | | | | | | |
| | | subunit TOM70 | | | | | | |
| | | (Mitochondrial | | | | | | |
| | | precursor proteins | | | | | | |
| | | import receptor) | | | | | | |
| | | (Translocase of | | | | | | |
| | | outer membrane | | | | | | |
| | | 70 kDa subunit) | | | | | | |
| | | [Source: UniProtKB/ | | | | | | |
| | | Swiss-Prot; | | | | | | |
| | | Acc: O94826] | | | | | | |
| | | [ENSCAFT00000014803] | | | | | | |
| ENSCAFT00000025695 | cf\|chr7: 42217617- | ens\|Protein enabled | 1.2 | 0.7 | 0.6 | 17.1 | 4.1 | 1.8 |
| | 42218255 | homolog | | | | | | |
| | | [Source: UniProtKB/ | | | | | | |
| | | Swiss-Prot; | | | | | | |
| | | Acc: Q8N8S7] | | | | | | |
| | | [ENSCAFT00000025695] | | | | | | |
| ENSCAFT00000014731 | cf\|chr31: 31152118- | ens\|Trifunctional | 1.1 | 0.6 | 0.5 | 32.7 | 3.1 | 4.7 |
| | 31152059 | purine biosynthetic | | | | | | |
| | | protein adenosine-3 | | | | | | |
| | | [Includes | | | | | | |
| | | Phosphoribosylamine-- | | | | | | |
| | | glycine | | | | | | |
| | | ligase(EC 6.3.4.13) | | | | | | |
| | | (Glycinamide | | | | | | |
| | | ribonucleotide | | | | | | |
| | | synthetase)(GARS) | | | | | | |
| | | (Phosphoribosyl- | | | | | | |
| | | glycinamide | | | | | | |
| | | synthetase); | | | | | | |
| | | Phosphoribosylformyl- | | | | | | |
| | | glycinamidine | | | | | | |
| | | cyclo- | | | | | | |
| | | ligase(EC 6.3.3 | | | | | | |
| ENSCAFT00000028950 | cf\|chr6: 31062633- | ens\|LisH domain- | 0.9 | 0.4 | 0.5 | 30.7 | 0.1 | 4.7 |
| | 31062692 | containing protein | | | | | | |
| | | C16orf63 | | | | | | |
| | | [Source: UniProtKB/ | | | | | | |
| | | Swiss-Prot; | | | | | | |
| | | Acc: Q96NB1] | | | | | | |
| | | [ENSCAFT00000028950] | | | | | | |
| GABPA | cf\|chr31: 24266843- | gb\|PREDICTED: | 1.1 | 0.5 | 0.5 | 39.5 | 0.0 | 4.1 |
| | 24266902 | Canis familiaris | | | | | | |
| | | similar to GA | | | | | | |
| | | binding protein | | | | | | |
| | | transcription | | | | | | |
| | | factor, alpha | | | | | | |
| | | subunit (60 kD) | | | | | | |
| | | (LOC478394), mRNA | | | | | | |
| | | [XM_535570] | | | | | | |
| DN745532 | cf\|chr30: 21180756- | gb\|GL-Cf-2324 | 1.1 | 0.5 | 0.5 | 22.0 | 0.1 | 1.8 |
| | 21180697 | GLGC-LIB0001-cf | | | | | | |
| | | Canis familiaris | | | | | | |
| | | Normalized | | | | | | |
| | | Mixed Tissue | | | | | | |
| | | cDNA Library | | | | | | |
| | | Canis familiaris | | | | | | |
| | | cDNA, mRNA | | | | | | |
| | | sequence | | | | | | |
| | | [DN745532] | | | | | | |
| UCHL3 | cf\|chr22: 32344465- | ens\|LIM domain only | 1.1 | 0.5 | 0.5 | 40.8 | 0.0 | 2.6 |
| | 32344524 | protein 7 | | | | | | |
| | | (LOMP)(F-box only | | | | | | |
| | | protein 20) | | | | | | |
| | | [Source: UniProtKB/ | | | | | | |
| | | Swiss-Prot; | | | | | | |
| | | Acc: Q8WWI1] | | | | | | |
| | | [ENSCAFT00000038963] | | | | | | |
| A_11_P0000041905 | cf\|chr11: 065475115- | Unknown | 1.1 | 0.5 | 0.5 | 28.6 | 1.3 | 4.7 |
| | 065475174 | | | | | | | |
| ARPP19 | cf\|chr30: 21184681- | tc\|Q6IAM2_HUMAN | 0.9 | 0.4 | 0.4 | 34.7 | 0.1 | 4.1 |
| | 21184622 | (Q6IAM2) ARPP-19 | | | | | | |
| | | protein, | | | | | | |
| | | partial (87%) | | | | | | |
| | | [TC47534] | | | | | | |
| A_11_P0000025712 | cf\|chrX: 074778180- | Unknown | 1.0 | 0.5 | 0.4 | 43.0 | 0.2 | 4.1 |
| | 074778238 | | | | | | | |
| PTPN4 | cf\|chr19: 33112790- | ens\|Tyrosine-protein | 1.0 | 0.4 | 0.4 | 38.1 | 0.0 | 2.6 |
| | 33107660 | phosphatase | | | | | | |
| | | non-receptor | | | | | | |
| | | type 4 (EC 3.1.3.48) | | | | | | |
| | | (Protein-tyrosine | | | | | | |
| | | phosphatase | | | | | | |
| | | MEG1)(PTPase- | | | | | | |
| | | MEG1)(MEG) | | | | | | |
| | | [Source: UniProtKB/ | | | | | | |
| | | Swiss-Prot; | | | | | | |
| | | Acc: P29074] | | | | | | |
| | | [ENSCAFT00000007849] | | | | | | |
| AKR7A2 | cf\|chr2: 82235958- | ens\|Aflatoxin | 1.2 | 0.5 | 0.4 | 26.4 | 0.2 | 3.0 |
| | 82236969 | B1 aldehyde | | | | | | |
| | | reductase member 2 | | | | | | |
| | | (EC 1.—.—.—) | | | | | | |
| | | (AFB1-AR1) | | | | | | |
| | | (Aldoketoreductase 7) | | | | | | |
| | | [Source: UniProtKB/ | | | | | | |
| | | Swiss-Prot; | | | | | | |
| | | Acc: O43488] | | | | | | |
| | | [ENSCAFT00000024096] | | | | | | |
| DCUN1D2 | cf\|chr22: 63733602- | ens\|DCN1-like | 0.8 | 0.3 | 0.4 | 8.7 | 0.1 | 2.6 |
| | 63733543 | protein 2 | | | | | | |
| | | (Defective in | | | | | | |
| | | cullin neddylation | | | | | | |
| | | protein 1-like | | | | | | |
| | | protein 2)(DCUN1 | | | | | | |
| | | domain-containing | | | | | | |
| | | protein 2) | | | | | | |
| | | [Source: UniProtKB/ | | | | | | |
| | | Swiss-Prot; | | | | | | |
| | | Acc: Q6PH85] | | | | | | |
| | | [ENSCAFT00000010286] | | | | | | |
| ENSCAFT00000008933 | cf\|chr2: 36968326- | ens\|La-related | 0.7 | 0.3 | 0.3 | 26.4 | 0.1 | 0.0 |
| | 36968385 | protein 5 (La | | | | | | |
| | | ribonucleoprotein | | | | | | |
| | | domain family | | | | | | |
| | | member 5) | | | | | | |
| | | [Source: UniProtKB/ | | | | | | |
| | | Swiss-Prot; | | | | | | |
| | | Acc: Q92615] | | | | | | |
| | | [ENSCAFT00000008933] | | | | | | |
| TC55617 | cf\|chr32: 41133223- | tc\|BC017382 USP53 | 1.2 | 0.4 | 0.3 | 38.1 | 0.1 | 2.6 |
| | 41133282 | protein | | | | | | |
| | | {Homo sapiens} | | | | | | |
| | | (exp = −1; | | | | | | |
| | | wgp = 0; | | | | | | |
| | | cg = 0), partial | | | | | | |
| | | (29%) | | | | | | |
| | | [TC55617] | | | | | | |
| DN880809 | cf\|chr9: 51530055- | gb\|nae36d08.y1 | 1.4 | 0.5 | 0.3 | 24.1 | 0.4 | 4.7 |
| | 51530114 | Dog eye | | | | | | |
| | | eye minus | | | | | | |
| | | lens and cornea. | | | | | | |
| | | Unnormalized (nae) | | | | | | |
| | | Canis familiaris | | | | | | |
| | | cDNA clone | | | | | | |
| | | nae36d08 5′, | | | | | | |
| | | mRNA sequence | | | | | | |
| | | [DN880809] | | | | | | |
| A_11_P0000014210 | cf\|chr2: 084527822- | Unknown | 1.3 | 0.4 | 0.3 | 28.6 | 0.1 | 4.7 |
| | 084527763 | | | | | | | |
| DN756656 | cf\|chr17: 54528470- | gb\|GL-Cf-13452 | 1.3 | 0.4 | 0.3 | 26.4 | 0.2 | 4.7 |
| | 54528529 | GLGC-LIB0001-cf | | | | | | |
| | | Canis familiaris | | | | | | |
| | | Normalized | | | | | | |
| | | Mixed Tissue | | | | | | |
| | | cDNA Library | | | | | | |
| | | Canis familiaris | | | | | | |
| | | cDNA, mRNA | | | | | | |
| | | sequence | | | | | | |
| | | [DN756656] | | | | | | |
| UBE2D1 | cf\|chr4: 13753844- | gb\|PREDICTED: | 1.0 | 0.2 | 0.2 | 36.5 | 0.0 | 2.6 |
| | 13753905 | Canis familiaris | | | | | | |
| | | similar to | | | | | | |
| | | ubiquitin- | | | | | | |
| | | conjugating | | | | | | |
| | | enzyme E2D1, UBC4 | | | | | | |
| | | [XM_845637] | | | | | | |
| MPHOSPH8 | cf\|chr25: 21403649- | ref\|Canis lupus | 1.3 | 0.2 | 0.2 | 24.1 | 0.1 | 0.0 |
| | 21403590 | familiaris | | | | | | |
| | | M-phase | | | | | | |
| | | phosphoprotein 8 | | | | | | |
| | | (MPHOSPH8), mRNA | | | | | | |
| | | [NM_001145981] | | | | | | |
| A_11_P0000041686 | cf\|chr34: 019918957- | Unknown | 2.2 | 0.4 | 0.2 | 6.9 | 0.9 | 4.7 |
| | 019918898 | | | | | | | |

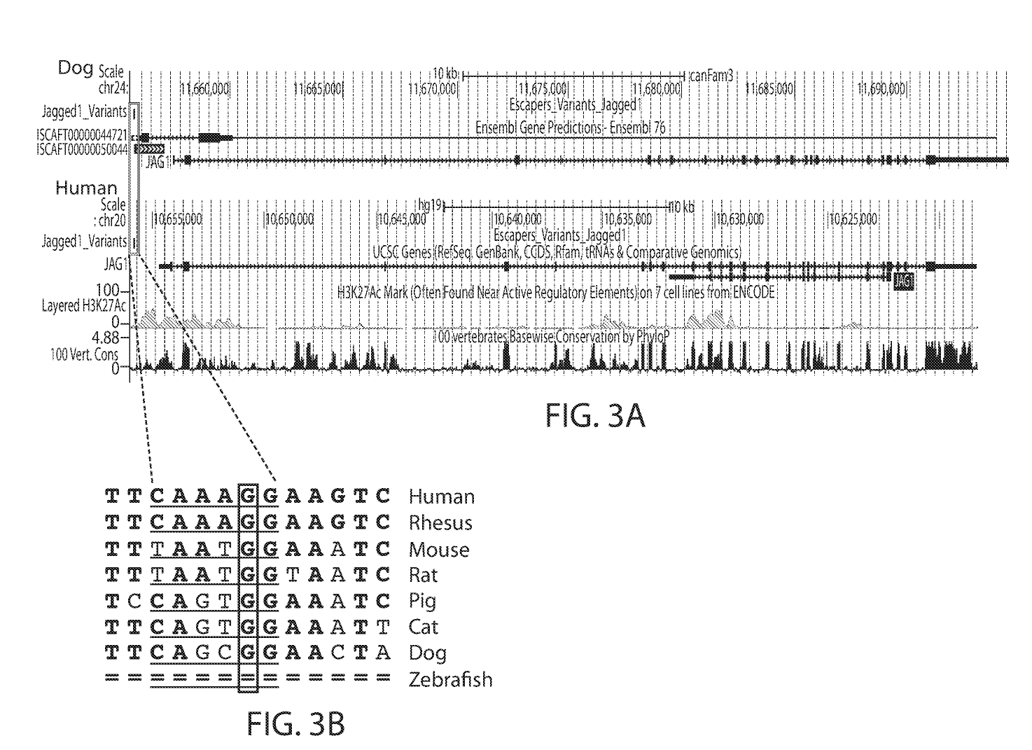

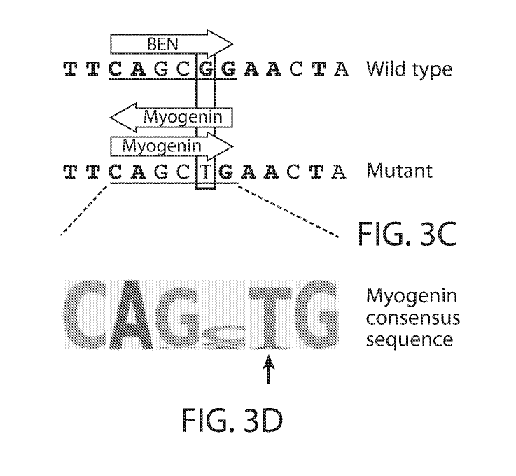

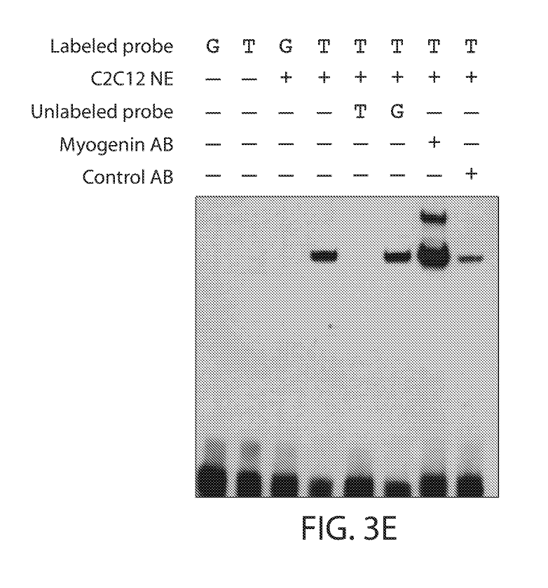

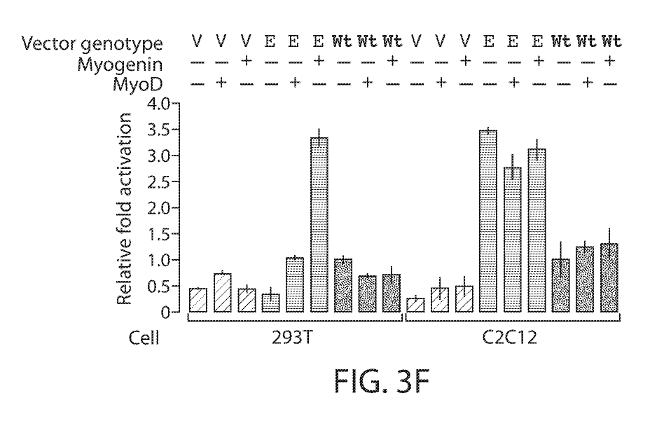

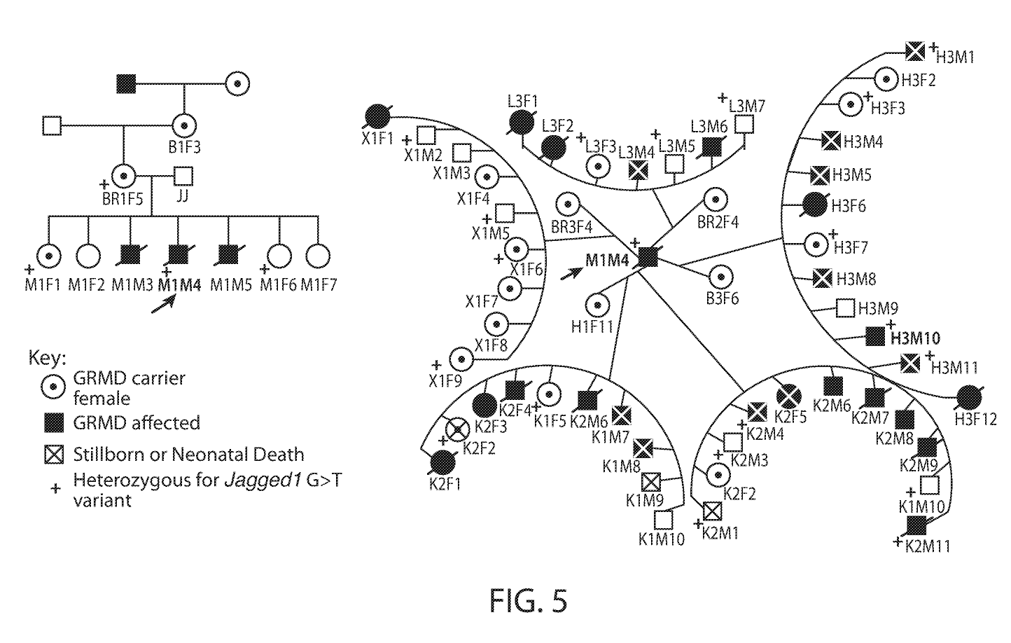

To identify potentially causative variants behind the differential gene expression observed in the escaper dogs, whole-genome sequencing of 3 dogs (2 escapers and 1 severely affected related dog) was performed. Variants within the Jagged1 locus (including 3KB upstream and downstream of the gene) were filtered, aiming to find a new mutation present only in the escaper GRMD dogs and not in the affected sibling. A total of ˜1300 variants followed the haplotype. After filtering for SNPs seen in previous data sets [ref. 17], variants were lifted over to the human genome and analyzed based on muscle enhancer marks near the promoters of the two isoforms of Jagged1 expressed in skeletal muscle (PMC3953330). Only a single point mutation was found after filtering, a G>T change at dog chr24:11644709 (canfam3.1) (FIG. 3A). Sanger sequencing of the three GRMD dogs confirmed that the candidate mutation was unique to the two escapers. Sequences of dogs related to the escaper dog showed that the variant segregated in the expected frequency. The low incidence of escapers in the colony was due to low frequency of dogs carrying both mutations: the GRMD dystrophin mutation and the new variant at Jagged1 promoter. All affected dogs lacked the Jagged1 variant, while both escapers were heterozygous. Thus, the novel Jagged1 mutation segregates with the escaper phenotype in this family. Five GRMD offspring were found to carry both mutations, three were stillborn, one died at six months-old of a common Golden Retriever gastric problem and one was the second escaper (FIG. 5). The candidate variant is conserved across 29 mammals (FIG. 3B). Transcription factor binding site analysis (TRAP and TRANSFAC) showed that the variant present in the escaper dogs created a Myogenin binding site (FIG. 3C) and that the mutated base (T) was important in the binding (FIG. 3D). Myogenin is a muscle specific transcription factor involved in muscle differentiation and repair [ref. 18]. To determine whether the variant affects DNA binding by transcription factor Myogenin, electrophoretic mobility shift assays (EMSA) were carried out using muscle cell nuclear extracts and biotin labeled oligonucleotide probes containing both genotypes (FIG. 3E). The oligonucleotide probe containing the escaper T allele robustly bound the Myogenin protein, whereas an oligonucleotide probe containing the wild-type G allele did not bind at all. A competition assay with unlabeled T probe efficiently competed with the binding of the labeled T probe. In contrast, the unlabeled G probe had no effect on the binding activity of the labeled T probe, indicating a specific interaction between the T allele and Myogenin.

To evaluate if the new Myogenin binding site was driving the overexpression of Jagged1 we performed a luciferase reporter assay using Jagged1 upstream promoter sequences containing the wild type sequence and escaper variant. Luciferase vectors containing either wildtype or escaper were transfected into muscle cells and embryonic kidney cells along with constructs that overexpress either Myogenin or MyoD as control. Muscle cells transfected with the escaper vector show luciferase activity that is three times higher than the Wt vector, notwithstanding the presence of overexpression vectors. In the myogenin-deficient cell line, luciferase activity was present only in the presence of both the escaper vector and Myogenin, but not when MyoD was added, indicating that Jagged1 overexpression is specifically driven by Myogenin (FIG. 3F).

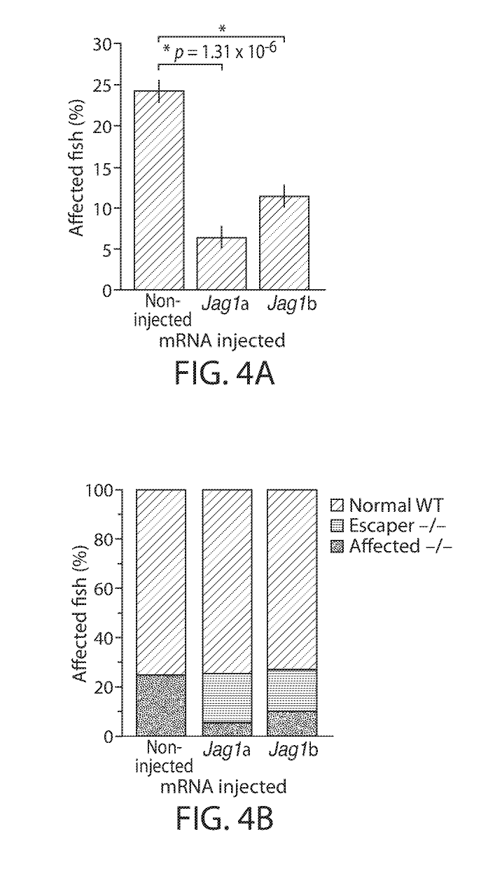

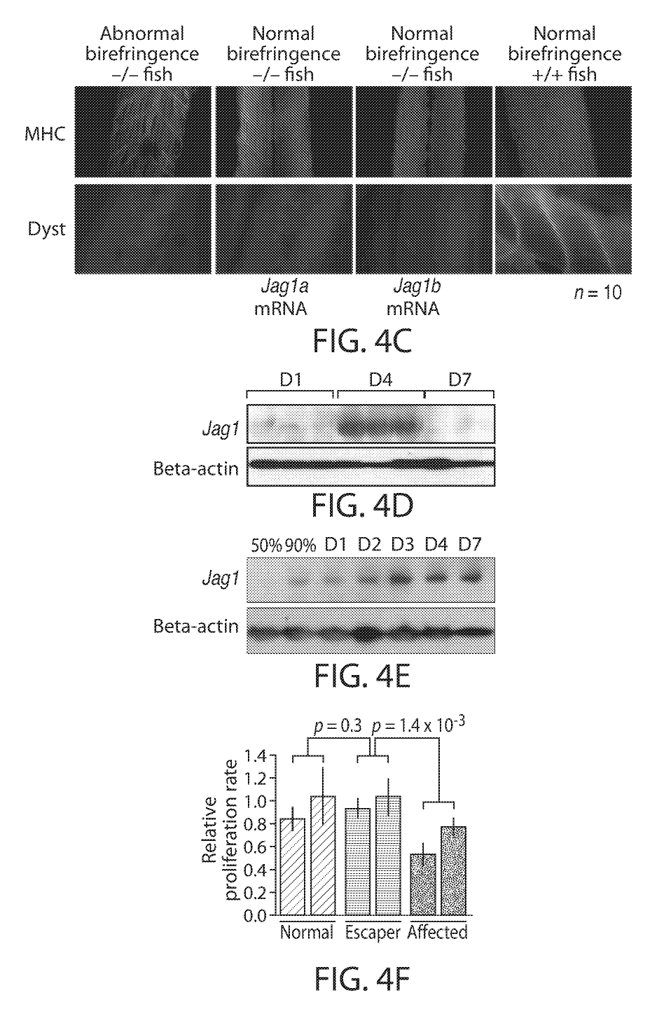

To evaluate if overexpression of jagged1 could improve the dystrophic muscle phenotype, the zebrafish DMD model (sapje) was used. In four separate experiments, ˜200 fertilized one-cell stage eggs, from a sapje heterogygous fish mating, were injected with 200 pg of mRNA of either one of the zebrafish jagged1 gene copies: jagged1 a or jagged1b. In all experiments, an average of 24% of the non-injected sapje fish exhibited a typical dystrophic patchy birefringence phenotype. This proportion was within the 21-27% expected range of affected fish on a heterozygous sapje mate. The fish injected with jagged1a and jagged1b both showed a significantly lower percentage of fish exhibiting poor birefringence (FIG. 4A). Genotyping analysis revealed that about 75% of sapje homozygous fish injected with jagged1a and 60% of jagged1b had normal birefringence, which demonstrated a common “recovery” from the muscle lethality phenotype (FIG. 4B). All phenotypically unaffected fish were confirmed to be WT or jagged1 heterozygous fish by genotyping. These results indicate that many of the dystrophin-null fish with overexpression of jagged1 failed to develop the abnormalities typically seen in dystrophin-null fish. To further evaluate the jagged1a and jagged1b overexpression fish, immunostaining of individual fish bodies was performed with myosin heavy chain (MHC) antibody. In the normal fish, MHC was clearly expressed and highlighted that muscle fibers were normal. Interestingly, MHC staining of jagged1 mRNA injected recovered dystrophin-null fish showed normal myofiber structure, similar to that of the normal fish, whereas affected dystrophin-null fish showed clear abnormalities of muscle (FIG. 4C).

The role of Jagged1 in skeletal muscle is not clear. Jagged1 is a Notch ligand. The notch signaling pathway represents a central regulator of gene expression [ref. 19] and is critical for cellular proliferation, differentiation and apoptotic signaling during all stages of embryonic muscle development [ref. 20, 21]. Notch signaling in mammalian cells is initiated by the ligation of the extracellular ligands (Delta and Jagged) [ref. 21, 22] to the Notch family of transmembrane receptors. It results in the cleavage of Notch intracellular domain (NICD) and subsequent activation of gene expression in the nucleus. Transcription factors known to be activated by Notch include the Hairy and Enhancer of Split (Hes) and Hes-related (Hey) families that are sequestered to the nucleus [ref. 23-26]. The regulation of Hes and Hey by Notch in skeletal muscle is still not clear, however it is known that they play a role in satellite cell activation and differentiation during muscle regeneration [ref. 27], and the upregulation of Hes and Hey via activation of Notch1 promotes satellite cell proliferation [ref. 21]. Recently, it was also shown that the dystrophin-deficient mdx mice and the severely dystrophic mdx:utrn double knockout mouse model had reduced overall Notch signaling expression levels (Jiang, C. et al. Dis Model Mech 7, 997-1004 (2014); Church, J. E. et al. Exp Physiol 99, 675-87 (2014)). The Notch pathway also plays an important role in regeneration after myotoxic injury [ref 21, 26, 28, 29] and overexpression of Notch improves muscle regeneration in aged mice [ref. 30]. Jagged1 expression is upregulated at day 4 after cardiotoxin-induced tibialis in anterior muscle injury cells. Day 4 is known by myoblast proliferation and fusion during the muscle regeneration [ref. 31] (FIG. 4D). Additionally Jagged1 is upregulated during myoblast muscle differentiation in vitro (FIG. 4E). To evaluate if muscle cells from escaper dogs proliferated faster than cells from severely affected dogs, a proliferation assay (MTTA) was performed. Muscle cells from escaper dogs divided at the same rate as normal dog cells but divided significantly faster than those from severely affected dogs (FIG. 4F), showing that Jagged1 overexpression may be involved in muscle cell proliferation and repair.

There is no cure for DMD, and existing therapies are ineffective. The study herein shows that overexpression of Jagged1 is likely to modulate the phenotype in GRMD dogs. Overexpression of Jagged1 rescued the dystrophic phenotype of the zebrafish model of muscular dystrophy. Based on the study herein, therefore, it is believed that increasing jagged1 in muscle is an effective therapy for DMD. Additionally, because increasing jagged1 expression affected muscle phenotype, it is also believed to be useful in treating various muscular dystrophies.

### Material and Methods

### GRMD Dogs

All animals were housed and cared and genotyped at birth as previously described [ref. 32]. GRMD dogs were identified by microchips. Animal care and experiments were performed in accordance with an animal research ethics committee.

### GWAS

DNA was isolated from blood and genotyped using the Illumina 170K canine HD array. Insufficiently genotyped individuals and lowly genotyped SNPs were filtered out using Plink (PLINK pngu.mgh.harvard.edu/purcell/plink/), leaving 2 escapers, 31 affected. EMMAX was used to calculate the principal components of the whole-genome SNP genotype data per individual by the EIGENSTRAT method. The threshold for genome-wide significance for each association analysis was defined based on the 95% confidence intervals (CIs) calculated from the beta distribution of observed p values, a method adopted from the study by the Wellcome Trust Case Control consortium [ref 4]. Identity By Descent (IBD) was calculated using Beagle.

mRNA Expression Profile

Sample labelling and array hybridization were performed according to the Two-Color Microarray-Based Gene Expression Analysis—Low Input Quick Amp Labeling—protocol (Agilent Technologies) using the SurePrint Canine 4×44K (Agilent Technologies) Microarray (GEO Platform GPL11351). Samples were labelled with Cy5 and a single RNA from a normal individual was labeled with Cy3 and used as a common reference on all arrays. A dye-swap technical replicates approach was also applied, where all samples were labeled with cy3 and the reference RNA was labeled with cy5. Labeled cRNA was hybridized using Gene Expression Hybridization Kit (Agilent). Slides were washed and processed according to the Agilent Two-Color Microarray-Based Gene Expression Analysis protocol (Version 5.5) and scanned on a GenePix 4000 B scanner (Molecular Devices, Sunnyvale, Calif., USA). Fluorescence intensities were extracted using Feature Extraction (FE) software (version 9.0; Agilent). Averaged values of dye-swap technical replicates were used for further analysis. Genes differentially expressed normal, mild and affected animals were identified with the Significance Analysis of Microarray (SAM) statistical approach [ref. 33], using the following parameters: one-class unpaired responses, t-statistic, 100 permutations. False discovery rate (FDR) was 5%. The raw data has been deposited in Gene Expression Omnibus (GEO) http://www.ncbi.nlm.nih.gov/geo/ database under accession number.

### Whole Genome Sequencing and Variant Calling

Whole-genome sequencing was performed to 30× depth of 3 dogs (2 escapers and 1 severely affected related dog). Samples were sequenced on an Illumina HiSeq 2000, sequencing reads were aligned to the CanFam 3.1 reference sequence using BWA [ref. 34]. Following GATK [ref. 35] base quality score recalibration, indel realignment (PMID: 21478889), duplicate removal, SNP and INDEL discovery was performed. Variant discovery across the 3 samples was performed using standard hard filtering parameters or variant quality score recalibration according to GATK Best Practices recommendations [ref. 36]. Variants were called in the jagged1 gene including the 3KB regions upstream and downstream of the gene (chr24:11654000-11696000, canfam3). Variants were filtered aiming to find a new mutation present only in the escaper GRMD dogs and not in the affected siblings or previous canine SNP data sets [ref. 17] using a custom perl script. Variants were lifted-over to human genome using UCSC Genome Browser. Variants present in muscle regulatory regions (ENCODE) were considered of interest.

### Electrophoretic Mobility Shift Assay (EMSA)

The duplex DNAs obtained by annealing of complementary oligonucleotides were either biotin labeled or unlabeled competitors. Probe sequences were: WT: CTCCTTTATTTCAGCGGAACTAAAGAAGTCTC (SEQ ID NO: 9) and for the variant CTCCTTTATTTCAGCTGAACTAAAGAAGTCTC (SEQ ID NO: 10). Biotin-labeled probes (0.5 pmol) were incubated with C2C12 nuclear extract (Active Motif) on ice for 20 min in the reaction buffer (10 mM Tris (pH 7.5), 50 mM KCl, mM EDTA, 1 mM DTT, 50% glycerol, 50 ng/ul of poly (dI.dC) and 10 ug/ul of bovine serum albumin). For competition experiments, unlabeled competitor DNAs in 100-fold molar excess over the labeled probe were included in the binding reactions. Supershift assay was performed with 5 ug of anti-myogenin F5D (Santa Cruz Biotechnology) mouse monoclonal antibody incubated 30 min at room temperature. Anti-mouse IgG (ABCAM) was used as control antibody. Samples were loaded onto 6% DNA Retardation Gels (Invitrogen) and separated at 10 V/cm in 0.5 TBE (45 mM Tris, 45 mM borate, 1 mM EDTA). Transfer to positive charged nylon membrane (GE-Hybond-N⁺) was performed in 0.5 TBE at 5° C. for 1 hour at 380 mA. Membrane crosslink was performed at 120 mJ/cm². Detection of the biotin-labeled DNA was carried out by chemiluminescence using LightShift Chemoluminescent EMSA kit (Thermo Scientific) following manufacturer's instructions.

### Zebrafish Lines

Zebrafish were housed and maintained as breeding stocks as previously described [ref. 37].

### Sapje Genotyping

Genomic DNA extracted from injected fish [ref. 38] and controls was amplified with for sapje mutation: forward primer 5′-CTGGTTACATTCTGAGAGACTTTC-3′ (SEQ ID NO: 11) and reverse primer 5′-AGCCAGCTGAACCAATTAACTCAC-3′ (SEQ ID NO: 12), with a 52° C. annealing temperature and 35 cycles. PCR products were purified using ExoSap (Affymetrix) and sequenced.

Zebrafish jagged1 Overexpression

Fertilized one-cell stage eggs from a sapje heterogygous fish mating were injected with 20 pg of mRNA of either one of the zebrafish jagged1 gene copies: jagged1a or jagged1b. Plasmid constructs were kindly provided [ref. 39] and linearized by Not1 restriction digestion. mRNA was synthesized with SP6 message machine kit (AMBION) and purified with mini Quick Spin Columns (Roche). Overexpression of zebrafish jagged1 was confirmed by western blot for HA tag (data not shown). Zebrafish injected with either mRNA and non-injected controls and assessed for phenotypic changes at 4 days post fertilization (4dpf), each injection was performed four times. Approximately 200 embryos were injected at each dosage.

### Zebrafish Birefringence Assay

The muscle phenotype was detected by using a birefringence assay, a technique used to analyze muscle quality due to the unique ability of highly organized sarcomeres to rotate polarized light. The birefringence assay was performed by placing anesthetized embryos on a glass polarizing filter and covering them with a second polarizing filter. The filters were placed on an underlit dissecting scope and the top polarizing filter was twisted until the light refracting through the zebrafish's striated muscle was visible.

### Zebrafish Immunostaining

Immunostaining was performed in 4 dpf embryos. Embryos were fixed in 4% paraformaldehyde (PFA) in PBS at 4° C. overnight and dehydrated in 100% methanol. After rehydration, 4 dpf embryos were incubated in 0.1% collagenase (Sigma) in PBS for 60 min. Blocking solution containing 0.2% saponin was used for 4 dpf embryos. Anti-slow muscle myosin heavy chain antibody (F59, Developmental Studies Hybridoma Bank; 1:50) was used. The embryos were placed in 3% methyl cellulose or mounted on a glass slide and observed with fluorescent microscopes (Nikon Eclipse E1000 and Zeiss Axioplan2).

### Western Blot

Muscle sample proteins were extracted using RIPA buffer with proteinase inhibitor tablets (Roche). Samples were centrifuged at 13,000 g for 10 minutes to remove insoluble debris. Soluble proteins were resolved by 6% sodium dodecyl sulfate-polyacrylamide gel electrophoresis (SDS-PAGE), and transferred to nitrocellulose membranes (Hybond; Amersham Biosciences). All membranes were stained with Ponceau (Sigma-Aldrich) to evaluate the amount of loaded proteins. Blots were blocked for 1 hour in Tris-buffered saline Tween (TBST) containing 5% powdered skim milk and reacted overnight with the following primary antibodies: anti-jagged1 (C-20, Santa Cruz Biotechnology, 1:1000). Horseradish peroxidase (HRP)-conjugated secondary antibody (Santa Cruz Biotechnology, 1:1000) was used to detect immunoreactive bands with Pierce ECL 2 (Thermo). Anti-beta actin antibody (HRP-ABCAM) was used as a loading control.

### Cell Growth Assay

The GRMD and normal myoblasts were plated at 96-well plates in three different concentrations: 100, 1000 and 10000 cells/well. All samples were plated in triplicate. Cells were maintained in DMEM-HG (Dulbecco's modified Eagle's medium with high glucose; Gibco) supplemented with 20% (v/v) FBS (fetal bovine serum; Gibco) and anti-anti (Gibco) at 37° C. and 5% CO₂ for one week. Cell growth was measured using the CellTiter 96 Non-Radioactive Cell Proliferation Assay (MTT—Promega) following manufacture's protocol. Absorbance was recorded using the Synergy2 plate reader (BioTek).

### Cardiotoxin Injury.

Mice were injured using an injection of 40 μl of 10 μM cardiotoxin isolated from Naja mossambica mossambica snake venom (Sigma-Aldrich, C-9759) into the right TA muscle. The left, contralateral TA muscle served as an uninjured control.

### REFERENCES

1. Monaco, A. P., Neve, R. L., Colletti-Feener, C., Bertelson, C. J., Kurnit, D. M, and Kunkel, L. M. Isolation of candidate cDNAs for portions of the Duchenne muscular dystrophy gene. Nature 323, 646-650 (1986).
2 Koenig, M. et al. Complete cloning of the Duchenne muscular dystrophy (DMD) cDNA and preliminary genomic organization of the DMD gene in normal and affected individuals. Cell 50, 509-517, doi:0092-8674(87)90504-6 [pii] (1987).
3 Hoffman, E. P., Brown, R. H., Jr. & Kunkel, L. M. Dystrophin: the protein product of the Duchenne muscular dystrophy locus. Cell 51, 919-928, doi:0092-8674(87)90579-4 [pii] (1987).
4 Norwood, F. L. M., Sutherland-Smith, A. J., Keep, N. H. & Kendrick-Jones, J. The structure of the N-terminal actin-binding domain of human dystrophin and how mutations in this domain may cause Duchenne or Becker muscular dystrophy. Structure 8, 481-491 (2000).
5 Monaco, A. P., Bertelson, C. J., Liechti-Gallati, S., Moser, H. & Kunkel, L. M. An explanation for the phenotypic differences between patients bearing partial deletions of the DMD locus. Genomics 2, 90-95 (1988).
6 Gussoni, E. et al. Long-term persistence of donor nuclei in a Duchenne muscular dystrophy patient receiving bone marrow transplantation. The Journal of Clinical Investigation 110, 807-814 (2002).
7. Kornegay, J. N., Tuler, S. M., Miller, D. M. & Levesque, D. C. Muscular dystrophy in a litter of golden retriever dogs. Muscle and Nerve 11, 1056-1064, doi:10.1002/mus.880111008 (1988).
8 Chapman, V. M., Miller, D. R., Armstrong, D. & Caskey, C. T. Recovery of induced mutations for X chromosome-linked muscular dystrophy in mice. Proceedings of the National Academy of Sciences of the United States of America 86, 1292-1296 (1989).
9 Bassett, D. & Currie, P. D. Identification of a zebrafish model of muscular dystrophy. Clin Exp Pharmacol Physiol 31, 537-540, doi:10.1111/j.1440-1681.2004.04030.x CEP4030 [pii] (2004).
10 Im, W. B. et al. Differential Expression of Dystrophin Isoforms in Strains of mdx Mice with Different Mutations. Human Molecular Genetics 5, 1149-1153, doi:10.1093/hmg/5.8.1149 (1996).
11 Bassett, D. I. et al. Dystrophin is required for the formation of stable muscle attachments in the zebrafish embryo. Development 130, 5851-5860, doi:10.1242/dev.00799 130/23/5851 [pii] (2003).
12 Zucconi, E. et al. Ringo: discordance between the molecular and clinical manifestation in a golden retriever muscular dystrophy dog. Neuromuscul Disord 20, 64-70, doi:50960-8966(09)00664-6 [pii]10.1016/j.nmd.2009.10.011 (2009).
13 Fairclough, R. J., Wood, M. J. & Davies, K. E. Therapy for Duchenne muscular dystrophy: renewed optimism from genetic approaches. Nat Rev Genet 14, 373-378, doi:10.1038/nrg3460 nrg3460 [pii] (2013).
14 Kerkis, I. et al. Early transplantation of human immature dental pulp stem cells from baby teeth to golden retriever muscular dystrophy (GRMD) dogs: Local or systemic? J Transl Med 6, 35, doi:10.1186/1479-5876-6-35 1479-5876-6-35 [pii] (2008).
15 Kang, H. M. et al. Variance component model to account for sample structure in genome-wide association studies. Nat Genet 42, 348-354, doi:10.1038/ng.548 ng.548 [pii] (2010).
16 Browning, S. R. & Browning, B. L. Rapid and accurate haplotype phasing and missing-data inference for whole-genome association studies by use of localized haplotype clustering. Am J Hum Genet 81, 1084-1097, doi:S0002-9297(07)63882-8 [pii] 10.1086/521987 (2007).
17 Axelsson, E. et al. The genomic signature of dog domestication reveals adaptation to a starch-rich diet. Nature 495, 360-364, doi:10.1038/nature11837 nature11837 [pii] (2013).
18 Wright, W. E., Sassoon, D. A. & Lin, V. K. Myogenin, a factor regulating myogenesis, has a domain homologous to MyoD. Cell 56, 607-617, doi:0092-8674(89)90583-7 [pii] (1989).
19 Fischer, A. & Gessler, M. Delta-Notch—and then? Protein interactions and proposed modes of repression by Hes and Hey bHLH factors. Nucleic acids research 35, 4583-4596, doi:gkm477 [pii] 10.1093/nar/gkm477 (2007).
20 Artavanis-Tsakonas, S., Rand, M. D. & Lake, R. J. Notch signaling: cell fate control and signal integration in development. Science 284, 770-776 (1999).
21 Conboy, I. M. & Rando, T. A. The regulation of Notch signaling controls satellite cell activation and cell fate determination in postnatal myogenesis. Dev Cell 3, 397-409, doi:S153458070200254X [pii] (2002).
22 Lai, E. C. Notch signaling: control of cell communication and cell fate. Development 131, 965-973, doi:10.1242/dev.01074 131/5/965 [pii] (2004).
23 Jarriault, S. et al. Signalling downstream of activated mammalian Notch. Nature 377, 355-358, doi:10.1038/377355a0 (1995).
24 Shinin, V., Gayraud-Morel, B., Gomes, D. & Tajbakhsh, S. Asymmetric division and cosegregation of template DNA strands in adult muscle satellite cells. Nat Cell Biol 8, 677-687, doi:ncb1425 [pii] 10.1038/ncb1425 (2006).
25 Carlson, M. E. & Conboy, I. M. Regulating the Notch pathway in embryonic, adult and old stem cells. Curr Opin Pharmacol 7, 303-309, doi:S1471-4892(07)00064-1 [pii] 10.1016/j.coph.2007.02.004 (2007).
26 Wen, Y. et al. Constitutive Notch activation upregulates Pax7 and promotes the self-renewal of skeletal muscle satellite cells. Mol Cell Biol 32, 2300-2311, doi:10.1128/MCB.06753-11 MCB.06753-11 [pii] (2012).
27 Hansson, E. M., Lendahl, U. & Chapman, G. Notch signaling in development and disease. Semin Cancer Biol 14, 320-328, doi:10.1016/j.semcancer.2004.04.011 S1044579X04000288 [pii] (2004).
28 Sun, H. et al. Stra13 regulates satellite cell activation by antagonizing Notch signaling. J Cell Biol 177, 647-657, doi:jcb.200609007 [pii] 10.1083/jcb.200609007 (2007).
29 Kitamoto, T. & Hanaoka, K. Notch3 null mutation in mice causes muscle hyperplasia by repetitive muscle regeneration. Stem Cells 28, 2205-2216, doi:10.1002/stem.547 (2010).
30 Conboy, I. M., Conboy, M. J., Smythe, G. M. & Rando, T. A. Notch-mediated restoration of regenerative potential to aged muscle. Science 302, 1575-1577, doi:10.1126/science.1087573 302/5650/1575 [pii] (2003).
31 Couteaux, R., Mira, J. C. & d'Albis, A. Regeneration of muscles after cardiotoxin injury. I. Cytological aspects. Biol Cell 62, 171-182, doi:0248-4900(88)90034-2 [pii] (1988).
32 Honeyman, K., Carville, K. S., Howell, J. M., Fletcher, S. & Wilton, S. D. Development of a snapback method of single-strand conformation polymorphism analysis for genotyping Golden Retrievers for the X-linked muscular dystrophy allele. American Journal of Veterinary Research 60, 734-737 (1999).
33 Tusher, V. G., Tibshirani, R. & Chu, G. Significance analysis of microarrays applied to the ionizing radiation response. Proc Natl Acad Sci USA 98, 5116-5121, doi:10.1073/pnas.091062498 091062498 [pii] (2001).
34 Li, H. & Durbin, R. Fast and accurate long-read alignment with Burrows-Wheeler transform. Bioinformatics 26, 589-595, doi:10.1093/bioinformatics/btp698 btp698 [pii] (2010).
35 McKenna, A. et al. The Genome Analysis Toolkit: a MapReduce framework for analyzing next-generation DNA sequencing data. Genome Res 20, 1297-1303, doi:10.1101/gr.107524.110 gr.107524.110 [pii] (2010).
36 DePristo, M. A. et al. A framework for variation discovery and genotyping using next-generation DNA sequencing data. Nat Genet 43, 491-498, doi:10.1038/ng.806 ng.806 [pii] (2011).
37 Lawrence, C. & Mason, T. Zebrafish housing systems: a review of basic operating principles and considerations for design and functionality. ILAR J 53, 179-191, doi:10.1093/ilar.53.2.179 ilar.53.2.179 [pii] (2012).
38 Meeker, N. D., Hutchinson, S. A., Ho, L. & Trede, N. S. Method for isolation of PCR-ready genomic DNA from zebrafish tissues. Biotechniques 43, 610, 612, 614, doi:000112619 [pii] (2007).
39 Yamamoto, M. et al. Mib-Jag1-Notch signalling regulates patterning and structural roles of the notochord by controlling cell-fate decisions. Development 137, 2527-2537, doi:10.1242/dev.051011dev.051011 [pii] (2010).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” 0 or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of treating muscular dystrophy (MD), the method comprising
administering to a subject having or suspected of having MD an effective amount of a composition that increases the expression of JAG1.

2. The method of claim 1, wherein the composition increases the expression of JAG1 to restore a muscle function or phenotype.

3. The method of claim 1, wherein the composition comprises a vector for recombinant expression of JAG1.

4-5. (canceled)

6. The method of claim 1, wherein the composition comprises a transcription factor or a vector for recombinant expression of the transcription factor, wherein the transcription factor increases the expression of JAG1.

7. The method of claim 6, wherein the transcription factor is selected from the group consisting of myogenin, MyoD, Myf5, MRF4, and Rbp-J.

8-9. (canceled)

10. A method of treating muscular dystrophy (MD), the method comprising
administering to a subject having or suspected of having MD an effective amount of a composition comprising a JAG1 agonist.

11. (canceled)

12. A method of treating muscular dystrophy (MD), the method comprising
administering to a subject having or suspected of having MD an effective amount of a composition that promotes JAG1 signaling.

13-14. (canceled)

15. The method of claim 12, wherein the composition comprises a transcription factor or a vector for recombinant expression of the transcription factor, wherein the transcription factor increases the expression of JAG1.

16. The method of claim 15, wherein the transcription factor is selected from the group consisting of myogenin, MyoD, Myf5, MRF4, and Rbp-J.

17. (canceled)

18. A method of treating muscular dystrophy (MD), the method comprising
administering to a subject having or suspected of having MD an effective amount of a composition comprising a compound provided in Table 2.

19. The method of claim 1, wherein the MD is Duchenne muscular dystrophy (DMD).

20. A method of increasing the proliferation of a muscle cell or a muscle progenitor cell, the method comprising increasing the expression of JAG1 in a muscle cell or a muscle progenitor cell, wherein increasing the expression of JAG1 increases the proliferation of the cell.

21. A method of increasing and/or enhancing myofiber structure in a muscle cell or muscle progenitor cell, the method comprising increasing the expression of JAG1 in a muscle cell or a muscle progenitor cell, wherein increasing the expression of JAG1 increases and/or enhances the myofiber structure of the cell.

22-25. (canceled)

26. A method of prognosing muscular dystrophy (MD), the method comprising
selecting a subject having or suspected of having MD;
measuring an expression level of JAG1 in a sample obtained from the subject; and o identifying the subject as having a more favorable prognosis when the expression level of JAG1 is higher than a control level.

27-30. (canceled)

31. A method of prognosing muscular dystrophy (MD), the method comprising
selecting a subject having or suspected of having MD;
detecting a variant in a JAG1 gene, a JAG1 promoter, or a JAG1 regulatory element in a sample obtained from the subject; and
identifying the subject as having a more favorable prognosis when the variant in the JAG1 gene, JAG1 promoter, or JAG1 regulatory element is detected.

32. The method of claim 31, wherein the variant comprises one or more of a mutation, a SNP, or a haplotype in the JAG1 gene, JAG1 promoter, or JAG1 regulatory element.

33. A method of evaluating responsiveness to treatment for muscular dystrophy (MD), the method comprising measuring an expression level of JAG1 in a sample obtained from a subject having MD prior to the subject receiving a treatment for MD;
measuring an expression level of JAG1 in a sample obtained from the subject after the subject has received a treatment for MD; and
comparing the expression levels of JAG1 measured prior to and after treatment;
wherein an increase in JAG1 expression after the subject has received the treatment identifies the subject as responsive to treatment; or
wherein a decrease or no change in JAG1 expression after the subject has received the treatment identifies the subject as unresponsive to treatment.

34-35. (canceled)

36. The method of claim 31, wherein the MD is Duchenne muscular dystrophy (DMD).

37. A method for identifying a compound for the treatment of muscular dystrophy (MD), the method comprising
contacting a cell with a candidate compound;
measuring an expression level of JAG1 in the cell; and
identifying the candidate compound as a compound for the treatment of MD if the expression level of JAG1 is higher than a control level.

38-39. (canceled)

40. A method for identifying a JAG1-modulating compound for the treatment of muscular dystrophy (MD), the method comprising
contacting a zebrafish with a candidate compound;
assessing the muscle phenotype of the zebrafish; and
identifying the candidate compound as a JAG1-modulating compound for the treatment of MD if the muscle phenotype of the zebrafish is improved compared to a control muscle phenotype.

41-48. (canceled)


SEQUENCE LISTING (20 sequences)

SEQ ID NO: 1 - DNA, 32767 bp, Homo sapiens
ctgcccggcgtgctgggtagaggtggccagccccggccgctgctgccagacgggctctccgggtccttctccgagagccgggcgggcacgcgtcattgtgttacctgcggccggcccgcgagctaggctggtttttttttttctcccctccctcccccctttttccatgcagctgatctaaaagggaataaaaggctgcgcataatcataataataaaagaaggggagcgcgagagaaggaaagaaagccgggaggtggaagaggagggggagcgtctcaaagaagcgatcagaataataaaaggaggccgggctctttgccttctggaacgggccgctcttgaaagggcttttgaaaagtggtgttgttttccagtcgtgcatgctccaatcggcggagtatattagagccgggacgcggcggccgcaggggcagcggcgacggcagcaccggcggcagcaccagcgcgaacagcagcggcggcgtcccgagtgcccgcggcgcgcggcgcagcgatgcgttccccacggacgcgcggccggtccgggcgccccctaagcctcctgctcgccctgctctgtgccctgcgagccaaggtaggagcccttctccgggcctccctcccagccgtcctctccctcccctcgcggcgccctgggcggttgagccccgcgagcaggctcggcagccctcgagcgcccgactcctcgcccctgcgcccgagcggctgcacctgggctgagcgcgccaactcccccacgggagcccctgcctcctgagggtgacccctccaggccggacaggggcgcgttcgctccgctgttgccccccgggaagtcgtcggatttcttccgcactcgtgcatttttttttttttttgccccggaatcatgttgagtccttgaacgtagtttcgcaaaattattttcactcggcgaggagggctcgtcggggcggggagtgggagggagtcgccacctctatactcgaagaaaacagctttctcccgcgctgacctacctccttccctcgccggcaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccggtgagtgactactcgggaaggaggccgggcggagcctcacacccgcgcctggccgagccctgttatcccttgcgagagggatttaaagggctttggcttgaaactaggcgctaactggggaaatctctaggggtggcgggtgagggagccgggacactatagttagatctcggctggggtagtcgaggtgggtcccattcctgctgctatttgcttagtgcacaaaagtggggatcccagagggtttatgggaccaggctcctaggaaggccggtgtaaacttgcaacttgaagttcccagagtaactctcccggccccaagccaagagatcgttgtagctgatctctttgcttaactaattaaacctggtgcgccgtggaaggcgactcggcgcagttctggcctgcgaacgccgtccaaattggcaacctcccctttcccgtgaacggccgggagggcgggtgtgtccttcctggagggtgtgggggagtccgtttcccgctgctccccgggagattttagtgtgtgcgggggcgctctccaggttcggcttaagggccgggggcggccgccgcaggaggaggttcttggggccctcacaggagctgtagacctgggaacccccgcccgctgctggccccttcctgcccccgcccccgagtggtgtagctcttgtggcctcacttccagcggttcccgggctctggaacagctgtgtttgcagacttccccgggaagggcgggtgcacggcccccgcaagccttcgggggccgtgccaaggcttggaaggggtggtcttctggaggtagtgcctgagggtgtaagtgataggccctcatccccccgccccattccgcccccaaaggagctcgaggcccgggcccagctggcagatcctgcagcgaggcccttgcaacatcccaaaatgggtggaaggaagatgggtgcgattgcagaaggggaggaaagggaaatgttgggggtgtgccctctgcctggctcctgctgtccaggaggcatgactcattgccaagactggggcttggcctagcctgtgctgggagggtgggtggtgagagggggagctcggtgccccagagcattgctttccaccacgtgctctaatggggtgcactctgctgcctcctgtccgccggcttaggttgtgattctgggaggagacagaggggagactatctggctgtgtctgtgtatgaagcttggggtggctcttaaaatccacgattacaggagctggcattataaagtgccctctgctgtgggtgagataagacagttatcaaaaactgccatgaaaaggggtttataaaaagaaaaattaaaaccaacgctgtctgtgaagctctcttgaggtccaagggctgtttctgttgtgggatttcatttttaccaagagtatttatcttgtttgttaatcccctattagggagaactttaagctaaagagtctggctgcgtttaaatctggctggcccaagctgactgtgaatactcaaactgcccttgaacgtgtgtttggagtttagggggaaggggcctgcaggtggttccaccctcctgccggaggatctgtctggagcccctcgcttttcacaagaccataaaaggaatcttattagtgatgtggaaaagcagcagcctggctgaggctcattttggagagtagggtcagagggatggctaacatgaaaacaaggaggcttgctccctcgctggccaaggaaaagtgacctagaaaagtgcgtctgtccgcttttgacacttggtcagagtcctcaatggaaaacgagcggaaatagtgtgggagggtaggagaggcgattgatttgcatttgctggtgctcttctgtggtttcaggccccacgggtcaccaaccatgaaatagactctcggcttttctcgaatgggggaagaccgatggggcaccactgaaaatgtggttcttttgaagttgggacaaacgcatgtatcactcataaacaagtctccaccctgatgccccagaaaggcctgctgtgaaacataagatttaattctcaagagaaattggagtttaggtgtatttgtttttctgtatgtggttggagaaacggcacagtccgtaactttcaagaatgattccaggctgagtagaaacgtaactgaaacacttaagtagctgagcgagtatttaactagagggcacagcttcatttagtttggcaattaactatgtcttttaaaactgatttccttgaatgggaaataaatgaacttagtacagaattcaagagatagcaagaaaaggatatatggttaaaaacaaaacaaaactccaaaccaccttgggctcccaacccctagcaggcacaattaccatttgcatgagtcctcccaaatttgctgacgctgggtatcagcaaagggtgcaggggtaggtcagtgtagaaggtcatgtagttcatgctttttgtttggaattatgacccctatccaagcaactgttcatgttctagagaggacgaggaggaaacgatttggtcagatatgaaatacagtccatgttctggtgtgcatgtgaaaggctttgtagaggtggcccttcctgtcatatatcatatctgagtatgtcccacgtggtgtcccctgccccggctctggtcttggggtagtttttcaccactggcttctaattctcatactgttgctgctctaaggggcttaacttgcagcaactatctggattaatgattctcaaattgggggaccaagtgataagaaccattattgtgggaagtaattgggcttaacaggaaataatttaggaattaccacttataaatcactgaaacggttaatctgtacctcccaaaattagtgctaaatcccagtagtgcctgcttattaaaaatacattagtgtaattaccactactcatctccaaaagctttttgggaacttcagaaccccagttagctgtattgctgcatcggtggccccagagtgaaatgaatctagagaaacgttaggggaagaaattggggtatctggcttattagcctcatatttaaattccattgatcctattaaaagacttgctgagatcaactttttttttttttttttttttttttttttttttttttttttggaaaatccaatgaaacgtgattcttctgtgtgagggatgccagctttggcccccttcatgcttcaggcctgaatgaccaattgccacgatgctaatcacagaatgtaattgacaggcccttacttttatgccttttgctgtcttttccagtccctgagcttctgtagatatggtgtgggcacatggagggcttaaggatggggttctgatttgacctgcctcctttctgcagccaggatttgcatcctgcccgtgtagtcagcatgtatacaaagaaagagcatgtgtgtacgtatgtgctcagagcaagggactttctgcatgtggagaaaacgccttttgccctaagaagtctacctcaaaccaaagggatgaagtgatgtatctgtgttgcaacactgggcttgggggaaagtgaagctgtgcttgctttgaaagagctgtgctaagtatctgcagaatagctgctccaaaaatggaatctttattccaggtacttggtttctaatttttttttctcttgcaggtgtaaccttctgtgttttctttctccttgttgtcttgtttcttagcaacttattcaaacctccagtacttataggaatttgcaacccaccagttccacacccgtaattctacagggacttaagatgtttttagaaaacggtgaaactttggcgtggaggctcttgggggagttcagtgggttaaaaggagagtcagcagcaacttacaggcatgcaacctcccagaaaatcagcaatctaagtgctgctcaaaatcttcacaccaaatagtggtgcctttggggaccttttagatagaagacgtggaaagtttagcatggagaaaaaagttacttaaaaaaaaaaaaacagtgtcaaggtaacaaggagccttcacagttcctgatctgaaaacaggaatgtgaaccagaggccttggctggattcatatcacccactggccccattgtagaagggccagaagttggtcccaatgtccggctttcaaggttagtgacaatgtaagaatgcatgatctctggatgtgcatacaattagcgttgtaatccataaatgcaaatactcctgcaaattcttccatggtaaacttctgtaaggcatgttgctcttccacctacccccatccttcaagcctaggatctaggctgctgagagaagtgactcttggcttcagcttttaaatatatatatatctctcaaaacttaatcttttcttccaactgttggaagatacattgggatgaatggaaagtggtgattctaaaatggtggtggaaatggaggagggtcctgggttcagaaaaatcccacctttactttggtgcatggcactgggtggctgtgttgtgacttctttgctgatttctgattaccgcatggaccacagcagccctgtgactgctgttttttttttttttttttttttttatggtgccagtgacatctaccattggtgcagtgcgtgtgctgggaagaaattccgcaaggtggctcccagtagttaaaggagtacataaatagtcaaacatgcagagtcctctaccagctcaacccagtttttatgcacttttggtaaataaacagaaaggcttcttgttcaggcttgctctgtgtgaaccagaccgttgtgcttggctggcttcttggtattctatctattaacctcgccacccttttgtaactggtgcatttaattatcagtgggatgcttttcaaacaggtaagggtaatgatgggaaagggggtggggatgtctcattgagagtcacaaatctggatatgagctttggggccccttggtgagcctttgagtgggctttgatgagaaaatcaccctttgtaaccagatcggactctattgacatgtcaagccctacagacttgattgctttctgtgcctgaagtcttagcctagatgctataggatagttgtcctttcttatctcactttcccaaaacccactcgcattgtgtgcatgagtacgtgtgcatgcgccacacatggactccctgcaaccttttctgcttaatatgttttgtgactggaatattttatactctggccacccagacacaggcaggacaatagagttggccgaattgactgaaatgaggattatggagaataatagggcctctccaaaaataatgctaacacaggacaagcgacaggaagcaacagttgagtcagcagagctcagggcccctgacgaaccctgggagccgactgactgtgcacaatggccgcgttggcacgtcgggcattcccaggatgtgcgtgccgccggcgggcctgcctgtcgcagcagcctggggttttggacgccgcacgcgtgccgtttggatggcggtttatttgtgtccgtcagcttggcaggaggggaggcagccgcttctcagggcttggtttggctgtcactgttgccaggaaaaggaagctcccggctgccaacacaattacctttgtttcttacatccccccttcttaatgtggcaattaagtgcttaccgctctgcccagtgccaaggtaaccgccatggagtgggacagggcagagaacacaaccttggcacagcagcctaagtgtggagggcgggaggaggtgcaggggccccagccttgctgggaacctggtggcccttggatgccttggggctttgatgccaggactctcctctaggtagaattccacagaaatcttccaaaaagcaattccgaaggctgaccattcctctttgatgccacaccagaatgggaaccagctgccaggtttccagatttccttgggcttggagctgggaaggggctgaagtgactcactttgcaaatccagactaaagatctgtttgctacccctccttcctgtgtggccttcacagtactgttctggaaaacataaggaaagatgaaagccaggtgactcttcggcagagtcttagttctgtagtctgaaatcaagaaaaacccaggtatttgcttttgttgttgttgttttgctgtataaacttaagtttgaacacttgcctttccaagccctcctgggatggaggactccgcctctactgggggtcagagttgcagtggctgcttgtatgtgacttcggcatagtggatttgctgctctgtggcctggtccctgctttgatgtggggtgcatgatctgggatctcagtgcctgggtgtatgttcagggatgtacagaggtcaagtgtgggcctgggaaaaattaaagttctctagcaactaataatcattgttaaattatcttgattgctgatctatagtgtggggatggagtgggaggttgagtgcctcctaataggaaccttggagggaatcctttttctctgtagggctgggcaggaggaatcctcctgccccaggagaccctaatcccctccctgaaacagacaggaaaaaaagtccaggctttgttgccgtcactgtagctccctgccctcaccctgtctctgcctctgagaaagaagggcagttcatggaaaatcacagagccaggcacgtagggctgtgtgcaattcttttctctccaggtggggtgatgtcggtcagcaacttctgaggttttcccagttctggcgaggatgagcctggtagactgcttggtcgtccaagttcttgtgcaagacctaaggatgctttcctctttgagtattaaactgagttagtactgccatgccagctcagtcccggcaccttcccttgcagtggggtttgtgggtattatgtgtttagcgcttgtgcaagtctgtcaaaactcctggtatcaatgcatttaatcagaggattaattacaacaatactgttgaagtggaggacgtgggcctcaacgtgaccagcaaatgaagggcttaattatacagaatcagccacacatctggcctcattttgactgtgctcttacctcaaatcagtagagaaatataatttcttgctgctcttagggaagagtcctgtaatttgcaggaagtcctaagaaacttaaagaggcagtaaataaggtataactttcgctaataaaatagtaacaacagcaatgaatttggacctgtggccactaacaggagagggctgggtatgtgccctggcttgcccggttttgcttgcttttctgagcattttcccacagttatcagtgaacgaaatggaatcctactctgctttgagtatgaaacatatttcggctgaattccaaggcagggtctatcacaagatatttttatattcagagatgacctgctgtgtcactctgagagcgctaagctggccttgagttatgaatgaaactcccttttaccctccctttttctctttttcctttatcttataggtgctttctcttctcttggcagcagtgtgcttctggtggcacagcacggtattatttaatctatacacgcttctatttcctatggcttggtttctgacacagcaaagcagggattcataacctagttcatgttccttgtctaccccacctgccttgagtacatttcttcagtgccaagatagcagaagcttcatttaaacgctggtgttttgtgagactgaccttactgtaaattacgaaggaaaattttagaaaaggagggaatttgctaatagcttaggtttggctgggggtggggaggtggaattctgagataagcttggaagataaaaggtgccttcagtttaacagctggcatcttgccagtatccagggaatttgtttagttagtagactctgattattcttggaatgttagatcttattaacagataagttcatcaataaatataacccatgggtgtgcggttgtttctccctttgtactttcttccctgggatctgcccccaactccaccgcccaagtgaggttgacttctttgtcttgcagagtcactttgcatctaccccctcctgtttcctagagttacccgatacttgtgggagccaggatggtctcacatagacagagttttcaaaggggttctaaaaaggaaaagggggcctccctagagactgcctcaccagggcccaaagtgtaaccttagcaataccccctcggtacaaatacctggttaggttagcttagaataaattctttgtgtgtgtgtgtgtgtgtgtgtgtgtgtgtgtgtgtgtttcctcatgcactagggtcagggtctggaaagttttgggaaactatatgctaaatactctcatatcatatagtgaggtggcatttcctgatctcaactttgagctgcctgcatgcatgttgactccagtgcgtccctgccaggtgaaaggagtcatttgtcaacctgggaagagtgctgacaaaggaaggggagttggtttggatttaaacatccaggaactcaatgcaacaaccaacagaatttttattttggcattatggttttgtttggtggggtattctgggaggccaagaatgtacgagattttggaaagtcgaccctgtttctgaattgctaaattgtttgtttgtcttgcagaggtcctatacgttgcttgtggaggcgtgggattccagtaatgacaccgttcgtaagtatcgcttctgtggacttttctctaaacacacgtggaagtgtggggcttggccaaccagttggagctggatctgtctccttagtacttagcatttctgccacctcttgccttctgttctcccctgtaccccagggtgaaccttcagtggaatgtattccttaacttttctaggatttttgcttggaatattaagtggccagaacagaatcccagttaactcattccctgattatgaaacctagattatcttcgggatagagatcttaatggttttacaactttaaaaccttgtagtctgctaaaggatcaaagtagtgactttttaaaagtccatttaaactttaagtgagtgtttaatctctcatcagaaagataaagccataaaggtggtgctgggctctagagaccgaccttgaatttcacttggtgggcaggtgagcttctagtctctccgggtttcccacactgctccacaaacctttcattgaagcgcaattccagagcttcacagccattacctgccttgtccctttctgaaagatggctctgttttcagtgcaggttgcctttcaggactgaggggttggttttggtggataagccatggaggatgtgattgaaatgacatagtctggcttacaaagtagtcttgctctttaattgtaactttacagtgttttcgctcacgagccgaaacttcaagttccctttttgccagcagagtttgggttgcggtcgctggaaaacttgatgctcactgaaatctcttggtctgtgcatgaggaagagtggatccagcttcttgtcttggctctctgttctctgaatgccttgtactttttgcttttctatcttaccatttttttttgtttgctttcactgtgaataatatattttcatctttccttgccttgcttttttttgctgacagaaaatggtgaatgcacaccgtgggtgcagtgagggcttggtgcctgctacgtgggcagcttccacctgtggctggcttattgggctacttggagccacagaatcattgctgagctccgatgtgccagagagtcccaggccatttgggtcactcttctagcttgggaatcttaagtccagctttttggatgtcatcctccctggagggggagctcatgaaagtccaaggttcgagtcttcctccccagagggctaccctgtaatgccgtgggggggtgtgtgtgggggtggcagggaagctctgtcagccttggctatggctgatgccagtcagggtcatagacacctgtttgttctcccctccctgccacaaacgttgaggactgtgaacattatgtcactgtgacctgcttacaatggtaactaagcgttaggcagaaggggttgaggaggggggagacataggctcttctgtaatagtatggtgtcgacatggtgttctttcccctcccttttagctattgaaacaatccacccggtagagtgaacagcttgaggttgatcctggcctgtgataagagccaggtcagtatacttgttagggacatgtgagagacctccccgccacactgcagcattcacagcacctttcctggacctcctgtcacctcgcaggtcagggagttatgtcccctgggaacccagggcttgtccagagctacccacagttgtctgcatttccaggtagcccgattttggggttctaggatgtttcatctcctgggggggtaatcaaatccctggaagaggacaagaaagactgaggtaggaataaagttcttttaaacctcaagggtgcccattgcaggttatcaaaactcacttggtgaaccctgaagagggagatgggtttggtggaaatggtttctccacttgcctatttggcctcttttatgatgcttccaaggaatcttgaattcagcacagtcaaaaccaaactcaggatcttctcttgccaaaagcagtcccttcccagggccccttctgggtgtctggcaccgatatttgaacactcattgccataagccagaaatatagaaggcgatctgtgtaccccctctctgttagcatccatacctagtccacctccaagttctgaaaaatctctccagctcttcacttgaatctgcctgccttctgccactgccctggtccaagtcaccatctgctcatgactgaacttagagtagccttcgtctggtccacctttatctgctctgactcacctctagtgctttctccattcttctgccatcctggtcttttcagagccataacatttgcttgtcacccttattatgaaaaaccaaccacttggtggccttctgttgctcttgggacagatcttcatgtggcccacaaggctctgtgcagcccagctttgtctccctgccagccttgtcttacacaccgtgttgtccatcgtatctgtagcctggccataccctagcagttgccttcctatcctcaactacatcacatgctttctggcctcaggacctttgcacatgctgtttatgctacttggagtgttttgttcctttcttcaccctcagccactccctctggactgcactccacagggcagaagctgaggatgttcagaagcccaaacggagttggctgccctgtgggacccaagcgagttgtaaacattctggcttcagatgtaaatcaaaggcagagccctgagttttagggcagagaattccttctatcagctctgcagtgagccctcacaggcagactcgggcccaaatatagcctaggtgctgtttatgtatttgaaagtatttaaggctggtccttctgtcatcggtcctccaaagtcttttattacattttgggactgtggtatatagagttccaaatttctttctcccctagagcaaatggtttcagtttactgtaatgcataataaacatgtaaacataaataggcacacttcagaccaggttttccctgtagcttagctttctcggctaaggcccccttccaggtttttgggtcggtgtggtcccaggttatgctcagactcgcccttcatcacctcctccttggcctgcgaggcggtcatggcttcttcgtgactcatcttcgtggcactggggattgcagggaggcatggtgatgtccttttccagtcacaaggctggactgccaaacgaactgccacagattcctttccagtgccccaagctgaaggaaagcgtgatcaggaagcaggcagcagacgtatttgaacccagacatgctcgaccccctccctgagcaggtgtgaaattatgtatgcagctccatagctccactgaggattctgaagtgatcctctgcacgacacttcccggaaataagtggaaagcttactgcatgactgaaaagtacatatcagtcctgcacccctaggattgccctggactcttgtctaaactgtttgttgttgataccagcctcagaagctggatgcctttaagccatggctagtgtgtttaaccgatcccttttatgaagatcttgtaagcgcgtggtaatagcccactatgctttttaactgaaccagcagagcaaacatagttaatggagagacatttttgtcattccttggcctctgtttatttgcaaaaacgagtaaagtgttacctgcctgcaagtggttcagtgcgtgccggggtgtcaggctgcaggtatgtgagcttgttcaaggcttgtcttgcccacgcagcagtttgagagccctagagggcgaacgctggggccctgttggaagcggtgtgtggatgaagccacccaggagtcccttctctcctggtcacttatagggcttgcatcactttttaaaggagcactggcccgaggcctagaaaaacagccatgtgggtgggtaagaatcacaaagacattgcaagtcagtcctaactgtctctccaagatggtcttgaatttgacaaggtgatgaagagttggtgctggcaggtttaagaaaacaaacacaaaaccaggttggggtgctgatttagtgccttgctgccttttcagatccctcccctgaactgctggcacctgatgtttgagctatttttgtacctgtctcttctcaaactagatgataagtggcctcagggcagggactacatactcctgagagctgcttgagctcgaggtgtttattatcctcagtaagcattttttttcaggagcatgacttggctcaaaaaaacaaaaagaaaaaagaaaccctcccctcaatttacctgtgtctaaactttggtgaattattctcccatctgccttctgggcagaggagaaaggtggaatgcatcaagttcaaggtcttggtatttaagagctggctttaaaggttgccatgttaacaattgtaaaaaaaaaaaaaaaaaaaaaaaaaaaagtaacttaatcgtctccttggcttccagttaagagttttgaaatggacaaaaagcttttctgagcaagacataaaaccctaaaaccagaaatgattgctgacctttatctggaataatgaaatttcttcaagagcaacattccatagggagctactgtctctcatatgtaaagagcacttgcaagtcagtatggcaatgtaatagaaaagttggcagagaagagaagctgttgctgttcacaggaacaaatataaatggttttgtaactaccagaaagggctcttgccatcacttagttaaatgctaattaaatgagatacctctttacctatcaggcatagattagaaggtttgataatgtacaacgttggcagtgttattgggaaacacagcttcctgggtggtgggaggacagactggtagtttgagactgtggacagaggttgatttgttgtgcacggactctgattcagcacttccatttctagagtatctcattgtgggaaccatacacaggcagatacgttcaaggaatgctcactgtggctttgtttaatagtaagaagcccaaacgactcacatgcacacatgtcagggactaggtaaattatttattggtgtttccagacaatgaaatactgtgtacccattaaagaaggtaaacaagtggtaacaggttgtctctgggtaggggcataaacttttcatttctttgccttttttacaacagatagaattttgtagcaggtgcatatgtttttattttaaaaagccaaaatggtcagggaagaaggctgcaatgtgaatattaacatgattcatcactttgtctctagaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccccgaatgtaacagaggtatgtgtgtgtgtgtgcatgtgcacgtgtgcctgttgcttttagtgtccatttatcaccccaccagcaggcaggtggcatgcggctatctcaggtggggtgggattttggctgttattctgggcctgcagtatgaagatgcggagtgtagaagatgctgttggtgccccatccagatcccctttactggcatccccatcccccaggtgctgtgttggctgccaacagctcacagctgctccttctcaggagactcgtccttggccaaaccagtgcatgccccattcctccagctcacagtcagtgattgacagacatagggctacaaagcccagcttcctttgccttaaaacagtagtacaatttgcactctagagcttaccctgggattaggctgaaatcagactccagttgagaccatgttcttgctcagctctttcccctaccccatctgcttccctcaattccttctacctatgtccagatccccttctcagactctgcttctaaggaacttgacccaaaatagatccagacaggactaggtcagcccagttgttgtctgaacaggctatcctataggaaatgatagcccctgcattcagggtagccttgcagcaagttttcactaggtgtctttgggggcatggctttgatggggagaggtatgtggttagctcaagctttcctattggtgtaggagtgggcccacttttagtctctgtgctgtctactgtcccagtccctttatagaattcagtgattccaattcatagcaacttgatgggtaattctgttaactgtataatatcccctcatgagcaataatgtactctgcttattttacatgataattaggtaaattgttttgatagactcaatttcaaagtgagtttttcaaccatgagagaggggtgggtcatgggggaaggaatttacagctctcccgggtagtactactgctttcttaaataattggagagaagagtttaaaaaggatggcatttctggaattcaaatgaaaaggggaattcattaacatattttagtgtgttttaaaaattaaacgacctaacacttagtgtgggttttttgtttggtgtttttttttttttttaagataaggtgctcactttgtcacccatgatggcacaatcatggctcactggagccttgaactcctgggttcaagtgatcctcctgcctcagcctcccaagtagctgggactataggcacatgccaccacacccagcttttttattttgtgtagacatagtcttgctatgttgttgaggctggtctcgaactcctggcctcaagctatccttctgcctcaatctcccaaagtgttgggattataggcatgggccactgttgcctggtcttagaaactgacattttttagaagttacttttttcttttttttaaacgaagtagagaattgctctgcaaggatactttataaaagtctcaccagatctggccaccagattagcctgcccaatgagtccagcctggctggctctgttagtcccatggaattttcaaaccaggcatttggtatgggttgtttttttttttttttttttttttaaaggctgaggctcatctgaatgtgtcctggtgggatggggaaggattatctatacctttttttcttctaatgaaaagggtgtgggcagaatggaaactgaccaagctctgctcaaggagccagggtagctgtggtttagagaggctggacgaaggtcccccttccctcgtcaacttgtctgcattttgtccccaaacaatatcatgttttcagaatctgctaacaaggccttgaagtttaaagctggttttgttatttgggcccctcccatcttcctctccttacaagggacttgatatgagacctgttaaaatcgtggaatttgcagactttaatgcaatgggccattctcatgaccggggcgatgcaaggaacaggcagtgtgctgacacgcccttctctgttttttacagctatttgccgacaaggctgcagtcctaagcatgggtcttgcaaactcccaggtgactgcaggtaaatcaactggtcttttgtgagatttcttttaattttcctttatgtaagagaggaactgactttattgtgactatgcctcttttttccttggagaacaagaaagcttttttaaaaaaaaaattcagatagacacatccgaagcttattaaattataccagctgtctggtggaatgcacacaccacttctctcttggaataaaaagaaactctttgggcatagccagtgacttcatgtattaactgttctgtaaattgataacacatttttataatcttcccttatagtgctgatgggctagatggagggatgtgggcttgtttctttgcgtctctctcctcttccaccaaggctttcttctcccagccccctccccgacttttcccatcaataggcgaaagtccagcattctttagaatgggattagtccaatctcctggcccctttgcaggcgggagctgggttggtgctgagaggttaatggctgccctggaatcttgtcagttgagcctgatgaatgtagacttttgctgcagtcctttgaagtctgttcctttatggtccaggaacaatgcggaagccaaagggaggcctgccctgaccacttcacgatggcgggttttggaacttcttgaaatttatattacgcccgtttggaaaaggcttattttgggaaatattgttggtgtctgtggtgtttttggcagtcttaatgctttgctctccctcctcccaaattttaaggagtaacgctaaatggatcctttgagcctgtcacttttgcccccaaagctactctgacatgagtaaagggaatcttatgaaacatagtctaaaccagtatgtcccaaactaatgttatttggggctcttattaaaacgcaggtcctgattctataggtctgggctgagcccaggtttctgcatttctaacaagctcccaagcgatgctgctgatggaaccatcagcaatcactgcccccagacctcagtactcaaagtgtaacttccagaattaacaagggagctgttaaaatgcaggttcgtgggctccgtcccacatccatagaatcctagtccttggtttggcccaggagtctgcatttgacaagctggccacatgactttgctgcttagtaaattttgaggctactcctttaaagagaactatctgtggcctttacctttatgattccaccgctccgtctgttcccctcccatgcaagggggaacatggaactaaattaagaactggttgtcccgaattgtctcttcctacacttcagaaagatagtttatttcctggaaggctgaggggcttctcctgctgtctcccattggtggcgatggactgtagtatttaacagcattgctgtccaatgcggagcaggaatccacctaccttaactcttcctaattaagggtgttctggagaaagtcccaatggcagtgctattagtgattccaagcctgctgcctgctcagatggatatcagcggaggccgactgtgatttgtgctgctgtcatcagaacacacatacagctatacaggggagggccctcccagccagaacaacaggctttgtttccagaatttagccctttctctttcgaagtcccacctccctctccctgcccagacacgcactgatggccttatttacttaaggtgcgactttcccacacatcatttgcatagtctcccagcccagttttccctccctaccttgaaagaaagtcaaaacagaaccttcacgctgaggaagttcaggttgtgtcctgctgcagaaatagttgaatggcaagttacctgcaagttgtcttttggattatgtattacacaggttattttttttttcacccccacatgagaaaatttactggtttttgttgttcccaagtagactgagtccctttccctgggtatacatgcttgcttattgcaagcctgccaataatatcaaacttaattatgaatcatcatctgaggaaagtcacttcaatttctcatgagaatcttaatcatatattattaaactgggataacttggttggtgcctcttttagggtcagtatttttatttgttttcttttgaagtcatccatggcctcttgtggttgcttttttatgcttggggattgaggtctctttaattaggtgaggttgggtcctcagctgcctctccttgcctttatgaggcaacttgaagagggctagtgatcctgaatttggaagagaataagggaagacggtgagaatattgccatttcgtaatcagttccctcttactatctttcaggctaagaatatcagccttgacagaatggctacacccttacctcccactaacgtgggtgtccgcttttgagaaattatttagattgtaattttgttcttatcttactcctttagtcacttgtcacgttgacattccattttacataaaccagaatccagtgtctaactttaaaccacacattctagtgatttctttgcatgtgggtgtgcatagcttggtttcagaagttaggaagtggttagtcagtattctcaggggttaagttgactctcagccgtgggtgattagctattagctagatccctataattttcccatgacaaggatttgctagcctgcctcccgctcttgggaaaccgaaagcctttccttctggggagctagagggggcgggggttgttttccagagttacagctgagcagcttagggggttggcgctgatcgtggcttttcccaggtcccgttctggaaacatgttgcccatcccacccgcaccccaccctccaccctccaccctccacgctccaccgggaggctagttgatgtttctccctgccttcccccacccttgttaaagcttggctcttgagaaggcaattaaagtttcagagggcttgaaaattcaagggagagtggtttgagacgctgccccctctcccaccccagatgaaagttgttgtatttgtttttgtttttgcttggggatcagaggcactatttggccccaaaagagtatgattaatcaaggtgcttatataaaagaacctcaactccgccaatactagataaattaagcagactgctttcttttttttttcttttcttggcaaagcaagaaatcactataaattagcacacagtaatgggacacaaattatggagttggttccaagtgttgtcatttccttctccttccctatagaagtgggtttagagagcctactctggctttggccaagggagggtgcccctttctctccttgtttttttaaataaagcgtcatcgtgaggcagtttcctgctgcacttggtggcttgttgcatttctgtgcactggggtcatgactttctgtgcagacaaagagcagagtggcaagtgcagcgtctgcagagactttggcatagctctcttgtcaccgagagagtcctttcctctatagtctgtcgcaactaggtgcacgggttgatttattccatgccttcacctttggctcactcgtgtgagcctgagaagagagtggctttgtgaagggcatccccatgctggcctcttcccaaacagccctccttttgtcaggagtcggctgggaggggacctcagctcaactgtgaaaatgtttgtttatacggagcccctgatgagccttgggagtctaccttcctgtccccagaaacaaaacagaaaaagcctggactttgcagtcttccatttgaattgtacaggtgtctcttaacgttcaaaaggctaacctggaggtgtgctgtgtgtctccaggtgccagtacggctggcaaggcctgtactgtgataagtgcatcccacacccgggatgcgtccacggcatctgtaatgagccctggcagtgcctctgtgagaccaactggggcggccagctctgtgacaaaggtatggcccttagggatgagacccagggtgggatctgggggtgggatggcactaatttggtttctaattttattggcaggggcttttattctgaattctccttgaatgctgtgtgggttttctggtttttctttttaaaagacgactggggctagaaacatggtgggttcgccatcttcacaggcgtctcttagtgactccaggatctttttggcagatctcaattactgtgggactcatcagccgtgtctcaacgggggaacttgtagcaacacaggccctgacaaatatcagtgttcctgccctgaggggtattcaggacccaactgtgaaattggtaagtggtccaagatgaatgagagccctttgtctaatttttctgtactcagcttttaaaaaatatacttaaggacttgtttaaggaaaaaaaagtgaaaatagcttccaaagataggccttatcccaaccattctataagcctttgtaactttcaagtctctaaagttttttgggtgatgctgattcgtataggaggcagcgggagatgaaatgaccccagctctgagatgtcttggaatgtggttagagacagctcagcgagaaagatgacttcctttctttcctagacacagcagggttctatcacacgcttgcttttggtttttgaaatatgcttgcactagtggctgtggtgtgggattcggttggaggggggtgtggggaggtaacggtgtgcaggcatccctctctgactgccatcctggtttttgcagctgagcacgcctgcctctctgatccctgtcacaacagaggcagctgtaaggagacctccctgggctttgagtgtgagtgttccccaggctggaccggccccacatgctctacaagtaagtccaattttcagcctgtgtccattctaaggatccttgtgccaagataccacgctggggagaggtacctaggcttgtccagtgtgaatgtctcggtgagagaggggcgggtgcaggcctaagtctggtaagcagtctggtggcttgtgtgtttggaagtctcccagcacccccatcctgtcccactaaggtagcgcatgtatctgttcttggaaggaacgtcctcgtggaatcgcttgttcctttgcatctgcttcctggagctggccaccttccaattgtggttattcttcttgcagccatggagaggctgccttcaggggaggttgtcacttactactggattctgtcatagggccagggggccccagagaagaacctggcctgcagagttcctttggctgtgagacagggctgtatatcagaatcacctgggctgctttacagtgtaaatctctgggtcccccagattccctccagaatcggggagtccatggtctttgataaacttctggaattctgcagtgcagccaggtctgagaaccgctgtcatcagcaccccaggtgtggttatggatgtggatttctggggtcagattgcctgtgctggtcctaggcccccacctgtcacttgcggaactccagcaggccacagaagctccccgggccctctttaaatggcacagtagcaattcccatctccgaggttgtcataaagacacaaaagtgaatacttcataaattgttaggagttgtctctggtcagagcagaagtggagaatgcagcctctaggtgtgctattttgggcaaactctgtaagcaaatgagaggaataaggttttccacctgtaaagaactatttttcctgacttcttagctacttctgagccatgatgaaacttttctttaaaaataaagattaggcagatagagatgtcctgtgttcctcctgggcggatgctccgtcagtatccagctgtctttctcccccactgcttaggttggggattacatgaactggggcagtgggggatttaaagctaaaatcaaggcttcggccttatcgtcaccgtcccacttgtcctgctgtagctttccttgtagcaggtgtctggctcttcaatgacagcccctcatgtaagaggtgatgttaaaggatctaattgtcagatggattgaattaaattgtcaaccccctccttttcttgttgaccagacattgatgactgttctcctaataactgttcccacgggggcacctgccaggacctggttaacggatttaagtgtgtgtgccccccacagtggactgggaaaacgtgccagttaggtaagaacatccctgtgactgtaatttttataccaaggttggctttgattgtcaaagccattacagaagacgagcgtgtggccggcagtgtgagttcacactccatcatgctaagaagtaatttattatcaagagtctagcaggcctgggagaaataacttgaatgcaaatggactagctcttttaggccctgtgggaaagtcgtgggattccttgaaccatgtcaatcgtctgtccgaggagtgttcagctgggttccaaaaggggatgatctcatgaggaatgagctgtctgcattctttttgccacatttaaatcccagcaactaggctgcctgggttttgcacagaaatgcctggtggctttctgctgttttgttgggggctggttaactagcagctggaaagttatcaattagggttcttattggataatagctagggagagggatcagatgcttaaaatagagggaacctctagtatcttttatttagctagcttattaaaatgtctcatgtctcagacaaactctggcctgttctttttttttccaaggtgggggagatgggggttgaacttcatttctcatgctcatccccatctcctttcagatgcaaatgaatgtgaggccaaaccttgtgtaaacgccaaatcctgtaagaatctcattgccagctactactgcgactgtcttcccggctggatgggtcagaattgtgacataagtgagtgactttgtttccattttgattttcatgaaacttgggtagccgacttgctggtccgtactggggctccagtcctaggacttgagcagtagaaggaaaaccttagagtgggaatggttttttgagagggagtggtcagggcagcagtgaaaacatacactttatcaggctcattagctgcgaaaaccagccatattctgataatgtggctgaaacaatgatgtgaagtttcactccctcattctggaagaattagggagggtccccacccagcgtaataaccttatctcacggaaagcacacaggaaatctgatttgaaacttatctatcctgagctgagaaggcttttcaattaagcccaatttcactgtaaattacctctttaaaatgatgacttatttattttttagatattaatgactgccttggccagtgtcagaatgacgcctcctgtcgggtatgtaaatctttgcttaaatcaaaactttgacaattgacaacaaatctttagatttgtgcgacactaggagagctctgtccctgtgagatttaaaaatttgggtgaaaaaagagtctcttggaagtttcgctagggcttatgttaccccagcctcgtgggagtcattttgaaggcatgcacatagaaattcctctaaccactttcagaggtttagcatttggctatttcagtgaaccagctaaaccgcaacagtcatgctaaaccgctgaagccctgtgtttgtggaatacatagaaaaaaagcatgtgtctctctctccccttctcctcttctaggatttggttaatggttatcgctgtatctgtccacctggctatgcaggcgatcactgtgagagagacatcgatgaatgtgccagcaacccctgtttgaatgggggtcactgtcagaatgaaatcaacagattccagtgtctgtgtcccactggtttctctggaaacctctgtcaggtgagtggtggcaaaacctcagatggccaagtttttagagcagagccgctgcctcctccgcttccctttacttttccctcagctcttgtgtctggaatttcctaacatctattaacatcctattgatgagagagtctctgctagacaaatgctggtggaaggagatactgacgaattattttcttcttagtgtcttcgtctttgtgttctttttggttccctccaccccctcttgtgctccctctcccactctgccccgttgtttcaaatactggacattggcagtccctacagcatagagatgtatcttacagggtgtctcctaatagaaaccgaaggaactgtcagattccagtgcctgctgccccagagaagttatcgtgacacctttgttttactctgatccctcccccttttcgctgttcctgatatttgcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaaggtacctcccttctcccgggacctggctgtctccagacttgctccttttgtccccacttactaccactgccccttgttacacttgagaagtaatgtattggtgaggaggacctctatagtgaaatgatcctgcctgtgtagcgggccgcaacacgtggaaacaatctattccggcctgccagtgggagagccgtgtctacaagtagcccaggggagagaatgtgaaatccatttagcattcctgttctggggtgggctgatgtgcaacgctgaggcaaatgcctgagaacaacactgtcaacagaacttcctgcaacggcagacagagggggatgttctgtatctgcacggtcaggtatgggagccccaggccatgtggactgtgactagtgtaactagaaactgattttatttcattttagttcatttaaatgtaaatagccacatgtggccggtggctgctgtattaggcagcacaacatttaaagcttctataagattgtccttgagccttgcttaagatcaaaaaggaagtaaataaagcaggtccttttgccttagcaagtgtttaccaaattgggtagatgtcaaaggtaggatgaaggggatccacaagcatgtgggttttcctggttgtgtggagggagaaaatgctttaaaactctttgcctggagtctcaactttgcctgtcataatgtgatgtggcacgttgccagtatcttggcttccctggggtgtgtctcattagccagagggtccctgtctccaggaaagtgggcttggcccaggcccttggcttaggaatgccgcatctgtgggtgcatggctgtgatctgatcccaagcctatcttctagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaagtaagactccctcatccgggaatggaccagtgctccccagcctgcccctgcccctgcctctgcccctggcccaccctgggatcattggtgttgaaagttttttttttttttttgagaccgaatttcgctgttgttgcccaggctagagtgcagtggcacgatctcggctcaccacaacctcggcctcccaggttcaagcgattctcctgcctgcctcagccttcctgagtagctgggattacaggcatgcgcaccacgcccaccacgcccagctaattttgtatttttagtagagatggggtttccccatgttggtcaggctggtctcgaactcccagcctcagatgatccaccccccgacccccaacttggcctcccaaagtgctgggattacaggtgtgagccaccatgcccagcagtgttgaaattgttttaatcagaaacagtaaggctggggagactggttgagacttatccttacatctgggatctaagactgaggtcgtgttcaatttaccagatcactctgcctcggagcctccatatctatgttggaacattctggaatgttgctaggttgggtgattcctcttttctgtaggagtttcttccccactttcacctagtaccggtgactaaatgcccatgtgagggcattaagtgggtaaggcactctctggaagcttctgcttgataagccgcctgtctaatttgagaaacagccctgatggagggtagcaggtggaggtgccaggtgcattgtgtcaggagggagccatgaaaactgctgaacagtgcttgtctatggcttcacaagtccttcagttctttttttgctttactttgtttcatagatattaatgactgtgagagcaacccttgtagaaacggtggcacttgcatcgatggtgtcaactcctacaagtgcatctgtagtgacggctgggagggggcctactgtgaaaccagtgagtctgccactctttggggagctctgggatgtatgggtcatgttggagggatcccacttcaacgtgggaaaactttctttgtactggaaatctctggaagcaagaccgcagtgatttctgcactgggacagtatcacttgtgtctcatttgcatttatgagtcctgatgatagcttatggggaggagcttctgctgaggacctatagaattagactgtgtaataaatattcctgcttgcaaggaaagtcatatttctactttcatttccatgggacttccagaggcccaccccacatcttgcctaacttgtggcattcaaagtaacccagttgggagctcctttattcctggaaaagcctgattgtgttaaagtactacgactgtggctgttctcagaataggatgttggatgtaagcagtaggaaaggcttggctctcgcctgtcgtgaatggtcctggatctcgtcttcagccgattgtctcccctttgattctagatattaatgactgcagccagaacccctgccacaatgggggcacgtgtcgcgacctggtcaatgacttctactgtgactgtaaaaatgggtggaaaggaaagacctgccactcacgtaagtggtagttggccttggatgctatctctgggacccttccctctgtcttctgtgggagggggccactgggactcacactgaaatattttcctccaggtgacagtcagtgtgatgaggccacgtgcaacaacggtggcacctgctatgatgagggggatgcttttaagtgcatgtgtcctggcggctgggaaggaacaacctgtaacataggtaactttatgccccacgtgggatctgcagtgggcatggccacctgggcgtgtctgctggttggcccctggagcttatgatatttctctgggcctggttcttgcccctccagactgtttctggatactcagggagggtggtcgtgtctgctcttcttttatagcccgaaacagtagctgcctgcccaacccctgccataatgggggcacatgtgtggtcaacggcgagtcctttacgtgcgtctgcaaggaaggctgggaggggcccatctgtgctcagagtgagtgtcctccccccttgaactctcccagggctgtcggagcagatcctgacacccttggggacttgcaatggaaaccagaagcagagctgtttgatgtctgcttttgtatcacctgggtctgcattaatgacttgatgggatacagccctgacctgggcatgagaaagtcgccaataagataagtggagacagatggcgtctcagttcccagctctgggtctgaccagcagcctgtctccttccattctcacccccacccctatcccctaccagggcctcttcctgtgtacatgtgtcccacagcaaatgaaactaagtgtggtgcgggcctggttccaatttagcaaaggtattttgctggtgttaactacgactttatcatttgggtattaggaaaataagtggtggttttacttagggctaagaccgctttccctgttgaagtcctcactattaagttgtcttttctttgcagataccaatgactgcagccctcatccctggtaagtgtgacatccttttaagccagcactcatccactatcgtgtgtgtgtgtgtgtgtgtgtgtgtgtgtgtgtgtgtgagaatcacccagggattgtgtttaaaatgcaagttctgagtcaggaggtctggggtggggcctcagactgcttttctaacaagctcccaggtgaagcgatcctgctgccccatggaccacactttaattagccaggttgtgtcctcatttagagcagtgatttttcaaccttggctgcacaccagaatcacctgggagcttttaaaaatcccgctgcctgggctgcaaccaagacctgtgaaattagactttctgggggtgggacccaggcatcagtattcttttaaaattccccaggcaacggccatggggtgagagccactgactgtaactggtgtggtgacatgtgagtgattggcagccagagtaatggactgggaggttggtaaccaaggtggtctttgctttcttagttacaacagcggcacctgtgtggatggagacaactggtaccggtgcgaatgtgccccgggttttgctgggcccgactgcagaataagtaaggactgtctccgtctggttttcccaatggctttaccgcttcattccatgcctcaccccaagactttcatctttaaaaacaaagccaccattccctttaagtagctctcatagagcaaaggatctctctcctccagctgggattaagatatgccaggctggatcctcccaggtacctgctggtagttccctgggtggtgtagccctccttgttcttggggagagagtgccagtcacagaatctgatgattaatctacagtggggtcctgaagaaagtatgtgcatttccccatgactaacatgaaattatgggcaagcagatgttaaccacagctacactggtataggataaagctaatacaaacggaagggaggacgactgtggcttccagaggcttgtctgtgctgggtctgatcaggccctggagaaatgactaaaacaggaaaataggggcaggcaacgcagtttttcaagaggcttaaagcatattattttgagccttttctctacttttttacaagccctttctcttacagaacaaaaaaggtgctagagtagttatggtttgattcttgtacccaaattgtctgaaattttactggtgcttgaaatccaagtctgggattaactagtcttcagcactagatgggggcaggttgtatctgtttcatgtttctaaataaacagttggcaaactgtggcccaggggccgtatccagcccactgtctgtttttataaatacagttttcttggcacacagccacacattcatttatgtattgtctatggctactttcatgataccacaagagttggatagttgtgacagagactatataccccacaaagcctaagatgtttactgtctgaccccttccaggaaactttgccaggcttgctgtaaattgcactgtcaaaggaatcaaattaaaaattgctgcctcccaaaatggttctccactgttagaactcaggatggtggtggtctttgggagccagtgattggaggtttttgcaatagatctgtttcttcttttggatgctgcttacctgagtgaaaattcatcaagcttatgatgatatgcttttcttgtatgttatgtcaataaaaacaatctcaaaacattgccacacaccatcagtccctaaacttgaactccatttctcctagacatcaatgaatgccagtcttcaccttgtgcctttggagcgacctgtgtggatgagatcaatggctaccggtgtgtctgccctccagggcacagtggtgccaagtgccaggaaggtatgtgtgccaggcttcagctgcccatgggtcttctggggtgagcgagctttgcagtcaccatttgacttttcacaaataagccactgtgctcagggaagggaacacacagtgggactacaagggaaagggaacgtgaagagtcttccccaaaacaggtagctgttgtttttggtcagctgttgccacaaaactgtgtaccaaaccatcccaaactgctgtggcttgcagcaagcattatttttattgctcatgatatgcagcatgggcagagagattgcgctgtgggttgggttcagttcgaccagtggctgtgtctggggcatattcctttcatcgccagtgggtaggtgtgcaagaggctagtcaaaggctctgctcacatcacatctgccaatgttacattggccagagcaagtcttgtggccaagcgcagagccagcaaaggcaggaagtacacatgctgttactgtgggaagaatgatcttaacatggcaaagggcacaggcataaccatttcataacagggaccaaagaattgaaccccgatccggttcacagaaaaataaactgctcacccgtctccacctgtcagtttcagggagaccttgcatcaccatggggagtgtgataccagatggggccaaatgggatgatgactgtaatacctgccagtgcctgaatggacggatcgcctgctcaaaggtaggacatgatggctgccgcagttcacctgtgttctggaatcagggatgagcatgcctgctaagctgccagcttctgtttttctccaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccagtatgtaacaacctttgttttttttttgaatggtggatgtctgcttgcttgctttaaaggaggaatgccatgagctaggatctacttctccctagcttacattcctgttttataataaagtaagcttgaacttagccagcctcaaagagaacatctcagccttttgttcttcctctcaat

SEQ ID NO: 2 - DNA, 6048 bp, Homo sapiens
gggaggggcgtgcccagggtgagcacgccctctcatgaatattaataagcgcgcatgcgccctgcccggcgtgctgggtagaggtggccagccccggccgctgctgccagacgggctctccgggtccttctccgagagccgggcgggcacgcgtcattgtgttacctgcggccggcccgcgagctaggctggtttttttttttctcccctccctcccccctttttccatgcagctgatctaaaagggaataaaaggctgcgcataatcataataataaaagaaggggagcgcgagagaaggaaagaaagccgggaggtggaagaggagggggagcgtctcaaagaagcgatcagaataataaaaggaggccgggctctttgccttctggaacgggccgctcttgaaagggcttttgaaaagtggtgttgttttccagtcgtgcatgctccaatcggcggagtatattagagccgggacgcggcggccgcaggggcagcggcgacggcagcaccggcggcagcaccagcgcgaacagcagcggcggcgtcccgagtgcccgcggcgcgcggcgcagcgatgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccccgaatgtaacagagctatttgccgacaaggctgcagtcctaagcatgggtcttgcaaactcccaggtgactgcaggtgccagtacggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttgaatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattatgagaacaagaactccaaaatgtctaaaataaggacacacaattctgaagtagaagaggacgacatggacaaacaccagcagaaagcccggtttgccaagcagccggcgtacacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttattttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaatttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatcttactttgtaaattctgatttcttttcaccattcgtacataatactgaaccacttgtagatttgatttttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaaatttttttgtatgaaaacatggaacagtgtggcctcttctgagcttacgtagttctaccggctttgccatgtgcttctgccaccctgctgagtctgttctggtaatcggggtataataggctctgcctgacagagggatggaggaagaactgaaaggcttttcaaccacaaaactcatctggagttctcaaagacctggggctgctgtgaagctggaactgcgggagccccatctaggggagccttgattcccttgttattcaacagcaagtgtgaatactgcttgaataaacaccactggattaatggcc

SEQ ID NO: 3 - DNA, 5988 bp, Homo sapiens
ctgcccggcgtgctgggtagaggtggccagccccggccgctgctgccagacgggctctccgggtccttctccgagagccgggcgggcacgcgtcattgtgttacctgcggccggcccgcgagctaggctggtttttttttttctcccctccctcccccctttttccatgcagctgatctaaaagggaataaaaggctgcgcataatcataataataaaagaaggggagcgcgagagaaggaaagaaagccgggaggtggaagaggagggggagcgtctcaaagaagcgatcagaataataaaaggaggccgggctctttgccttctggaacgggccgctcttgaaagggcttttgaaaagtggtgttgttttccagtcgtgcatgctccaatcggcggagtatattagagccgggacgcggcggccgcaggggcagcggcgacggcagcaccggcggcagcaccagcgcgaacagcagcggcggcgtcccgagtgcccgcggcgcgcggcgcagcgatgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccccgaatgtaacagagctatttgccgacaaggctgcagtcctaagcatgggtcttgcaaactcccaggtgactgcaggtgccagtacggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttgaatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattatgagaacaagaactccaaaatgtctaaaataaggacacacaattctgaagtagaagaggacgacatggacaaacaccagcagaaagcccggtttgccaagcagccggcgtacacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttattttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaatttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatcttactttgtaaattctgatttcttttcaccattcgtacataatactgaaccacttgtagatttgatttttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaaatttttttgtatgaaaacatggaacagtgtggcctcttctgagcttacgtagttctaccggctttgccatgtgcttctgccaccctgctgagtctgttctggtaatcggggtataataggctctgcctgacagagggatggaggaagaactgaaaggcttttcaaccacaaaactcatctggagttctcaaagacctggggctgctgtgaagctggaactgcgggagccccatctaggggagccttgattcccttgttattcaacagcaagtgtgaatactgcttgaataaacaccactggattaatggcca

SEQ ID NO: 4 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 5 - protein, 1185 aa, Homo sapiens
QFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 6 - DNA, 32767 bp, Canis familiaris
atgcggtccccacggacgcgcgaccggcccgggcgccccctgagcctcctgctcgccctgctctgcgccctgcgagccaaggtaggagcctgccgggcctccctcccggcccgccctctcctttccctcgcagctccctggacgcctgcggctgagcgcctcgagcgggcgcgggagcccccgggcgccccgctccccggctgggtcccccgcgcccggggggctgcacctgggccgggaagtcccggacctcctacgtgtcccctccaccctccgggcggggccgggggctgtgttctctccgctcctcgcccccgggagcactcgtcggatttctccggcactcgtgcattttgtgctcgggaatcacgttgagtccttgcacccagttttgcaaaatccttttcactcggcgccgggggcccgtcggggcgggcggggagggagtcgccgcttccaccctcggagcaaacccctctcccccgcgctgacctccctcccccctcgccggcaggtgtgcggcgcctcgggccagttcgagctggagatcctgtccatgcagaacgtgaacggggagctgcagaacgggcactgctgcggcggcgtccggagcccgggggaccgcaagtgctctcacgacgagtgtgacacgtacttcaaagtgtgcctcaaggagtaccagttccgcgtcacggccggggggccctgtagcttcgggtcgggctccacgccagtcctcgggggcaacaccttcaatctcaaggccggccgtggcagcgaacgcaaccgcatcgtgctgcctttcagtttcgcctggccggtgagtgcgccacgcggggagggcggcccgccggggtcccagcccgcgccggcggagccccgcggcctcgccagagggacggcgggtctgggctggagcctgcgcgccggctggcaaagccttgccgggtgcggcgtgaggcggctgcgactccggttacggtctccgcggcctcttgcctagcgcgcgacagtggggagcccgcgggggctcgcgggggctcgcggggcaaagctcccagggaggcgggcttattaaacctgcatctagaaggccccagagtgaccctacccccccccccccccagctgggcgtctttatggacgatctctctttgcttaacgaattgaacctgatgcgccgtggaaggcgacgcgcagttctggccttcgaagccgtccaaatggtcactcccccctttctcgtgagctgccgcgagggcgggtgtgcccttcctggagggcgtgggggagccagtttcccgccgctgcccgggagactttggggcgtgcggggacgcgctccgggctggacgggagggtgcgggggcggccgccgcctgggaggtgggtgggggttgactctgggccgaggctggagccgggagcccgagagcctccgccccctgccggccccttcctgccctcgcccccgcgcggtgtagcccttgtggcctcacttccagcggttcccgggctctggaaccagctgtgtttgcaaacttccccgggaagggcgggggtgcgccctcgtccggcgggctccggcccccgctagcctccgggggcgtgtcaaggcctggagggggcggcccgctggaggcggctgcggagaaagtgccagggctcggacccctgccccgggcgccgctcgaggccccgggcccggctggcccgccctgcaaccaggcccttgcaacatcccaaactgggtagaaggaagatgtgtgtgattgcagaagcggaggaaaggggaaaacgctggtgccctctgctccgctcctgccgtccaggaggcgtggctcatcgccaggactggggcttggcccggcctgtgcggcgagggtgggcggtgagggggggaggggaaagctccgtgcccgggggaatactctccgccacgtgctctgatggggtgcgctctgctgcctcctatccgcgggctggggctgtgattcgcagggccgggaggagagggaggggggagagtgcctcggtgtgggcgtggggggggggcacgtgtgattgtgagtgtgtgtgtgtgtgtgtgtgtgtgtgtatcttggggtggctctcaagccgcacattgactggagctggcgttataaagtgccctctgccatgggtgagataagacagttatcggaaacatgccattaaaagggacttataaaaacccatggtatgtagagttctgagaatggagggctgcgtctgttgtgagatttcaattttacaaagactgtttatcttgtttgttaatcccctactagggagaactttaaagaggctggccactttgtaatctgcccgtgcccaagttcactgtgaatactgcccgcccttgaacttgtgtttagagtttaggggggaaggggcctgcaggtggttccaccctcctgaggatcagtccggagccccttgcttttcacaagaccataaaaggaatcttattagtgatgtggaaaagctgcaggcctggctgaggctcactttggagagtagggtcagaggatggcttggatgagaacagggagccttgctcaccagactggccccggaaaagtgacccagaaggtgtgtctgtccctatctaggactctgccaaatgtcaaatggggaatgagcagaagcggtgtgtggggcagggagacgattgatttgctggtgctcttctgtggtttcagggcccacgggtcaccaaccatgaaatagactctcggcttttctagaatagggagactgctggggctacacagagtgtagttcttttgaagttgggccaaatgcatgtatcactcataaatgagtctccaccctgccccccctaaaagacctgctgtgaaacataagctttgcttcccaagagaaagtgaagtttaggtgtattgttttagtttattttccgagtgtggttagagaaacaccacagtctgtaacttttaaagaatgattccagggtgaatgttaacctgaagtgaaagcatttaggtatcgaagaacatagaacagtctcattcagtttggcaataaaatattaataagcacgtctatttaagactgactttcttaaataggtaataactgaacttggtacagaattcaagatggaaggtgatacaatttttttttcttttttttttaatcaaaccacttgggacttccattggaggccctggcccttaagcagtcacaattacccttagcatgggtcttcccaaatgtgctgacagtggaagagctgtggttcagagggcacggtagaaggtcagtttagaaggtggtgtaattcatgtcttttgtttggaattatgactgtagtcaaaccaactgttcgtgttatagagaggaagaagaggaaatgagtggtcacatttgaaatctagtccacattccagtgtccctgtggggagctctgtagaggtgggtctgcctgttggattgtatctgatcaatgtgccactggttgtctgctgcccctgttccaggcttgtggttctcattggtggcttctaatccccaaaccagggctgttcacagggtcttaactggaagtaactggcctgattgatgaccctccaatcagacgcacccagtgataagaacatatgttatggaaggtaattgagctcaacgggaagtgatttgagaataaccatttgtaaatcattgaaatggttaagttgtacctctcagaattagtgctaactccccatagtacctgcttattgaaaatccattcatgcagttaccactaccaagcccccagagctttttaggaactccaggaccccagttcctgaactgatgctttgcggaccctgcagtgaaatgaatctagagaaacttggggtggggtggaggtagttgactatcaggtcttttggcgtcctatttaaaggccattgacagtgataaaaggcttgctgggagcagctgattttttttttttttttcaggaaaatccaatgaagtgtcagagattacagagggatgccagctttggcccctttcacacttagggcctgattgagcaactgcgggggcggggagggggggtttgaggaaatccgatgccactaggttcatcacagaatataattgatatgtccttgcctttatgccctctgctgtgtttctgggtccctgtgcttctgttaggtgcggtgtggacacatggatggggttctcctctgaactggctcctttctgcagccagtatgtacagcccatgtggtcatcatgcatgcagacaaccgcacacaggagttcaagtgctttgagcacaggccttttctcatgtggagaaaacctcttctgttctcagacccctgtgtcaaatgaaagtgatgaaatgacacatctgcgttgcaaaaccaggcttggggggaaaggggacctgtgcttacttggtgctaagtgtctgtaggatggctgctccccacatgaaatccctgttcagattacttggtttctaattttttcttctcctcaaggtgtaaacctctgtgttttcttcctccttgtcttgtttcttagcaacttaaacctccagtacttacatgagtttgcaacccaccaactccacacccataattcttcagcgacttcagacatttgtagacaacggtgaagcttttggcatcgaggcagtaggggtaaggaggagtggcagccacttaaacaggcatgtcacttccctgaaaaccaatgatctgaatactgcctacaacctttacatcaaggagcaggaaggggtcccttggtagaaggcatgtggaaagtttcgcatggatgaaaaatgttgttcctgaaaaacatgcagccaaggcaacaaaggagtcttcatgttcctgatctgaaaacaggaaagcaagcagaggtcttggctggattcatagcagccaccagtccattctaaaggcccagaacttggttccagtgtcaggcttgccatgtaggaatgcgtcctctctggatgtgcacacagttagcatggtagtccttaaggcaaatattcccaagagttcccccttggtaaacttctataagtgtgttccttctgagagaagtggcacatggctcgagcttttaaatacacttaaaaaaataaaataaaataaacataatccacttttcaacttcaggaagatgtattaggatgcatgagaagtgatgactcttgggtttcctagaacggtggcagaggtagaaccgggttctgcattcagaagtgttctacctttatcttggctcgtggtgctgggtggccggccgtgtggtgatttccttgctggtttctggtcactgcgtggactgccctgccctgtggtgacgtttgtcactggtgcagtgcatgttcagggaagaaattctgcgatgtggctcccagtggctaaagtggtacataaatagtcaaacattcagagtcctccaccagctaagcccagtttttctacacttttggtaaataagtagaaaggcttcttgttcaggcttgctctgtgtgaaccggacggttgtgcttggctggcttctcggtattctatctattaacctcgccacccttttgtaactggtgcatttaattatcagtgggatgcttttcaaacaggtaagggtaatgatgggaaaggcggtgggggcatctcattgagagtctccattctggatatgagctttggggcccacttggtgagtctttgaggtgggctttcatgagaaaatcaccctttgtaaccagatcggactctattgaaatgtcaagccctgcagactaaattgcagtccacggccgaagtcatgtcagctcggatgctgtggggcacttgtcctttcttcaggcctcccctggccacgacctggtgacacgtgaattgttcgtgcgagcgtgcgtgtgcccacacatgcgatcgctgaagcctttaatatgtttgtgactggaatattttataccgtggccacccagacgcaggcaggacaatagtgtggccgaattgactgaaatgaggattacagagaataatgccgcctctccaaaaatactgctaatacagggcccgcgacaggaagcgccagttgagtcagcagagcacagggccccaggcaaaccccgggccgaccgcctggcacagtggccccccgggcacgtcgggcatcccggggtctgtgctcctcggccgccccgtcgcagcggcctggggttttggacgccgcacgcgtgccgctcggacggcggtttgttgcgtccgtcagcttggcaggaatggaagcagcctcttctcgggcttggtttggccgccactgttgccaggaaaaggaagctcccagctgccaacacaattacctttgtttcttacatcccccttcttaatgtggcaattaagtgcttaccactctgcgcggtgccaaggcaaccaccgtggagtcgcccgcagcctccgtgccccgggtgctgggaggccccgggtgcctccgggggccttggggtttggaggccagaccctcctccccagaggtcactctgagagctggccattcttctttcctgctgcctgggaatggggactagctgctgtggccagatttccctgggcctggaggtgggaagggaggcacaaacccacactgaagtagcttcccatctacccatccatcctgctctgcccccaggctctcattctgggagagatttaagaaaagaaaaaaaaaaaaaaaagcaaggtgactcttcttctagtcttacttttgtagtctgaggccaagaaagatccaggtatttgctggttctgtttctcgtgggtggtatttttgtatgaacttgttaagttggacacttgcccatcatctggcctggcaccccctttccaagcccttaagagggtgcccagggacttggcctccgctgggggacagattggcagtgacaaattgggggtgacgtgatcagagcagattgcgtccaattcttggcctgatactggcctaacatgatgacacaaagtgtgcggtctggacctccgagtatggatatatattctgggatgtacagaggtcaagcgtgggcctgggaaaaattaaagttcctagcaactaataatcattaccaaattatcttgattgttgatctatagtgtggggatggagtgggagggaagagggcctcctaataggggccttggagggaatcctttcttctgaaggctgggtgggaggaatcctcctgccccaggaggtgaggcccagaaccatcagcacttctcagaaaccagggacagagacgatccagggctttttatttcttttgtttattttttttgacaaggccttgttgtcagcactgtagccccagccctcaccccgtgtccgtctctgagaaggcagttcacggaagattaggacacttggcgccccgagctcagtgctgccgggtcgctcttttccctcgaggttgggcagtgtcggtcagcacctcctgaggtcttcccggcaatggtggagatgaacctggtacacagcttgctcttcctggttcttttgcaagaactgagggatgccaccttctttgagcatcaagcagagttaatactgtcatgccagctcagtccaggcacgccttctcctcctgtggggtttgtggatattacatgtttagtgcttgtgcaagtctgtcagaaccgcgggtatcaatgcatttaatcagaggattaattacaatacgggaatgtatagaacgtggtctcgacatggccagccagcgatgggcttaattatacagaatcagccccacatcgggcctcatttggacttggttctcacctccaatcagtagagaaatgtaatttcttgctgctctcagggaagagtcctgtaatttgttgttaagtcccgggaaatttagtggctgtaaacaaggtgtaacttttactaataaaatattaacgatgacaacaatgaattgggacccatggccaccaaacaggagagggctgggtatgtgccctggcttgcctggttttgcttgcttttctgagcattttcccacagttatcagtgaacgaaatggaatcctactctgctttgagtatgaaacatatttcggctgaattccaaggcagggtctatcacaagatatttttatattcagagacaacatgctgtgtctctgtgcgagccataagcttgcctggagttatgaatgaagctccttttttctttcttttgctccatgtttgccttcaccttgtaggtctgcctcttcttggtgactgtgcttctggccacacggtgctctttgagctccgtgcgccactgtgtccagtgcgttggtatctgacagtgaccatgcagggatacagaggaggaaggcctcatgggccttttctccccacccccctcagatatatttctccagcgccaagatagcaggtttatttaaactgtggtgttttgtgagactgacctcattctaaattacaagtgaagatttgagtaaaagcgggaatttgctaatggtttggatttgggggggggggggaggtgtggaattctgagataagcctggaagataaaaaggtgccttcagtataatagctggcatcctgccagtgtccagggaatttgtttagttagtggactctgattattcctggaatgttagatcttattgacagataagtccatcaatagctataacccgtgggggtgtggtggtttctccctttttgtgctttcacccccagagtctggcccccaagcaccccaagttaggtcgccgcctttgtcctggtctctttgcacctaccccccccccctgcttcctagagtcacctgatacttgtgggagctgagatggtctcacagacagggctttcaaaggggttctggaaaggaaagggggcctccccaggggctgccccaccagggcccaaagtgtaaccttagcaatactcagccctcctcactggatgccctccccccgcccccatgcaaatacctggttaggttagcttagaagaaattctttgtgtgtgtttctcatgcactagagttaaggtctggaaagttttggaaaactgtactgtgttcacatatagggaggtggtacttcctgatgatgactttgggcagcttgcacagctgacagtgaccaatcgtggtgggcccctgccaggcggaaggagtcaacctggggatgggtaccaaggaggggcgttgggggatctctgctccacagcaaacaagagttttcattttggttttatggttctgttcggtggagtatgtgagggagcgcgagcaagtgtgacatgtgggagctgactcttatttctgaatactaagtggtggtttttctctgttgcagaggtcctacactttgctggtggaagcgtgggattccagtaatgacactctccgtaagtatcacagcaacgggctttactccaggtgtgcctttctggaagtgcggtgcttggggcacagtagtgttgggctggatctgtctcccgagtgctgcagagggcttgtcctacactccctgctgtctgtccccatcctgtgcccaccccaccatccccagggtgaactccctggagtcttttccataacttcccctagaatgtgtccacttggaacattaagtggccaggacagattcccagttacctcagtccctgaatatgagacctagattatctttgggatagagatcttaatggttttacaactttattttttttttaaagattttatttatttatgagagagagagagagagagagagagaggcagagacataggcagagagagaagcaggctgcatgcaggagcccgacgtgggactcgatcccagatcccgggatcatgccctgagctgaaggcagaaccgctgagccacccaggcatcccggttttacaactttaaaactgtgcagactgctaaaggatcaaagtagtgactttttaaaagtccatttaaactttaagttgagcgtttaatctctcatcagaaagataaagcctgcaaaggtggcgctgagctctaaatctgaagaccttgaatttcactcggtgggcaggtgagcctctagcggctcagggtttctttcccacactgtgcatgacagacctttcattgaagtggaatcccagagcctcacaggcaccgcagctgcctgtccccttctgaaagatgacggtttcaggggcaggttgcttttcagaggtgcgaggtggggaagtcacagggattgtgtctggaaatggcagcagcctggcttctatggtgtagtctttgctctttaacttaaaaatgttttccctccctcgctgaaactctgcacttccttttttgctagcaagatctggaaggttgtcctgggaaaacttgattcatgctgaaatcccttagtctgcatagggggagggtgatccaactctctttgccctccaccctctgaatgtctcctcccttcttccgtacctcactgaccctttctggttttggctactgataacatctcccctcatctcctcaccatgccattttcttttaagcacatctgaggatgctgaatgcccagagtggatgtggtgagggcttggcacttgtgaggaaggctactttgggctgtgtctggctcatcggctctgccctccagccccccacctccctaccctccgcccggggccgcataattactgctaagccctgtagtgttgaaggttccagtcaatttgggtcactcctcttgctctgatatccgaagcccagatctgtggactctctgagggccaccccgtcctgctgtggtgtggtggcagaggagggtggtaaccagcctcagttgtggcccatgcctgtcagggtcattagacaccttttcgttctcctccctcatgggccacaaacattggggattgtgtgagagttgtgtcactgtgaactggttaccatggtaactaagctttgggaagatggggagggggcagttagacacttccgatattctggtgcagatgtgatcttctccttcccttccctgttagatgttaaaacaatctggtagagtgaagatcctcgagattgattctgactcatggcaggagctggttcaggacacctgttagggtgtgcagcacagttagtgtctgtagggcccttcttgggccttccgtcaccccacaggtcagctagctgcgcccctgggaagctgttgaccatccagagctgcctgcggttgtttccatttccgagtagccctgttttggggttctgaggcccctcctcactgaggagggggtaaagaaatccccagaagggcacaaggaagacttgaggtagggagaaagagctttcagacctctactcttgaagaccggagttgccacaaagccctgaaatgcctactgcaggtcgctgaatctcaccgtgtgagccccaaagaggaggaagtggtttatacacttgtttattcagcctctctctttacagcacttccaagggatcttgaattcacgttcagaaccatatttggcatcttctctctccaaattagatcctgcctagggcccttcatggatgactggcaccagcatttcaacacctagtaccatgagccagaaatctggaagacaccctgtagtacttccctttctcttcccatccatatctaatccaccttcaggttctgaaaatcttatctctaatgcctcactggaatcctgccacctcccacatgtcccctgctggtccaagtcaccacctgctaacacctgaacccctatggtagtggttgaattggcccatccatatctactctggtccaccaccagtgctttttccatcctcttgccatcctggtctttccagagcctttccctttttttcatttaaaatgattgacctacaatatcctctgtttctggtttgcatcttgattcgatttgattgtcctcttcactaagagccacttggtggcttcctgttgctcttgggacagctctgtgtgtggccccaaaggccttgtacagctgttttgtttccttgctagctcctaactccatcttacatgctgcattgtcttctaccatatccaaatcctcatcctgttccaccagtccccattctgtcctcaaatgcatcatcaccttctgtcctgccgcagggcccttgcacatgctgtttgcactgcctggaatgttttgcttattactctttaccctctgtccctcccatgaggcagtactccatgagggcaaagcctaaggatgtgtagcaacccagatggagttggctgctctttgggggccaaacaagttgccaaaattctggtttcatatgagaggcagaaccctgagttttaggacagaggattccttctcacctaggctctgtagagagattggccccgacgaagcccaggtgctgtttgagtgttagactgttgcctaaagcctggccctcctggcattagtcctcttgagaccatttctcttctctttcagaactatataagacccacatttttcttttctcccccagtgcaaacggtttcagtttatggcagtgctcagtagatcaaataagtgttagtccaggttttccttctagcttaggcaccttgactaagctcctgtcctaggtttgtgggttggcgtggcctcaaggttatgctcagactcgccttattacctctgccttggcctgcgaggctgttgtgggggctacgtgctccatcttcacacagctgaagatcacaggagggtctgctaacatcctcttccggtcatagagctggacccccaggtgaaataccccctggatacacctctatgccccaagatgagaggacgtgcaattcaggtggcggcagcttggaagaagcaggtagcagagttgatgtctacaaccagataccagaaagtctgtcctgcacccatgactcccccccccaccccccccaccaccacctccagctgctgggaggaacctgccacacaggtgtgaggctggttctatggatctgttagccatggtgaggattccagaataatagttttcacattttctagacctaggtggaaagccaactgaactgttgaaaatgacctgtcagcctgtgtctttaggattaccctagactcttgtctgaactgtttgttgttacctgcccaagagatgctggaggcctttgagccgtgtgtttaaataattcccgtattaagatctcataatcacatggtaatagctcgctacagtttttaattgaatcacggagcaaacacacagttaatggagggacatttttgtcattccttgacctgtgttatttgtaagacttcatgttagtaaagcgttatctgcaggtggttctgtttgtgctggggtgtaagtctacaggtatgtgggcttgttcgacgcttgtcctgcccacacgacagttaaacccagagggtgaatgctggggcgaagctggaactcgtgtggatggagcccctcaggagctcctcctcccctggtcacttgttggctttgcataatcttgtagatgagcacaggcgctggtcctagaaaaacaggggtgtgggtgggtaagaatcacagacattgtgagtaggtcctaacacagtctcccgaaggtggccttgcccttgacaaggcagtgaaggcttggtgctggcaggtttaagaaaacaaacaccacatcaggctgtggtgctgacttactgccttgtttccctttcccgtccctcccctggactcctggcacctggcggttggatttatttttgaacctgtctcttctctgactagattataagtgacctcagggcagggaccacttcttactcctctcgagtgagagctggctgcttgagctctgggcgtgttcattttcaataggcaccttttaaagagcatgacttggctctgaaaaacccctagtctacctgtccctaaactggggtgtgttactttcccctctgccttctgggcaaaggtgaaaggtgaagcacatcaagttcgtggtacaagtgccagctgaaaaggtggcaaagtttataaatttttaaagacctaaaactctacccttggcttccagggaggagtttagaagtagaccaagtctttctagagcaagacccaaaactaaaaccagaagaggaggattgctaacctttacctggaataaggtgctttctctaatatgtaaaaggcttttgcaaattggtgtggccagcaacgcactagagcacttggcaaaggagagaaacaggttacagttcacaggaacagatgcagatggctttgtaaccacgtggaagggtgctcaccatcacttaattaaatgctaattaaagtaagagatccgtctttacctatcaggcaaagatcagaaagtttggtaacctatactgtgctggcatcgccgtcgggaaagccagtttcctgtgcgggtggcaggacaggctgacaggcctttgttcgtggtcgtctgtcgatggatgtgggttgtgtgcactgtcctctgacccagcgcttccctttctaggaggtctcttacactagaaccacgcacatgggcagtcggttcaaggatgttcgttatggctttgctcactggtaaaaggtcaaaatagcctaagtgcacatatgtcaggaactaggtgaacgcgtttcactgtattcccagacagggaaatactacacagccgtttaaaagggtaaatagggcagcccccggtggctcagcggtttagcgcctcctgcagcccagggcctgatcctggagacccgggatcgagtcccacgtcgggttccccgcgtggagcctgcttctccctctgcctgtgtctctgcctctctctccctctctgtgcctctcatgaatgaataaataaaacctttaaaaaaataaaagggtaaataaacgtaaaggtggctgcctctggggagagggacagacttttcacttttctgcctgtttgtagtgtttgaattttgtactatgtgcgtttgttttcattgttaaaaataggcaaaatggtcaggggagaagcttctggtgcaaatgttaatacaaatcaatacttttttttttctttttttgttttgtctctagagcccgacagcattattgaaaaggcctctcactcaggcatgatcaaccccagcaggcagtggcagacactgaagcagaacacaggggttgcccactttgagtatcagatccgtgtcacctgtgatgactattactatggctttggctgcaacaagttctgccgacccagagatgacttctttggacactatgcctgtgaccagaatggcaacaaaacttgcatggagggctggatgggcccagaatgtaacaaaggtgtgtgggcaggcgtgtgctgggtgggttaaccagctctgagtggctggcactgttggtgtcaggttctctccccacccccacccagtcaagtggctatctcacatggggggatatttgggctgttttctaggcctgaagcacaaagatgctgaagatgctgttggtactcccctttcccccagctgctgagtgttggctgctaacggctcacagctgttccttctctagagccttgtccttggccaaacagccactccatgcctcttccctccagctcacagtctgcgaatgactggatgggggtacaaaggccagccaccacccccaccactccccaccccccgttggccttgcactctgtgctacagttcacacgccagggcttcccctgggatcacgttaaagccagattccagctgagacctctgcttggctccttccccaccccatctgctgccttcaactcctttctcctgtacccaaacccctgtctcaggctctgcttctaaggaacctgacctaaagcagatgctaattgggacagggttagctcagtgcctgaatatgtgatcctgcagcaaatgacagcccctagttcacggccatctgctggcaagcttttacgaggtgttttcgggggctgtggtgttgagggggatggggtgagtggtgaaccagagctttaacacttgcatagagtcgaacccatcaccgcagcataagctctcagatgtgctgtctgttgtcccagccttctcctgggattcagggagttaactgcatagtcacagatgttcacagaattatctgtgctttgtacctttacaaagcttctaattcatagcaagtgggtaattctagttgaacagtgtaggccctcacgaacattagtgtactctgcttatttcacctaatttggtaaattctttcaagatacactcaaatttagaaagggaggttttgaattgactgaagggcaagacacagaggaatttgcaggtctcctgagcttttcttaaatcactagagttttaaagggatggcatttccggtatccaaatgaaagaattagcaaacgtacctaaagtagttggtattctgaaaataagtaatgattactgggatacgatgttgagtattttttttttatctgttttttttttttttttttgttttgtttttgttttttttttttttaatgaaaggaaaaagaattcctttgcaaggatactttgcagaagtaccaccagatctggcctctggattagcctgccaaatgaggcctgcctcattggctttgttagtcccatgggatttttgagacattgggcattctagggggtggggatgagggggttaaggatgctggggctcatcttgaatgtgccctcatgggacagggtggtgagatgaggaaggatcacagctcctgttcttttggagagggtgggaatcagggagctcgctagctattgtggagagcctcagaatgaagatccccctcccccatttagtccctgagtagtatcatgttttgaagagctgctagcatggccttgaagtttaaagctggatttgctatttgggcacctcccatcttccttcccttacaatggacttgacatgagatctcttaaagtcatggattttgaggcttaatgtactgggccattctcgtggtggtggtggtggtggtggtggtggtggtggtggtgcaagaaaccatgtgcttgactcatctttgtgtgtgttttgttgttgtttttatagctatttgccgacagggctgtagtcccaagcatggatcttgcaaactcccaggtgactgcaggtaaatgactaaacttttttttttttttttaatcctttaatgcaagagggggactgaatttattgtgaacatgcctcttttcctttggaggacaagaaagcttaaaaaaaaaaaaaaaaccaaataggcacatccgaagcttattaaattataccagctgtctggtggaatgtgcacacttctctcttggaaggaaaaaaaaaaaactcttatgggcatagtcggtgacttcatgtattaactgttctgtaaattgataacacgtttttataatcttccccttaaagtgctaatggatgagatggagggatgtgggtttctttgagtctgtctcctcccccaacctggttttcggtctcccaaccctcccccacttttcccatcactaggggacagccgagcattctttagaatgggattagtacaatctcctggcccctttgcaggcgggagccaggccggtgctgagaggttaatggctgcgctggaatcttgtcagttgagcctgatgaatgtagacttttgctgctggcctttgaagtctgttcttttatggtcccggacaatgcagaagccaaacagagacccaccccaactgcttcatgattacagtttttggaaccttcttgaaatgtacattatgccaatttgggaaagacttgcttcaggaaatggtgttggtgcctgtgacattctgacacagtctcacttgcctccccctcctctcagatctgaaggattagcagatttagtgacactgagaggatccttggggcccagcagttttcccccaggagctaccctggttgagcccagggaggcaaacgaaacacactaaaccagcacatctcaagtggactctcttccaccgctctggttaccatgcagggtctgatcccataggtctggggggacctgaatgtgcatttccaataagtggcccgtccccccgcccggtgatgcccatgtcgctggcaggaccatactttgagaatcactgccccaaacctcagtgttcacgtccgtagttccctgaatgaactggggacttggacatgcagattcccgggtgctgcaccgcacccactgaatcccaggccagggttcggcccaggaatctgcatcttgacgaagcccaccgagcgatcttgctgttctctgtgtttgggggactgccactttgcagggaattctctggtctttactttgcggccccgccggcgcacgtctcttcccctcctctgcagcggggagcactgctctgaggcttgatgccaaagtgagaactgaggcagctggaagcaaactcaaaccaggttgaccgggcattgtctcttcctccatgtcagagaggtttattttctgttgggccgtgggtggtggcatgctaagccccattggtggcagctgagcattggagttgacagcattgctgtcaaagtgtgagcaggaatccacctaccttcgcccctcttaagggtgttctagagaaagtcagatgagcagcgctgttcgccactctgggcctgctctgctcagatgggtgtcgggaggccggccgccgcgatttcggatttctgctactgtcatcagaacggctgcgcagggagggccctcccagccagaacaacaggctttgtttccagaacggagccctttctctttcgaagtctcccctcccactccctgcccagacatgcaccggtgcctttatttacttaaggtgtgactttcccacacgtcatttgcatagtctcccagcctgtcctgttctgcctaccttgaaggaaaatcaaaacagaaccttctagctgaggaagctcaggttgtgtcctgctccagaaatagttgaatggccagttacctgtaagttgtcctatggattatgtattacagaggtggtgtttttttgttgtttggtttgtttttgctgccttttttttttttttttttaatccccgcatgagaaaacttcactggttttgttgttcccaagtagactcagattccttttcctgggtgcacatgcttgctttattgcaagcctgccaataatatcaaacttaatgatgaatcattgtaggaggaaagtcacttcaaattctcatgagaaccttaatcatgtattattaaactgggataactttgtagatgcctcttttagggtcagtatttttatttgttttcttttgaagtcatccatggcctcttgtggtttcttctttatgcttggggatccagctctcgttaattaagaggtgaggttgggtcctcagctgcctcttcttttattaggcaaagtgaagaggtgtcgccatccatattttggaaggaagagaagacagtaaggatactgctatttccgaatcagtttcctcttaatagctctcaagctaagatgatcttttcctgaatgaaaccccctcccccaactcccactaacttgagtgtccactttaaaaaaaaaaaaaaaatttctaccagatttttttttttttttcttactgctctgaatggtcacggtcatttagactgctgttctgttatacacgaagccaaaccctctgtctgattaattttaaaccacgcattctggtggttctttgaattggggtgtggatggcttgtttgtggaagttaggaagtggttagtcagtgttctcaggggttaagttgactcttggccgtgggtgattagctattagctggatccctataattttcccatgataaggatttgctagcctgcctccggctctctggaaacggaaagcctttccttcttgggggtgcttgttttccagagttacagctgagtggctgggggtgggggagggcgctggtcgttcttactgtggcttttcccaggtccagttccatgctcgccccaccttctctgggctttctcctgtttactaaagcttgagttttaagagggcagttaaagtttctgagggcttaaaacttgaggggagagtgagtgagaccctgccctctctcccaccccaggtgaaggtttatgtttgtgtttctgtttggggatcaaaggcaccgttggcctctaaagagtccgattaatgagacgcttatgaaggaaacttgactctgtttcccaatactagataaaccgagttgacctcttcctccccccccccacccaccccgagtgagaaatcactataaaatgaattagcttaagcagccccggtagtgggccataaattatctgtgggattggttccaagtgctgcggctccgctacctgtcctgcagaagtgggttccgagatctttgcctcctcttggccagaggagggtgccccccccctttgatttcctaaagcctcccagggaggtggcttcctgcctcactgggtgtcttgttgcgtttctgtgcactggtgacttgctgtttagacaaagaaccaggcctccgtggcaaagtggccgacggtgccgtggttgcgacgactttcactggctcttgtcacccgagagtccttcctgtgcctccgcagccgctcgccggggccctggactggtttattccacgcccccaccgtcgcgcagccctgtgagcctgagaagagagtggctttgtgaggtcagcccccgccggcctctccccagacagccctgcttttgtcggcagccgcctgggaggggacctcggcccaaggaccgaaaatgtttgtttagccggagcccccgccggagcccccgcacaagccttccttgcgagtccactgtcctgtcaccagaaatgaaacaaggcaggaaaggcctggacttcttagccttgcacttaaatggtgcagatcgccccgaatgttctaaggactaaagggggggtgcgtgctgtccgtccccaggtgccagtacggctggcaaggcctgtactgcgacaagtgcatcccgcaccccgggtgtgtccacggcacctgtaacgagccctggcagtgcctctgcgagaccaactggggcggccagctctgcgacaaaggtaggctcctgggggtgggaccctgggcgggacggggcctcggggttcacgggagagttaaggagtgcggccgctgtcgtggggctttcacttttattttatttttatttctagggacttaattctgaatctgttcgaatttgcctttaaggccgtggcttggggtcttttttttctggttttagtctttaaagaggaacggtgggggttggggcccccccgctagaaacgtggcagccgagcagcctgtgccgcctgcgagccactgaccccgcggtctctctggcagatctcaactactgcgggactcggcagccctgcctcaacgggggcacctgcagcaacacgggccctgacaagtaccagtgctcctgccccgagggctactcaggccccaactgtgagatcggtgagcggtccggggggaccgagagccctcggcttaatttctttttttttttttttcttttggtactctgagtttacaaaaaaataacaatacccttaaggcctcataaaaggacgaaaatatcctccaaagtgaagacctgttaccccagcggttctgtgggtcccctaaacttccttcagggtgatgccgcccacgcgggggggcgtgaccccggctctgaggctctgagccgccttggaatgtggctggggacacttaggccgaaagccggcttcctgccttccctggacacagcgggcctatagcccgggctcgctcccgcttgtccgaggcgagctggagctgcggcccaggcgtgggactggctgcggggagagaggacgtaggtcgaaacacgcgggtgccccttgctcactgccctccgggtttcgcagcggagcacgcctgcctgtccgacccctgccacaacagaggcagctgcagggagacctcgctgggcttcgagtgtgagtgctctccgggctggacgggccccacgtgttccacaagtaagtctggggttgggcccgtgtcttttccagaggatcactgcattgaggtggggcttgggggcaggtctcagtgaggagtcttgtctcggtgggaggggggaggtgccatcagcagtctgatggggagtctggcttgggcttccagcccttgcacccgaagtctctgggcaccctcctcctaccaagccaagtttgcatggtgtggctgctaccagaagggacttctccgcgggatacccccgtcctctgtgagtgctccctggagctctgctgcctccaaatcaagtcctagctcttctccttggagccatggagaggctgccggcaagggaggtagccactcctcaatttggtcatgggtggagggtggccccaaaagagagacccaggccacagagtggtcctggctctgaggcctggctgcgcatcagaacccccaaagtgctggacagtgcagatgagggggtcctcacaattcccctcaggactggggagtccacattctttcacgagcacctgggcctctcaggtgcaacctggcctaagaaccactgtcctcagcaccctgaaagtagagcatgcctggttaagaatgtgggttctggggttgggtcacaggggctggcaccctggccccacctgcctcttgtaggaccactcgttacctctctgtgccctgttgaacgggacagtggcaatgcccgtctcttagggttgtgtagaatcaatgagcagatcacaagagagcctggtacttcctaagtgggcagcagaggaagaggctactagagacaagtgcagaagtgggatgtggggcccccagctatgtggttttgggaaaactctgagaaaattagaagaatgaagttttctaccagaaaagagtcagttttccttacctcttcttagctacctctgagccacgctgaaacttcgctttaaaaataaagagtaggtagggtgcccaggtggctcagtcagcctttggctcgggtcatgatcctggagatccaggatcagctgcatcgggctccctgctctgcggggagtctgcttctccctctactcctaaacaccctcctcccatgcactttctctctcaaataaataaaatctttaaaaatagagagtagacagagctagaggtcccatgttagcagtgtggtgatgctcttggggatccagctggcttttcctgctgcttagatgggggattaaatgaactggggcagtggaggatttaaagctaaaatcaggccttatcgtcatagcagtcacttagagcaggtgtctggctcttaagtgaaggcccctcatgtgagggttggcattcaattatctaattgtcaaacggactgagattaaattgccaaccctcttcccttttgtccaacagacattgatgactgttctccaaataactgttcccacgggggcacctgccaggacttggttaatggattcaagtgcgtgtgcccaccccagtggactggcaaaacgtgccagttaggtaaggaagttcgggcacccaggttggcccttatggatccgagttgttactggagatgagcctgtggctagtggtgtgagtttgcacaccatcatactaaaaagtaatttattgtcaggagtccagcgggcctgggagagggtacttgaatgctaatggcctagctctttaggccctgtgggaaagtcgtgggattccttgaaccatgtcaatcgtctgtccgaggagtgttcagctgggttccacggggatggtctcatgaggaatgagctgtctgcattcttcctgccacatttaaatcccagcaactaggctgcgccgcgctcgcacagcaatgcctggtggctttttgctctcctgttgggggccagttgttaaccaccagccagaaagttatcaattagcattccttattgggtaatggctagggagagggatcagctgttccaaatagtgggaacctagtctctgatttagcgaggttattaaaacgtctcctagttcagactattgatagggtgtgtttgggcgggggtgggggagtggggggtaaatctcccatttctcaatgcccctccacattttctttcagatgcaaatgaatgtgaggccaaaccttgtgtaaatgccaagtcctgtaagaacctgattgcgagctactactgtgattgcctccctggctggacgggtcagaattgtgacataagtgagtccctttgtttcgattttgatttttgttgaaacttgggtattaaaggctttcctcagcctctatgtcagctccagctctaagacttgagcggtaaaaggaaaacctcagagagggaatgtttttggggagggagcggtcagtgcagaaatgaaaacatgcactttatcagctcattagccggtaaaacagccgtatgctgataaggtggctaaaacaatgatgtgaagtttcactcgctcagtctggaggaatgagggagggttcccacccagtgtaataaccgcatgtcaagagaagcacacaggacatctgatttgaggcttttatctgtcctgagcggagaacgcttttcaattaagcccaatttcaatgtaaattgcctctttaatacgacaacctgtttatttttcagatattaatgactgccttggccagtgtcagaatgatgcctcctgtcgggtatgtgaatctttgcttaagtgaaaaccttactgattgacacaaatctgttaagatacatgtgaccctgggagagcttgatccctctgagattctaaaaatgcaggtgaaaagtgattctccgcaagtttcagttgggctcctgttaccttcccagccccctgggagccatctcgaaggcatgcctatagaaattcctctctcacgttgcgagaggctgggcatttagcgatgtcagggattcagtggaagggcagcgggtgtgctagactgccaaggccccatgtttgtggaatacagaggaaaaagcatgtgtttctccccccctccttctcctcttccaggatttggttaatggttatcgctgtatctgtccacctggctatgcaggcgatcactgtgagagagacatcgatgaatgcgccagcaacccctgtttgaatgggggtcactgtcagaatgaaatcaacagattccagtgtctgtgtcccactggtttctctggaaacctctgtcaggtgagtggggctagaacctccaaaggccaagtttctacagcagagcagctccccacccctgcccagttccctcctgatctctggacttgccatgaaatctgttaccatccagtgaccagggacttctgctcggtgaacacaggtggaaggacaccaaggagtgggttttgttcacagtgtcggcagcctgtccatttgtgcccccaactccgtgctctcccactctgccctgttttgccgtgcatccctggacagtctctaggacaaaactgtctattatgtagggtgttctccaaatggaaggaggtgaaggggtcagattccagcaactgctgccccagagcacttctaatcacacctctgtttcctctcccccttccctgctcctggcagctggacatagattattgtgagcccaatccctgccagaacggtgcccagtgctacaaccgtgccagcgactatttctgcaagtgtcccgaggactacgaaggcaagaactgctcccacctcaaagaccattgccgcacgactccctgcgaaggtacctgccccacctgtggtcctggctggcctccgaggctcactcctctacccctatttgtcagtaccccccaatattctcagcagctggctgggtgagacggtaacgccctaacctgcctcgctaggggagccagaacacgtggaaaccctctgtccgggcctgccagtgggagaaccttgtccacacgtagcattcctgttctggggtgggctgatttgcaaccctgagaccttccccaaagcagtgctgccggaagaactgtctgcagcactggagatgtgtgctgtctgcactgtcgggtgtgctagccacaggagcatttagtgtgaccactacgactggaaacttccttgactccagttcattcaaatctaaataaccccacgtgtctcatggtcgttgtattaggtggcctggctctgcgggttctctgtccttcctgcttgcttaggtaggaagtaaagctcctcctccctgtagcaagcttctgcccgactgaggagatgtcagaggtaggaggagaggagcccacagacccagaggcttcttagccacgtagggcgaaaatgccttaaaaacaaaaacaaaaaaaaaaacagagcttctgtctgtctggagtcttggctttgcctgtcacagtgtgacgtggcacatcgccagagtcttggccccggggtgcctcttgatttcttaccctcattagccaaagggtgcctgtttccaggaaagcgagcttgggcggaggtcctcagcttaggaatgccatccatgccttatgggtctgtggccctgatctgaccccgagtctgccttctagtgattgacagctgcacagtggccatggcttccaacgacacacctgagggggtgcggtacatctcctcgaacgtctgtggtccccatgggaagtgcaagagtcagtcgggaggcaaattcacctgtgactgtaacaaaggcttcactggaacgtactgccatgaaagtaagagccctcttcggggaatggggtgtggtgccccccacccctcctctgcccagttcaagggagcatttgagcttgattcgtttcagtcgagaccagtgacggtttaccattacatctaagatctaagaccgagttgtgtttaatttattgagatcacagacacctctgaccccttcagcacgtttatgttctggaatcttactagtttgggtggtcctgctttctgtaggcttttcttccctactccatccgagtagcttagcctccaaggcattaaataaaacagacaacgtatcctctggaggcttctgcctggtaagctgcttgctgaagtcggaaaaaccttcctgatggagttgaggatgttgaggtgcctggctgcccattttctcaggaggtggcctttgggccagtgcctggctatggcatcggttgtcctttacttaccagcttttttgtcttcatttcacagatattaatgactgcgagagcaatccctgtaaaaacggcggcacttgcatcgatggggtcaactcctacaaatgcatctgcagtgacggctgggagggggcctactgtgaaaccagtgagtgtgtggtctccctgggccagacagctctgggatgctggggtaggaggtatcatcagggaagcttgccttttcaatgggaattagatagtaagaccccaaccgttcctgtcctgggacagcattgcctgtgtcccgtctgtatttgtgaatgctgaggacggccaaaggggaggaacttctgctgaggagctgtggaaacagactgtcatcaggctaaatatccctgcttgcagagaaagtagtcttctgttttaatagtaatttgtgcctgtcctgatttttaacttccatgggagtgacagaggcctgcccttgtcttgccaccttgtggggtttaacccagctgaggagaagagaaggtgtttaagtcccaggatggatggctcagctggtctcggaccttagagtggaaagctgtgggtggaagtggccataggaagggtgtggctctgacacgccgtggatggccctagaagcttctggagctgggtgtctcctctttgattctagacattaacgactgcagccagaatccctgccacaatggtggctcatgtcgtgacctcgtcaacgacttctactgtgactgtaaaaacgggtggaaaggaaagacctgccattcgcgtaagtggtagctggctggggccccctctccgcatccccttcccaccaccctgtcttctgtgaagggacctcacatggagatccccctcctccaggtgacagccagtgtgatgaggccacgtgcaacaacggtggcacctgctatgacgaaggggatgctttcaagtgcatgtgtcctggaggctgggaagggacgacctgcaacataggtagctttctgccccacatggggtcggcagtgggtgtggccatctcaacacctaggaccgctttcctggcggtctgctgctcggctctcctactctctttggatcctcagggagggtggtcctgtcttgtcttgtcttacagcccgaaatagtagctgcctacccaacccttgccacaacgggggcacctgtgtggtcaacggggactccttcacgtgtgtctgcaaagaaggctgggaggggccgatctgcgctcagagtgagtgtcccctgccccacccgccttgaactccacagagggctgcaggagttcatcctgacaccctcagggactcgcgatggaaaccagaagcagggctgtttgatgtctgcttctgcatcacctgggtctgttctaatgcacttgatggaccgcagccctgacctgggcatgagaaagtcgccgatgagataagtggagacagatggtgtctctgttcccagcgtgggtctgaccagcagcctgtctccttccattctcacagcccccttcccgccctgagcggggcctcttcctgtgtaatgtcccacggcacacagaacaaagtgtggtgcaggcctggttccaattgagcagaggtcttaagcgcattttgccggtgtaaactacagtgtgatcattggggtattaatggaaataagtggtgcttctacttagggttaaaactgctttgcccggtgaaggcctcgctgttaagtcgtctttttactttgcagataccaatgactgcagtcctcatccttggtaagcatgagcgccttggaagccagcccttgctggctacgatgtgcatgcgcatcacctgggggtcctgtctaagatgcaggttctgaggcaggaggtctgggtggggctgcagacccttcctgtctaacaagctcccaggtcctgctggtccgtggaccacactctgattagccaggtcatgtcttggttgggggctgtgattattctcaacgattcccaaccttggctgtgcataagaatcacctggggagcttttaggagtcccaatgcctggattgcagcacagccccatgaaatgagactctctggggttgggacccaggcaccattattttgaaatctccccaggctacaggggtgtgtggccaggggtgagagctgctgaatggaactggcgtggccgcctgtggtcaactggctggtggagtcgtggatcaggaggtcggtaactgaggccgtctttgctttcttcttagttacaacagtggcacgtgcgtggatggagacaactggtaccggtgtgagtgtgcccctggttttgctggacccgactgcagaataagtaaggaccagccctggttggtttccccagtggccttcttccccctgattcagccctcagcctgagacttttcatcatgaaaagttaaggttactgctctctgtagctctgagaggcacagaacctcccacctccagctgggattaggatagcctggctggaatgtcccaggtccttgctgtcccttccccaggggacacagccctcttccaaggaagagaggagcagccacaggatctgatgattaatctgttctgaggccccaaggaaagggtgcttttcctcatgactacctgaaattaggggcgagtggacatgtgccacagaggaccgagctaaggacagtggccaggactagggttgtggttcccaaaggattgatccgaccctagtgaaattaaaatgggagacaggcagggtgcctaaagtctattttgagccttttcctactttcttatatgccttttagtttgggaggagggagtgatacttgtattttttgttattctcatccccaaattatttggaattttactagtgtttgagatcccccaagtctggggctgactggtgttcaccaaaatggggtacatttggtctatcccatgtttctaaattgtgttggcaaactgtagccttggcccactgcctgtttttgtaaataaagttttattggcacacagctactcttttatatactatctaaagctgctttcatgctataacaagggtagtcatgacaaagcctacaaggcctgcaagggttaaaatatttgacctttcccaagaactttacccttgctcttaagttgtagtatctgatgaaaaggaacttacttaattaaaaagtgccgcctcctgaaatgattctcagcagcttgaagtcaggctaatgatgatctttgggaggaagggactagtggaaatttgggggctactgatcggtttatttgggtgttggttacagaattcatcaagcttatacctagggtttacacgctcttctatctgtatgttagattagtaaaaacttagaagaatgtcattgcttcccattggagcctaaagttggtctccatttctcctagacatcaatgaatgccagtcctcaccgtgtgcctttggggcaacctgtgtggatgagattaatggctaccggtgcgtctgccccccagggcacagcggtgccaagtgccaggaaggtatgtgtgggtcaggctgcagctgcccatgtgtcttgtgggggcagtgggcactgcagtcaccacggttaccctttgactgttcctgagtaggcaaaggagaacatggcggggatatgcacagggggccacagggtcggggaaacttagggggtcccctacaaacaggatagctgttactagtcaactcccgaagttttgccgtgtgacaaaccacccccaaacctgcggcttacagcaggcttttatttgcattgctcatgggcacgcaggctgggctcagttctgttctgtgtgcttcgtcaccctccttgggccagcggccatatctcaactgcgagaggctgcccacatcacatcccctagtgctgcacaggtcccacaggccccacaggtcctgaggccacgtgcaaaggcagcaggtgagggaagaacacctattgccccgtggcacagggcaaaggtacagcccttccctgacagggagggagtgaagaatagaacggtcaatagaaaaggaaatctgttccactcatgcgctctacctttcagtttcagggaaaccttgcatcactatgggaagtgtgatcccagatggggccaagtgggacgatgactgcaatacctgccagtgcctgaacggaaggatcgcctgctccaaggtacagcatgagggccctctgcctctcacctgggttctagattgctgctgggtgttttctcctgagctgcccgctccatctcctctccccctgctccccaggtctggtgcggccctcgaccttgcctgctccacaaagggcacagcgagtgccccagcgggcagagctgcatccctatcctggaggaccagtgtttcgtccgcccctgcacgggcgtgggcgagtgtcggtcttccagtctccagccggtgaagaccaagtgtacctctgacccctactaccaggataactgtgccaacatcacattcaccttcaacaaagagatgatgtcaccggtacgaataacttctcattcagtttgggctggcggtgtgtgtctcctggttccaaagcaggagcattgcaagctaggatcttccccttagcttaaattcctaacttgcaaggaaacgtcagccttttttatttttttcctgttcctcgcaatatcttacatgtgtggtttttacatgtgtttctagggtctaaccacagagcatatctgcagcgaattgaggaatctgaatatcttgaagaacgtttctgctgaatattcaatctacatagcttgtgagccttccccttctgcgaacaacgaaatacatgtggccattgtaagtatgagacccagtcacgcctcatgactcgactgcagggtagttctcaaagtccagttagccggtgtccagacagagcaaggacactgaaacagcttggcacacgggctaagtcactttcagtgatggctgctgtgtagtgttgccctgatcccacatctggcccttctttttccagtctgctgaagacatacgggacgatggaaaccctatcaaggaaatcactgacaaaatcatagacctcgttagtaaacgtgacgggaatagctcgctgatcgctgctgtggcggaagtgcgagtacagaggcggcctctgaagaacagaacaggtaggtggcaggtggggacagtcctttcgcgtgaggactgtccgacggatggctcctcggctcaccgttcagggagggcattagctgacagcttctctcaaggaaactccccaccccccccacccccatttaactggcggaaaaactgggaccagctgccatgaccaaggtcccgtggtgatgggcagaagtacc

SEQ ID NO: 7 - DNA, 6906 bp, Canis familiaris
atgcggtccccacggacgcgcgaccggcccgggcgccccctgagcctcctgctcgccctgctctgcgccctgcgagccaaggtgtgcggcgcctcgggccagttcgagctggagatcctgtccatgcagaacgtgaacggggagctgcagaacgggcactgctgcggcggcgtccggagcccgggggaccgcaagtgctctcacgacgagtgtgacacgtacttcaaagtgtgcctcaaggagtaccagttccgcgtcacggccggggggccctgtagcttcgggtcgggctccacgccagtcctcgggggcaacaccttcaatctcaaggccggccgtggcagcgaacgcaaccgcatcgtgctgcctttcagtttcgcctggccgaggtcctacactttgctggtggaagcgtgggattccagtaatgacactctccagcccgacagcattattgaaaaggcctctcactcaggcatgatcaaccccagcaggcagtggcagacactgaagcagaacacaggggttgcccactttgagtatcagatccgtgtcacctgtgatgactattactatggctttggctgcaacaagttctgccgacccagagatgacttctttggacactatgcctgtgaccagaatggcaacaaaacttgcatggagggctggatgggcccagaatgtaacaaagctatttgccgacagggctgtagtcccaagcatggatcttgcaaactcccaggtgactgcaggtgccagtacggctggcaaggcctgtactgcgacaagtgcatcccgcaccccgggtgtgtccacggcacctgtaacgagccctggcagtgcctctgcgagaccaactggggcggccagctctgcgacaaagatctcaactactgcgggactcggcagccctgcctcaacgggggcacctgcagcaacacgggccctgacaagtaccagtgctcctgccccgagggctactcaggccccaactgtgagatcgcggagcacgcctgcctgtccgacccctgccacaacagaggcagctgcagggagacctcgctgggcttcgagtgtgagtgctctccgggctggacgggccccacgtgttccacaaacattgatgactgttctccaaataactgttcccacgggggcacctgccaggacttggttaatggattcaagtgcgtgtgcccaccccagtggactggcaaaacgtgccagttagatgcaaatgaatgtgaggccaaaccttgtgtaaatgccaagtcctgtaagaacctgattgcgagctactactgtgattgcctccctggctggacgggtcagaattgtgacataaatattaatgactgccttggccagtgtcagaatgatgcctcctgtcgggatttggttaatggttatcgctgtatctgtccacctggctatgcaggcgatcactgtgagagagacatcgatgaatgcgccagcaacccctgtttgaatgggggtcactgtcagaatgaaatcaacagattccagtgtctgtgtcccactggtttctctggaaacctctgtcagctggacatagattattgtgagcccaatccctgccagaacggtgcccagtgctacaaccgtgccagcgactatttctgcaagtgtcccgaggactacgaaggcaagaactgctcccacctcaaagaccattgccgcacgactccctgcgaagtgattgacagctgcacagtggccatggcttccaacgacacacctgagggggtgcggtacatctcctcgaacgtctgtggtccccatgggaagtgcaagagtcagtcgggaggcaaattcacctgtgactgtaacaaaggcttcactggaacgtactgccatgaaaatattaatgactgcgagagcaatccctgtaaaaacggcggcacttgcatcgatggggtcaactcctacaaatgcatctgcagtgacggctgggagggggcctactgtgaaaccaacattaacgactgcagccagaatccctgccacaatggtggctcatgtcgtgacctcgtcaacgacttctactgtgactgtaaaaacgggtggaaaggaaagacctgccattcgcgtgacagccagtgtgatgaggccacgtgcaacaacggtggcacctgctatgacgaaggggatgctttcaagtgcatgtgtcctggaggctgggaagggacgacctgcaacatagcccgaaatagtagctgcctacccaacccttgccacaacgggggcacctgtgtggtcaacggggactccttcacgtgtgtctgcaaagaaggctgggaggggccgatctgcgctcagaataccaatgactgcagtcctcatccttgttacaacagtggcacgtgcgtggatggagacaactggtaccggtgtgagtgtgcccctggttttgctggacccgactgcagaataaacatcaatgaatgccagtcctcaccgtgtgcctttggggcaacctgtgtggatgagattaatggctaccggtgcgtctgccccccagggcacagcggtgccaagtgccaggaagtttcagggaaaccttgcatcactatgggaagtgtgatcccagatggggccaagtgggacgatgactgcaatacctgccagtgcctgaacggaaggatcgcctgctccaaggtctggtgcggccctcgaccttgcctgctccacaaagggcacagcgagtgccccagcgggcagagctgcatccctatcctggaggaccagtgtttcgtccgcccctgcacgggcgtgggcgagtgtcggtcttccagtctccagccggtgaagaccaagtgtacctctgacccctactaccaggataactgtgccaacatcacattcaccttcaacaaagagatgatgtcaccgggtctaaccacagagcatatctgcagcgaattgaggaatctgaatatcttgaagaacgtttctgctgaatattcaatctacatagcttgtgagccttccccttctgcgaacaacgaaatacatgtggccatttctgctgaagacatacgggacgatggaaaccctatcaaggaaatcactgacaaaatcatagacctcgttagtaaacgtgacgggaatagctcgctgatcgctgctgtggcggaagtgcgagtacagaggcggcctctgaagaacagaacagatttcctggtccctctgctgagctctgtcttaacggtggcttggatctgttgcctggtgacggccttctactggtgcgtgcggaagcggcggaagcccagcagccacgcgcgctcggcctccgaggacaacaccaccaacaacgtgcgggagcagctgaaccagatcaagaaccccatcgagaagcatggggccaacacggtccccgtcaaggactacgagaacaaaaactccaaaatgtcaaaaataaggacacacaactcagaggtggaggaggacgacatggataagcaccagcagaaagcccggtttgccaagcagcccgcatacacgctggtagaccgagaggagaagcctcccaacggcacgccagccaaacacccaaactggacaaacaaacaggacaacagagacctggaaagtgcccagagcctaaaccggatggagtacatcgtatagcagacagcaggcgctgccgctaggtagagtcggagggtgctgggggcttgtagttcagttcttcagctgtcctgtcctcttccagtctgaggctgtcgttgacttagaatcctgtgttaatttttgtttttgacaagctggcttacactggcaatggtagtttctgtggttggctgggaaatccaatgctgcagctcacagctatgcaaaaaccagtccaaagtgccgcccccccccccccccaccgccgcccctgccccacagctgacacctcttggaccaggctcccaggagaatgcccggccccggggccttgagcttccacctctgccagatgtcccgatggtgatgcagtcttaggatcatagtttttatttatatttattgactcttgagttgtttttgtatattggttttatgatgacgtacaagtagttctgtatttgaaagtgccttttgcagctcagaaccacagcaactatcacaaatgagtttattatttattttttttattgtattttgttgttgggggaggggggaccttgatgtcagcagttgctggtaaaaatgaagaatttaaagaggaaaaatgtgtcaaaagtagaattttgtatagttatgtaaataattcttttttattaatcactgtgtatatttgatttattaacttaataatcaagagccttaaaacatcattcctttttatttatatgtacgtgtttagaattgaaggtttttgatagcattgttaagtgtatggctttatttttttgaacttattttcttattacgtgttgcctataagccaaaactaaggtgtttgaaaatagtttaaaacaataggatgggcttcagtgcctggaatactggtggaatttttttttttttttttttgtacgacgtcagatgtttaaaacaccttctatagcatcactttaaaacacgttttaaggactgaggcagtttgaaaattagtctagaaaagcaggtggtttttttgtttttttatgctttagacttgaaaagagacaggcaggtgatcggctacacagcagtttaagagaacatgttgagcttcaacttaacgtagccaaaatgtgagtggttgaatatcattaaaaatatcaaattgtgtgaagttggaagcacaccaatcttattttgtaaattctgatttcttttcaccattcgtatgtaatactgaaccacttgtagattttttttgtttgtttgttatctactgcatttagggagtattcttataagctagttgaatacttgaaccttaaaatgtccagtaagatcactgtttagatttgccatagaatacactgcctgccttaagtgaggaaataaaaatgctatttacaaagttgaagatcaaaacggcttctaaaacagttcacgtcgtcggctcactacggagaccgtgaagatactctgtattgtcctattagtgttatgaacataaaaatgcatcttcgatgtgttgttcctggcaataaattttgaaaagtactatttattaaattttttgtatgaaacctggaacagtgtggcctcttctgagcttctgtagttttgcttggctctaccttgtgcctttgccaccccaccgaggccgttctggtaatcagggtgtgctaaacaggctgtgcttgacaggggtgctgaggaagactgaaagccttttcagccacaaaatttcagctgaagttctcaaaaaaccatgggctgctgtgaagccagagctgtgagagccccagctcgggggtcctggattcccttgttattcaacagcaagtgtgaatactgcttgaataaacaccactggattaatggcctgtagtgtggaggtgaattgtttgtgagcactcacgggaaatgcccaccgcactaaaaggggttttaaaaagcttggaattagtgttgggggaggaagagggagatttggtgctttcggtctttttttttttttttttcccctggctgcttggattaatattaatgtcctgccgtcattttagaccacaccttcgtggcttctgtgcacgcttacgcctccagggcggtttcctcgctgcacaggaggtggtagttacaagcttctggggttagagccatgtactcatcaccttgttccaagggaatccgtgcggctgtggggtgccaggtatctggccttcctggaggttccagaggccaaccatcgccacctgttcaggacaggtgaactgtggttctccttgtggctgctggtgccagaggccagaatggccctgcccgcacatcacgggtggggctgggaaagctttgcctctggcacctcagtagtcagagatgagccagagtgacgggcacatggatggcttccagcagcttcatggagttgccaggagttccctgcttctgatgagtgagccaaagcctaacctccctggagctgtcccctagcgctgcacattcaggccatccgtccacctctgattttcatagtatgcttttactctttcttgggaaaattctcttctactggactaggaactgtgttctctgatgggcatctgcttaagaatgggcaatgggctttaaaaatatcccaagtttcctgtgcatgagcagagtactgaggagtcagaatccctaactatgcgttcgggcttgctgggaaagcagcccgaaggacctggaatcccgtgacgactccctgccctggtgtagcaatactttctgtccctaatctctgctgtgggacagctggtttctgcaaggtggggtcacagggagaagtgctctggtacagacccagggagccaggacacagtccgatttagcagcactcaaacaccatcccgaggcaatatcttgctccggtggtttgtgaacttggattcttcaggaccccagaattccttgggtagttgccaagtgcttgcttttgtgccaagaggaaaaaaaaaaaaaagtttaacgagcctctgcaattcttgattccaggggggaagtgatcttgagattcacatttgttgttttaagaagccatctcagtagtgagaaggcagagatcaggctcctctgggcatggctcaaagccaggaccttaatcataatgggcagttcccctcttttccaggtagagggaatccggcaagggagccctggttatcgtgggaacaagctggaaaagaagggactccatacctttcccattccccagcaatgttctgagccctcgtttaagcagaccct

SEQ ID NO: 8 - protein, 1218 aa, Canis familiaris
MRSPRTRDRPGRPLSLLLALLCALRAKVCGASGQFELEILSMQNVNGELQNGHCCGGVRSPGDRKCSHDECDTYFKVCLKEYQFRVTAGGPCSFGSGSTPVLGGNTFNLKAGRGSERNRIVLPFSFAWPRSYTLLVEAWDSSNDTLQPDSIIEKASHSGMINPSRQWQTLKQNTGVAHFEYQIRVTCDDYYYGFGCNKFCRPRDDFFGHYACDQNGNKTCMEGWMGPECNKAICRQGCSPKHGSCKLPGDCRCQYGWQGLYCDKCIPHPGCVHGTCNEPWQCLCETNWGGQLCDKDLNYCGTRQPCLNGGTCSNTGPDKYQCSCPEGYSGPNCEIAEHACLSDPCHNRGSCRETSLGFECECSPGWTGPTCSTNIDDCSPNNCSHGGTCQDLVNGFKCVCPPQWTGKTCQLDANECEAKPCVNAKSCKNLIASYYCDCLPGWTGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCKNGGTCIDGVNSYKCICSDGWEGAYCETNINDCSQNPCHNGGSCRDLVNDFYCDCKNGWKGKTCHSRDSQCDEATCNNGGTCYDEGDAFKCMCPGGWEGTTCNIARNSSCLPNPCHNGGTCVVNGDSFTCVCKEGWEGPICAQNTNDCSPHPCYNSGTCVDGDNWYRCECAPGFAGPDCRININECQSSPCAFGATCVDEINGYRCVCPPGHSGAKCQEVSGKPCITMGSVIPDGAKWDDDCNTCQCLNGRIACSKVWCGPRPCLLHKGHSECPSGQSCIPILEDQCFVRPCTGVGECRSSSLQPVKTKCTSDPYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCVRKRRKPSSHARSASEDNTTNNVREQLNQIKNPIEKHGANTVPVKDYENKNSKMSKIRTHNSEVEEDDMDKHQQKARFAKQPAYTLVDREEKPPNGTPAKHPNWTNKQDNRDLESAQSLNRMEYIV

SEQ ID NO: 9 - DNA, 32 bp, Artificial Sequence
  Synthetic Polynucleotide
ctcctttatttcagcggaactaaagaagtctc

SEQ ID NO: 10 - DNA, 32 bp, Artificial Sequence
  Synthetic Polynucleotide
ctcctttatttcagctgaactaaagaagtctc

SEQ ID NO: 11 - DNA, 24 bp, Artificial Sequence
  Synthetic Polynucleotide
ctggttacattctgagagactttc

SEQ ID NO: 12 - DNA, 24 bp, Artificial Sequence
  Synthetic Polynucleotide
agccagctgaaccaattaactcac

SEQ ID NO: 13 - DNA, 13 bp, Homo sapiens
ttcaaaggaagtc

SEQ ID NO: 14 - DNA, 13 bp, Macaca mulatta
ttcaaaggaagtc

SEQ ID NO: 15 - DNA, 13 bp, Mus musculus
tttaatggaaatc

SEQ ID NO: 16 - DNA, 13 bp, Rattus rattus
tttaatggtaatc

SEQ ID NO: 17 - DNA, 13 bp, Sus scrofa domesticus
tccagtggaaatc

SEQ ID NO: 18 - DNA, 13 bp, Felis catus
ttcagtggaaatt

SEQ ID NO: 19 - DNA, 13 bp, Canis familiaris
ttcagcggaacta

SEQ ID NO: 20 - DNA, 13 bp, Artificial Sequence
  Synthetic Polynucleotide
ttcagctgaacta